(19) United States
(12) Patent Application Publication
(10) Pub. No.: US 2025/0019374 A1
(43) Pub. Date: Jan. 16, 2025

(54) PROTEIN TYROSINE PHOSPHATASE INHIBITORS AND METHODS OF USE THEREOF

(71) Applicants: Calico Life Sciences LLC, South San Francisco, CA (US); AbbVie Inc., North Chicago, IL (US)

(72) Inventors: Andrew Bogdan, Evanston, IL (US); Elliot P. Farney, Evanston, IL (US); Jennifer M. Frost, Gurnee, IL (US); Philip R. Kym, Libertyville, IL (US); Zhaoming Xiong, Cambridge, MA (US); Naomi Anne Barton, Nottingham (GB); Claire Anne Marie Cariou-Mumford, Nottingham (GB); Christopher Patrick McMahon, Macclesfield (GB); Giuseppe Nano, Wishaw, Scotland (GB)

(21) Appl. No.: 18/709,214

(22) PCT Filed: Nov. 10, 2022
(86) PCT No.: PCT/US2022/049584

§ 371 Date: May 10, 2024

Related U.S. Application Data
(60) Provisional application No. 63/278,339, filed on Nov. 11, 2021.

(51) Int. Cl. C07D 417/04; A61K 31/433; A61K 31/454; C07D 417/14

(52) U.S. Cl. CPC C07D 417/04; A61K 31/433; A61K 31/454; C07D 417/14

(57) ABSTRACT

The present invention provides for compounds of Formula (I) (I); wherein R², X, and Z have any of the values defined herein, and pharmaceutically acceptable salts thereof, that are useful as agents in the treatment of non-small cell lung cancer.

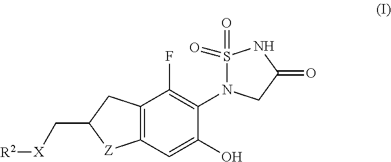

### CROSS-REFERENCE TO RELATED APPLICATION

This application claims the benefit of and priority to U.S. Provisional Application No. 63/278,339, filed on Nov. 11, 2021, the entire disclosure of which is hereby incorporated by reference herein in its entirety for all purposes.

### BACKGROUND

Protein tyrosine phosphatase non-receptor type 2 (PTPN2), also known as T cell protein tyrosine phosphatase (TC-PTP), controls multiple cellular regulatory processes by removing phosphate groups from tyrosine substrates. PTPN2 is ubiquitously expressed, but expression is highest in hematopoietic and placental cells (Mosinger, B. Jr. et al., Proc Natl Acad Sci USA 89:499-503; 1992). In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif (Tillmann U. et al., Mol Cell Biol 14:3030-3040; 1994). The 45 kDa isoform can passively transfuse into the cytosol under certain cellular stress conditions. PTPN2 negatively regulates signaling of non-receptor tyrosine kinases (e.g. JAK1, JAK3), receptor tyrosine kinases (e.g. INSR, EGFR, CSF1R, PDGFR), transcription factors (e.g. STAT1, STAT3, STAT5a/b), and Src family kinases (e.g. Fyn, Lck). As a critical negative regulator of the JAK-STAT pathway, PTPN2 functions to directly regulate signaling through cytokine receptors, including IFNγ.

Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade (Manguso R. T. et al., Nature 547:413-418; 2017). Loss of Ptpn2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosine phosphatase-1B (PTP1B), has been shown to play a key role in insulin and leptin signaling and is a primary mechanism for down-regulating both the insulin and leptin receptor signaling pathways (Kenner K. A. et al., J Biol Chem 271: 19810-19816, 1996). Animals deficient in PTPN1 have improved glucose regulation and lipid profiles and are resistant to weight gain when treated with a high fat diet (Elchebly M. et al., Science 283: 1544-1548, 1999). Thus, there is a medical need for PTPN1 inhibitors.

### BRIEF SUMMARY

The present disclosure is directed, at least in part, to compounds, for the inhibition of protein tyrosine phosphatase, e.g., protein tyrosine phosphatase non-receptor type 2 (PTPN2) and/or protein tyrosine phosphatase non-receptor type 1 ((PTPN1), also known as protein tyrosine phosphatase-1B (PTP1B)).

For example, disclosed herein is a compound represented by Formula (I):

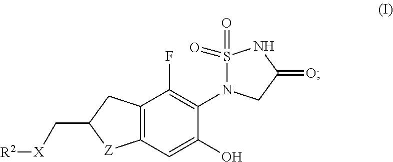

or a pharmaceutically acceptable salt thereof, wherein:
X is —O— or —N(R^{a})—;
Z is —O— or —N(R¹)—;
R¹ is hydrogen or C₁₋₆alkyl;
R² is selected from the group consisting of hydrogen, —C₁₋₆alkyl, —C₁₋₆alkylene-C₃₋₆cycloalkyl, —C₁₋₆alkylene-4-6 membered heterocyclyl, and —C₁₋₆alkylene-phenyl;
   wherein —C₁₋₆alkyl, —C₁₋₆alkylene-C₃₋₆cycloalkyl, —C₁₋₆alkylene-4-6 membered heterocyclyl, and —C₁₋₆alkylene-phenyl may optionally be substituted on one or more available carbons by one, two, three or more substituents each independently selected from R^{g};
   wherein if —C₁₋₆alkylene-4-6 membered heterocyclyl contains a substitutable ring nitrogen atom, that ring nitrogen atom may optionally be substituted by R^{h};
R^{g} is independently selected, for each occurrence, from the group consisting of halogen, hydroxyl, C₁₋₆alkyl, phenyl, and C₁₋₆alkoxy; wherein C₁₋₆alkyl, phenyl, and C₁₋₆alkoxy may optionally be substituted by one, two three or more substituents each independently selected from R^{P};
R^{h} is C₁₋₆alkyl-O—C(O)—;
R^{P} is independently selected, for each occurrence, from the group consisting of halogen, and hydroxyl; and
R^{a} is selected from the group consisting of hydrogen and C₁₋₆alkyl; wherein C₁₋₆alkyl may optionally be substituted by one or more substituents each independently selected from the group consisting of halogen, cyano, oxo, hydroxyl, and C₃₋₆cycloalkyl.

### BRIEF DESCRIPTION OF THE SEQUENCE LISTING

Incorporated herein by reference in its entirety is a Sequence Listing entitled, “CLS-029PR_ABV21373USL1 SEQ ID List_ST25.txt”, comprising SEQ ID NO: 1 through SEQ ID NO: 3, which includes the amino acid sequence disclosed herein. The Sequence Listing has been submitted herewith in ASCII text format via EFS. The Sequence Listing was first created on Jun. 22, 2021 and is 7,282 bytes in size.

### DETAILED DESCRIPTION

For example, disclosed herein is a compound represented by Formula (I):

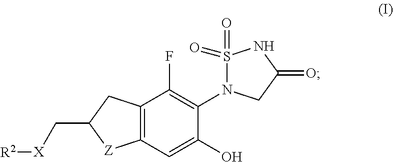

or a pharmaceutically acceptable salt thereof, wherein:
X is —O— or —N(R)—;
Z is —O— or —N(R¹)—;
R¹ is hydrogen or C₁₋₆alkyl;
R² is selected from the group consisting of hydrogen, —C₁₋₆alkyl, —C₁₋₆alkylene-C₃₋₆cycloalkyl, —C₁₋₆alkylene-4-6 membered heterocyclyl, and —C₁₋₆alkylene-phenyl;
   wherein —C₁₋₆alkyl, —C₁₋₆alkylene-C₃₋₆cycloalkyl, —C₁₋₆alkylene-4-6 membered heterocyclyl, and —C₁₋₆alkylene-phenyl may optionally be substituted on one or more available carbons by one, two, three or more substituents each independently selected from R^{g};
   wherein if —C₁₋₆alkylene-4-6 membered heterocyclyl contains a substitutable ring nitrogen atom, that ring nitrogen atom may optionally be substituted by R^{h};
R^{g} is independently selected, for each occurrence, from the group consisting of halogen, hydroxyl, C₁₋₆alkyl, phenyl, and C₁₋₆alkoxy; wherein C₁₋₆alkyl, phenyl, and C₁₋₆alkoxy may optionally be substituted by one, two three or more substituents each independently selected from R^{P};
R^{h} is C₁₋₆alkyl-O—C(O)—;
R^{P} is independently selected, for each occurrence, from the group consisting of halogen, and hydroxyl; and
R^{a} is selected from the group consisting of hydrogen and C₁₋₆alkyl; wherein C₁₋₆alkyl may optionally be substituted by one or more substituents each independently selected from the group consisting of halogen, cyano, oxo, hydroxyl, and C₃₋₆cycloalkyl.

The present disclosure includes compounds represented by Formula (I-a) or Formula (Ib):

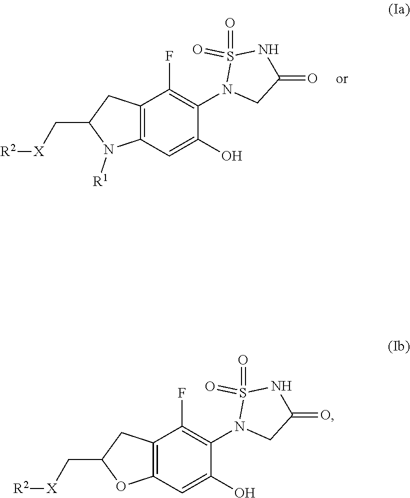

or a pharmaceutically acceptable salt thereof, wherein R¹ and R², are defined herein.

The present disclosure includes compounds represented by Formula (Ia-1) or Formula (Ia-2):

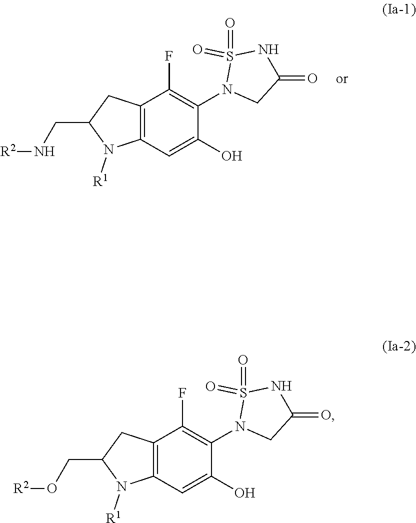

or a pharmaceutically acceptable salt thereof, wherein R¹ and, R² are defined herein.

In some embodiments, R¹ is hydrogen or C₁₋₆alkyl. In some embodiments, R¹ is hydrogen. In some embodiments, R¹ is methyl.

The present disclosure includes compounds represented by Formula (Ib-1) or Formula

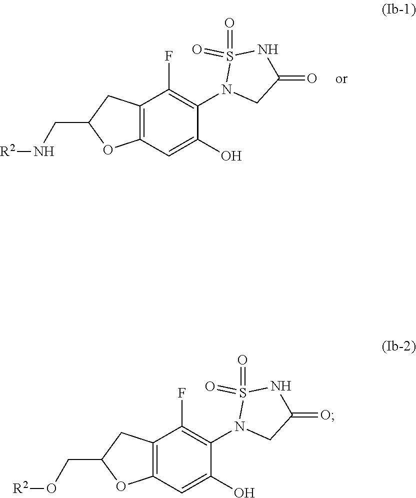

or a pharmaceutically acceptable salt thereof, wherein R² is defined herein.

In some embodiments, R² is selected from the group consisting of hydrogen, —C₁₋₆alkyl, —C₁₋₆alkylene-C₃₋₆cycloalkyl, —C₁₋₆alkylene-4-6 membered heterocyclyl, and —C₁₋₆alkylene-phenyl;
wherein —C₁₋₆alkyl, —C₁₋₆alkylene-C₃₋₆cycloalkyl, —C₁₋₆alkylene-4-6 membered heterocyclyl, and —C₁₋₆alkylene-phenyl may optionally be substituted on one or more available carbons by one, two, three or more substituents each independently selected from R^{g};
wherein if —C₁₋₆alkylene-4-6 membered heterocyclyl contains a substitutable ring nitrogen atom, that ring nitrogen atom may optionally be substituted by R^{h}.

In some embodiments, R² is —C₁₋₆alkyl optionally substituted with one, two, or three instances of R^{g}. In some embodiments, R² is —C₁₋₆alkyl optionally substituted with one, two, or three instances of R^{g}, wherein R^{g} is selected from halogen, hydroxyl, C₁₋₆alkyl, and C₁₋₆alkoxy wherein R^{g} is optionally substituted by one, two, three or more halogen. In some embodiments, R² is —C₁₋₆alkyl may be substituted with one, two, or three instances of R^{g}, wherein R^{g} is selected from halogen, hydroxyl, C₁₋₆alkyl, and C₁₋₆alkoxy, wherein C₁₋₆alkyl, and C₁₋₆alkoxy are optionally substituted by one, two, three or more substituents each independently selected from R^{P}; and R^{P} is independently selected, for each occurrence, from halogen.

In some embodiments, R² is selected from the group consisting of

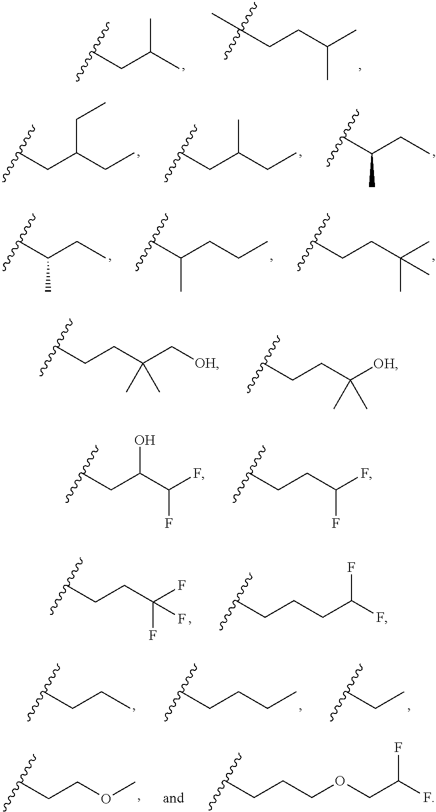

In some embodiments, R² is —C₁₋₆alkylene-C₃₋₆cycloalkyl, wherein R² may optionally be substituted by one, two, three or more substituents each independently selected from R^{g}. In some embodiments, R² is —C₁₋₆alkylene-C₃₋₆cycloalkyl, wherein R² is substituted by one, two, three or more substituents each independently selected from R^{g}. In some embodiments, R² is —C₁₋₆alkylene-C₃₋₆cycloalkyl, wherein R² may optionally be substituted by one, two, three or more substituents each independently selected from the group consisting of fluorine, hydroxyl, C₁₋₆alkyl, and phenyl, wherein C₁₋₆alkyl may optionally be substituted by one, two or three hydroxyl or fluorine. In some embodiments, R² is —C₁₋₆alkylene-C₃₋₆cycloalkyl, wherein R² may optionally be substituted by one, two, three or more substituents each independently selected from R^{g}, wherein R^{g} is independently selected, for each occurrence, from the group consisting of fluorine, C₁₋₆alkyl, and phenyl, wherein C₁₋₆alkyl and phenyl may optionally be substituted by one, two or three substituents each independently selected from R^{P}; and R^{P} is independently selected, for each occurrence, from hydroxyl or fluorine.

In some embodiments, R² is selected from the group consisting of

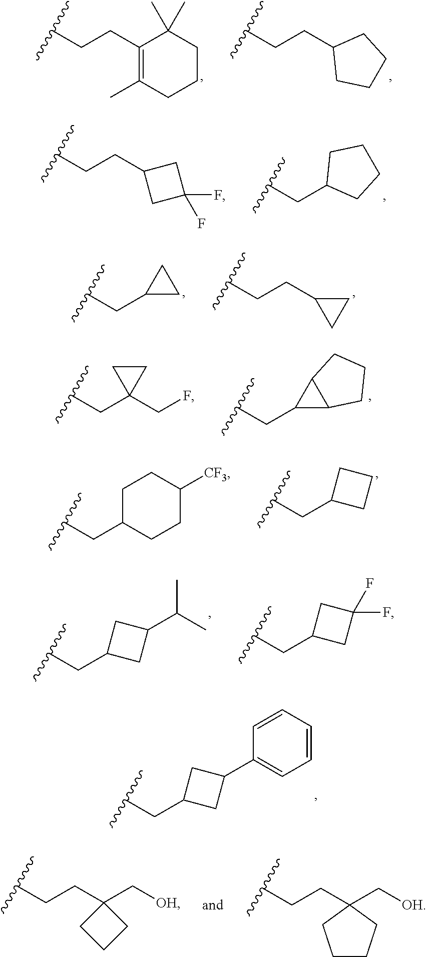

In some embodiments, R² is —C₁₋₆alkylene-4-6 membered heterocyclyl, wherein R² may optionally be substituted by one, two, three or more substituents each independently selected from R^{g}, wherein if the 4-7 membered heterocyclyl contains a substitutable ring nitrogen atom, that ring nitrogen atom may optionally be substituted by R^{h}. In some embodiments, R² may optionally be substituted by R^{h}, and R^{h} is C₁₋₆alkyl-O—C(O)—. In some embodiments, R² is selected from the group consisting of

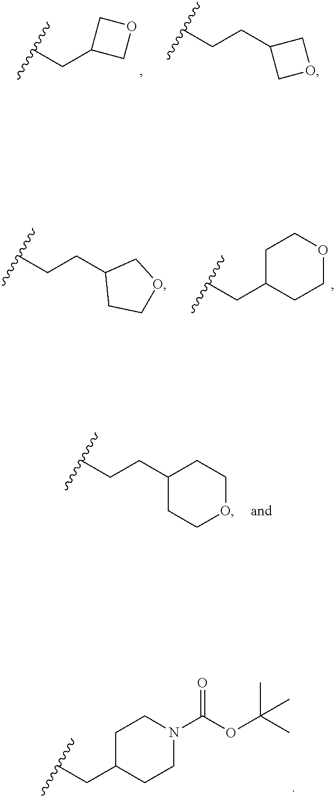 [SMILES: *CC1COC1 ; *CCC1COC1 ; *CCC1CCOC1 ; *CC1CCOCC1 ; *CCC1CCOCC1 ; *CC1CCN(C(=O)OC(C)(C)C)CC1]

In some embodiments, R² is hydrogen.

The present disclosure includes a compound selected from the group consisting of
5-[(2R)-4-fluoro-6-hydroxy-2-{[(2-methylpropyl)amino]methyl}-2,3-dihydro-1-benzofuran-5-yl]-1λ⁶,2,5-thiadiazolidine-1,1,3-trione;
5-[(2S)-4-fluoro-6-hydroxy-2-{[(2-methylpropyl)amino]methyl}-2,3-dihydro-1-benzofuran-5-yl]-1λ⁶,2,5-thiadiazolidine-1,1,3-trione;
5-(4-fluoro-6-hydroxy-2-{[(2-methylpropyl)amino]methyl}-2,3-dihydro-1-benzofuran-5-yl)-1λ⁶,2,5-thiadiazolidine-1,1,3-trione;
5-[(2R)-2-{[(2-cyclopentylethyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-{[(4-hydroxy-3,3-dimethylbutyl)amino]methyl}-2,3-dihydro-1-benzofuran-5-yl]-1λ⁶,2,5-thiadiazolidine-1,1,3-trione;
5-[(2R)-4-fluoro-6-hydroxy-2-{[(3-hydroxy-3-methylbutyl)amino]methyl}-2,3-dihydro-1-benzofuran-5-yl]-1λ⁶,2,5-thiadiazolidine-1,1,3-trione;
5-[(2R)-2-({[2-(3,3-difluorocyclobutyl)ethyl]amino}methyl)-4-fluoro-6-hydroxy-2,3-dihydro-1-benzofuran-5-yl]-1λ⁶,2,5-thiadiazolidine-1,1,3-trione;
5-[(2R)-2-{[(4,4-difluorobutyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-({[2-(oxolan-3-yl)ethyl]amino}methyl)-2,3-dihydro-1-benzofuran-5-yl]-1λ⁶,2,5-thiadiazolidine-1,1,3-trione;
5-{(2R)-4-fluoro-6-hydroxy-2-[(propylamino)methyl]-2,3-dihydro-1-benzofuran-5-yl}-1λ⁶,2,5-thiadiazolidine-1,1,3-trione;
5-{(2R)-2-[(dipropylamino)methyl]-4-fluoro-6-hydroxy-2,3-dihydro-1-benzofuran-5-yl}-1λ⁶,2,5-thiadiazolidine-1,1,3-trione;
5-{(2R)-2-[(butylamino)methyl]-4-fluoro-6-hydroxy-2,3-dihydro-1-benzofuran-5-yl}-1λ⁶,2,5-thiadiazolidine-1,1,3-trione;
5-{(2R)-2-[(ethylamino)methyl]-4-fluoro-6-hydroxy-2,3-dihydro-1-benzofuran-5-yl}-1λ⁶,2,5-thiadiazolidine-1,1,3-trione;
5-[(2R)-2-{[(cyclopentylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-({[(oxan-4-yl)methyl]amino}methyl)-2,3-dihydro-1-benzofuran-5-yl]-1λ⁶,2,5-thiadiazolidine-1,1,3-trione;
5-[(2R)-4-fluoro-6-hydroxy-2-{[(3-methylbutyl)amino]methyl}-2,3-dihydro-1-benzofuran-5-yl]-1λ⁶,2,5-thiadiazolidine-1,1,3-trione;
5-[(2R)-4-fluoro-6-hydroxy-2-{[(2-methylbutyl)amino]methyl}-2,3-dihydro-1-benzofuran-5-yl]-1λ⁶,2,5-thiadiazolidine-1,1,3-trione;
5-[(2R)-2-{[(cyclopropylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-2-{[bis(cyclopropylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-2-{[(cyclobutylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-({[(oxetan-3-yl)methyl]amino}methyl)-2,3-dihydro-1-benzofuran-5-yl]-1λ⁶,2,5-thiadiazolidine-1,1,3-trione;
5-{(2R)-4-fluoro-6-hydroxy-2-[({2-[1-(hydroxymethyl)cyclobutyl]ethyl}amino)methyl]-2,3-dihydro-1-benzofuran-5-yl}-1λ⁶,2,5-thiadiazolidine-1,1,3-trione;
5-{(2R)-4-fluoro-6-hydroxy-2-[({2-[1-(hydroxymethyl)cyclopentyl]ethyl}amino)methyl]-2,3-dihydro-1-benzofuran-5-yl}-1λ⁶,2,5-thiadiazolidine-1,1,3-trione;
5-[(2R)-2-({[3-(2,2-difluoroethoxy)propyl]amino}methyl)-4-fluoro-6-hydroxy-2,3-dihydro-1-benzofuran-5-yl]-1λ⁶,2,5-thiadiazolidine-1,1,3-trione;
tert-butyl 4-[({[(2R)-4-fluoro-6-hydroxy-5-(1,1,4-trioxo-1λ⁶,2,5-thiadiazolidin-2-yl)-2,3-dihydro-1-benzofuran-2-yl]methyl}amino)methyl]piperidine-1-carboxylate;
5-[(2R)-4-fluoro-6-hydroxy-2-({[2-(oxan-4-yl)ethyl]amino}methyl)-2,3-dihydro-1-benzofuran-5-yl]-1λ⁶,2,5-thiadiazolidine-1,1,3-trione;
5-{(2R)-2-[({[(1RS,5SR)-bicyclo[3.1.0]hexan-6-yl]methyl}amino)methyl]-4-fluoro-6-hydroxy-2,3-dihydro-1-benzofuran-5-yl}-1λ⁶,2,5-thiadiazolidine-1,1,3-trione;
5-[(2R)-4-fluoro-6-hydroxy-2-{[(3-phenylpropyl)amino]methyl}-2,3-dihydro-1-benzofuran-5-yl]-1λ⁶,2,5-thiadiazolidine-1,1,3-trione;
5-[(2R)-4-fluoro-6-hydroxy-2-({[2-(2,6,6-trimethylcyclohex-1-en-1-yl)ethyl]amino}methyl)-2,3-dihydro-1-benzofuran-5-yl]-1λ⁶,2,5-thiadiazolidine-1,1,3-trione;
5-[(2R)-4-fluoro-6-hydroxy-2-({[(3-phenylcyclobutyl)methyl]amino}methyl)-2,3-dihydro-1-benzofuran-5-yl]-1λ⁶,2,5-thiadiazolidine-1,1,3-trione;
5-{(2R)-4-fluoro-6-hydroxy-2-[({[4-(trifluoromethyl)cyclohexyl]methyl}amino)methyl]-2,3-dihydro-1-benzofuran-5-yl}-1λ⁶,2,5-thiadiazolidine-1,1,3-trione;
5-{(2R)-4-fluoro-2-[({[1-(fluoromethyl)cyclopropyl]methyl}amino)methyl]-6-hydroxy-2,3-dihydro-1-benzofuran-5-yl}-1λ⁶,2,5-thiadiazolidine-1,1,3-trione;
5-[(2S)-4-fluoro-6-hydroxy-2-{[(2-methylpropyl)amino]methyl}-2,3-dihydro-1H-indol-5-yl]-1λ⁶,2,5-thiadiazolidine-1,1,3-trione;
5-[(2R)-4-fluoro-6-hydroxy-2-{[(2-methylpropyl)amino]methyl}-2,3-dihydro-1H-indol-5-yl]-1λ⁶,2,5-thiadiazolidine-1,1,3-trione;
5-[(2R)-2-{[(2-ethylbutyl)amino]methyl}-4-fluoro-6-hydroxy-2,3-dihydro-1H-indol-5-yl]-1λ⁶,2,5-thiadiazolidine-1,1,3-trione;
5-{(2R)-4-fluoro-6-hydroxy-2-[(propylamino)methyl]-2,3-dihydro-1H-indol-5-yl}-1λ⁶,2,5-thiadiazolidine-1,1,3-trione;
5-[(2R)-4-fluoro-6-hydroxy-2-{[(2-methoxyethyl)amino]methyl}-2,3-dihydro-1H-indol-5-yl]-1λ⁶,2,5-thiadiazolidine-1,1,3-trione;
5-[(2R)-2-{[(cyclobutylmethyl)amino]methyl}-4-fluoro-6-hydroxy-2,3-dihydro-1H-indol-5-yl]-1λ⁶,2,5-thiadiazolidine-1,1,3-trione;
5-[(2R)-4-fluoro-6-hydroxy-2-({[(oxan-4-yl)methyl]amino}methyl)-2,3-dihydro-1H-indol-5-yl]-1λ⁶,2,5-thiadiazolidine-1,1,3-trione;
5-[(2R)-4-fluoro-6-hydroxy-2-{[(3-methylbutyl)amino]methyl}-2,3-dihydro-1H-indol-5-yl]-1λ⁶,2,5-thiadiazolidine-1,1,3-trione;
5-[(2R)-2-{[(3,3-difluoropropyl)amino]methyl}-4-fluoro-6-hydroxy-2,3-dihydro-1H-indol-5-yl]-1λ⁶,2,5-thiadiazolidine-1,1,3-trione;
5-[(2R)-4-fluoro-6-hydroxy-2-{[(3,3,3-trifluoropropyl)amino]methyl}-2,3-dihydro-1H-indol-5-yl]-1λ⁶,2,5-thiadiazolidine-1,1,3-trione;
5-[(2R)-4-fluoro-6-hydroxy-2-{[(2-methylbutyl)amino]methyl}-2,3-dihydro-1H-indol-5-yl]-1λ⁶,2,5-thiadiazolidine-1,1,3-trione;
5-[(2R)-2-({[(3,3-difluorocyclobutyl)methyl]amino}methyl)-4-fluoro-6-hydroxy-2,3-dihydro-1H-indol-5-yl]-1λ⁶,2,5-thiadiazolidine-1,1,3-trione;
5-[4-fluoro-6-hydroxy-2-({[2-(oxetan-3-yl)ethyl]amino}methyl)-2,3-dihydro-1H-indol-5-yl]-1λ⁶,2,5-thiadiazolidine-1,1,3-trione;
5-(2-{[(4,4-difluorobutyl)amino]methyl}-4-fluoro-6-hydroxy-2,3-dihydro-1H-indol-5-yl)-1λ⁶,2,5-thiadiazolidine-1,1,3-trione;
5-[(2R)-2-{[(cyclopentylmethyl)amino]methyl}-4-fluoro-6-hydroxy-2,3-dihydro-1H-indol-5-yl]-1λ⁶,2,5-thiadiazolidine-1,1,3-trione;
5-[(2R)-2-{[(cyclopropylmethyl)amino]methyl}-4-fluoro-6-hydroxy-2,3-dihydro-1H-indol-5-yl]-1λ⁶,2,5-thiadiazolidine-1,1,3-trione;
5-[(2R)-4-fluoro-6-hydroxy-2-{[(pentan-2-yl)amino]methyl}-2,3-dihydro-1H-indol-5-yl]-1λ⁶,2,5-thiadiazolidine-1,1,3-trione;
5-[(2R)-2-({[(2R)-butan-2-yl]amino}methyl)-4-fluoro-6-hydroxy-2,3-dihydro-1H-indol-5-yl]-1λ⁶,2,5-thiadiazolidine-1,1,3-trione;
5-{(2R)-2-[(butylamino)methyl]-4-fluoro-6-hydroxy-2,3-dihydro-1H-indol-5-yl}-1λ⁶,2,5-thiadiazolidine-1,1,3-trione;
5-[(2R)-4-fluoro-6-hydroxy-2-(hydroxymethyl)-2,3-dihydro-1H-indol-5-yl]-1λ⁶,2,5-thiadiazolidine-1,1,3-trione;
5-[(2R)-2-{[(2-cyclopropylethyl)amino]methyl}-4-fluoro-6-hydroxy-2,3-dihydro-1H-indol-5-yl]-1λ⁶,2,5-thiadiazolidine-1,1,3-trione;
5-[(2R)-2-{[(3,3-dimethylbutyl)amino]methyl}-4-fluoro-6-hydroxy-2,3-dihydro-1H-indol-5-yl]-1λ⁶,2,5-thiadiazolidine-1,1,3-trione;
5-{(2R)-4-fluoro-6-hydroxy-2-[({[3-(propan-2-yl)cyclobutyl]methyl}amino)methyl]-2,3-dihydro-1H-indol-5-yl}-1λ⁶,2,5-thiadiazolidine-1,1,3-trione;
5-[(2R)-4-fluoro-6-hydroxy-2-{[methyl(2-methylpropyl)amino]methyl}-2,3-dihydro-1H-indol-5-yl]-11λ⁶, 2,5-thiadiazolidine-1,1,3-trione;
5-[(2R)-4-fluoro-6-hydroxy-1-methyl-2-{[(2-methylpropyl)amino]methyl}-2,3-dihydro-1H-indol-5-yl]-1λ⁶,2,5-thiadiazolidine-1,1,3-trione; and
5-[(2R)-2-{[(3,3-difluoro-2-hydroxypropyl)amino]methyl}-4-fluoro-6-hydroxy-2,3-dihydro-1H-indol-5-yl]-1λ⁶,2,5-thiadiazolidine-1,1,3-trione;
   or a pharmaceutically acceptable salt thereof.

In some embodiments, a compound disclosed herein is formulated as a pharmaceutically acceptable composition comprising a disclosed compound and a pharmaceutically acceptable carrier. In some embodiments, disclosed herein is a method of treating non-small cell lung cancer in a patient in need thereof comprising administering to the patient an effective amount of a compound disclosed herein.

Table of Exemplary Compounds

| No. | Structure |
|---|---|
| 100 | 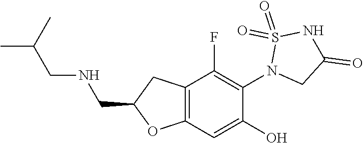 [SMILES: CC(C)CNC[C@H]1Cc2c(cc(O)c(N3CC(=O)NS3(=O)=O)c2F)O1] |
| 101 | 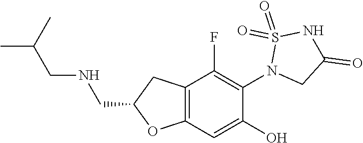 [SMILES: CC(C)CNC[C@@H]1Cc2c(cc(O)c(N3CC(=O)NS3(=O)=O)c2F)O1] |
| 102 | 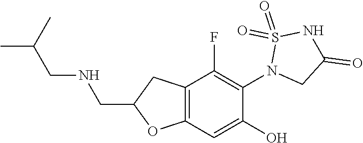 [SMILES: CC(C)CNCC1Cc2c(cc(O)c(N3CC(=O)NS3(=O)=O)c2F)O1] |
| 103 | 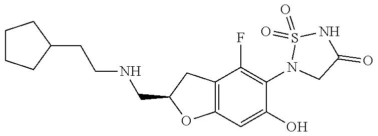 [SMILES: O=C1CN(c2c(O)cc3c(c2F)C[C@H](CNCCC2CCCC2)O3)S(=O)(=O)N1] |
| 104 | 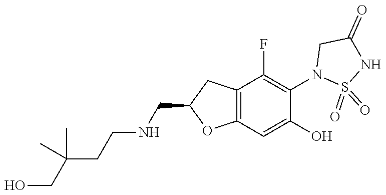 [SMILES: CC(C)(CO)CCNC[C@H]1Cc2c(cc(O)c(N3CC(=O)NS3(=O)=O)c2F)O1] |
| 105 | 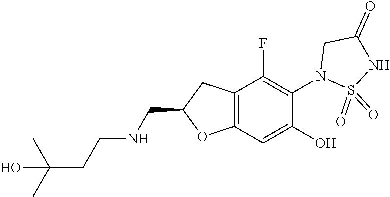 [SMILES: CC(C)(O)CCNC[C@H]1Cc2c(cc(O)c(N3CC(=O)NS3(=O)=O)c2F)O1] |
| 106 | 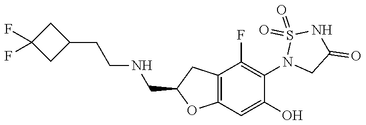 [SMILES: O=C1CN(c2c(O)cc3c(c2F)C[C@H](CNCCC2CC(F)(F)C2)O3)S(=O)(=O)N1] |
| 107 | 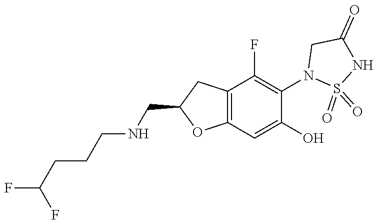 [SMILES: O=C1CN(c2c(O)cc3c(c2F)C[C@H](CNCCCC(F)F)O3)S(=O)(=O)N1] |
| 108 | 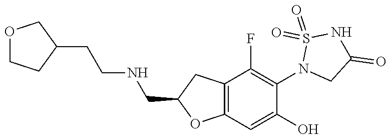 [SMILES: O=C1CN(c2c(O)cc3c(c2F)C[C@H](CNCCC2CCOC2)O3)S(=O)(=O)N1] |
| 109 | 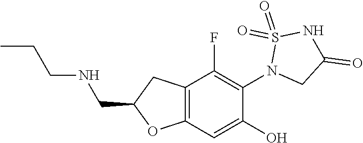 [SMILES: CCCNC[C@H]1Cc2c(cc(O)c(N3CC(=O)NS3(=O)=O)c2F)O1] |
| 110 | 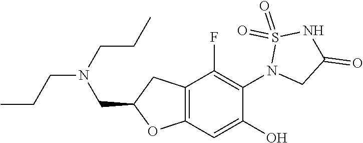 [SMILES: CCCN(CCC)C[C@H]1Cc2c(cc(O)c(N3CC(=O)NS3(=O)=O)c2F)O1] |
| 111 | 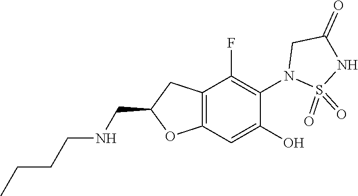 [SMILES: CCCCNC[C@H]1Cc2c(cc(O)c(N3CC(=O)NS3(=O)=O)c2F)O1] |
| 112 | 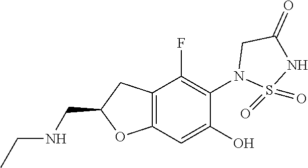 [SMILES: CCNC[C@H]1Cc2c(cc(O)c(N3CC(=O)NS3(=O)=O)c2F)O1] |
| 113 | 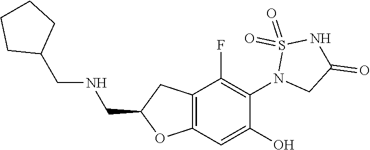 [SMILES: O=C1CN(c2c(O)cc3c(c2F)C[C@H](CNCC2CCCC2)O3)S(=O)(=O)N1] |
| 114 | 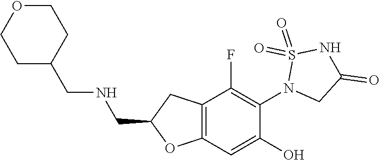 [SMILES: O=C1CN(c2c(O)cc3c(c2F)C[C@H](CNCC2CCOCC2)O3)S(=O)(=O)N1] |
| 115 | 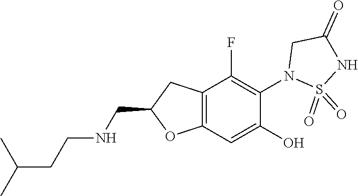 [SMILES: CC(C)CCNC[C@H]1Cc2c(cc(O)c(N3CC(=O)NS3(=O)=O)c2F)O1] |
| 116 | 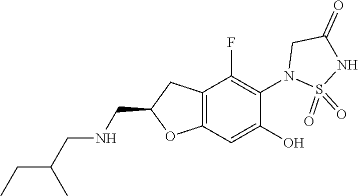 [SMILES: CCC(C)CNC[C@H]1Cc2c(cc(O)c(N3CC(=O)NS3(=O)=O)c2F)O1] |
| 117 | 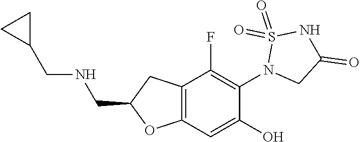 [SMILES: O=C1CN(c2c(O)cc3c(c2F)C[C@H](CNCC2CC2)O3)S(=O)(=O)N1] |
| 118 | 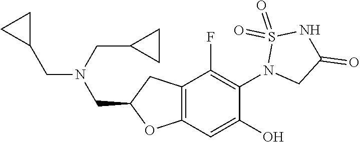 [SMILES: O=C1CN(c2c(O)cc3c(c2F)C[C@H](CN(CC2CC2)CC2CC2)O3)S(=O)(=O)N1] |
| 119 | 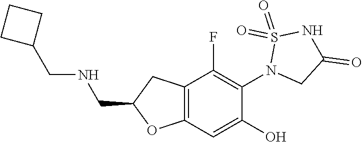 [SMILES: O=C1CN(c2c(O)cc3c(c2F)C[C@H](CNCC2CCC2)O3)S(=O)(=O)N1] |
| 120 | 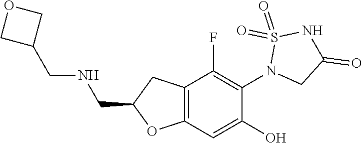 [SMILES: O=C1CN(c2c(O)cc3c(c2F)C[C@H](CNCC2COC2)O3)S(=O)(=O)N1] |
| 121 | 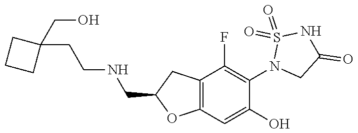 [SMILES: O=C1CN(c2c(O)cc3c(c2F)C[C@H](CNCCC2(CO)CCC2)O3)S(=O)(=O)N1] |
| 122 | 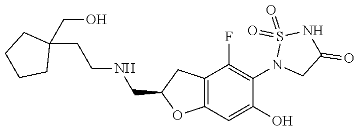 [SMILES: O=C1CN(c2c(O)cc3c(c2F)C[C@H](CNCCC2(CO)CCCC2)O3)S(=O)(=O)N1] |
| 123 | 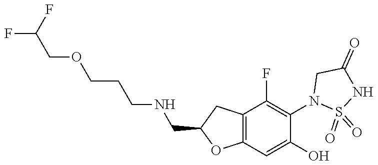 [SMILES: O=C1CN(c2c(O)cc3c(c2F)C[C@H](CNCCCOCC(F)F)O3)S(=O)(=O)N1] |
| 124 | 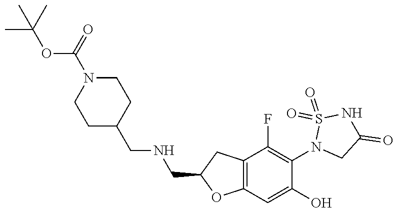 [SMILES: CC(C)(C)OC(=O)N1CCC(CNC[C@H]2Cc3c(cc(O)c(N4CC(=O)NS4(=O)=O)c3F)O2)CC1] |
| 125 | 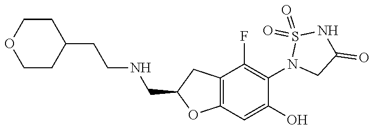 [SMILES: O=C1CN(c2c(O)cc3c(c2F)C[C@H](CNCCC2CCOCC2)O3)S(=O)(=O)N1] |
| 126 | 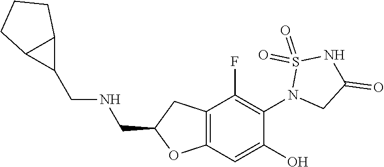 [SMILES: O=C1CN(c2c(O)cc3c(c2F)C[C@H](CNCC2C4CCCC42)O3)S(=O)(=O)N1] |
| 127 | 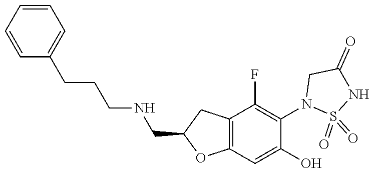 [SMILES: O=C1CN(c2c(O)cc3c(c2F)C[C@H](CNCCCc2ccccc2)O3)S(=O)(=O)N1] |
| 128 | 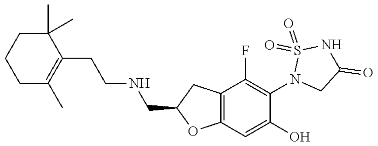 [SMILES: CC1=C(CCNC[C@H]2Cc3c(cc(O)c(N4CC(=O)NS4(=O)=O)c3F)O2)C(C)(C)CCC1] |
| 129 | 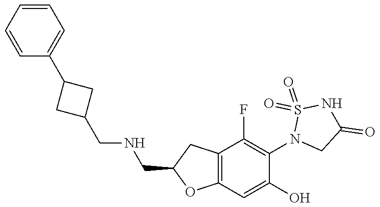 [SMILES: O=C1CN(c2c(O)cc3c(c2F)C[C@H](CNCC2CC(c4ccccc4)C2)O3)S(=O)(=O)N1] |
| 130 | 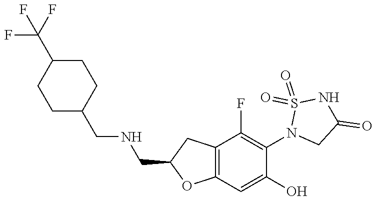 [SMILES: O=C1CN(c2c(O)cc3c(c2F)C[C@H](CNCC2CCC(C(F)(F)F)CC2)O3)S(=O)(=O)N1] |
| 131 | 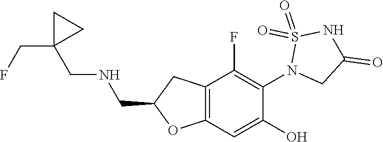 [SMILES: O=C1CN(c2c(O)cc3c(c2F)C[C@H](CNCC2(CF)CC2)O3)S(=O)(=O)N1] |
| 132 | 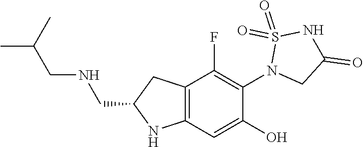 |
| 133 | 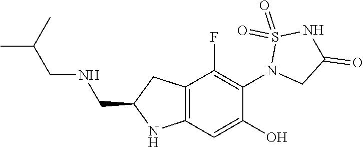 |
| 134 | 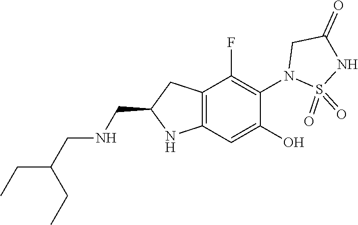 [SMILES: CCC(CC)CNC[C@H]1Cc2c(cc(O)c(N3CC(=O)NS3(=O)=O)c2F)N1] |
| 135 | 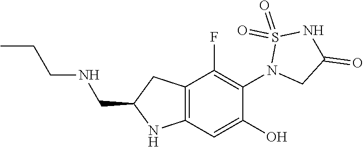 [SMILES: CCCNC[C@H]1Cc2c(cc(O)c(N3CC(=O)NS3(=O)=O)c2F)N1] |
| 136 | 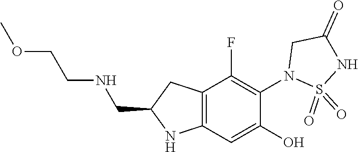 [SMILES: COCCNC[C@H]1Cc2c(cc(O)c(N3CC(=O)NS3(=O)=O)c2F)N1] |
| 137 | 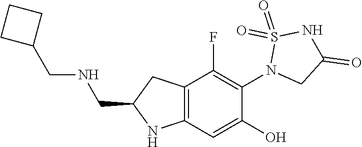 [SMILES: O=C1CN(c2c(O)cc3c(c2F)C[C@H](CNCC2CCC2)N3)S(=O)(=O)N1] |
| 138 | 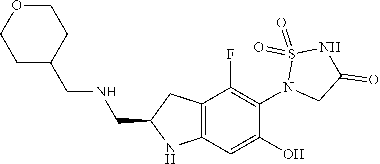 [SMILES: O=C1CN(c2c(O)cc3c(c2F)C[C@H](CNCC2CCOCC2)N3)S(=O)(=O)N1] |
| 139 | 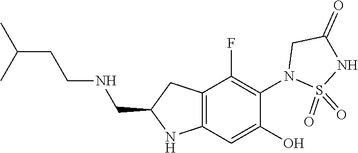 [SMILES: CC(C)CCNC[C@H]1Cc2c(cc(O)c(N3CC(=O)NS3(=O)=O)c2F)N1] |
| 140 | 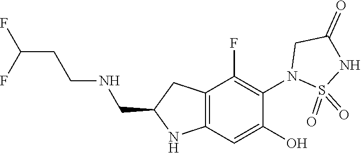 [SMILES: O=C1CN(c2c(O)cc3c(c2F)C[C@H](CNCCC(F)F)N3)S(=O)(=O)N1] |
| 141 | 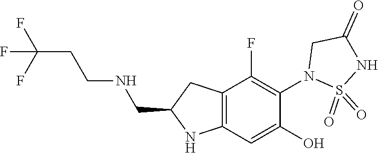 [SMILES: O=C1CN(c2c(O)cc3c(c2F)C[C@H](CNCCC(F)(F)F)N3)S(=O)(=O)N1] |
| 142 | 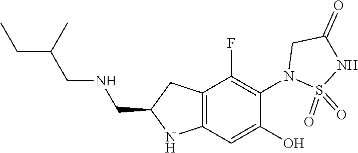 [SMILES: CCC(C)CNC[C@H]1Cc2c(cc(O)c(N3CC(=O)NS3(=O)=O)c2F)N1] |
| 143 | 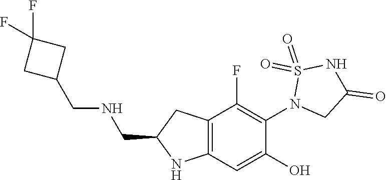 [SMILES: O=C1CN(c2c(O)cc3c(c2F)C[C@H](CNCC2CC(F)(F)C2)N3)S(=O)(=O)N1] |
| 144 | 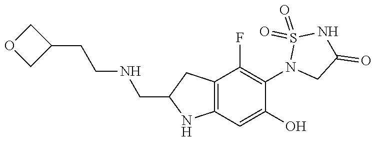 [SMILES: O=C1CN(c2c(O)cc3c(c2F)CC(CNCCC2COC2)N3)S(=O)(=O)N1] |
| 145 | 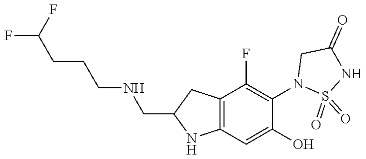 [SMILES: O=C1CN(c2c(O)cc3c(c2F)CC(CNCCCC(F)F)N3)S(=O)(=O)N1] |
| 146 | 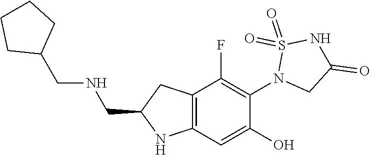 [SMILES: O=C1CN(c2c(O)cc3c(c2F)C[C@H](CNCC2CCCC2)N3)S(=O)(=O)N1] |
| 147 | 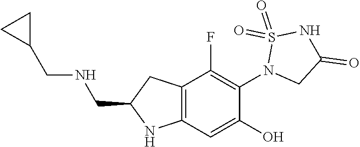 [SMILES: O=C1CN(c2c(O)cc3c(c2F)C[C@H](CNCC2CC2)N3)S(=O)(=O)N1] |
| 148 | 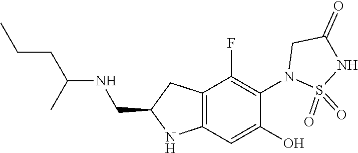 [SMILES: CCCC(C)NC[C@H]1Cc2c(cc(O)c(N3CC(=O)NS3(=O)=O)c2F)N1] |
| 149 | 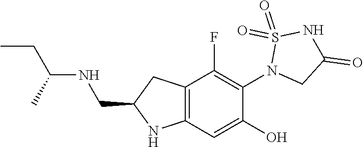 [SMILES: CC[C@@H](C)NC[C@H]1Cc2c(cc(O)c(N3CC(=O)NS3(=O)=O)c2F)N1] |
| 150 | 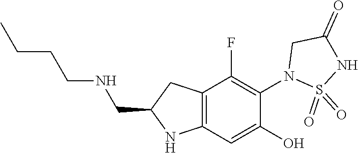 [SMILES: CCCCNC[C@H]1Cc2c(cc(O)c(N3CC(=O)NS3(=O)=O)c2F)N1] |
| 151 | 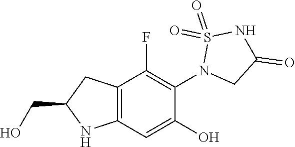 [SMILES: O=C1CN(c2c(O)cc3c(c2F)C[C@H](CO)N3)S(=O)(=O)N1] |
| 152 | 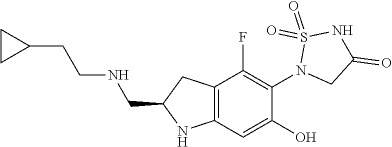 [SMILES: O=C1CN(c2c(O)cc3c(c2F)C[C@H](CNCCC2CC2)N3)S(=O)(=O)N1] |
| 153 | 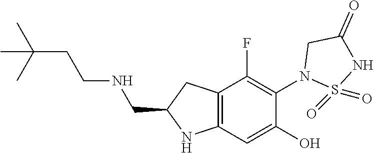 [SMILES: CC(C)(C)CCNC[C@H]1Cc2c(cc(O)c(N3CC(=O)NS3(=O)=O)c2F)N1] |
| 154 | 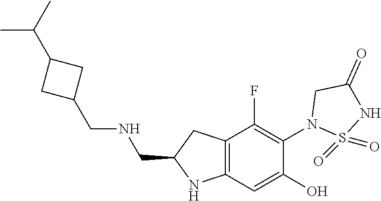 [SMILES: CC(C)C1CC(CNC[C@H]2Cc3c(cc(O)c(N4CC(=O)NS4(=O)=O)c3F)N2)C1] |
| 155 | 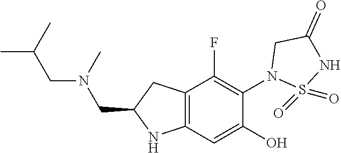 [SMILES: CC(C)CN(C)C[C@H]1Cc2c(cc(O)c(N3CC(=O)NS3(=O)=O)c2F)N1] |
| 156 | 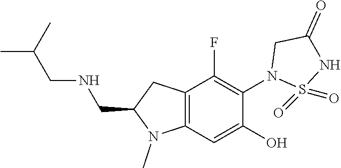 [SMILES: CC(C)CNC[C@H]1Cc2c(cc(O)c(N3CC(=O)NS3(=O)=O)c2F)N1C] |
| 157 | 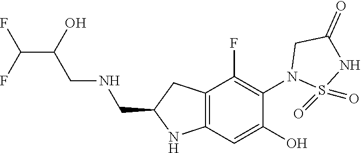 [SMILES: O=C1CN(c2c(O)cc3c(c2F)C[C@H](CNCC(O)C(F)F)N3)S(=O)(=O)N1] |

### Methods of Making Exemplary Compounds

The compounds of the present disclosure may be better understood in connection with the following synthetic schemes and methods which illustrate a means by which the compounds can be prepared. The compounds of the present disclosure can be prepared by a variety of synthetic procedures. Representative synthetic procedures are shown in, but not limited to, Schemes 1-2.

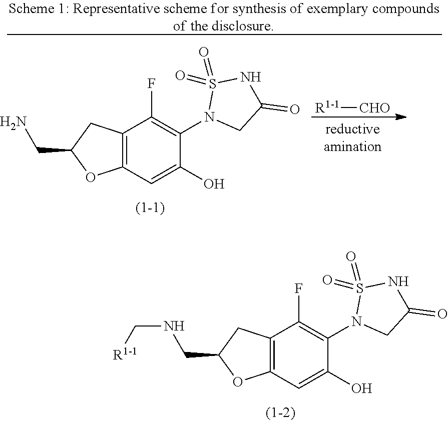

As shown in Scheme 1, compounds of formula (1-2) can be prepared from compounds of formula (1-1). Compounds of formula (1-1) can be reductively aminated with aldehydes, R¹⁻¹—CHO, wherein R¹⁻¹ is optionally substituted C₁₋₆alkyl, optionally substituted —C₁₋₆alkylene-C₃₋₆cycloalkyl, optionally substituted —C₁₋₆alkylene-phenyl, or optionally substituted —C₁₋₆alkylene-4-6 membered heterocyclyl, to give compounds of formula (1-2). Compounds of formula (1-2) can initially be reacted with aldehydes, R¹⁻¹—CHO, in the presence of triethylamine in a solvent such ethanol or a mixture of ethanol and dichloromethane. Subsequent treatment with a reductant such as sodium borohydride can complete the reductive amination. Compounds of formula (1-2) are representative of compounds of Formula (I).

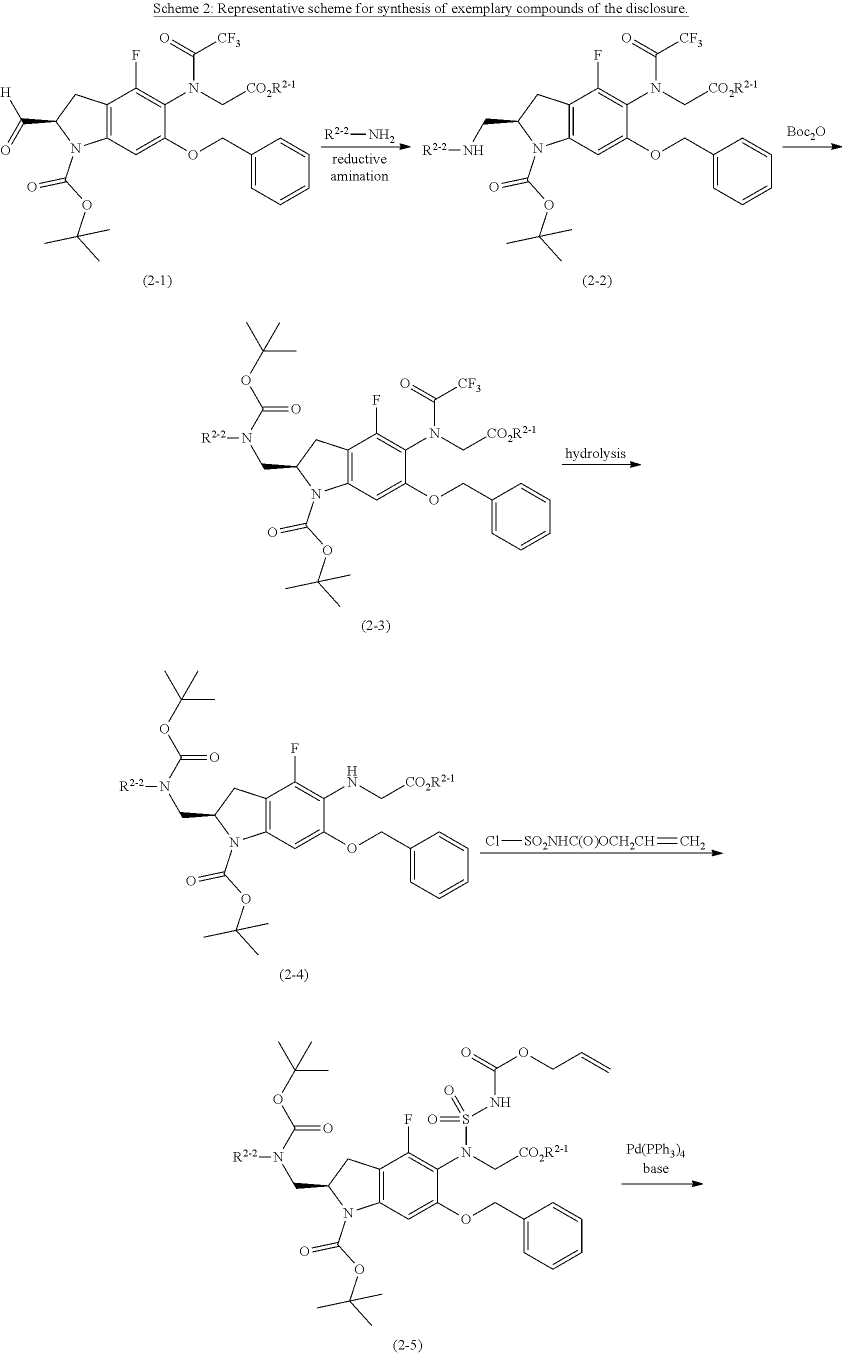

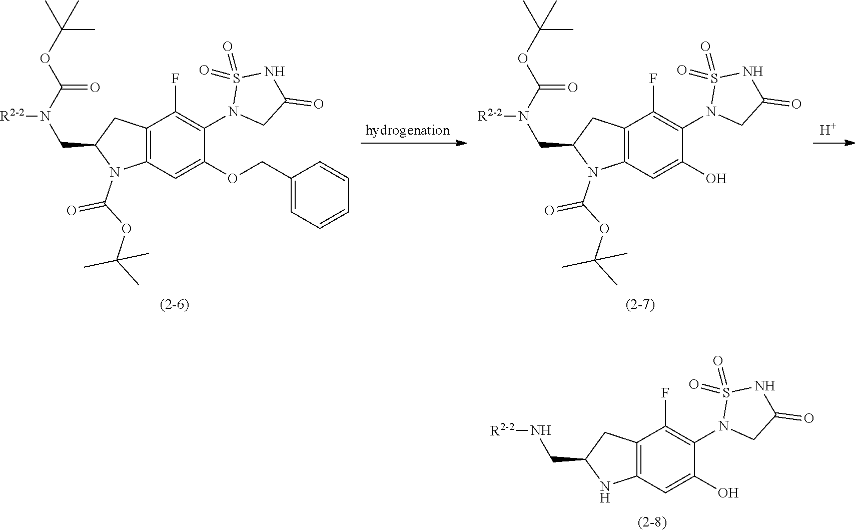

As shown in Scheme 2, compounds of formula (2-8) can be prepared from compounds of formula (2-1). Compounds of formula (2-1), wherein R²⁻¹ is methyl or t-butyl, can be reductively aminated with amines, R²⁻²—NH₂, wherein R²⁻² is optionally substituted C₁₋₆alkyl, optionally substituted —C₁₋₆alkylene-C₃₋₆cycloalkyl, optionally substituted —C₁₋₆alkylene-phenyl, or optionally substituted —C₁₋₆alkylene-4-6 membered heterocyclyl, to give compounds of formula (2-2). Compounds of formula (2-1) are initially combined with amines, R²⁻²—NH₂, in a solvent such as 1,2-dichloroethane in the presence of acetic acid and optionally in the presence of triethylamine and then subsequently treated with a reductant such as sodium triacetoxyborohydride to perform the reductive amination. The amines of formula (2-2) can be protected upon treatment with di-tert-butyl dicarbonate in the presence of N,N-diisopropylethylamine in a solvent such as acetonitrile, dichloromethane, or a mixture thereof, to give compounds of formula (2-3). The trifluoroacetyl moiety of compounds of formula (2-3) can be removed by hydrolysis with either sodium methoxide in warmed methanol or aqueous lithium hydroxide in optionally warmed methanol or a mixture of methanol and tetrahydrofuran to give compounds of formula (2-4). Compounds of formula (2-4) can be treated with Cl—SO₂—NC(O)OCH₂CH═CH₂ in the presence of a base such as triethylamine in dichloromethane at approximately 0° C. to give compounds of formula (2-5). Compounds of formula (2-5) can be converted to compounds of formula (2-6) by treatment with a catalyst such as tetrakis(triphenylphosphine)palladium(0) and a base such as potassium carbonate or sodium methoxide in heated methanol. The benzyl moiety of compounds of formula (2-6) can be removed by hydrogenation in the presence of a catalyst such as 10% palladium on carbon in a solvent such as a mixture of water and ethanol to give compounds of formula (2-7). Compounds of formula (2-7) can be treated under acidic conditions such as with trifluoroacetic acid or hydrochloric acid optionally in the presence of propan-1-amine in a solvent such as dichloromethane, acetonitrile, or a mixture thereof, to give compounds of formula (2-8). Compounds of formula (2-8) are representative of compounds of Formula (I).

### Pharmaceutical Compositions

The present disclosure provides pharmaceutical compositions comprising a compound disclosed herein, e.g., a compound of Formula (I). In some embodiments, the pharmaceutical composition further comprises a pharmaceutically acceptable excipient. In some embodiments, a compound disclosed herein, e.g., a compound of Formula (I), is provided in an effective amount in the pharmaceutical composition. In some embodiments, the effective amount is a therapeutically effective amount. In certain embodiments, the effective amount is a prophylactically effective amount.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., inhibiting the activity of a target molecule (e.g. PTPN2 and/or PTPN1), and/or reducing, eliminating, or slowing the progression of disease symptoms. Determination of a therapeutically effective amount of a compound disclosed herein is well within the capabilities of those skilled in the art, especially in light of the detailed disclosure herein.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present disclosure.

“Alkyl” refers to a radical of a straight-chain or branched saturated hydrocarbon group having from 1 to 20 carbon atoms (“C₁-C₂₀ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅, 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₆ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present disclosure.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include, but are not limited to, phenyl, naphthyl, indenyl, and tetrahydronaphthyl. Each instance of an aryl group may be independently optionally substituted, e.g., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

“Halo” or “halogen,” independently or as part of another substituent, mean, unless otherwise stated, a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

Additionally, terms such as “haloalkyl” are meant to include monohaloalkyl and polyhaloalkyl. For example, the term “halo-C₁-C₆ alkyl” includes, but is not limited to, fluoromethyl, difluoromethyl, trifluoromethyl, 2,2,2-trifluoroethyl, 4-chlorobutyl, 3-bromopropyl, and the like.

“Heteroaryl” refers to a radical of a 5-10 membered monocyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits.

“Cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. Each instance of a cycloalkyl group may be independently optionally substituted, e.g., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

In some embodiments, “cycloalkyl” is a monocyclic, saturated cycloalkyl group having from 3 to 10 ring carbon atoms (“C₃-C₁₀ cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 5 to 6 ring carbon atoms (“C₅-C₆ cycloalkyl”). In some embodiments, a cycloalkyl group has 5 to 10 ring carbon atoms (“C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is substituted C₃-C₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione.

Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Amino” refers to the radical —NR⁷⁰R⁷¹, wherein R⁷⁰ and R⁷¹ are each independently hydrogen, C₁-C₈ alkyl, C₃-C₁₀ cycloalkyl, 4-10 membered heterocyclyl, C₆-C₁₀ aryl, and 5-10-membered heteroaryl. In some embodiments, amino refers to NH₂.

“Cyano” refers to the radical —CN.

“Hydroxy” or “hydroxyl” refers to the radical —OH.

In some embodiments one or more of the nitrogen atoms of a disclosed compound if present are oxidized to the corresponding N-oxide.

Alkyl, alkenyl, alkynyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

### Other Definitions

“Treating” or “treatment” refers to a method of alleviating or abrogating a disease and/or its attendant symptoms. In certain embodiments, the compounds disclosed herein are useful in the treatment of non-small cell lung cancer. In certain embodiments, a method of treating a human subject with non-small cell lung cancer comprising administering to the human subject in need thereof a therapeutically effective amount of a compound of formula (I) is provided.

The phrase “therapeutically effective amount” refers to an amount of a compound, or a pharmaceutically acceptable salt thereof, sufficient to prevent the development of or to alleviate to some extent one or more of the symptoms of the condition or disorder being treated when administered for treatment in a particular subject or subject population. The term “subject” as used herein, refers to a human. The terms “human,” patient,” and “subject” are used interchangeably herein.

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor.

“Patient” or “subject” in need thereof refers to a living organism suffering from or prone to a disease or condition that can be treated by administration of a compound or pharmaceutical composition, as provided herein. In some embodiments, a patient is human.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient.

The term “PTPN2” as used herein refers to protein tyrosine phosphatase non-receptor type 2. The term “PTPN1” refers to protein tyrosine phosphatase non-receptor type 1 (PTPN1), also known as protein tyrosine phosphatase-1B (PTP1B),

In some embodiments, a compound disclosed herein is formulated as a pharmaceutically acceptable composition comprising a disclosed compound and a pharmaceutically acceptable carrier.

### EXEMPLIFICATION

In order that the present disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General schemes relating to methods of making exemplary compounds of the invention are additionally described in the section entitled Methods of Making Exemplary Compounds.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

APCI for atmospheric pressure chemical ionization; Boc for tert-butoxycarbonyl; BrettPhos for 2-(dicyclohexylphosphino)3,6-dimethoxy-2′,4′,6′-triisopropyl-1,1′-biphenyl; BrettPhos Pd G3 for [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate; DMSO for dimethyl sulfoxide; ee for enantiomeric excess; ESI for electrospray ionization; HPLC for high performance liquid chromatography; i.d. for internal diameter; MS for mass spectrum; NMR for nuclear magnetic resonance; Ph for phenyl; ppm for parts per million; psi for pounds per square inch; PTFE for polytetrafluoroethylene; RockPhos for 2-di(tert-butyl)phosphino-2′,4′,6′-triisopropyl-3-methoxy-6-methylbiphenyl; RockPhos Pd G3 for [(2-di-tert-butylphosphino-3-methoxy-6-methyl-2′,4′,6′-triisopropyl-1,1′-biphenyl)-2-(2-aminobiphenyl)]palladium(II) methanesulfonate; RuPhos Pd G3 for (2-dicyclohexylphosphino-2′,6′-diisopropoxy-1,1′-biphenyl)[2-(2′-amino-1,1′-biphenyl)]palladium(II) methanesulfonate; SFC for supercritical chromatography; and v/v for volume/volume.

### Example I-1: 5-[(2R)-4-fluoro-6-hydroxy-2-{[(2-methylpropyl)amino]methyl}-2,3-dihydro-1-benzofuran-5-yl]-1λ⁶,2,5-thiadiazolidine-1,1,3-trione (Compound 100)

### Example I-1A-1 and Example I-1A-2: tert-butyl {[(2S)-4-fluoro-6-hydroxy-5-(1,1,4-trioxo-1λ⁶,2,5-thiadiazolidin-2-yl)-2,3-dihydro-1-benzofuran-2-yl]methyl}(2-methylpropyl)carbamate (I-1A-1) and tert-butyl {[(2R)-4-fluoro-6-hydroxy-5-(1,1,4-trioxo-1λ⁶,2,5-thiadiazolidin-2-yl)-2,3-dihydro-1-benzofuran-2-yl]methyl}(2-methylpropyl)carbamate (I-1A-2)

Triethylamine (42.5 mg, 0.420 mmol) was added to a suspension of the product of Example I-3 (43 mg, 0.115 mmol) in acetonitrile (1 mL) at 23° C. giving a clear solution. Di-tert-butyl dicarbonate (22.90 mg, 0.105 mmol) was then added, and the reaction mixture was stirred at ambient temperature for 1 hour. The reaction mixture was diluted with ethyl acetate, washed with 0.2 M HCl and brine, and dried over Na₂SO₄. The mixture was filtered and concentrated. The crude residue was dissolved in methanol (5 mL), K₂CO₃ (200 mg, 1.45 mmol) was added, and the mixture was stirred at ambient temperature for 14 hours. The reaction mixture was diluted with 0.2 M HCl (20 mL) and extracted with ethyl acetate. The ethyl acetate fraction was washed with brine and dried over Na₂SO₄. The mixture was concentrated, and the residue was purified by SFC to give two peaks. The first peak (Example I-1A-1) was arbitrarily assigned as the (S)-enantiomer (15.9 mg, >99% ee, 29% yield); MS (APCI⁻) m/z 472.6 [M−H]⁻. The 2^{nd} peak (example I-1A-2) was arbitrarily assigned as the (R)-enantiomer (24.5 mg, 87% ee, 45% yield); MS (APCI⁻) m/z 472.8 [M−H]⁻.

Preparative SFC was performed on a Waters SFC80Q SFC running under ChromScope software control. The preparative SFC system was equipped with a CO₂ pump, modifier pump with 4-port solvent selection valve, automated back pressure regulator (ABPR), UV detector, and 6-position fraction collector. The mobile phase was comprised of supercritical CO₂ supplied by a Dewar of bone-dry non-certified CO₂ pressurized to 350 psi with a modifier of methanol (0.1% diethylamine) at a flow rate of 80 g/minute. The column was held at ambient temperature and the backpressure regulator was set to maintain 150 bar. The sample was dissolved in methanol at a concentration of 8 mg/mL. The sample was loaded into the modifier stream in 0.2 mL (2 mg) injections. The mobile phase was held isocratically at 20% modifier. Fraction collection was time triggered. The instrument was fitted with a CHIRALPAK® IC column with dimensions 31 mm i.d.×250 mm length with 5 μm particles.

The ee % was determined by Analytical Ultra-High Performance SFC which was performed on a Waters UPC² SFC system running under Empower software control. The SFC system included a 6-way column switcher, sample manager autosampler running with partial loop injections, CO₂ pump, binary modifier pump, column oven, and convergence manager (ABPR). The mobile phase comprised of bulk-delivered bone-dry CO₂ with a modifier mixture of methanol (0.1% diethylamine)-CO₂ at a flow rate of 1 mL/minute. The oven temperature was at 35° C. and the outlet pressure at 150 bar. The mobile phase was 20% isocratic of modifier over 20 minutes. The retention times of the enantiomers were retention time=11.114 minutes for example I-1A-1 and retention time=12.581 minutes for example I-1A-2.

### Example I-1B: 5-[(2R)-4-fluoro-6-hydroxy-2-{[(2-methylpropyl)amino]methyl}-2,3-dihydro-1-benzofuran-5-yl]-1λ⁶,2,5-thiadiazolidine-1,1,3-trione-trifluoroacetic acid

A mixture of the product of Example I-1A-2 (24 mg, 0.051 mmol) (87% ee) and trifluoroacetic acid (148 mg, 1.298 mmol) in dichloromethane (1 mL) was stirred at room temperature for 30 minutes. The reaction mixture was concentrated in vacuo. The residue was triturated with acetonitrile to give the title compound as a trifluoroacetic acid salt (15 mg, 0.031 mmol, 60.7% yield). ¹H NMR (600 MHz, DMSO-d₆) δ ppm 9.27 (s, 1H), 8.60 (s, 1H), 8.50 (s, 1H), 6.21 (s, 1H), 5.17 (dq, J=12.3, 7.0 Hz, 1H), 3.88 (s, 2H), 3.27 (d, J=6.3 Hz, 2H), 2.94 (dd, J=15.2, 6.8 Hz, 1H), 2.83 (ddt, J=19.9, 12.1, 6.1 Hz, 2H), 1.99 (hept, J=6.8 Hz, 1H), 0.95 (t, J=6.6 Hz, 6H); MS (APCI⁻) m/z 372.4 [M−H]⁻.

### Example I-2: 5-[(2S)-4-fluoro-6-hydroxy-2-{[(2-methylpropyl)amino]methyl}-2,3-dihydro-1-benzofuran-5-yl]-1λ⁶,2,5-thiadiazolidine-1,1,3-trione (Compound 101)

The title compound was synthesized as a trifluoroacetic acid salt from Example I-1A-1 using the same procedure described for Example I-1B in 39% yield. ¹H NMR (400 MHz, DMSO-d₆) δ ppm 9.28 (s, 1H), 8.51 (s, 2H), 6.21 (d, J=1.1 Hz, 1H), 5.22-5.10 (m, 1H), 3.88 (s, 2H), 3.26 (d, J=6.3 Hz, 2H), 2.93 (dd, J=15.3, 6.7 Hz, 1H), 2.89-2.76 (m, 2H), 1.98 (hept, J=6.8 Hz, 1H), 0.95 (dd, J=6.7, 4.3 Hz, 6H); MS (APCI⁺) m/z 374.6 [M+H]⁺.

### Example I-3: 5-(4-fluoro-6-hydroxy-2-{[(2-methylpropyl)amino]methyl}-2,3-dihydro-1-benzofuran-5-yl)-1λ⁶,2,5-thiadiazolidine-1,1,3-trione (Compound 102)

### Example I-3A: 1-(benzyloxy)-5-bromo-3-fluoro-2-nitrobenzene

Benzyl alcohol (2.2 mL, 21.16 mmol) was added to a solution of 5-bromo-1,3-difluoro-2-nitrobenzene (5 g, 19.96 mmol) and cesium carbonate (13 g, 39.9 mmol) in N,N-dimethylformamide (10 mL) at 0° C. The reaction mixture was stirred at room temperature for 24 hours. The reaction mixture was diluted with ethyl acetate (100 mL) and water (100 mL). The aqueous layer was separated and extracted with ethyl acetate (2×50 mL). The organic layers were combined, washed with brine (4×50 mL), dried over magnesium sulfate, filtered, and concentrated under reduced pressure. The crude residue was subjected to column chromatography (SiO₂, dry loaded with silica, 0-30% ethyl acetate in isohexane) to afford the title compound (5.7 g, 16.25 mmol, 81% yield, 93% purity). ¹H NMR (400 MHz, DMSO-d₆) δ ppm 7.71-7.51 (m, 2H), 7.48-7.31 (m, 5H), 5.37 (s, 2H).

### Example I-3B: 2-(benzyloxy)-4-bromo-6-fluoroaniline

To a suspension of zinc (5.2 g, 80 mmol) and the product of Example I-3A (5.7 g, 16.25 mmol) in methanol/tetrahydrofuran (1:1, 75 mL) at 5° C. was added a saturated aqueous solution of ammonium chloride (28 mL) over a period of 15 minutes. The reaction mixture was allowed to warm to room temperature over 2 hours and stirred at room temperature for 20 hours. The reaction mixture was cooled to 5° C., more zinc (3.2 g, 48.9 mmol) was added, and the reaction mixture was stirred at 5° C. for 5 minutes. A saturated aqueous solution of ammonium chloride (17 mL) was added over a period of 10 minutes. The reaction mixture was warmed to room temperature over 1 hour and stirred at room temperature for 100 hours. Diatomaceous earth (5 g) was added to the reaction mixture and the suspension was filtered through a pad of diatomaceous earth. The filter cake was washed with ethyl acetate (2×200 mL). The filtrate was concentrated under reduced pressure and diluted with ethyl acetate (150 mL) and water (50 mL). The aqueous layer was extracted with ethyl acetate (2×75 mL). The organic layers were combined, dried over magnesium sulfate, filtered, and concentrated under reduced pressure to afford the title compound (4.87 g, 14.80 mmol, 91% yield). ¹H NMR (400 MHz, DMSO-d₆) δ ppm 7.53-7.45 (m, 2H), 7.43-7.36 (m, 2H), 7.35-7.29 (m, 1H), 7.00-6.92 (m, 2H), 5.16 (s, 2H), 4.84 (s, 2H); MS (ESI⁺) m/z 298, 296 [M+H]⁺.

### Example I-3C: N-[2-(benzyloxy)-4-bromo-6-fluorophenyl]-2,2,2-trifluoroacetamide

Trifluoroacetic anhydride (2 mL, 14.16 mmol) was slowly added to a solution of the product of Example I-3B (4.2 g, 13.47 mmol) and pyridine (1.6 mL, 19.78 mmol) in dichloromethane (20 mL) at 0° C. The reaction mixture was stirred for 15 minutes at 0° C., warmed to room temperature, and stirred for 18 hours. The reaction mixture was quenched with water (10 mL) and diluted with dichloromethane (25 mL) and water (20 mL). The aqueous layer was separated and extracted with dichloromethane (2×30 mL). The organic layers were combined, dried over magnesium sulfate, filtered, and concentrated under reduced pressure. The crude residue was subjected to column chromatography (SiO₂, dry loaded with diatomaceous earth, 0-40% ethyl acetate in isohexane) to afford the title compound (5.0 g, 11.93 mmol, 89% yield). ¹H NMR (400 MHz, DMSO-d₆) δ ppm 11.04 (s, 1H), 7.50-7.20 (m, 7H), 5.24 (s, 2H); MS (ESI⁻) m/z 390, 392 [M−H]⁻.

### Example I-3D: N-[6-(benzyloxy)-4-bromo-2-fluoro-3-(prop-2-en-1-yl)phenyl]-2,2,2-trifluoroacetamide

n-Butyllithium (2.5 M in hexane) (8.8 mL, 22.00 mmol) was added dropwise to a solution of diisopropylamine (3.2 mL, 22.83 mmol) in tetrahydrofuran (20 mL) at −78° C. The reaction mixture was stirred at −78° C. for 45 minutes. A solution of the product of Example I-3C (4.12 g, 9.98 mmol) in tetrahydrofuran (3 mL) was added dropwise over 10 minutes, and the reaction mixture was stirred at −78° C. for 30 minutes. Allyl bromide (0.9 mL, 10.40 mmol) was added dropwise over 10 minutes, and the mixture was stirred at −78° C. for 45 minutes. The reaction mixture was quenched with 1 M aqueous hydrochloric acid (50 mL) and diluted with ethyl acetate (100 mL) and water (30 mL). The aqueous layer was separated and extracted with ethyl acetate (2×70 mL). The organic layers were combined and dried over magnesium sulfate, filtered, and concentrated under reduced pressure. The crude residue was subjected to column chromatography (SiO₂, dry loaded with diatomaceous earth, 0-10% ethyl acetate in isohexane) to afford the title compound (2.38 g, 4.41 mmol, 44% yield, 80% purity). ¹H NMR (400 MHz, DMSO-d₆) δ ppm 11.06 (s, 1H), 7.52-7.20-(m, 6H), 5.97-5.79 (m, 1H), 5.31-5.16 (m, 2H), 5.07 (d, J=10.1 Hz, 1H), 4.95 (d, J=17.0 Hz, 1H), 3.53-3.37 (m, 2H); MS (ESI⁻) m/z 430, 432 [M−H]⁻.

### Example I-3E: N-{6-(benzyloxy)-4-bromo-2-fluoro-3-[(oxiran-2-yl)methyl]phenyl}-2,2,2-trifluoroacetamide

3-Chloroperoxybenzoic acid (4 g, 17.38 mmol) was added portion-wise to a solution of the product of Example I-3D (2.38 g, 4.41 mmol) in dichloromethane (20 mL) at 0° C. The reaction mixture was stirred at 0° C. for 1 hour then warmed to room temperature and stirred for 18 hours. A saturated aqueous solution of sodium thiosulfate (100 mL), a saturated aqueous solution of sodium hydrogen carbonate (50 mL) and dichloromethane (50 mL) were added, and the mixture was rapidly stirred for 48 hours. The reaction mixture was diluted with dichloromethane (100 mL), and the aqueous layer was separated. The aqueous layer was extracted with dichloromethane (2×100 mL). The organic layers were combined, dried over magnesium sulfate, filtered, and concentrated under reduced pressure. The crude residue was subjected to column chromatography (SiO₂, 0-55% ethyl acetate in isohexane) to afford the title compound (1.9 g, 4.03 mmol, 91% yield). ¹H NMR (400 MHz, DMSO-d₆) δ ppm 11.08 (s, 1H), 7.49-7.24-(m, 6H), 5.24 (s, 2H), 3.20-2.87-(m, 3H), 2.80-2.68 (m, 1H), 2.47-2.41 (m, 1H); MS (ESI⁻) m/z 446, 448.1 [M−H]⁻.

### Example I-3F: 1-[3-amino-4-(benzyloxy)-6-bromo-2-fluorophenyl]-3-[(2-methylpropyl)amino]propan-2-ol

2-Methylpropan-1-amine (6.00 mL, 60.4 mmol) was added to a solution of the product of Example I-3E (1.9 g, 4.03 mmol) in methanol (15 mL). The reaction mixture was stirred at room temperature for 20 hours and then stirred at 45° C. for 30 hours. The reaction mixture was cooled to room temperature and concentrated under reduced pressure. The crude residue was subjected to column chromatography (SiO₂, 0-15% methanol in dichloromethane) to afford the title compound (1.33 g, 2.97 mmol, 74% yield). ¹H NMR (400 MHz, DMSO-d₆) δ ppm 7.49 (d, J=7.1 Hz, 2H), 7.45-7.28-(m, 3H), 6.98 (d, J=1.7 Hz, 1H), 5.14 (s, 2H), 4.85-4.54 (m, 3H), 3.86-3.68 (m, 1H), 2.82-2.62 (m, 2H), 2.53-2.36 (m, 2H), 2.29 (d, J=6.7 Hz, 2H), 1.62 (hept, J=6.7 Hz, 1H), 0.84 (d, J=6.7 Hz, 6H); MS (ESI⁺) m/z 425, 427 [M+H]⁺.

### Example I-3G: tert-butyl {3-[3-amino-4-(benzyloxy)-6-bromo-2-fluorophenyl]-2-hydroxypropyl}(2-methylpropyl)carbamate

Di-tert-butyl dicarbonate (0.7 g, 3.21 mmol) was added to a solution of the product of Example I-3F (1.33 g, 2.97 mmol) in dichloromethane (15 mL). The reaction mixture was stirred at room temperature for 1 hour and then concentrated under reduced pressure to afford the title compound (1.57 g, 2.83 mmol, 95% yield). ¹H NMR (400 MHz, DMSO-d₆) δ ppm 7.55-7.23 (m, 5H), 6.97 (s, 1H), 5.13 (s, 2H), 4.94 (d, J=6.2 Hz, 1H), 4.79 (s, 2H), 3.88 (s, 1H), 3.31-3.19 (m, 1H), 3.19-2.56 (m, 5H), 1.85 (dt, J=13.6, 6.7 Hz, 1H), 1.30 (d, J=57.5 Hz, 9H), 0.80 (dd, J=23.0, 6.7 Hz, 6H); MS (ESI⁺) m/z 425, 427 [M-C(O)OC(CH₃)₃+H]⁺.

### Example I-3H: tert-butyl {[5-amino-6-(benzyloxy)-4-fluoro-2,3-dihydro-1-benzofuran-2-yl]methyl}(2-methylpropyl)carbamate

Cesium carbonate (850 mg, 2.61 mmol) and RockPhos Pd G3 (70 mg, 0.083 mmol) were added to a solution of the product of Example I-3G (500 mg, 0.856 mmol) in N,N-dimethylformamide (2 mL). The reaction mixture was purged with nitrogen for 15 minutes, stirred at 100° C. for 25 minutes, cooled to room temperature, and then diluted with ethyl acetate (25 mL), water (20 mL) and brine (15 mL). The aqueous layer was separated and extracted with ethyl acetate (2×20 mL). The organic layers were combined, washed with brine (3×20 mL), dried over magnesium sulfate, filtered, and concentrated under reduced pressure. The crude residue was subjected to column chromatography (SiO₂, dry loaded with silica, 0-20% ethyl acetate in isohexane) to afford the title compound (307 mg, 0.622 mmol, 73% yield). ¹H NMR (400 MHz, DMSO-d₆) δ ppm 7.54-7.21 (m, 5H), 6.37 (s, 1H), 5.09 (s, 2H), 4.92 (s, 1H), 4.18-3.94 (m, 2H), 3.47 (d, J=14.4 Hz, 1H), 3.26-2.91 (m, 4H), 2.81 (dd, J=15.3, 6.5 Hz, 1H), 1.94-1.79 (m, 1H), 1.38 (d, J=13.0 Hz, 9H), 0.81 (d, J=6.6 Hz, 6H); MS (ESI⁺) m/z 345 [M-C(O)OC(CH₃)₃+H]⁺.

### Example I-3I: tert-butyl {[6-(benzyloxy)-4-fluoro-5-(2,2,2-trifluoroacetamido)-2,3-dihydro-1-benzofuran-2-yl]methyl}(2-methylpropyl)carbamate

Trifluoroacetic anhydride (85 μL, 0.602 mmol) was added dropwise to a solution of pyridine (70 μL, 0.865 mmol) and the product of Example I-3H (287 mg, 0.581 mmol) in dichloromethane (1.5 mL) at room temperature. The reaction mixture was stirred at room temperature for 30 minutes, quenched with water (10 mL) and diluted with dichloromethane (10 mL). The aqueous layer was separated and extracted with dichloromethane (2×10 mL). The organic layers were combined, dried over magnesium sulfate, filtered, and concentrated under reduced pressure to afford the title compound (410 mg, 0.531 mmol, 91% yield, 70% purity). ¹H NMR (400 MHz, DMSO-d₆) δ ppm 7.47-7.23 (m, 6H), 6.57 (s, 1H), 5.19-5.00 (m, 3H), 3.48-3.21 (m, 3H), 3.18-2.80 (m, 3H), 1.96-1.76 (m, 1H), 1.39 (d, J=18.4 Hz, 9H), 0.82 (d, J=6.5 Hz, 6H); MS (ESI⁺) m/z 441 [M-C(O)OC(CH₃)₃+H]⁺.

### Example I-3J: methyl {[6-(benzyloxy)-2-{[(tert-butoxycarbonyl)(2-methylpropyl)amino]methyl}-4-fluoro-2,3-dihydro-1-benzofuran-5-yl](trifluoroacetyl)amino}acetate

Methyl 2-bromoacetate (95 μL, 1.000 mmol) was added to a solution of N,N-diisopropylethylamine (170 μL, 0.973 mmol) and the product of Example I-3I (377 mg, 0.488 mmol) in N,N-dimethylformamide (1 mL). The reaction mixture was stirred at 60° C. for 1 hour and then cooled to room temperature. Additional methyl 2-bromoacetate (95 μL, 1.000 mmol) was added, and the reaction mixture was stirred at 60° C. for 18 hours before cooling to room temperature. Additional N,N-diisopropylethylamine (170 μL, 0.973 mmol) and methyl 2-bromoacetate (95 μL, 1.000 mmol) were added, and the reaction mixture was stirred at 60° C. for 2 hours. The reaction mixture was cooled to room temperature and diluted with ethyl acetate (20 mL) and water (10 mL). The aqueous layer was separated and extracted with ethyl acetate (2×10 mL). The organic layers were combined, washed with brine (4×10 mL), dried over magnesium sulfate, filtered, and concentrated under reduced pressure. The crude residue was subjected to column chromatography (SiO₂, 0-20% ethyl acetate in isohexane) to afford the title compound (305 mg, 0.398 mmol, 82% yield, 80% purity). ¹H NMR (400 MHz, DMSO-d₆) δ ppm 7.44-7.26 (m, 5H), 6.64 (s, 1H), 5.21-5.02 (m, 3H), 4.52 (d, J=17.4 Hz, 1H), 4.13 (d, J=16.8 Hz, 1H), 3.61 (s, 3H), 3.53-3.36 (m, 2H), 3.28-3.17 (m, 1H), 3.14-2.80 (m, 3H), 1.94-1.80 (m, 1H), 1.36 (d, J=32.4 Hz, 9H), 0.90-0.71 (m, 6H); MS (ESI⁺) m/z 513 [M-C(O)OC(CH₃)₃+H]⁺.

### Example I-3K: methyl {[6-(benzyloxy)-2-{[(tert-butoxycarbonyl)(2-methylpropyl)amino]methyl}-4-fluoro-2,3-dihydro-1-benzofuran-5-yl]amino}acetate

Sodium methoxide (5.4 M in methanol) (80 μL, 0.431 mmol) was added to a solution of the product of Example I-3J (300 mg, 0.392 mmol) in methanol at room temperature. The reaction mixture was stirred at room temperature for 30 minutes and then stirred at 60° C. for 1 hour. Additional sodium methoxide (5.4 M in methanol) (80 μL, 0.431 mmol) was added, and the reaction mixture was stirred at 60° C. for 1 hour. The reaction mixture was cooled to room temperature and stirred for 18 hours. Additional sodium methoxide (5.4 M in methanol) (80 μL, 0.431 mmol) was added, and the reaction mixture was stirred at 60° C. for 1 hour. Additional sodium methoxide (5.4 M in methanol) (145 μL, 0.784 mmol) was added, and the reaction mixture was stirred at 60° C. for 2 hours. The reaction mixture was cooled to room temperature, quenched with a saturated aqueous solution of ammonium chloride (1.5 mL) and concentrated under reduced pressure to remove methanol. The aqueous suspension was diluted with ethyl acetate (10 mL) and water (5 mL). The aqueous layer was extracted with ethyl acetate (2×10 mL). The combined organic layers were dried over magnesium sulfate, filtered, and concentrated to afford the title compound (200 mg, 0.390 mmol, 100% yield). ¹H NMR (400 MHz, DMSO-d₆) δ ppm 7.51-7.26 (m, 5H), 6.42 (s, 1H), 5.10 (s, 2H), 4.95 (d, J=7.9 Hz, 1H), 4.51 (s, 1H), 3.91 (d, J=6.7 Hz, 2H), 3.60 (s, 3H), 3.52-3.43 (m, 1H), 3.39-3.24 (m, 1H), 3.23-2.92 (m, 3H), 2.82 (d, J=16.3 Hz, 1H), 1.93-1.81 (m, 1H), 1.38 (d, J=17.6 Hz, 9H), 0.81 (d, J=6.8 Hz, 6H); MS (ESI⁺) m/z 517 [M+H]⁺.

### Example I-3L: methyl {[6-(benzyloxy)-2-{[(tert-butoxycarbonyl)(2-methylpropyl)amino]methyl}-4-fluoro-2,3-dihydro-1-benzofuran-5-yl]({[(prop-2-en-1-yl)oxy]carbonyl}sulfamoyl)amino}acetate

To a solution of chlorosulfonyl isocyanate (0.09 mL, 1.037 mmol) in dichloromethane (1 mL) at 0° C. was added allyl alcohol (0.07 mL, 1.029 mmol) dropwise. The resulting solution was stirred for 20 minutes at 0° C. before the product of Example I-3K (0.240 g, 0.390 mmol) and triethylamine (0.2 mL, 1.435 mmol) in dichloromethane (1 mL) was added dropwise. The resulting mixture was allowed to stir at room temperature for 30 minutes. The reaction mixture was quenched with water (3 mL) and the layers were separated. The aqueous layer was extracted with dichloromethane (2×5 mL). The organic layers were combined, dried over magnesium sulfate, filtered, and concentrated under reduced pressure. The crude residue was subjected to column chromatography (SiO₂, 0-40% ethyl acetate in isohexane) to afford the title compound (170 mg, 0.213 mmol, 55% yield, 85% purity). ¹H NMR (400 MHz, DMSO-d₆) δ ppm 11.55 (s, 1H), 7.55-7.22 (m, 5H), 6.69-6.32 (m, 2H), 6.01-5.64 (m, 2H), 5.34-4.96 (m, 4H), 4.80 (d, J=17.9 Hz, 1H), 4.43 (dt, J=5.4, 1.6 Hz, 2H), 4.37-4.11 (m, 2H), 3.59 (s, 3H), 3.53-3.17 (m, 1H), 3.17-2.75 (m, 3H), 1.94-1.79 (m, 1H), 1.40 (s, 9H), 0.74-0.92 (m, 6H); MS (ESI⁻) m/z 678 [M−H]⁻.

### Example I-3M: tert-butyl {[6-(benzyloxy)-4-fluoro-5-(1,1,4-trioxo-1λ⁶,2,5-thiadiazolidin-2-yl)-2,3-dihydro-1-benzofuran-2-yl]methyl}(2-methylpropyl)carbamate ammonia

Potassium carbonate (35 mg, 0.253 mmol) was added to a suspension of tetrakis(triphenylphosphine)palladium(0) (11 mg, 9.52 μmol) and the product of Example I-3L (95 mg, 0.119 mmol) in methanol (1 mL). The mixture was stirred at 60° C. for 40 minutes. After cooling to room temperature, ammonium chloride (95 mg, 1.782 mmol) was added to the reaction mixture and the volatiles were removed in vacuo. The crude residue was subjected to column chromatography (Reveleris® reversed phase (C18) flash cartridge, dry loaded with diatomaceous earth, 5-70% methanol in 10 mM ammonium bicarbonate) to afford the title compound as an ammonium salt (41 mg, 0.067 mmol, 57% yield). ¹H NMR (400 MHz, DMSO-d₆) δ ppm 7.49 (d, J=7.2 Hz, 2H), 7.37-7.25 (m, 3H), 7.00 (s, 3H), 6.42 (s, 1H), 5.15-4.99 (m, 3H), 3.89 (s, 2H), 3.49 (d, J=14.4 Hz, 1H), 3.41-3.19 (m, 2H), 3.15-3.06 (m, 1H), 3.04-2.94 (m, 1H), 2.89-2.79 (m, 1H), 1.94-1.81 (m, 1H), 1.39 (d, J=18.1 Hz, 9H), 0.87-0.76 (m, 6H); MS (ESI⁻) m/z 562 [M−H]⁻.

### Example I-3N: tert-butyl {[4-fluoro-6-hydroxy-5-(1,1,4-trioxo-1λ⁶,2, 5-thiadiazolidin-2-yl)-2,3-dihydro-1-benzofuran-2-yl]methyl}(2-methylpropyl)carbamate ammonia

To a solution of the product of Example I-3M (40 mg, 0.067 mmol) in degassed water (0.5 mL) and ethanol (0.5 mL) was added 10% Pd/C (5 mg). The resulting suspension was allowed to stir under hydrogen (2 bar) for 1 hour. The reaction mixture was filtered through a diatomaceous earth pad, and the pad was then washed with ethanol (50 mL) and water (50 mL). The volatiles were removed under reduced pressure to afford the title compound as an ammonium salt (32 mg, 0.062 mmol, 92% yield). ¹H NMR (400 MHz, DMSO-d₆) δ ppm 9.14 (s, 1H), 7.09 (s, 3H), 6.12 (s, 1H), 5.03 (s, 1H), 3.87 (s, 2H), 3.51-3.25 (m, 2H), 3.19 (dd, J=15.1, 9.1 Hz, 1H), 3.06 (d, J=36.2 Hz, 2H), 2.80 (d, J=15.5 Hz, 1H), 1.95-1.83 (m, 1H), 1.40 (s, 9H), 0.82 (d, J=6.6 Hz, 6H); MS (ESI⁻) m/z 472 [M−H]⁻.

### Example I-3O: 5-(4-fluoro-6-hydroxy-2-{[(2-methylpropyl)amino]methyl}-2,3-dihydro-1-benzofuran-5-yl)-1λ⁶,2,5-thiadiazolidine-1,1,3-trione

Trifluoroacetic acid (25 μL, 0.324 mmol) was added to a solution of the product of Example I-3N (28 mg, 0.054 mmol) in dichloromethane (1 mL). The reaction mixture was stirred at room temperature for 2 hours. 0.7 M NH₃ in methanol in water (95:5) was added until the reaction mixture became basic. The solvent was removed under reduced pressure at room temperature. The crude residue was subjected to column chromatography (Reveleris® reversed phase (C18) flash cartridge, dry loaded with diatomaceous earth, 5-15% methanol in 10 mM ammonium bicarbonate) to afford the title compound (5.46 mg, 0.014 mmol, 26% yield). ¹H NMR (400 MHz, DMSO-d₆) δ ppm 9.26 (s, 1H), 8.48 (s, 1H), 6.21 (s, 1H), 5.22-5.08 (m, 1H), 3.88 (s, 2H), 3.38-3.21 (m, 3H), 2.94 (dd, J=15.3, 6.8 Hz, 1H), 2.88-2.75 (m, 2H), 1.97 (hept, J=6.8 Hz, 1H), 0.95 (dd, J=6.7, 5.3 Hz, 6H); MS (ESI⁺) m/z 374 [M+H]⁺.

### Example I-4: 5-[(2R)-2-{[(2-cyclopentylethyl)amino]methyl}-4-fluoro-6-hydroxy-2,3-dihydro-1-benzofuran-5-yl]-1λ⁶,2,5-thiadiazolidine-1,1,3-trione (Compound 103)

### Example I-4A: 1-(benzyloxy)-3,5-difluorobenzene

To a solution of 3,5-difluorophenol (24.1 g, 185 mmol) in N,N-dimethylformamide (370 mL) was added potassium carbonate (38.3 g, 277 mmol) and benzyl bromide (22.0 mL, 185 mmol), and the reaction mixture was stirred at room temperature. After 17 hours, water (800 mL) was added, and the mixture was extracted into heptane (400 mL). The organic extract was washed with saturated aqueous ammonium chloride (3×100 mL), dried over sodium sulfate, filtered, and concentrated to afford 43.8 g of a residue that was dissolved in heptane (20 mL) and purified by flash chromatography on silica gel [220 g SiO₂, isocratic heptanes for 2 column volumes then ramp to 11% methyl tert-butyl ether/heptanes to elute product, monitored/collected at 210 nm] to afford the title compound (32.1 g, 146 mmol, 79% yield). MS (APCI⁺) m/z 221 [M+H]⁺, but no ionization was observed.

### Example I-4B: (2R)-1-[4-(benzyloxy)-2,6-difluorophenyl]-3-[(2-methoxyethoxy)methoxy]propan-2-ol

Three reactions were set up and carried out in an identical manner, as follows. A solution of the product of Example I-4A (12.8 g, 58.3 mmol) in tetrahydrofuran (259 mL) was cooled to an internal temperature of −75° C. and a 1.9 M solution of phenyllithium in dibutyl ether (33.7 mL, 64.1 mmol) was added dropwise at a rate such that the internal temperature did not exceed −72° C. After stirring for 2 hours at −75° C., a solution of (2R)-2-{[(2-methoxyethoxy)methoxy]methyl}oxirane (9.45 g, 58.3 mmol) in tetrahydrofuran (65 mL) was added at a rate such that the internal temperature did not exceed −66° C. Thereafter, a solution of boron trifluoride diethyl etherate (11.1 mL, 87.0 mmol) in tetrahydrofuran (65 mL) was added at a rate such that the internal temperature did not exceed −66° C. Thereafter, the reaction mixture was stirred an additional 10 minutes, the cooling bath was removed, and the reaction mixture was allowed to warm to room temperature. After stirring for 1 hour at room temperature, the reaction was quenched via addition of saturated aqueous sodium bicarbonate (30 mL). The mixture was extracted with ethyl acetate (3×50 mL), and the combined organic extracts were washed with water (1×100 mL) and brine (1×100 mL), dried over sodium sulfate, and filtered. The filtrates from the 3 reactions were combined and concentrated. The crude residue was dissolved in dichloromethane (20 mL) and purified by flash chromatography on silica gel [220 g SiO₂, heptanes→25.6% methyl tert-butyl ether/heptanes over 15 minutes, hold 15 minutes, then ramp to 50% methyl tert-butyl ether/heptanes over 15 minutes, monitored/collected at 210 nm] to afford the title compound (18.9 g, 49.5 mmol, 28% yield). MS (APCI⁺) m/z 383 [M+H]⁺.

### Example I-4C: (2R)-6-(benzyloxy)-4-fluoro-2-{[(2-methoxyethoxy)methoxy]methyl}-2,3-dihydro-1-benzofuran

To a solution of the product of Example I-4B (18.93 g, 49.5 mmol) in tetrahydrofuran (330 mL) was added a 1 M solution of potassium tert-butoxide in tetrahydrofuran (49.5 mL, 49.5 mmol), and the reaction mixture was heated to an internal temperature of 60° C. After 75 minutes, the mixture was cooled to room temperature, quenched with saturated aqueous ammonium chloride (100 mL), stirred 5 minutes, and then extracted into ethyl acetate (2×100 mL). The combined organic extracts were washed with brine (1×200 mL), dried over sodium sulfate, filtered, and concentrated to afford 17.9 g of crude material that was dissolved in toluene (10 mL) and purified via flash chromatography [220 g SiO₂, heptanes→18.4% methyl tert-butyl ether/heptanes, monitored/collected at 210 nm] to afford the title compound (14.9 g, 40.1 mmol, 81% yield). MS (APCI⁺) m/z 363 [M+H]⁺.

### Example I-4D: (2R)-6-(benzyloxy)-5-bromo-4-fluoro-2-{[(2-methoxyethoxy)methoxy]methyl}-2,3-dihydro-1-benzofuran

To a solution of the product of Example I-4C (13.8 g, 38.2 mmol) in acetonitrile (255 mL) was added freshly recrystallized N-bromosuccinimide (6.80 g, 38.2 mmol), and the reaction mixture was stirred at room temperature. After 49 minutes, the mixture was directly concentrated, dissolved in toluene (25 mL), filtered to removed phthalimide, and immediately purified by flash chromatography on silica gel [220 g SiO₂, heptanes→31% methyl tert-butyl ether/heptanes, monitored/collected at 210 nm] to afford the title compound (15.6 g, 35.4 mmol, 93% yield). MS (APCI⁺) m/z 442 [M+H]⁺.

### Example I-4E: [(2R)-6-(benzyloxy)-5-bromo-4-fluoro-2,3-dihydro-1-benzofuran-2-yl]methanol

To the product of Example I-4D (9.61 g, 21.8 mmol) was added a 4 M solution of hydrochloric acid in dioxane (109 mL 436 mmol), and the reaction mixture was stirred at room temperature. After 51 minutes, a 0.5 M solution of zinc chloride in tetrahydrofuran (17.8 g zinc chloride, 131 mmol) was added. After 38 minutes, concentrated hydrochloric acid (18.2 mL, 218 mmol) was added. After 97 minutes, quantitative conversion was observed. The mixture was poured very slowly into saturated aqueous sodium bicarbonate (570 mL, 653 mmol), and after effervescence had ceased, the mixture was extracted into ethyl acetate (200 mL). The organic layer was dried over sodium sulfate, filtered, and concentrated. The crude material was dissolved in toluene (10 mL) and purified by flash chromatography on silica gel [120 g SiO₂, heptanes→45% methyl tert-butyl ether/heptanes, monitored/collected at 210 nm]. The 8.59 g of obtained material was contaminated with methoxymethyl-related impurities and was thus dissolved in toluene/dichloromethane (2:1, 15 mL) and again purified by flash chromatography on silica gel [120 g SiO₂, heptanes→8.7% acetone/heptanes, monitored/collected at 210 nm] to afford the title compound (4.95 g, 14.0 mmol, 64% yield). MS (APCI⁺) m/z 354 [M+H]⁺.

### Example I-4F: benzyl {[(2R)-6-(benzyloxy)-5-bromo-4-fluoro-2,3-dihydro-1-benzofuran-2-yl]methyl}carbamate

A solution of the product of Example I-4E (4.95 g, 14.0 mmol) and triethylamine (3.91 mL, 28.0 mmol) in anhydrous dichloromethane (93 mL) was cooled to an internal temperature of −5° C., and methanesulfonyl chloride (1.31 mL, 16.8 mmol) was added dropwise over 5 minutes. After 10 minutes, the reaction was quenched with saturated aqueous sodium bicarbonate (100 mL), the layers were separated, and the organic layer was dried over sodium sulfate, filtered, and concentrated to afford [(2R)-6-(benzyloxy)-5-bromo-4-fluoro-2,3-dihydro-1-benzofuran-2-yl]methyl methanesulfonate (6.01 g, 13.94 mmol, 99% yield) that was used in the subsequent step without further purification. To a solution of the crude intermediate in anhydrous N,N-dimethylformamide (70 mL) was added sodium azide (2.72 g, 41.8 mmol), and the reaction mixture was heated to an internal temperature of 80° C. After 144 minutes, the reaction mixture was cooled to room temperature and partitioned between water (2×300 mL) and ethyl acetate (200 mL). The organic fraction was washed with brine (1×200 mL), dried over sodium sulfate, filtered, and concentrated to afford (2R)-2-(azidomethyl)-6-(benzyloxy)-5-bromo-4-fluoro-2,3-dihydro-1-benzofuran (5.24 g, 13.86 mmol, 99% yield) that was used in the subsequent step without further purification. To a solution of the crude product in tetrahydrofuran (56 mL) and water (28 mL) was added triphenylphosphine (4.41 g, 16.8 mmol), and the reaction mixture was stirred at room temperature. After 65 minutes, the internal temperature was increased to 57° C. After 58 minutes, the internal temperature was increased to 61° C. After 22 minutes, quantitative conversion was achieved. The internal temperature was adjusted to 17° C. and water (28 mL) and 1 M aqueous sodium hydroxide (14.0 mL, 14.0 mmol) were added followed by benzyl chloroformate (2.06 mL, 14.4 mmol), and the reaction mixture was stirred at room temperature. After 10 minutes, the mixture was diluted with water (100 mL) and extracted into ethyl acetate (150 mL). The organic layer was washed with brine (1×100 mL), dried over sodium sulfate, filtered, and concentrated to afford 12.7 g of a residue that was dissolved in toluene (15 mL) and purified by flash chromatography on silica gel [120 g SiO₂, heptanes→18.5% acetone/heptanes over 25 minutes, monitored/collected at 210 nm] to afford the title compound (5.82 g, 12.0 mmol, 85% yield over four steps). MS (APCI⁺) m/z 487 [M+H]⁺.

### Example I-4G: tert-butyl {[(2R)-6-(benzyloxy)-2-({[(benzyloxy)carbonyl]amino}methyl)-4-fluoro-2,3-dihydro-1-benzofuran-5-yl]({[(prop-2-en-1-yl)oxy]carbonyl}sulfamoyl)amino}acetate

A stock solution of catalyst was prepared as follows: A 100 mL round-bottomed flask was charged with 2-methyl-2-butanol (36 mL), and the solvent was deoxygenated via sub-surface nitrogen sparging for 10 minutes. Thereafter, solid sodium tert-butoxide (27.5 mg, 0.289 mmol), tris(dibenzylideneacetone)dipalladium(0) (132 mg, 0.144 mmol), and RockPhos (150 mg, 0.318 mmol) was added, and the resulting mixture was heated to an internal temperature of 80° C. After 30 minutes, the homogeneous solution was cooled to room temperature. In a separate vessel, a mixture of the product of Example I-4F (5.34 g, 10.98 mmol), sodium trifluoroacetate (1.79 g, 13.2 mmol), tert-butyl-2-aminoacetate (1.73 g, 13.2 mmol) and 1,5,7-triazabicyclo[4.4.0]dec-5-ene (TBD, 1.83 g, 13.2 mmol) in 2-methyl-2-butanol (41 mL) was deoxygenated via sub-surface nitrogen sparging for 20 minutes. Subsequently, 13.7 mL of the catalyst stock solution prepared above was added via cannula, the solution was further deoxygenated for 15 minutes, and the reaction mixture was heated to an internal temperature of 70° C. An additional charge of catalyst stock solution (13.7 mL) was added after 3 hours. After 12 hours of total reaction time, proton NMR indicated quantitative conversion. Thereafter, the mixture was cooled to room temperature and partitioned between ethyl acetate (150 mL) and 0.5 M aqueous hydrochloric acid (200 mL). The organic extract was washed with brine (1×80 mL), dried over sodium sulfate, and filtered. Toluene (70 mL) was added to the filtrate. Due to the acute sensitivity of the product towards air oxidation in concentrated form, the flask containing the diluted product was attached to a rotary evaporator, and the apparatus was evacuated and backfilled with nitrogen three times before concentrating in the presence of nitrogen. Upon concentration, the rotary evaporator was backfilled with nitrogen, the product-containing flask was quickly removed and placed on a Schlenk line under nitrogen to afford 10.43 g of tert-butyl {[(2R)-6-(benzyloxy)-2-({[(benzyloxy)carbonyl]amino}methyl)-4-fluoro-2,3-dihydro-1-benzofuran-5-yl]amino}acetate partially dissolved in toluene. The crude material was used in the subsequent step without purification.

To a solution of chlorosulfonyl isocyanate (1.39 mL, 16.0 mmol) in dichloromethane (336 mL) at an internal temperature of −10° C. was added allyl alcohol (1.09 mL, 16.0 mmol) at a rate such that the internal temperature did not exceed 0° C. After 30 minutes, a preformed solution of the crude tert-butyl {[(2R)-6-(benzyloxy)-2-({[(benzyloxy)carbonyl]amino}methyl)-4-fluoro-2,3-dihydro-1-benzofuran-5-yl]amino}acetate and N,N-diisopropylethylamine (3.72 mL, 21.3 mmol) in dichloromethane (17.7 mL) was added at a rate such that the internal temperature did not exceed 0° C. After 15 minutes, the reaction was quenched with water (50 mL), and the mixture was stirred for 5 minutes. Then the layers were separated. The organic layer was dried over sodium sulfate, filtered, and concentrated. The residue was dissolved in dichloromethane/toluene (1:1, 10 mL) and purified by flash chromatography on silica gel [120 g SiO₂, heptanes→30% acetone/heptanes, monitored/collected at 210 nm] to afford the title compound (5.13 g, 7.33 mmol, 69% yield over two steps). MS (APCI⁺) m/z 700 [M+H]⁺.

### Example I-4H: benzyl {[(2R)-6-(benzyloxy)-4-fluoro-5-(1,1,4-trioxo-1λ⁶,2,5-thiadiazolidin-2-yl)-2,3-dihydro-1-benzofuran-2-yl]methyl}carbamate

To a solution of the product of Example I-4G (2.67 g, 3.82 mmol) in anhydrous methanol (21.2 mL) was added a solution of sodium tert-butoxide (5.72 mL, 11.5 mmol, 2 M in tetrahydrofuran), and the solution was deoxygenated via sub-surface nitrogen sparging for 15 minutes. Thereafter, tetrakis(triphenylphosphine)palladium(0) (22.0 mg, 0.0190 mmol) was added, and the reaction mixture was heated to an internal temperature of 50° C. After 3 hours, the mixture was cooled to an internal temperature of −9° C., quenched by adding a solution of hydrochloric acid (4.07 mL, 12.2 mmol, 3 M in cyclopentyl methyl ether (CPME)) and stirred for 5 minutes, after which water (30 mL) was added. After stirring for 5 minutes, the mixture was partitioned between ethyl acetate (120 mL) and water (50 mL). The organic extract was washed with brine (1×50 mL), dried over sodium sulfate, filtered, and concentrated. The residue (4.19 g) was dissolved in a mixture of methanol/N,N-dimethylformamide/water (1:1:1, 15 mL), filtered through a glass microfiber frit, and purified by HPLC [Waters XBridge™ BEH C18 OBD Prep Column, 130A, 5 μm, 30 mm×100 mm, flow rate 40 mL/minute, 5-100% gradient (5 →35% over 10 minutes) of acetonitrile in buffer (0.025 M aqueous ammonium bicarbonate, adjusted to pH 10 with ammonium hydroxide)]. The fractions were concentrated to afford 2.22 g of the ammonium salt of the title compound. The material was suspended in ethyl acetate (70 mL) and washed with 6 M HCl (50 mL). The organic phase was dried over sodium sulfate, filtered, and concentrated to afford the title compound (1.98 g, 3.60 mmol, 48.2% yield). MS (APCI⁺) m/z 559 [M+H]⁺.

### Example I-4I: 5-[(2R)-2-(aminomethyl)-4-fluoro-6-hydroxy-2,3-dihydro-1-benzofuran-5-yl]-1λ⁶,2,5-thiadiazolidine-1,1,3-trione hydrogen chloride (1/1)

A suspension of the product of Example I-4H (1.66 g, 3.07 mmol) and 5% palladium on alumina (0.371 g, 0.174 mmol [Pd]) in tetrahydrofuran (46 mL) and water (15.3 mL) was pressurized with hydrogen gas to 50 psi and stirred at room temperature. After 16 hours, 1 M sodium hydroxide (3.07 mL, 3.07 mmol) was added, the solution was stirred for 1 hour, additional water/tetrahydrofuran (1:3, 20 mL) was added, stirred for 15 minutes, then filtered through a 0.45 μM Whatman PTFE filter, and the catalyst bed was rinsed with additional water/tetrahydrofuran (1:3, 20 mL). The mixture was concentrated to afford 1.31 g of a residue that was suspended in a solution of hydrochloric acid (3 M in cyclopentyl methyl ether, 20.4 mL, 61.3 mmol) and stirred at room temperature for 10 minutes. The resulting precipitate was isolated on a fritted funnel, washed with cyclopentyl methyl ether (2×20 mL) and dried to constant mass to afford the title compound (1.05 g, 2.89 mmol, 94% yield). ¹H NMR (500 MHz, DMSO-d₆) δ ppm 10.4 (br s, 1H), 8.31 (t, J=6.0 Hz, 3H), 6.33 (s, 1H), 5.12 (m, 1H), 4.28 (s, 2H), 3.31 (dd, J=15.4, 9.4 Hz, 1H), 3.15 (m, 1H), 3.07 (m, 1H), 2.98 (dd, J=15.4, 6.8 Hz, 1H); MS (APCI⁻) m/z 353 [M]⁻.

### Example I-4J: 5-[(2R)-2-{[(2-cyclopentylethyl)amino]methyl}-4-fluoro-6-hydroxy-2,3-dihydro-1-benzofuran-5-yl]-1λ⁶,2,5-thiadiazolidine-1,1,3-trione

In a 4 mL vial was added the product of Example I-4I (25 mg, 0.074 mmol) and triethylamine (31 μL, 0.22 mmol, 3 equivalents) in 3:2 v/v ethanol/dichloromethane (0.75 mL). 2-Cyclopentylacetaldehyde (16.5 mg, 0.15 mmol, 2.0 equivalents) was added, and the reaction mixture was stirred at room temperature for 2 hours. Sodium borohydride (11.2 mg, 0.30 mmol, 4.0 equivalents) was added in one portion and the mixture was stirred for 30 minutes at room temperature. The reaction mixture was cooled to 0° C. in an ice bath, and aqueous hydrochloric acid (2.0 M, 0.74 mL, 1.47 mmol, 20 equivalents) was slowly added, and the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A), to afford the title compound (11.9 mg, 39.1% yield). ¹H NMR (500 MHz, DMSO-d₆) δ ppm 9.26 (s, 1H), 8.56 (s, 2H), 6.20 (s, 1H), 5.16-5.07 (m, 1H), 3.88 (s, 2H), 3.37-3.24 (m, 3H), 2.99-2.89 (m, 3H), 1.84-1.69 (m, 3H), 1.67-1.43 (m, 6H), 1.14-1.03 (m, 2H); MS (APCI⁺) m/z 414.3 [M+H]⁺.

### Example I-5: 5-[(2R)-4-fluoro-6-hydroxy-2-{[(4-hydroxy-3,3-dimethylbutyl)amino]methyl}-2,3-dihydro-1-benzofuran-5-yl]-1λ⁶,2,5-thiadiazolidine-1,1,3-trione (Compound 104)

### Example I-5A: methyl 2,2-dimethylpent-4-enoate

The title compound was synthesized from methyl isobutyrate using the procedure described for Example I-23A in 71.8% yield. ¹H NMR (400 MHz CDCl₃) δ ppm 5.57-5.89 (m, 1H), 4.91-5.16 (m, 2H), 3.56 (s, 3H), 2.27 (m, 2H), 1.17 (s, 6H).

### Example I-5B: 2,2-dimethylpent-4-en-1-ol

The title compound was synthesized from Example I-5A in the same way as described for Example I-23B in 95% yield. ¹H NMR (400 MHz, CDCl₃) δ ppm 5.65-5.93 (m, 1H), 4.89-5.11 (m, 2H), 3.19-3.36 (m, 2H), 3.28 (s, 2H), 1.95 (d, J=7.63 Hz, 2H), 0.82 (s, 6H).

### Example I-5C: tert-butyl[(2,2-dimethylpent-4-en-1-yl)oxy]dimethylsilane

The title compound was synthesized from Example I-5B in a same way as described for Example I-23C in 74% yield. ¹H NMR (400 MHz, CDCl₃) δ ppm 5.63-6.07 (m, 1H), 4.86-5.21 (m, 2H), 3.21 (s, 2H), 2.00 (d, J=7.50 Hz, 2H), 0.91 (s, 9H), 0.84 (s, 6H), 0.01 (s, 6H).

### Example I-5D: 4-{[tert-butyl(dimethyl)silyl]oxy}-3,3-dimethylbutanal

The title compound was synthesized from Example I-5C in a same way as described for Example I-23D in 66% yield. ¹H NMR (400 MHz, CDCl₃) δ ppm 9.84 (t, J=3.q Hz, 1H) 3.35 (s, 2H) 2.28 (d, J=3.1 Hz, 2H) 1.02 (s, 6H) 0.89 (s, 9H) 0.04 (s, 6H); MS (ESI⁺) m/z 231 [M+H]⁺.

### Example I-5E: 5-[(2R)-4-fluoro-6-hydroxy-2-{[(4-hydroxy-3,3-dimethylbutyl)amino]methyl}-2,3-dihydro-1-benzofuran-5-yl]-1λ⁶,2,5-thiadiazolidine-1,1,3-trione

In a 4 mL vial was added the product of Example I-4I (50 mg, 0.14 mmol) and triethylamine (59 μL, 0.42 mmol, 3 equivalents) in 3:2 v/v ethanol/dichloromethane (1.4 mL). The product of Example I-5D (65.4 mg, 0.42 mmol, 2.0 equivalents) was added, and the reaction mixture was stirred at room temperature for 2 hours. Sodium borohydride (21.4 mg, 0.57 mmol, 4.0 equivalents) was added in one portion and the resultant mixture was stirred for 30 minutes at ambient temperature. The reaction mixture was cooled to 0° C. in an ice bath, aqueous hydrochloric acid (2.0 M, 1.4 mL, 2.8 mmol, 20 equivalents) was slowly added, and the reaction mixture was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (21.7 mg, 36.8% yield). ¹H NMR (500 MHz, DMSO-d₆) δ ppm 6.17 (s, 1H), 5.09-5.00 (m, 1H), 3.88 (s, 2H), 3.28 (dd, J=15.1, 9.2 Hz, 1H), 3.14-3.08 (m, 4H), 2.95-2.81 (m, 3H), 1.48 (tt, J=9.8, 4.9 Hz, 2H), 0.82 (s, 6H); MS (APCI⁺) m/z 418.3 [M+H]⁺.

### Example I-6: 5-[(2R)-4-fluoro-6-hydroxy-2-{[(3-hydroxy-3-methylbutyl)amino]methyl}-2,3-dihydro-1-benzofuran-5-yl]-1λ⁶,2,5-thiadiazolidine-1,1,3-trione (Compound 105)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). 3-Hydroxy-3-methylbutanal (43.3 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the reaction mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (7.9 mg, 13.8% yield). ¹H NMR (400 MHz, DMSO-d₆) δ ppm 6.18 (s, 1H), 5.08 (dq, J=9.5, 6.4 Hz, 1H), 4.08 (d, J=5.3 Hz, 1H), 3.88 (s, 2H), 3.18 (dd, J=11.9, 4.8 Hz, 3H), 3.06-2.87 (m, 4H), 1.67 (td, J=7.2, 2.4 Hz, 2H), 1.13 (s, 6H); MS (APCI⁺) m/z 404.3 [M+H]⁺.

### Example I-7: 5-[(2R)-2-({[2-(3,3-difluorocyclobutyl)ethyl]amino}methyl)-4-fluoro-6-hydroxy-2,3-dihydro-1-benzofuran-5-yl]-1λ⁶,2,5-thiadiazolidine-1,1,3-trione (Compound 106)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). 2-(3,3-Difluorocyclobutyl)acetaldehyde (56.9 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (7.2 mg, 11.6% yield). ¹H NMR (400 MHz, DMSO-d₆) δ ppm 9.26 (s, 1H), 8.82-8.37 (m, 2H), 6.21 (s, 1H), 5.11 (dq, J=9.4, 6.3 Hz, 1H), 3.88 (s, 2H), 3.35 (d, J=9.3 Hz, 1H), 3.26 (d, J=6.1 Hz, 2H), 2.92 (dt, J=15.8, 6.9 Hz, 3H), 2.77-2.61 (m, 2H), 2.32-2.05 (m, 3H), 1.81 (q, J=7.8 Hz, 2H); MS (APCI⁺) m/z 436.3 [M+H]⁺.

### Example I-8: 5-[(2R)-2-{[(4,4-difluorobutyl)amino]methyl}-4-fluoro-6-hydroxy-2,3-dihydro-1-benzofuran-5-yl]-1λ⁶,2,5-thiadiazolidine-1,1,3-trione (Compound 107)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). 4,4-Difluorobutanal (45.8 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (10.8 mg, 18.7% yield). ¹H NMR (500 MHz, DMSO-d₆) δ ppm 9.28 (s, 1H), 8.72-8.58 (m, 2H), 6.26-5.95 (m, 2H), 5.12 (dq, J=9.3, 6.4 Hz, 1H), 3.88 (s, 2H), 3.33-3.26 (m, 3H), 3.02 (t, J=7.9 Hz, 2H), 2.94 (dd, J=15.3, 6.8 Hz, 1H), 1.98-1.81 (m, 2H), 1.81-1.67 (m, 2H); MS (APCI⁺) m/z 410.3 [M+H]⁺.

### Example I-9: 5-[(2R)-4-fluoro-6-hydroxy-2-({[2-(oxolan-3-yl)ethyl]amino}methyl)-2,3-dihydro-1-benzofuran-5-yl]-1λ⁶,2,5-thiadiazolidine-1,1,3-trione (Compound 108)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). 2-(Tetrahydrofuran-3-yl)acetaldehyde (48.4 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (8.8 mg, 15% yield). ¹H NMR (400 MHz, DMSO-d₆) δ ppm 9.26 (s, 1H), 8.85-7.98 (m, 2H), 6.20 (s, 1H), 5.11 (dq, J=9.4, 6.4 Hz, 1H), 3.88 (s, 2H), 3.82-3.67 (m, 2H), 3.67-3.56 (m, 1H), 3.29-3.21 (m, 4H), 2.95 (h, J=7.0, 6.2 Hz, 3H), 2.15 (h, J=7.1 Hz, 1H), 2.06-1.93 (m, 1H), 1.75-1.56 (m, 2H), 1.53-1.39 (m, 1H); MS (APCI⁺) m/z 416.3 [M+H]⁺.

### Example I-10: 5-{(2R)-4-fluoro-6-hydroxy-2-[(propylamino)methyl]-2,3-dihydro-1-benzofuran-5-yl}-1λ⁶,2,5-thiadiazolidine-1,1,3-trione (Compound 109)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Propionaldehyde (24.6 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (2.4 mg, 4.8% yield). ¹H NMR (400 MHz, DMSO-d₆) δ ppm 6.20 (s, 1H), 5.16-5.04 (m, 1H), 3.88 (s, 2H), 3.25-3.14 (m, 3H), 2.98-2.84 (m, 3H), 1.60 (qt, J=8.1, 4.0 Hz, 2H), 0.95-0.83 (m, 3H); MS (APCI⁺) m/z 360.3 [M+H]⁺.

### Example I-11: 5-{(2R)-2-[(dipropylamino)methyl]-4-fluoro-6-hydroxy-2,3-dihydro-1-benzofuran-5-yl}-1λ⁶,2,5-thiadiazolidine-1,1,3-trione (Compound 110)

Example I-11 was isolated as a by-product in the synthesis of Example I-10 (5.9 mg, 10.4% yield). ¹H NMR (400 MHz, DMSO-d₆) δ ppm 9.38-9.11 (m, 2H), 6.19 (s, 1H), 5.29 (s, 1H), 3.88 (s, 2H), 3.54-3.34 (m, 4H), 3.11 (s, 3H), 2.89 (dd, J=15.3, 6.6 Hz, 1H), 1.64 (s, 4H), 0.90 (t, J=7.5 Hz, 6H); MS (APCI⁺) m/z 402.3 [M+H]⁺.

### Example I-12: 5-{(2R)-2-[(butylamino)methyl]-4-fluoro-6-hydroxy-2,3-dihydro-1-benzofuran-5-yl}-1λ⁶,2,5-thiadiazolidine-1,1,3-trione (Compound 111)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Butyraldehyde (30.6 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (3.8 mg, 7.3% yield). ¹H NMR (400 MHz, DMSO-d₆) δ ppm 6.20 (s, 1H), 5.11 (dq, J=8.7, 6.3 Hz, 1H), 3.88 (s, 2H), 3.24 (d, J=6.2 Hz, 3H), 2.98-2.88 (m, 3H), 1.64-1.51 (m, 2H), 1.33 (h, J=7.4 Hz, 2H), 0.90 (t, J=7.4 Hz, 3H); MS (APCI⁺) m/z 374.3 [M+H]⁺.

### Example I-13: 5-{(2R)-2-[(ethylamino)methyl]-4-fluoro-6-hydroxy-2,3-dihydro-1-benzofuran-5-yl}-1λ⁶,2,5-thiadiazolidine-1,1,3-trione (Compound 112)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Acetaldehyde (10.3 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (10.3 mg, 21.1% yield). ¹H NMR (600 MHz, DMSO-d₆) δ ppm 9.25 (s, 1H), 8.67-8.45 (m, 2H), 6.20 (s, 1H), 5.14-5.06 (m, 1H), 3.88 (s, 2H), 3.37-3.28 (m, 1H), 3.26 (d, J=6.2 Hz, 2H), 3.00 (q, J=7.3 Hz, 2H), 2.93 (dd, J=15.3, 6.8 Hz, 1H), 1.19 (t, J=7.2 Hz, 3H); MS (APCI⁺) m/z 346.3 [M+H]⁺.

### Example I-14: 5-[(2R)-2-{[(cyclopentylmethyl)amino]methyl}-4-fluoro-6-hydroxy-2,3-dihydro-1-benzofuran-5-yl]-1λ⁶,2,5-thiadiazolidine-1,1,3-trione (Compound 113)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Cyclopentanecarbaldehyde (41.6 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (17.0 mg, 30.2% yield). ¹H NMR (400 MHz, DMSO-d₆) δ ppm 9.27 (s, 1H), 8.55 (s, 2H), 6.21 (s, 1H), 5.22-5.10 (m, 1H), 3.88 (s, 2H), 3.38-3.24 (m, 3H), 3.02-2.88 (m, 3H), 2.17 (hept, J=7.7 Hz, 1H), 1.84-1.72 (m, 2H), 1.67-1.45 (m, 4H), 1.30-1.14 (m, 2H); MS (APCI⁺) m/z 400.3 [M+H]⁺.

### Example I-15: 5-[(2R)-4-fluoro-6-hydroxy-2-({[(oxan-4-yl)methyl]amino}methyl)-2,3-dihydro-1-benzofuran-5-yl]-1λ⁶,2,5-thiadiazolidine-1,1,3-trione (Compound 114)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Tetrahydro-2H-pyran-4-carbaldehyde (48.4 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (17.6 mg, 30% yield). ¹H NMR (400 MHz, DMSO-d₆) δ ppm 6.18 (s, 1H), 5.07 (dt, J=13.3, 6.2 Hz, 1H), 3.91-3.80 (m, 4H), 3.33-3.22 (m, 3H), 3.12 (d, J=6.1 Hz, 2H), 2.92 (dd, J=15.2, 6.9 Hz, 1H), 2.76 (d, J=6.9 Hz, 2H), 1.92-1.78 (m, 1H), 1.69-1.57 (m, 2H), 1.26-1.12 (m, 2H); MS (APCI⁺) m/z 416.3 [M+H]7.

### Example I-16: 5-[(2R)-4-fluoro-6-hydroxy-2-{[(3-methylbutyl)amino]methyl}-2,3-dihydro-1-benzofuran-5-yl]-1λ⁶,2,5-thiadiazolidine-1,1,3-trione (Compound 115)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Isovaleraldehyde (36.5 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (7.2 mg, 13.1% yield). ¹H NMR (400 MHz, DMSO-d₆) δ ppm 6.20 (s, 1H), 5.17-5.05 (m, 1H), 3.88 (s, 2H), 3.37-3.33 (m, 1H), 3.25 (d, J=6.2 Hz, 2H), 2.99-2.88 (m, 3H), 1.61 (dh, J=12.9, 6.7 Hz, 1H), 1.49 (ddd, J=14.0, 6.7, 2.2 Hz, 2H), 0.89 (d, J=6.6 Hz, 6H); MS (APCI⁺) m/z 388.3 [M+H]⁺.

### Example I-17: 5-[(2R)-4-fluoro-6-hydroxy-2-{[(2-methylbutyl)amino]methyl}-2,3-dihydro-1-benzofuran-5-yl]-1λ⁶,2,5-thiadiazolidine-1,1,3-trione (Compound 116)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). 2-Methylbutanal (36.5 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (10.8 mg, 19.7% yield). ¹H NMR (600 MHz, DMSO-d₆) δ ppm 9.26 (s, 1H), 8.71-8.36 (m, 2H), 6.21 (s, 1H), 5.21-5.13 (m, 1H), 3.88 (s, 2H), 3.29-3.25 (m, 3H), 2.99-2.89 (m, 2H), 2.83-2.73 (m, 1H), 1.79-1.74 (m, 1H), 1.49-1.35 (m, 1H), 1.23-1.12 (m, 1H), 0.94 (t, J=6.5 Hz, 3H), 0.87 (td, J=7.4, 1.3 Hz, 3H); MS (APCI⁺) m/z 388.3 [M+H]⁺.

### Example I-18: 5-[(2R)-2-{[(cyclopropylmethyl)amino]methyl}-4-fluoro-6-hydroxy-2,3-dihydro-1-benzofuran-5-yl]-1λ⁶,2,5-thiadiazolidine-1,1,3-trione (Compound 117)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Cyclopropanecarbaldehyde (29.7 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (11.1 mg, 21.1% yield). ¹H NMR (400 MHz, DMSO-d₆) δ ppm 9.25 (s, 1H), 8.61 (s, 1H), 6.21 (s, 1H), 5.15-5.11 (m, 1H), 3.88 (s, 2H), 3.30-3.24 (m, 4H), 3.00-2.77 (m, 3H), 1.10-0.99 (m, 1H), 0.62-0.53 (m, 2H), 0.39-0.31 (m, 2H); MS (APCI⁺) m/z 372.3 [M+H]⁺.

### Example I-19: 5-[(2R)-2-{[bis(cyclopropylmethyl)amino]methyl}-4-fluoro-6-hydroxy-2,3-dihydro-1-benzofuran-5-yl]-1λ⁶,2,5-thiadiazolidine-1,1,3-trione (Compound 118)

Example I-19 was isolated as a by-product in the synthesis of Example I-18 (11.5 mg, 19.1% yield). ¹H NMR (400 MHz, DMSO-d₆) δ ppm 6.18 (s, 1H), 4.08 (q, J=5.3 Hz, 1H), 3.88 (s, 2H), 3.70-3.44 (m, 2H), 3.24-3.09 (m, 4H), 2.96 (dd, J=15.2, 6.8 Hz, 2H), 1.30-0.85 (m, 2H), 0.83-0.12 (m, 8H); MS (APCI⁺) m/z 426.4 [M+H]⁺.

### Example I-20: 5-[(2R)-2-{[(cyclobutylmethyl)amino]methyl}-4-fluoro-6-hydroxy-2,3-dihydro-1-benzofuran-5-yl]-1λ⁶,2,5-thiadiazolidine-1,1,3-trione (Compound 119)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Cyclobutanecarbaldehyde (35.7 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (10.1 mg, 18.6% yield). ¹H NMR (400 MHz, DMSO-d₆) δ ppm 9.23 (s, 1H), 8.49-7.91 (m, 2H), 6.19 (s, 1H), 5.16-5.06 (m, 1H), 3.88 (s, 2H), 3.30-3.26 (m, 1H), 3.18 (dd, J=8.4, 5.6 Hz, 2H), 3.02-2.87 (m, 3H), 2.66-2.54 (m, 1H), 2.10-2.00 (m, 2H), 1.93-1.71 (m, 4H); MS (APCI⁺) m/z 386.3 [M+H]⁺.

### Example I-21: 5-[(2R)-4-fluoro-6-hydroxy-2-({[(oxetan-3-yl)methyl]amino}methyl)-2,3-dihydro-1-benzofuran-5-yl]-1λ⁶,2,5-thiadiazolidine-1,1,3-trione (Compound 120)

In a 4 mL vial was added the product of Example I-4I (50 mg, 0.14 mmol) and triethylamine (57 μL, 0.41 mmol, 3 equivalents) in 3:2 v/v ethanol/dichloromethane (1.4 mL). Oxetane-3-carbaldehyde (36.5 mg, 0.42 mmol, 3.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (13.1 mg, 24% yield). ¹H NMR (600 MHz, DMSO-d₆) δ ppm 9.27 (s, 1H), 8.76-8.45 (m, 2H), 6.20 (s, 1H), 5.10 (dtd, J=9.4, 6.9, 5.3 Hz, 1H), 4.68-4.61 (m, 2H), 4.38-4.33 (m, 2H), 3.88 (s, 2H), 3.36-3.27 (m, 4H), 3.27-3.23 (m, 2H), 2.93 (dd, J=15.2, 6.8 Hz, 1H); MS (APCI⁺) m/z 388.3 [M+H]⁺.

### Example I-22: 5-{(2R)-4-fluoro-6-hydroxy-2-[({2-[1-(hydroxymethyl)cyclobutyl]ethyl}amino)methyl]-2,3-dihydro-1-benzofuran-5-yl}-1λ⁶,2,5-thiadiazolidine-1,1,3-trione (Compound 121)

### Example I-22A: tert-butyl(dimethyl){[1-(prop-2-en-1-yl)cyclobutyl]methoxy}silane

To a solution of (1-allylcyclobutyl)methanol (prepared according to Bioorganic and Medicinal Chemistry, 2002, 10 (4), 1093-1106) (2.5 g, 15.85 mmol, 80% purity) in anhydrous tetrahydrofuran (70 mL) was added imidazole (2.158 g, 31.7 mmol) and then tert-butyldimethylchlorosilane (3.58 g, 23.77 mmol) in portions at 0° C. The reaction mixture was stirred at 20° C. for 3 hours. One additional reaction on 500 mg scale was set up and run as described above. These two reaction mixtures were combined and extracted with water (200 mL). The organic phase was separated, and the aqueous phase was extracted with ethyl acetate (60 mL). The combined organic phases were washed with brine (100 mL), dried over anhydrous sodium sulfate, and concentrated under reduced pressure. The residue was then dissolved with petroleum ether and filtered through silica gel, and the filter cake was washed with petroleum ether (1500 mL). The filtrate was concentrated under reduced pressure to give the title compound (4 g, 90% purity, 86% yield). ¹H NMR (400 MHz, CDCl₃) δ ppm 5.79 (ddt, J=17.07, 10.07, 7.32 Hz, 1H), 4.96-5.10 (m, 2H), 3.44 (s, 2H), 2.21 (d, J=7.25 Hz, 2H), 1.63-1.92 (m, 6H), 0.92 (s, 9H), −0.05 (s, 6H).

### Example I-22B: [1-({[tert-butyl(dimethyl)silyl]oxy}methyl)cyclobutyl]acetaldehyde

To a solution of the product of Example I-22A (3 g, 11.23 mmol, purity 90%) in dioxane (120 mL) and water (12 mL) was added a 0.2 M solution osmium tetroxide in t-butanol (220 mg, 0.865 mmol) dropwise at 20° C. After 15 minutes, the reaction mixture was cooled to 0° C. before sodium periodate (9.61 g, 44.9 mmol) was added in portions. After addition, the mixture was warmed up to 20° C. and stirred at that temperature for 3 hours. The mixture was diluted with ethyl acetate (200 mL) and filtered. The filtrate was added to saturated sodium thiosulfate aqueous solution (300 mL) and the resulting mixture was stirred at 20° C. for 1 hour. The mixture was transferred to a separatory funnel and the organic phase was separated, washed with brine (500 mL), dried over anhydrous sodium sulfate and concentrated under reduced pressure to give the title compound (3 g, purity 70%, yield 77%) which was used for the next step without further purification. ¹H NMR (400 MHz, CDCl₃) δ ppm 9.63 (t, J=2.75 Hz, 1H), 3.56 (s, 2H), 2.46 (d, J=2.63 Hz, 2H), 1.82-1.90 (m, 6H), 0.89 (s, 9H), 0.02 (s, 6H).

### Example I-22C: 5-{(2R)-4-fluoro-6-hydroxy-2-[({2-[1-(hydroxymethyl)cyclobutyl]ethyl}amino)methyl]-2,3-dihydro-1-benzofuran-5-yl}-1λ⁶,2,5-thiadiazolidine-1,1,3-trione

In a 4 mL vial was added the product of Example I-4I (50 mg, 0.14 mmol) and triethylamine (59 μL, 0.42 mmol, 3 equivalents) in 3:2 v/v ethanol/dichloromethane (1.4 mL). The product of Example I-22B (68.5 mg, 0.42 mmol, 2.0 equivalents) was added, and the reaction mixture was stirred at ambient temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, aqueous hydrochloric acid (2.0 M, 1.4 mL, 2.8 mmol, 20 equivalents) was slowly added, and the reaction was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-40% A, 8.0-8.1 minutes 40-100% A, 8.1-9.0 minutes 100% A, 9.0-9.1 minutes linear gradient 100-5% A, 9.1-10.0 minutes 5% A), to afford the title compound (17.2 mg, 28.3% yield). ¹H NMR (600 MHz, DMSO-d₆) δ ppm 9.26 (s, 1H), 8.80-8.35 (m, 2H), 6.20 (s, 1H), 5.15-5.07 (m, 1H), 3.88 (s, 2H), 3.34 (s, 2H), 3.28 (dd, J=9.1, 4.4 Hz, 3H), 2.97-2.89 (m, 3H), 1.87-1.73 (m, 6H), 1.72-1.64 (m, 2H); MS (APCI⁺) m/z 430.4 [M+H]⁺.

### Example I-23: 5-{(2R)-4-fluoro-6-hydroxy-2-[({2-[1-(hydroxymethyl)cyclopentyl]ethyl}amino)methyl]-2,3-dihydro-1-benzofuran-5-yl}-1λ⁶,2,5-thiadiazolidine-1,1,3-trione (Compound 122)

### Example I-23A: methyl 1-allylcyclopentanecarboxylate

To a solution of lithium diisopropylamide (122 mL, 243 mmol) in anhydrous tetrahydrofuran (720 mL) was added methyl cyclopentanecarboxylate (24 g, 187 mmol) dropwise at −65° C. under nitrogen. The reaction mixture was stirred at −65° C. under nitrogen for 40 minutes before 3-bromoprop-1-ene (29.4 g, 243 mmol) was added dropwise. The mixture was then allowed to warm up to at 20° C. and stirred at 20° C. for 16 hours before it was quenched by adding 500 mL of saturated aqueous NH₄Cl solution dropwise at 0° C. The resulting mixture was extracted with ethyl acetate (3×150 mL). The combined organic phases were washed with brine (500 mL), dried over anhydrous sodium sulfate, filtered, and concentrated. The crude product was chromatographed on silica gel eluting with petroleum ether to give the title compound (31.2 g, 167 mmol, yield 89%). ¹H NMR (400 MHz, CDCl₃) δ ppm 5.76-5.64 (m, 1H), 5.06-4.98 (m, 2H), 3.67 (s, 3H), 2.34 (d, J=7.3 Hz, 2H), 2.11-2.02 (m, 2H), 1.69-1.58 (m, 4H), 1.58-1.46 (m, 2H).

### Example I-23B: (1-allylcyclopentyl)methanol

To a solution of the product of Example I-23A (15.5 g, 77 mmol, purity 90%) in tetrahydrofuran (300 mL) was added LiAlH₄ (4.65 g, 122 mmol) in portions at 0° C. under nitrogen. Then the mixture was stirred at 0° C. under nitrogen for 2 hours. The reaction mixture was quenched by adding 4.65 mL of water dropwise at 0° C., followed by 4.65 mL of 15% aqueous NaOH solution and 13.95 mL of water. One additional reaction of the same type was run as described above on 15.5 g scale. These two reaction mixtures were combined and filtered through a pad of diatomaceous earth. The filter cake was washed with ethyl acetate (1000 mL) and tetrahydrofuran (1000 mL). The filtrate was concentrated under reduced pressure to give the title compound (22.5 g, 144 mmol, yield 94%, purity 90%). ¹H NMR (400 MHz, CDCl₃) δ ppm 5.86 (m, 1H), 5.13-5.03 (m, 2H), 3.40 (s, 2H), 2.17 (d, J=7.4 Hz, 2H), 1.67-1.51 (m, 4H), 1.56-1.50-1.38 (m, 4H).

### Example I-23C: tert-butyl(dimethyl){[1-(prop-2-en-1-yl)cyclopentyl]methoxy}silane

To a solution of the product of Example I-23B (10.4 g, 74.2 mmol, purity 90%) and imidazole (12.12 g, 178 mmol) in anhydrous dichloromethane (208 mL) was added tert-butyldimethylchlorosilane (12.30 g, 82 mmol) at 0° C. Then the mixture was stirred at 20° C. for 12 hours. Two additional reactions of the same type were run as described above on 2.5 g and 10.4 g scales. These three reaction mixtures were combined, diluted with water (300 mL), and extracted with dichloromethane (3×200 mL). The combined organic fractions were dried over anhydrous sodium sulfate and concentrated under reduced pressure. The residue was purified by column chromatography on silica gel eluted with petroleum ether to give the title compound (39.35 g, 155 mmol, yield 93%, purity 90%). ¹H NMR (400 MHz, CDCl₃) δ ppm 5.87-5.76 (m, 1H), 5.06-4.99 (m, 2H), 3.31 (s, 2H), 2.13 (d, J=7.3 Hz, 2H), 1.61-1.54 (m, 4H), 1.47-1.32 (m, 4H), 0.88 (s, 9H), 0.04 (s, 6H).

### Example I-23D: [1-({[tert-butyl(dimethyl)silyl]oxy}methyl)cyclopentyl]acetaldehyde

To a solution of the product of Example I-23C (14.5 g, 51.3 mmol, purity 90%) in water (60 mL) and tetrahydrofuran (300 mL) was added a solution of osmium tetroxide (107 mg, 0.421 mmol) in 2-methylpropan-2-ol (2 mL) at 20° C. The mixture was stirred for 15 minutes at 20° C. Then sodium periodate (43.9 g, 205 mmol) was added in portions at 0° C. The mixture was stirred for 2 hours at 20° C. Two additional reactions of the same type were run as described above on 14.5 g and 5 g scales. These three reaction mixtures were combined and extracted with ethyl acetate (400 mL). The combined organic fractions were filtered. The filtrate was quenched with saturated Na₂S₂O₃ aqueous solution (600 mL), and the mixture was extracted with ethyl acetate (3×200 mL). The combined organic phases were washed with brine (300 mL), dried over anhydrous sodium sulfate, filtered, and concentrated under reduced pressure. The residue was purified by column chromatography on silica gel eluted with petroleum ether to give the title compound (19 g, 70.4 mmol, yield 57.2%, purity 95%). ¹H NMR (400 MHz, CDCl₃) δ ppm 9.79 (t, J=2.9 Hz, 1H), 3.41 (s, 2H), 2.39 (d, J=2.9 Hz, 2H), 1.71-1.66 (m, 6H), 1.52-1.41 (m, 2H), 0.88 (s, 9H), 0.01 (s, 6H).

### Example I-23E: 5-{(2R)-4-fluoro-6-hydroxy-2-[({2-[1-(hydroxymethyl)cyclopentyl]ethyl}amino)methyl]-2,3-dihydro-1-benzofuran-5-yl}-1λ⁶,2, 5-thiadiazolidine-1,1,3-trione

In a 4 mL vial was added the product of Example I-4I (50 mg, 0.14 mmol) and triethylamine (59 μL, 0.42 mmol, 3 equivalents) in 3:2 v/v ethanol/dichloromethane (1.4 mL). The product of Example I-23D (72.5 mg, 0.42 mmol, 2.0 equivalents) was added, and the reaction mixture was stirred at room temperature for 2 hours. Sodium borohydride (21.4 mg, 0.57 mmol, 4.0 equivalents) was added in one portion, and the resultant mixture was stirred for 30 minutes at ambient temperature. The reaction mixture was cooled to 0° C. in an ice bath, and aqueous hydrochloric acid (2.0 M, 1.4 mL, 2.8 mmol, 20 equivalents) was slowly added. The mixture was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (14.8 mg, 23.6% yield). ¹H NMR (400 MHz, DMSO-d₆) δ ppm 9.25 (s, 1H), 8.74-8.35 (m, 2H), 6.20 (s, 1H), 5.16-5.05 (m, 1H), 3.88 (s, 2H), 3.30-3.24 (m, 3H), 3.16 (s, 2H), 3.03-2.88 (m, 3H), 1.67 (td, J=7.1, 3.3 Hz, 2H), 1.58-1.38 (m, 6H), 1.32-1.21 (m, 2H); MS (APCI⁺) m/z 444.4 [M+H]⁺.

### Example I-24: 5-[(2R)-2-({[3-(2,2-difluoroethoxy)propyl]amino}methyl)-4-fluoro-6-hydroxy-2,3-dihydro-1-benzofuran-5-yl]-1λ⁶,2,5-thiadiazolidine-1,1,3-trione (Compound 123)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). 3-(2,2-Difluoroethoxy)propanal (35.4 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (0.8 mg, 2.2% yield). ¹H NMR (500 MHz, DMSO-d₆:D₂O=9:1 (v/v)) δ ppm 6.29-5.99 (m, 2H), 5.20-5.11 (m, 1H), 3.97 (s, 2H), 3.74-3.60 (m, 4H), 3.42-3.25 (m, 3H), 3.10-3.04 (m, 2H), 2.96 (dd, J=15.4, 6.8 Hz, 1H), 1.99-1.87 (m, 2H); MS (APCI⁺) m/z 440.4 [M+H]⁺.

### Example I-25: tert-butyl 4-[({[(2R)-4-fluoro-6-hydroxy-5-(1,1,4-trioxo-1λ⁶,2,5-thiadiazolidin-2-yl)-2,3-dihydro-1-benzofuran-2-yl]methyl}amino)methyl]piperidine-1-carboxylate (Compound 124)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). tert-Butyl 4-formylpiperidine-1-carboxylate (54.3 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A), to afford the title compound (0.1 mg, 0.23% yield). ¹H NMR (400 MHz, DMSO-d₆:D₂O=9:1 (v/v)) δ ppm 6.27 (s, 1H), 5.21-5.14 (m, 1H), 4.02-3.93 (m, 3H), 3.44-3.32 (m, 1H), 3.30-3.24 (m, 2H), 3.01-2.86 (m, 4H), 2.74 (s, 2H), 2.01-1.84 (m, 1H), 1.81-1.68 (m, 2H), 1.43 (s, 9H), 1.17-1.01 (m, 2H); MS (APCI⁺) m/z 515.5 [M+H]⁺.

### Example I-26: 5-[(2R)-4-fluoro-6-hydroxy-2-({[2-(oxan-4-yl)ethyl]amino}methyl)-2,3-dihydro-1-benzofuran-5-yl]-1λ⁶,2,5-thiadiazolidine-1,1,3-trione (Compound 125)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). 2-(Tetrahydro-2H-pyran-4-yl)acetaldehyde (32.6 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (4 mg, 11% yield). ¹H NMR (400 MHz, DMSO-d₆:D₂O=9:1 (v/v)) δ ppm 6.25-6.20 (m, 1H), 5.15-5.03 (m, 1H), 3.94 (s, 2H), 3.87-3.78 (m, 2H), 3.37-3.23 (m, 3H), 3.16 (d, J=6.2 Hz, 2H), 2.97-2.87 (m, 3H), 1.60-1.49 (m, 5H), 1.24-1.09 (m, 2H); MS (APCI⁺) m/z 430.4 [M+H]⁺.

### Example I-27: 5-{(2R)-2-[({[(1RS,5SR)-bicyclo[3.1.0]hexan-6-yl]methyl}amino)methyl]-4-fluoro-6-hydroxy-2,3-dihydro-1-benzofuran-5-yl}-1λ⁶,2,5-thiadiazolidine-1,1,3-trione (Compound 126)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). Bicyclo[3.1.0]hexane-6-carbaldehyde (28.0 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A), to afford the title compound (4.2 mg, 12.1% yield). ¹H NMR (400 MHz, DMSO-d₆:D₂O=9:1 (v/v)) δ ppm 6.25 (s, 1H), 5.17-5.07 (m, 1H), 3.94 (s, 2H), 3.40-3.17 (m, 3H), 2.99-2.75 (m, 3H), 1.78-1.48 (m, 5H), 1.31 (t, J=3.2 Hz, 2H), 1.13-1.01 (m, 1H), 0.89-0.81 (m, 1H); MS (APCI⁺) m/z 412.3 [M+H]⁺.

### Example I-28: 5-[(2R)-4-fluoro-6-hydroxy-2-{[(3-phenylpropyl)amino]methyl}-2,3-dihydro-1-benzofuran-5-yl]-1λ⁶,2,5-thiadiazolidine-1,1,3-trione (Compound 127)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). 3-Phenylpropanal (34.1 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A), to afford the title compound (4.6 mg, 12.5% yield). ¹H NMR (400 MHz, DMSO-d₆:D₂O=9:1 (v/v)) δ ppm 7.36-7.28 (m, 2H), 7.27-7.18 (m, 3H), 6.22 (s, 1H), 5.12-5.08 (m, 1H), 3.93 (s, 2H), 3.45-3.29 (m, 1H), 3.25-3.21 (m, 2H), 2.97-2.87 (m, 3H), 2.69-2.60 (m, 2H), 2.00-1.84 (m, 2H); MS (APCI⁺) m/z 436.4 [M+H]⁺.

### Example I-29: 5-[(2R)-4-fluoro-6-hydroxy-2-({[2-(2,6,6-trimethylcyclohex-1-en-1-yl)ethyl]amino}methyl)-2,3-dihydro-1-benzofuran-5-yl]-1λ⁶,2,5-thiadiazolidine-1,1,3-trione (Compound 128)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). 2-(2,6,6-Trimethylcyclohex-1-en-1-yl)acetaldehyde (42.3 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A), to afford the title compound (5.4 mg, 13.7% yield). ¹H NMR (400 MHz, DMSO-d₆:D₂O=9:1 (v/v)) δ ppm 6.24 (s, 1H), 5.18-5.07 (m, 1H), 3.94 (s, 2H), 3.40-3.26 (m, 3H), 2.99-2.85 (m, 3H), 2.45-2.27 (m, 2H), 1.89 (t, J=6.2 Hz, 2H), 1.61 (s, 3H), 1.56-1.47 (m, 2H), 1.43-1.35 (m, 2H), 0.98 (s, 6H); MS (APCI⁺) m/z 468.4 [M+H]⁺.

### Example I-30: 5-[(2R)-4-fluoro-6-hydroxy-2-({[(3-phenylcyclobutyl)methyl]amino}methyl)-2,3-dihydro-1-benzofuran-5-yl]-1λ⁶,2,5-thiadiazolidine-1,1,3-trione (Compound 129)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). 3-Phenylcyclobutane-1-carbaldehyde (40.8 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the reaction mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 Å AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A), to afford the title compound (6.4 mg, 16.4% yield). ¹H NMR (400 MHz, DMSO-d₆:D₂O=9:1 (v/v)) δ ppm 7.35-7.13 (m, 5H), 6.22 (s, 1H), 5.15-5.07 (m, 1H), 3.91 (s, 2H), 3.62-3.54 (m, 1H), 3.41-3.26 (m, 2H), 3.26-3.13 (m, 2H), 3.07-3.00 (m, 1H), 2.97-2.86 (m, 1H), 2.68-2.55 (m, 1H), 2.49-2.41 (m, 2H), 2.28-2.19 (m, 1H), 1.90-1.78 (m, 1H); MS (APCI⁺) m/z 462.4 [M+H]⁺.

### Example I-31: 5-{(2R)-4-fluoro-6-hydroxy-2-[({[4-(trifluoromethyl)cyclohexyl]methyl}amino)methyl]-2,3-dihydro-1-benzofuran-5-yl}-1λ⁶,2,5-thiadiazolidine-1,1,3-trione (Compound 130)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). 4-(Trifluoromethyl)cyclohexane-1-carbaldehyde (45.8 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the resultant mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-80% A, 8.0-8.1 minutes 80-100% A, 8.1-9.0 minutes 100% A, 9.0-9.1 minutes linear gradient 100-5% A, 9.1-10.0 minutes 5% A), to afford the title compound (7.4 mg, 18.2% yield). ¹H NMR (400 MHz, DMSO-d₆:D₂O=9:1 (v/v)) 6 ppm 6.24 (s, 1H), 5.21-5.11 (m, 1H), 3.94 (s, 2H), 3.40-3.19 (m, 3H), 3.09-2.79 (m, 3H), 2.36-2.19 (m, 1H), 1.94-1.82 (m, 2H), 1.74-1.41 (m, 5H), 1.32-1.19 (m, 1H), 1.11-0.98 (m, 1H); MS (APCI⁺) m/z 482.2 [M+H]⁺.

### Example I-32: 5-{(2R)-4-fluoro-2-[({[1-(fluoromethyl)cyclopropyl]methyl}amino)methyl]-6-hydroxy-2,3-dihydro-1-benzofuran-5-yl}-1λ⁶,2,5-thiadiazolidine-1,1,3-trione (Compound 131)

In a 4 mL vial was added the product of Example I-4I (30 mg, 0.08 mmol) and triethylamine (36 μL, 0.25 mmol, 3 equivalents) in 3:2 v/v ethanol/dichloromethane (0.8 mL). 1-(Fluoromethyl)cyclopropane-1-carbaldehyde (26.0 mg, 0.25 mmol, 3.0 equivalents) was added, and the reaction mixture was stirred at room temperature for 2 hours. Sodium borohydride (13.5 mg, 0.34 mmol, 4.0 equivalents) was added in one portion, and the mixture was stirred for 30 minutes at ambient temperature. The reaction mixture was cooled to 0° C. in an ice bath, and saturated ammonium chloride (1.0 mL) was slowly added. Then the mixture was partially concentrated under a stream of nitrogen. Methanol (1 mL) was added, and the mixture was purified via reverse-phase preparative HPLC on a Phenomenex® Luna® C8(2) 5 μm 100 AXIA™ column (50 mm×30 mm). A gradient of methanol (A) and 25 mM ammonium bicarbonate buffer (pH 7) in water (B) was used, at a flow rate of 40 mL/minute (0-0.5 minutes 5% A, 0.5-8.0 minutes linear gradient 5-60% A, 8.0-8.1 minutes 60-100% A, 8.1-9.0 minutes 100% A, 9.0-9.1 minutes linear gradient 100-5% A, 9.1-10.0 minutes 5% A), to afford the title compound (2.0 mg, 5.9% yield). ¹H NMR (400 MHz, DMSO-d₆:D₂O=9:1 (v/v)) 6 ppm 6.20 (s, 1H), 5.04-4.92 (m, 1H), 4.43 (s, 1H), 4.30 (s, 1H), 3.99 (s, 2H), 3.30-3.20 (m, 1H), 3.01-2.77 (m, 3H), 2.65-2.60 (m, 2H), 0.57-0.52 (m, 4H); MS (APCI⁺) m/z 404.3 [M+H]⁺.

### Example II-1: 5-[(2S)-4-fluoro-6-hydroxy-2-{[(2-methylpropyl)amino]methyl}-2,3-dihydro-1H-indol-5-yl]-1λ⁶,2,5-thiadiazolidine-1,1,3-trione (Compound 132)

### Example II-1A: methyl (2R)-2-[(tert-butoxycarbonyl)amino]-3-{[tert-butyl(dimethyl)silyl]oxy}propanoate

To a solution of methyl (2R)-2-[(tert-butoxycarbonyl)amino]-3-hydroxypropanoate (33.6 g, 153 mmol) and tert-butyldimethylchlorosilane (25.4 g, 169 mmol) in tetrahydrofuran (260 mL) was added imidazole (25 g, 367 mmol) portionwise over 15 minutes at 0° C. The resulting mixture was warmed to room temperature and stirred for 18 hours. The reaction was quenched by addition of methanol (15 mL). The resulting mixture was stirred for 5 minutes before diluting sequentially with water (50 mL), brine (75 mL) and ethyl acetate (400 mL). The layers were separated, and the aqueous layer was extracted with ethyl acetate (2×100 mL). The organic layers were combined, washed with brine (200 mL), dried over sodium sulfate, filtered, and concentrated in vacuo to afford the title compound (59.8 g, 152 mmol, 99% yield). ¹H NMR (400 MHz, CDCl₃) δ ppm 5.34 (d, J=8.9 Hz, 1H), 4.35 (dt, J=9.0, 2.9 Hz, 1H), 4.04 (dd, J=10.1, 2.8 Hz, 1H), 3.82 (dd, J=10.1, 3.1 Hz, 1H), 3.74 (s, 3H), 1.45 (s, 9H), 0.86 (s, 9H), 0.02 (d, J=5.4 Hz, 6H).

### Example II-1B: tert-butyl [(2S)-1-{[tert-butyl(dimethyl)silyl]oxy}-3-hydroxypropan-2-yl]carbamate

To a solution of the product of Example II-1A (29.85 g, 76 mmol) in tetrahydrofuran (300 mL) at 0° C. was added lithium borohydride (2 M in tetrahydrofuran, 57 mL, 114 mmol) dropwise over 20 minutes. The resulting colorless solution was slowly warmed to room temperature and stirred for 80 hours. The mixture was diluted carefully with a saturated aqueous solution of ammonium chloride (20 mL), water (75 mL) and ethyl acetate (400 mL). The resulting biphasic mixture was stirred vigorously for 20 minutes and then separated. The aqueous layer was extracted with ethyl acetate (2×100 mL). 1 M Aqueous hydrochloric acid (100 mL) was slowly added to the rapidly stirred combined organic layers at 0° C. The mixture was stirred for an additional 10 minutes upon completion of the addition. The organic layer was separated, washed with brine (100 mL), dried over magnesium sulfate, filtered, and concentrated in vacuo. The residue was taken up in 20% tert-butyl methyl ether in isohexane (200 mL), filtered through a pad of a mixture of silica and diatomaceous earth (1:1, 5×5 cm) and washed with 20% tert-butyl methyl ether in isohexane (3×50 mL). The filtrate was concentrated in vacuo to afford the title compound (22.73 g, 63.2 mmol, 83% yield). ¹H NMR (400 MHz, CDCl₃) δ ppm 3.89-3.74 (m, 3H), 3.72-3.59 (m, 2H), 1.45 (s, 9H), 0.91 (d, J=6.4 Hz, 9H), 0.09 (d, J=9.4 Hz, 6H).

### Example II-1C: tert-butyl (4R)-4-({[tert-butyl(dimethyl)silyl]oxy}methyl)-2-oxo-1,2λ⁴,3-oxathiazolidine-3-carboxylate

To a solution of imidazole (17.2 g, 253 mmol) and triethylamine (26.5 mL, 190 mmol) in dichloromethane (200 mL) at −40° C. was added a solution of thionyl chloride, 1 M in dichloromethane (76 mL, 76 mmol) dropwise over 5 minutes. After 30 minutes, a solution of the product of Example II-1B (22.73 g, 63.2 mmol) in dichloromethane (45 mL) was added dropwise over 30 minutes with a syringe pump. The mixture was allowed to warm slowly to room temperature and stirred for 20 hours. The reaction was quenched with a saturated aqueous solution of sodium bicarbonate (125 mL). The aqueous layer was extracted with dichloromethane (2×125 mL). The combined organic layers were washed with brine (125 mL), dried over magnesium sulfate, filtered, and concentrated in vacuo. The crude residue was subjected to column chromatography (SiO₂, 0-30% ethyl acetate in isohexane) to afford the title compound (16.2 g, 41.5 mmol, 66% yield). ¹H NMR (400 MHz, CDCl₃) δ ppm 5.07-4.90 (m, 1H), 4.86-4.66 (m, 1H), 4.24-4.01 (m, 2H), 3.85-3.69 (m, 1H), 1.61-1.47 (m, 9H), 0.96-0.81 (m, 9H), 0.16-0.00 (m, 6H).

### Example II-1D: tert-butyl (4R)-4-({[tert-butyl(dimethyl)silyl]oxy}methyl)-2,2-dioxo-1,2λ⁶,3-oxathiazolidine-3-carboxylate

To a solution of the product of Example II-1C (16.2 g, 41.5 mmol) in acetonitrile (80 mL) and water (20 mL) at 0° C. was added ruthenium(III) chloride, H₂O (0.05 g, 0.222 mmol) followed by sodium periodate (13.30 g, 62.2 mmol). After 10 minutes, the mixture was filtered through a plug of diatomaceous earth (3×3 cm) and the solid was washed with ethyl acetate (3×50 mL). The filtrate was washed with a saturated aqueous solution of sodium thiosulfate (100 mL). The aqueous layer was then extracted with ethyl acetate (2×100 mL). The combined organic layers were washed sequentially with a saturated aqueous solution of sodium thiosulfate (100 mL) and brine (100 mL). The mixture was dried over sodium sulfate, filtered, and concentrated in vacuo. The crude residue was subjected to column chromatography (SiO₂, dry loaded with silica, 0-100% ethyl acetate in isohexane) to afford the title compound (12.5 g, 32.3 mmol, 78% yield). ¹H NMR (400 MHz, CDCl₃) δ ppm 4.67-4.55 (m, 2H), 4.31-4.21 (m, 1H), 3.86 (dd, J=10.2, 4.0 Hz, 1H), 3.83-3.70 (m, 1H), 1.55 (s, 9H), 0.89 (s, 9H), 0.08 (d, J=2.7 Hz, 6H).

### Example II-1E: tert-butyl [(2S)-1-[4-(benzyloxy)-6-bromo-2-fluoro-3-(2,2,2-trifluoroacetamido)phenyl]-3-{[tert-butyl(dimethyl)silyl]oxy}propan-2-yl]carbamate

To a solution of diisopropylamine (4.30 mL, 30.2 mmol) in tetrahydrofuran (65 mL) at −78° C. was added n-butyllithium 2.5 M in hexane (12.0 mL, 30.0 mmol) dropwise over 10 minutes. After 15 minutes, a solution of the product of Example I-3C (5.25 g, 13.12 mmol) in tetrahydrofuran (20 mL) was added dropwise over 45 minutes using a syringe pump keeping the temperature below −74° C. After 30 minutes, a solution of the product of Example II-1D (5.33 g, 13.78 mmol) in tetrahydrofuran (15 mL) was added dropwise over 45 minutes using a syringe pump, and the resulting solution was stirred for a further 90 minutes keeping the temperature below −74° C. The reaction was quenched slowly with 1 M aqueous hydrochloric acid (35 mL), and the mixture was allowed to warm to room temperature over 20 minutes. 1 M Aqueous hydrochloric acid (25 mL) was added, and the mixture was stirred at room temperature for a further 30 minutes. The mixture was diluted with ethyl acetate (200 mL), the layers were separated, and the aqueous layer was extracted with ethyl acetate (2×150 mL). The combined organic layers were washed with brine (150 mL), dried over magnesium sulfate, filtered, and concentrated in vacuo. The crude residue was subjected to column chromatography (SiO₂, dry loaded with silica, 0-10% ethyl acetate in isohexane) to afford the title compound (8.06 g, 10.67 mmol, 81% yield, 90% purity). ¹H NMR (400 MHz, CDCl₃) δ ppm 7.44-7.29 (m, 5H), 7.05 (d, J=1.8 Hz, 1H), 5.16-4.98 (m, 2H), 4.83 (d, J=9.6 Hz, 1H), 3.99 (s, 1H), 3.68 (d, J=3.7 Hz, 2H), 3.05 (ddd, J=12.7, 10.1, 2.4 Hz, 1H), 2.91 (d, J=14.1 Hz, 1H), 1.38-1.13 (m, 9H), 0.93 (s, 9H), 0.08 (d, J=1.7 Hz, 6H); MS (ESI⁺) m/z 579 (⁷⁹Br), 581 (⁸¹Br) [M-C(O)OC(CH₃)₃+H]⁺.

### Example II-1F: methyl [{6-(benzyloxy)-4-bromo-3-[(2S)-2-[(tert-butoxycarbonyl)amino]-3-{[tert-butyl(dimethyl)silyl]oxy}propyl]-2-fluorophenyl}(trifluoroacetyl)amino]acetate

Methyl bromoacetate (1.1 mL, 11.94 mmol) was added to a suspension of the product of Example II-1E (8.06 g, 10.67 mmol), potassium carbonate (2.95 g, 21.35 mmol) and potassium iodide (1.78 g, 10.72 mmol) in acetone (15 mL) at room temperature. The mixture was then stirred at 60° C. for 5 hours. The mixture was cooled to room temperature and concentrated in vacuo. The residue was partitioned between ethyl acetate (150 mL) and a saturated aqueous solution of ammonium chloride (150 mL). The aqueous layer was extracted with ethyl acetate (2×100 mL). The combined organic layers were washed with brine (100 mL), dried over magnesium sulfate, filtered, and concentrated in vacuo. The crude residue was subjected to column chromatography (SiO₂, dry loaded with silica, 0-40% ethyl acetate in isohexane) to afford the title compound (8.4 g, 9.50 mmol, 89% yield). ¹H NMR (400 MHz, CDCl₃) δ ppm 7.45-7.29 (m, 5H), 7.07 (d, J=7.8 Hz, 1H), 5.11-4.98 (m, 2H), 4.82-4.67 (m, 1H), 4.53 (dd, J=24.1, 16.9 Hz, 1H), 4.08-3.89 (m, 2H), 3.72-3.58 (m, 5H), 3.10-2.84 (m, 2H), 1.43-1.19 (m, 9H), 1.00-0.85 (m, 9H), 0.15-−0.05 (m, 6H); MS (ESI⁺) m/z 773 (⁷⁹Br), 775 (⁸¹Br) [M+Na]⁺.

### Example II-1G: tert-butyl (2S)-6-(benzyloxy)-2-({[tert-butyl(dimethyl)silyl]oxy}methyl)-4-fluoro-5-[(2-methoxy-2-oxoethyl)(trifluoroacetyl)amino]-2,3-dihydro-1H-indole-1-carboxylate

Nitrogen was bubbled through a mixture of the product of Example II-1F (4.2 g, 4.75 mmol) and cesium carbonate (4.6 g, 14.12 mmol) in tetrahydrofuran (20 mL) and water (2 mL) for 10 minutes. RuPhos Pd G3 (0.4 g, 0.478 mmol) was added, and nitrogen was bubbled through the mixture for a further 5 minutes before stirring at 80° C. for 8 hours. The mixture was cooled to room temperature, filtered through a pad of diatomaceous earth (3×3 cm) and washed with ethyl acetate (2×100 mL). The crude residue was subjected to column chromatography (SiO₂, dry loaded with silica, 0-25% ethyl acetate in isohexane) to afford the title compound (3.2 g, 4.77 mmol, 100% yield). ¹H NMR (400 MHz, CDCl₃) δ ppm 7.55-7.29 (m, 6H), 5.15-4.98 (m, 2H), 4.64 (dd, J=16.8, 6.1 Hz, 1H), 4.49 (s, 1H), 3.89 (dd, J=16.8, 13.1 Hz, 1H), 3.77-3.67 (m, 2H), 3.65 (d, J=6.2 Hz, 3H), 3.22-3.04 (m, 2H), 1.56 (s, 9H), 0.75 (d, J=20.6 Hz, 9H), 0.04-−0.10 (m, 6H); MS (ESI⁺) m/z 694 [M+H]⁺.

### Example II-1H: tert-butyl (2S)-6-(benzyloxy)-4-fluoro-2-(hydroxymethyl)-5-[(2-methoxy-2-oxoethyl)(trifluoroacetyl)amino]-2,3-dihydro-1H-indole-1-carboxylate

To a solution of the product of Example II-1G (3.5 g, 5.22 mmol) in tetrahydrofuran (25 mL) at room temperature was added tetra-N-butylammonium fluoride, 1 M in tetrahydrofuran (5.8 mL, 5.80 mmol). The resulting solution was stirred at room temperature for 1 hour. The reaction mixture was concentrated in vacuo. The crude residue was subjected to column chromatography (SiO₂, dry loaded with silica, 0-60% ethyl acetate in isohexane) to afford the title compound (2.41 g, 4.33 mmol, 83% yield). ¹H NMR (400 MHz, CDCl₃) δ ppm 7.53-7.18 (m, 6H), 5.14-4.98 (m, 2H), 4.73-4.53 (m, 2H), 3.92 (d, J=16.9 Hz, 1H), 3.87-3.71 (m, 2H), 3.65 (d, J=4.4 Hz, 3H), 3.27 (td, J=15.9, 10.1 Hz, 1H), 2.96 (d, J=15.8 Hz, 1H), 1.76-1.45 (m, 9H); MS (ESI⁻) m/z 555 [M−H]⁻.

### Example II-1I: tert-butyl (2S)-6-(benzyloxy)-4-fluoro-2-formyl-5-[(2-methoxy-2-oxoethyl)(trifluoroacetyl)amino]-2,3-dihydro-1H-indole-1-carboxylate

Dess-Martin periodinane (1,1,1-tris(acetyloxy)-1,1-dihydro-1,2-benzodioxol-3-(1H)-one) (300 mg, 0.707 mmol) was added to a solution of the product of Example II-1H (400 mg, 0.647 mmol) in dichloromethane (0.5 mL) at 0° C. The reaction mixture was then allowed to slowly warm to room temperature and stirred for 18 hours. Saturated aqueous solutions of sodium thiosulfate (15 mL) and sodium bicarbonate (15 mL) were added, and the biphasic mixture was diluted with dichloromethane (30 mL). The layers were separated, and the aqueous layer was extracted with dichloromethane (2×20 mL). The combined organic layers were washed with a saturated aqueous solution of sodium bicarbonate (3×15 mL) and brine (15 mL), dried over magnesium sulfate, filtered, and concentrated in vacuo to afford the title compound (398 mg, 0.574 mmol, 89% yield, 80% purity). ¹H NMR (400 MHz, CDCl₃) δ ppm 9.65 (d, J=19.1 Hz, 1H), 7.43-7.29 (m, 6H), 5.17-5.02 (m, 2H), 4.94-4.79 (m, 1H), 4.67 (d, J=16.8 Hz, 1H), 3.89 (d, J=17.8 Hz, 1H), 3.65 (d, J=1.3 Hz, 3H), 3.45-3.31 (m, 1H), 3.16 (dt, J=16.5, 6.3 Hz, 1H), 1.53 (s, 9H); MS (ESI⁺) m/z 455 [M-C(O)OC(CH₃)₃+H]⁺.

### Example II-1J: tert-butyl (2S)-6-(benzyloxy)-4-fluoro-5-[(2-methoxy-2-oxoethyl)(trifluoroacetyl)amino]-2-{[(2-methylpropyl)amino]methyl}-2,3-dihydro-1H-indole-1-carboxylate

To a degassed solution of the product of Example II-1I (398 mg, 0.574 mmol) in 1,2-dichloroethane (2 mL) was added 2-methylpropan-1-amine (110 μL, 1.107 mmol) followed by acetic acid (60 μL, 1.048 mmol). The resulting solution was stirred at room temperature for 20 minutes. Additional 2-methylpropan-1-amine (40 μL, 0.403 mmol) and then acetic acid (20 μL, 0.349 mmol) were added, and the reaction mixture was stirred at room temperature for 25 minutes. Sodium triacetoxyborohydride (180 mg, 0.849 mmol) was then added, and the mixture stirred for 45 minutes. The reaction mixture was diluted with water (15 mL) and dichloromethane (20 mL). The layers were separated, and the aqueous layer was extracted with dichloromethane (2×15 mL). The combined organic layers were dried over magnesium sulfate, filtered, and concentrated in vacuo. The crude residue was subjected to column chromatography (12 g SiO₂ cartridge, dry loaded with silica, 0-100% ethyl acetate in isohexane) to afford the title compound (190 mg, 0.249 mmol, 43% yield). ¹H NMR (400 MHz, CDCl₃) δ ppm 7.44-7.29 (m, 6H), 5.12-5.00 (m, 2H), 4.65 (dd, J=16.8, 2.5 Hz, 1H), 3.97-3.87 (m, 1H), 3.65 (d, J=4.8 Hz, 3H), 3.36-3.11 (m, 2H), 3.10-2.71 (m, 2H), 2.68-2.34 (m, 1H), 2.07 (s, 2H), 1.91-1.74 (m, 1H), 1.62-1.48 (m, 9H), 1.01-0.83 (m, 6H); MS (ESI⁺) m/z 613 [M+H]⁺.

### Example II-1K: tert-butyl (2S)-6-(benzyloxy)-2-{[(tert-butoxycarbonyl)(2-methylpropyl)amino]methyl}-4-fluoro-5-[(2-methoxy-2-oxoethyl)(trifluoroacetyl)amino]-2,3-dihydro-1H-indole-1-carboxylate

To a solution of the product of Example II-1J (220 mg, 0.306 mmol) in acetonitrile (3 mL) at room temperature was added N,N-diisopropylethylamine (0.16 mL, 0.916 mmol) and di-tert-butyl dicarbonate (130 mg, 0.596 mmol). The mixture was stirred at room temperature for 30 minutes. The reaction mixture was quenched with water (10 mL), diluted with dichloromethane (10 mL), and the layers were separated. The aqueous layer was extracted with dichloromethane (3×15 mL). The combined organic layers were washed with brine (10 mL), dried over magnesium sulfate, filtered, and concentrated in vacuo. The crude residue was subjected to column chromatography (SiO₂, dry loaded with silica, 12 g cartridge, 0-40% ethyl acetate in isohexane) to afford the title compound (210 mg, 0.280 mmol, 92% yield). ¹H NMR (400 MHz, CDCl₃) δ ppm 7.64-7.29 (m, 6H), 5.19-4.95 (m, 2H), 4.64 (t, J=16.3 Hz, 2H), 3.92 (dd, J=16.8, 11.0 Hz, 1H), 3.65 (s, 3H), 3.49 (s, 1H), 3.38-3.24 (m, 1H), 3.24-2.76 (m, 4H), 2.13-1.84 (m, 1H), 1.65-1.51 (m, 9H), 1.41 (d, J=12.3 Hz, 9H), 0.96-0.73 (m, 6H); MS (ESI⁺) m/z 613 [M-C(O)OC(CH₃)₃+H]⁺.

### Example II-1L: tert-butyl (2S)-6-(benzyloxy)-2-{[(tert-butoxycarbonyl)(2-methylpropyl)amino]methyl}-4-fluoro-5-[(2-methoxy-2-oxoethyl)amino]-2,3-dihydro-1H-indole-1-carboxylate

To a solution of the product of Example II-1K (520 mg, 0.694 mmol) in methanol (2.5 mL) at room temperature was added sodium methoxide (0.39 mL, 2.106 mmol). The mixture was then stirred at 50° C. for 1 hour, cooled to room temperature and diluted with water (10 mL) and methyl tert-butyl ether (15 mL). To the cloudy biphasic mixture was added a saturated aqueous solution of ammonium chloride (10 mL). The aqueous layer was extracted with methyl tert-butyl ether (3×20 mL). The combined organic layers were washed with brine (15 mL), dried over magnesium sulfate, filtered, and concentrated in vacuo to afford the title compound (460 mg, 0.523 mmol, 75% yield). ¹H NMR (400 MHz, CDCl₃) δ ppm 7.63-7.07 (m, 6H), 5.23-5.01 (m, 2H), 4.82-4.48 (m, 2H), 3.99-3.77 (m, 2H), 3.60-3.19 (m, 3H), 3.20-2.80 (m, 5H), 2.12-1.79 (m, 2H), 1.56 (d, J=4.2 Hz, 9H), 1.41 (s, 9H), 0.87 (s, 6H); MS (ESI⁺) m/z 517 [M-C(O)OC(CH₃)₃+H]⁺.

### Example II-1M: tert-butyl (2S)-6-(benzyloxy)-2-{[(tert-butoxycarbonyl)(2-methylpropyl)amino]methyl}-4-fluoro-5-[(2-methoxy-2-oxoethyl)({[(prop-2-en-1-yl)oxy]carbonyl}sulfamoyl)amino]-2,3-dihydro-1H-indole-1-carboxylate

To a solution of chlorosulfonyl isocyanate (0.11 mL, 1.267 mmol) in dichloromethane (1 mL) at 0° C. was added allyl alcohol (90 μL, 1.323 mmol) dropwise. The resulting solution was stirred for 20 minutes at 0° C. before a prepared solution of the product of Example II-1L (460 mg, 0.523 mmol) and triethylamine (0.25 mL, 1.794 mmol) in dichloromethane (2 mL) was added dropwise. The resulting solution was stirred at room temperature for 30 minutes. The reaction mixture was quenched with water (10 mL) and diluted with dichloromethane (20 mL). The layers were separated, and the aqueous layer was extracted with dichloromethane (3×20 mL). The combined organic layers were dried over magnesium sulfate, filtered, and concentrated in vacuo. The crude residue was subjected to column chromatography (SiO₂, dry loaded with silica, 24 g cartridge, 0-35% ethyl acetate in isohexane) to afford the title compound (277 mg, 0.249 mmol, 48% yield). ¹H NMR (400 MHz, CDCl₃) δ ppm 7.51-7.30 (m, 6H), 5.85-5.70 (m, 1H), 5.51-5.18 (m, 2H), 5.17-5.06 (m, 2H), 4.90-4.49 (m, 3H), 4.45-4.24 (m, 4H), 3.73-3.59 (m, 3H), 3.58-2.90 (m, 5H), 2.10-1.83 (m, 1H), 1.57 (d, J=15.8 Hz, 9H), 1.42 (s, 9H), 0.88 (t, J=6.2 Hz, 6H); MS (ESI⁺) m/z 801 [M+Na]⁺.

### Example II-1N: tert-butyl (2S)-6-(benzyloxy)-2-{[(tert-butoxycarbonyl)(2-methylpropyl)amino]methyl}-4-fluoro-5-(1,1,4-trioxo-1λ⁶,2,5-thiadiazolidin-2-yl)-2,3-dihydro-1H-indole-1-carboxylate-ammonia

To a solution of the product of Example II-1M (230 mg, 0.207 mmol) and potassium carbonate (110 mg, 0.796 mmol) in methanol (1.5 mL) was added tetrakis(triphenylphosphine)palladium(0) (20 mg, 0.017 mmol). The reaction mixture was stirred at 60° C. for 30 minutes. The mixture was cooled to room temperature and diluted with methanol (5 mL). Diatomaceous earth was added, and the mixture was concentrated in vacuo. The crude residue was subjected to column chromatography (Reveleris® 24 g reversed phase (C18) flash cartridge, dry loaded with diatomaceous earth, 5-70% methanol in 10 mM ammonium bicarbonate) to afford the title compound as an ammonium salt (133 mg, 0.147 mmol, 71% yield). ¹H NMR (400 MHz, DMSO-d₆) δ ppm 7.71-7.45 (m, 5H), 7.42-7.22 (m, 1H), 7.06 (s, 3H), 5.09 (s, 2H), 4.65 (s, 1H), 4.00-3.79 (m, 2H), 3.50-3.21 (m, 3H), 3.14-2.78 (m, 3H), 1.91 (s, 1H), 1.49 (s, 9H), 1.44-1.20 (m, 9H), 0.81 (d, J=6.6 Hz, 6H); MS (ESI⁺) m/z 563 [M-C(O)OC(CH₃)₃+H]⁺.

### Example II-10: tert-butyl (2S)-2-{[(tert-butoxycarbonyl)(2-methylpropyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a suspension of the product of Example II-1N (130 mg, 0.143 mmol) in degassed water (0.1 mL) and ethanol (2 mL) was added 10% Pd/C (15 mg). The resulting suspension was allowed to stir under hydrogen (5 bar) for 1.5 hours. Additional 10% Pd/C (23 mg) was added, and the reaction mixture was allowed to stir under hydrogen (5 bar) for 4 hours. Additional 10% Pd/C (23 mg, 0.022 mmol) was added, and the reaction mixture was allowed to stir under hydrogen (5 bar) for 18 hours. The reaction mixture was filtered through a pad of diatomaceous earth and washed with ethanol (3×20 mL). The filtrate was concentrated in vacuo. The crude residue was subjected to column chromatography (Reveleris® 24 g reversed phase (C18) flash cartridge, dry loaded with diatomaceous earth, 5-55% methanol in 10 mM ammonium bicarbonate) to afford the title compound as an ammonium salt (51 mg, 0.082 mmol, 57% yield). ¹H NMR (400 MHz, DMSO-d₆) δ ppm 7.22-6.78 (m, 4H), 4.61 (d, J=7.9 Hz, 1H), 3.97-3.77 (m, 2H), 3.47-3.13 (m, 3H), 3.11-2.87 (m, 3H), 2.87-2.62 (m, 1H), 1.91 (s, 1H), 1.50 (s, 9H), 1.34 (d, J=17.7 Hz, 9H), 0.81 (d, J=6.7 Hz, 6H); MS (ESI⁻) m/z 571 [M−H]⁻.

### Example II-1P: 5-[(2S)-4-fluoro-6-hydroxy-2-{[(2-methylpropyl)amino]methyl}-2,3-dihydro-1H-indol-5-yl]-1λ⁶,2,5-thiadiazolidine-1,1,3-trione

To a solution of the product of Example II-10 (50 mg, 0.081 mmol) in dichloromethane (1 mL) at room temperature was added trifluoroacetic acid (0.09 mL, 1.168 mmol). The reaction mixture was stirred at room temperature for 3 hours. The reaction mixture was neutralized with 0.7 M NH₃ in methanol:water (95:5) (5 mL) and concentrated in vacuo. The crude residue was subjected to column chromatography (Reveleris® 12 g reversed phase (C18) flash cartridge, dry loaded with diatomaceous earth, 5-20% methanol in 10 mM ammonium bicarbonate) to afford the title compound (7.48 mg, 0.020 mmol, 25% yield). ¹H NMR (400 MHz, DMSO-d₆) δ ppm 8.69 (s, 1H), 8.18 (s, 2H), 6.02 (s, 1H), 5.93 (s, 1H), 4.20-4.07 (m, 1H), 3.84 (s, 2H), 3.18-2.93 (m, 3H), 2.87-2.62 (m, 3H), 1.97 (hept, J=6.8 Hz, 1H), 0.95 (d, J=6.7 Hz, 6H); MS (ESI⁺) m/z 373 [M+H]⁺.

### Example II-2: 5-[(2R)-4-fluoro-6-hydroxy-2-{[(2-methylpropyl)amino]methyl}-2,3-dihydro-1H-indol-5-yl]-1λ⁶,2,5-thiadiazolidine-1,1,3-trione (Compound 133)

### Example II-2A: methyl (2S)-2-[(tert-butoxycarbonyl)amino]-3-{[tri(propan-2-yl)silyl]oxy}propanoate

To a solution of methyl (2S)-2-[(tert-butoxycarbonyl)amino]-3-hydroxypropanoate (50 g, 228 mmol) and triisopropylchlorosilane (53 mL, 250 mmol) in tetrahydrofuran (450 mL) was added imidazole (34.2 g, 502 mmol) portionwise at 0° C. The resulting mixture was warmed to room temperature and stirred for 18 hours. The reaction was quenched by addition of methanol (20 mL). The resulting mixture was stirred for 5 minutes before diluting sequentially with 1:1 water:brine (500 mL) and ethyl acetate (300 mL). The layers were separated, and the aqueous layer was extracted with ethyl acetate (2×300 mL). The organic layers were combined, washed with brine (200 mL), dried over sodium sulfate, filtered, and concentrated in vacuo. The crude residue was subjected to plug chromatography (SiO₂, 500 g, 10% ethyl acetate in isohexane) to afford the title compound (81.3 g, 195 mmol, 85% yield, 90% purity). ¹H NMR (400 MHz, DMSO-d₆) δ ppm 6.85 (d, J=8.2 Hz, 1H), 4.18 (dt, J=8.2, 5.6 Hz, 1H), 3.88 (d, J=5.6 Hz, 2H), 3.62 (s, 3H), 1.37 (s, 9H), 1.16-0.93 (m, 21H).

### Example II-2B: tert-butyl [(2R)-1-hydroxy-3-{[tri(propan-2-yl)silyl]oxy}propan-2-yl]carbamate

To a solution of the product of Example II-2A (81.3 g, 195 mmol) in tetrahydrofuran (500 mL) at 0° C. was added lithium borohydride (2 M in tetrahydrofuran, 136 mL, 273 mmol) dropwise over 1 hour 20 minutes. The resulting solution was slowly warmed to room temperature and stirred for 80 hours. The mixture was diluted carefully with a saturated aqueous solution of ammonium chloride (50 mL), water (100 mL) and ethyl acetate (200 mL). The resulting biphasic mixture was stirred vigorously for 20 minutes. The layers were separated. The aqueous layer was extracted with ethyl acetate (2×200 mL). 1 M Aqueous hydrochloric acid (100 mL) was slowly added to the rapidly stirred combined organic layers at 0° C. The mixture was stirred for an additional 10 minutes upon completion of the addition. The organic layer was separated, washed with brine (100 mL), dried over sodium sulfate, filtered, and concentrated in vacuo to afford the title compound (75.8 g, 195 mmol, 100% yield, 90% purity). ¹H NMR (400 MHz, DMSO-d₆) δ ppm 6.31 (d, J=8.2 Hz, 1H), 4.53 (t, J=5.6 Hz, 1H), 3.72-3.53 (m, 2H), 3.53-3.43 (m, 1H), 3.43-3.34 (m, 2H), 1.37 (s, 9H), 1.14-0.94 (m, 21H).

### Example II-2C: tert-butyl (4S)-2-oxo-4-({[tri(propan-2-yl)silyl]oxy}methyl)-1,2λ⁴,3-oxathiazolidine-3-carboxylate

To a solution of imidazole (21.5 g, 316 mmol) and triethylamine (87 mL, 622 mmol) in dichloromethane (300 mL) at −60° C. was added thionyl chloride (18.4 mL, 252 mmol) dropwise over 15 minutes. After 30 minutes, a solution of the product of Example II-2B (75.8 g, 196 mmol) in dichloromethane (100 mL) was added dropwise over 45 minutes. The mixture was allowed to warm slowly to room temperature and stirred for 18 hours. The reaction was quenched with a saturated aqueous solution of sodium bicarbonate (400 mL) and the layers were separated. The aqueous layer was extracted with dichloromethane (3×100 mL). The combined organic layers were washed with brine (125 mL), dried over sodium sulfate, filtered, and concentrated in vacuo. The crude residue was subjected to plug chromatography (SiO₂, 10% ethyl acetate in isohexane) to afford the title compound (76.8 g, 176 mmol, 89% yield). ¹H NMR (400 MHz, CDCl₃) δ ppm 5.13-4.92 (m, 1H), 4.88-4.71 (m, 1H), 4.27-4.13 (m, 1H), 4.13-4.04 (m, 1H), 3.89-3.76 (m, 1H), 1.52 (s, 9H), 1.18-0.93 (m, 21H).

### Example II-2D: tert-butyl (4S)-2,2-dioxo-4-({[tri(propan-2-yl)silyl]oxy}methyl)-1,2λ⁶,3-oxathiazolidine-3-carboxylate

To a solution of the product of Example II-2C (10 g, 24.14 mmol) in acetonitrile (100 mL) and water (24 mL) at 0° C. was added ruthenium(III) chloride, H₂O (0.027 g, 0.121 mmol) followed by sodium periodate (7.74 g, 36.2 mmol). After 45 minutes, the mixture was filtered through a plug of diatomaceous earth and the solid was washed with ethyl acetate (5×50 mL). The filtrate was stirred with a saturated aqueous solution of sodium thiosulfate (150 mL) for 10 minutes. The layers were then separated, and the aqueous layer extracted with ethyl acetate (2×200 mL). The combined organic layers were washed with brine (200 mL), dried over sodium sulfate, filtered, and concentrated under reduced pressure. The crude residue was subjected to column chromatography (SiO₂, 120 g, 0-100% dichloromethane in isohexane) to afford the title compound (9.518 g, 22.77 mmol, 94% yield). ¹H NMR (400 MHz, CDCl₃) δ ppm 4.69 (dd, J=9.2, 2.1 Hz, 1H), 4.61 (ddd, J=9.2, 5.9, 0.9 Hz, 1H), 4.29 (dddd, J=8.3, 6.1, 4.1, 2.1 Hz, 1H), 3.97 (ddd, J=9.9, 4.1, 0.9 Hz, 1H), 3.86 (dd, J=9.9, 8.7 Hz, 1H), 1.55 (s, 9H), 1.16-1.01 (m, 21H).

### Example II-2E: tert-butyl [(2R)-1-[4-(benzyloxy)-6-bromo-2-fluoro-3-(2,2,2-trifluoroacetamido)phenyl]-3-{[tri(propan-2-yl)silyl]oxy}propan-2-yl]carbamate

To a solution of diisopropylamine (7.5 mL, 53.1 mmol) in tetrahydrofuran (80 mL) was added butyllithium (1.73 M in hexanes) (30 mL, 51.9 mmol) dropwise at −78° C. over 15 minutes, keeping the temperature below −63° C. The solution was stirred at −78° C. for 30 minutes before a solution of the product of Example I-3C (10 g, 24.23 mmol) in tetrahydrofuran (40 mL) was added dropwise over 45 minutes with a syringe pump, keeping the temperature below −67° C. The resulting solution was stirred at −78° C. for 2 hours before a solution of the product of Example II-2D (10.6 g, 25.5 mmol) in tetrahydrofuran (40 mL) was added dropwise over 30 minutes with a syringe pump, keeping the temperature below −67° C. The resulting solution was left to stir at −78° C. for 1 hour, then quenched slowly with 1 M aqueous hydrochloric acid (120 mL, 120 mmol) and stirred vigorously for 5 minutes at room temperature. The biphasic mixture was diluted with ethyl acetate (100 mL) and stirred for 5 minutes. The layers were separated, and the aqueous layer was extracted with ethyl acetate (3×100 mL). The combined organic layers were dried over sodium sulfate, filtered, and concentrated under reduced pressure. The crude residue was subjected to column chromatography (Reveleris® 330 g reversed phase (C18) flash cartridge, dry loaded with diatomaceous earth, 50-100% acetonitrile in water 0.1% trifluoroacetic acid) to afford the title compound (9.75 g, 13.51 mmol, 53% yield). ¹H NMR (400 MHz, DMSO-d₆) δ ppm 11.03 (s, 1H), 7.43-7.28 (m, 6H), 6.55-6.45 (m, 1H), 5.20 (s, 2H), 3.97-3.81 (m, 1H), 3.62 (d, J=6.4 Hz, 2H), 2.99-2.82 (m, 1H), 2.79-2.63 (m, 1H), 1.28 (s, 9H), 1.10-0.95 (m, 21H); MS (ESI⁺) m/z 621/623 [M-C(O)OC(CH₃)₃+H]⁺.

### Example II-2F: tert-butyl [{6-(benzyloxy)-4-bromo-3-[(2R)-2-[(tert-butoxycarbonyl)amino]-3-{[tri(propan-2-yl)silyl]oxy}propyl]-2-fluorophenyl}(trifluoroacetyl)amino]acetate

To a solution of the product of Example II-2E (15.14 g, 19.30 mmol) in acetone (150 mL) were added tert-butyl 2-bromoacetate (3.4 mL, 23.02 mmol), potassium carbonate (6.0 g, 43.46 mmol) and potassium iodide (3.86 g, 23.26 mmol). The resulting suspension was stirred at 55° C. for 2 hours and then allowed to cool to room temperature. The solvent was removed, and the residue was partitioned between a saturated aqueous solution of ammonium chloride (100 mL) and ethyl acetate (100 mL). The layers were separated, and the aqueous layer was extracted with ethyl acetate (3×50 mL). The combined organic layers were dried over sodium sulfate, filtered, and the solvent was removed under reduced pressure. The crude residue was subjected to column chromatography (SiO₂, 220 g, 0-30% ethyl acetate in isohexane) to afford the title compound (12.82 g, 15.30 mmol, 77%). ¹H NMR (400 MHz, DMSO-d₆) δ ppm 7.47-7.29 (m, 6H), 6.52 (d, J=9.2 Hz, 0.4H), 6.37 (d, J=9.5 Hz, 0.6H), 5.29-5.14 (m, 2H), 4.42-4.28 (m, 1H), 4.00-3.91 (m, 1H), 3.91-3.77 (m, 1H), 3.68-3.51 (m, 2H), 2.98-2.84 (m, 1H), 2.81-2.64 (m, 1H), 1.39 (s, 4H), 1.38 (s, 5H), 1.30 (s, 4H), 1.26 (s, 5H), 1.05-0.96 (m, 21H); MS (ESI⁺) m/z 858 [M+Na]⁺.

### Example II-2G: tert-butyl (2R)-6-(benzyloxy)-5-[(2-tert-butoxy-2-oxoethyl)(trifluoroacetyl)amino]-4-fluoro-2-({[tri(propan-2-yl)silyl]oxy}methyl)-2,3-dihydro-1H-indole-1-carboxylate

To a solution of the product of Example II-2F (13.9 g, 15.30 mmol) in dioxane (40 mL) was added a solution of cesium carbonate (9.97 g, 30.6 mmol) in water (4 mL) and BrettPhos (1.232 g, 2.295 mmol). The resulting mixture was degassed under vacuum and backfilled with nitrogen three times. BrettPhos Pd G3 (1.387 g, 1.530 mmol) was added to the mixture, and the reaction mixture was degassed under vacuum and backfilled with nitrogen three times. The reaction mixture was then stirred at 70° C. for 16 hours. The reaction mixture was then cooled to room temperature and then filtered through a short pad of diatomaceous earth that was rinsed with ethyl acetate (3×30 mL). The filtrate was washed with water (30 mL) and then brine (30 mL). The organic layer was dried over sodium sulfate, filtered, and concentrated in vacuo. The crude residue was subjected to column chromatography (SiO₂, 220 g cartridge, 0-50% (toluene with 5% tert-butyl methyl ether) in isohexane). The resultant crude residue was re-subjected to column chromatography (SiO₂, 220 g cartridge, 0-10% tetrahydrofuran in isohexane) to afford the title compound (10.10 g, 13.38 mmol, 79% yield).

### Example II-2H: tert-butyl (2R)-6-(benzyloxy)-5-[(2-tert-butoxy-2-oxoethyl)(trifluoroacetyl)amino]-4-fluoro-2-(hydroxymethyl)-2,3-dihydro-1H-indole-1-carboxylate

To a solution of the product of Example II-2G (4.6 g, 5.61 mmol) in tetrahydrofuran (30 mL) at room temperature was added tetra-N-butylammonium fluoride, 1 M in tetrahydrofuran (6.2 mL, 6.17 mmol). The resulting solution was stirred at room temperature for 1 hour. The reaction mixture was concentrated in vacuo. The crude residue was subjected to column chromatography (SiO₂, 80 g, dry loaded with silica, 0-60% ethyl acetate in isohexane) to afford the title compound (3.42 g, 5.09 mmol, 91% yield, 89% purity). ¹H NMR (400 MHz, DMSO-d₆) δ ppm 7.52-7.28 (m, 6H), 5.21-5.04 (m, 2H), 4.98 (dt, J=11.3, 5.9 Hz, 1H), 4.51-4.35 (m, 2H), 4.09-3.91 (m, 1H), 3.60-3.42 (m, 2H), 3.24-3.11 (m, 2H), 1.50 (s, 9H), 1.38 (s, 9H); MS (ESI⁺) m/z 622 [M+Na]⁺.

### Example II-2I: tert-butyl (2R)-6-(benzyloxy)-5-[(2-tert-butoxy-2-oxoethyl)(trifluoroacetyl)amino]-4-fluoro-2-formyl-2,3-dihydro-1H-indole-1-carboxylate

Dess-Martin periodinane (1,1,1-tris(acetyloxy)-1,1-dihydro-1,2-benzodioxol-3-(1H)-one) (2.379 g, 5.61 mmol) was added to a solution of the product of Example II-2H (3.43 g, 5.10 mmol) in dichloromethane (25 mL) at 0° C. The reaction mixture was then allowed to slowly warm to room temperature and stirred for 3 hours. Saturated aqueous solutions of sodium thiosulfate (15 mL) and sodium bicarbonate (15 mL) were added, and the biphasic mixture was stirred for 15 minutes. It was then diluted with dichloromethane (30 mL). The layers were separated, and the aqueous layer was extracted with dichloromethane (2×20 mL). The combined organic layers were washed with a saturated aqueous solution of sodium thiosulfate (3×15 mL), a saturated aqueous solution of sodium bicarbonate (3×15 mL), and brine (15 mL). The organic fraction was dried over magnesium sulfate, filtered, and concentrated in vacuo to afford the title compound (3.44 g, 4.61 mmol, 90% yield, 80% purity). ¹H NMR (400 MHz, DMSO-d₆ at 90° C.) δ ppm 9.63 (dd, J=7.0, 1.3 Hz, 1H), 7.54-7.26 (m, 6H), 5.30-5.01 (m, 3H), 4.42 (dd, J=16.5, 3.3 Hz, 1H), 3.90 (dd, J=16.5, 3.9 Hz, 1H), 3.46-3.31 (m, 1H), 3.16 (ddd, J=15.9, 10.4, 4.9 Hz, 1H), 1.50 (s, 9H), 1.39 (d, J=4.4 Hz, 9H).

### Example II-2J: tert-butyl (2R)-6-(benzyloxy)-5-[(2-tert-butoxy-2-oxoethyl)(trifluoroacetyl)amino]-4-fluoro-2-{[(2-methylpropyl)amino]methyl}-2,3-dihydro-1H-indole-1-carboxylate

To a solution of the product of Example II-2I (3.92 g, 5.26 mmol) in 1,2-dichloroethane (24 mL) was added 2-methylpropan-1-amine (1.567 mL, 15.77 mmol) followed by acetic acid (903 μL, 15.77 mmol). The resulting solution was stirred at room temperature for 40 minutes. Sodium triacetoxyborohydride (1.671 g, 7.89 mmol) was then added, and the mixture was stirred for 1 hour. The reaction mixture was quenched with a saturated aqueous solution of sodium bicarbonate (10 mL) and stirred for 10 minutes. The mixture was then diluted with water (100 mL) and extracted with ethyl acetate (3×100 mL). The combined organic layers were washed with brine (50 mL), dried over sodium sulfate, filtered, and concentrated in vacuo to afford the title compound (4.01 g, 5.28 mmol, 100% yield, 86% purity). ¹H NMR (400 MHz, DMSO-d₆) δ ppm 7.50-7.24 (m, 6H), 5.23-5.03 (m, 2H), 4.54-4.35 (m, 2H), 3.96 (dd, J=16.7, 2.3 Hz, 1H), 3.90 (s, 2H), 3.23-3.08 (m, 1H), 3.07-2.95 (m, 1H), 2.81-2.70 (m, 1H), 2.41-2.23 (m, 2H), 1.66-1.55 (m, 1H), 1.50 (s, 9H), 1.38 (s, 9H), 0.96-0.74 (m, 6H); MS (ESI⁺) m/z 654 [M+H]⁺.

### Example II-2K: tert-butyl (2R)-6-(benzyloxy)-2-{[(tert-butoxycarbonyl)(2-methylpropyl)amino]methyl}-5-[(2-tert-butoxy-2-oxoethyl)(trifluoroacetyl)amino]-4-fluoro-2,3-dihydro-1H-indole-1-carboxylate

To a solution of product of Example II-2J (4.01 g, 5.28 mmol) in acetonitrile (30 mL) at room temperature was added N,N-diisopropylethylamine (2.76 mL, 15.83 mmol) and di-tert-butyl dicarbonate (2.303 g, 10.55 mmol). The mixture was stirred at room temperature for 30 minutes. The reaction was quenched with water (30 mL). The aqueous layer was extracted with ethyl acetate (3×30 mL). The combined organic layers were washed with brine (30 mL), dried over sodium sulfate, filtered, and concentrated under reduced pressure. The crude residue was subjected to column chromatography (SiO₂, 80 g cartridge, 0-20% ethyl acetate in isohexane) to afford the title compound (3.59 g, 4.52 mmol, 86% yield). ¹H NMR (400 MHz, DMSO-d₆ at 90° C.) δ ppm 7.54-7.24 (m, 6H), 5.24-5.06 (m, 2H), 4.78-4.63 (m, 1H), 4.44 (dd, J=16.5, 9.6 Hz, 1H), 3.98-3.80 (m, 1H), 3.52-3.28 (m, 2H), 3.24-3.10 (m, 1H), 3.06-2.84 (m, 3H), 1.95-1.85 (m, 1H), 1.54 (d, J=2.3 Hz, 9H), 1.46-1.29 (m, 18H), 0.94-0.78 (m, 6H); MS (ESI⁺) m/z 777 [M+Na]⁺.

### Example II-2L: tert-butyl (2R)-6-(benzyloxy)-2-{[(tert-butoxycarbonyl)(2-methylpropyl)amino]methyl}-5-[(2-tert-butoxy-2-oxoethyl)amino]-4-fluoro-2,3-dihydro-1H-indole-1-carboxylate

Lithium hydroxide (2 M aqueous) (5.71 mL, 11.41 mmol) was added to a solution of the product of Example II-2K (3.019 g, 3.80 mmol) in methanol (10 mL) and tetrahydrofuran (10 mL). The reaction mixture was stirred at room temperature for 1 hour. The reaction mixture was neutralized to pH 7 with concentrated hydrochloric acid. The reaction was diluted with ethyl acetate (50 mL) and water (50 mL). The layers were separated, and the aqueous layer extracted with ethyl acetate (2×50 mL). The combined organic layers were washed with brine (50 mL), dried over sodium sulfate, filtered, and concentrated in vacuo to afford the title compound (2.77 g, 3.71 mmol, 97% yield, 88% purity). ¹H NMR (400 MHz, DMSO-d₆ at 90° C.) δ ppm 7.54-7.24 (m, 5H), 7.18 (s, 1H), 5.09 (s, 2H), 4.61 (dddd, J=9.3, 7.5, 5.6, 2.1 Hz, 1H), 3.85 (d, J=2.0 Hz, 2H), 3.45-3.21 (m, 2H), 3.08-3.01 (m, 1H), 3.00 (d, J=7.4 Hz, 2H), 2.84 (dd, J=15.8, 2.1 Hz, 1H), 1.99-1.81 (m, 1H), 1.60-1.46 (m, 9H), 1.46-1.22 (m, 18H), 0.94-0.74 (m, 6H); MS (ESI⁺) m/z 658 [M+H]⁺.

### Example II-2M: tert-butyl (2R)-6-(benzyloxy)-2-{[(tert-butoxycarbonyl)(2-methylpropyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.6 mL, 6.91 mmol) in dichloromethane (18 mL) at 0° C. was added allyl alcohol (0.5 mL, 7.35 mmol) dropwise. The resulting solution was stirred for 20 minutes at 0° C. before a prepared solution of the product of Example II-2L (2.77 g, 3.71 mmol) and triethylamine (1.3 mL, 9.33 mmol) in dichloromethane (18 mL) was added dropwise. The resulting solution was stirred at room temperature for 30 minutes. The reaction mixture was quenched with water (50 mL) and diluted with dichloromethane (50 mL). The layers were separated, and the aqueous layer was extracted with dichloromethane (3×50 mL). The combined organic layers were dried over magnesium sulfate, filtered, and concentrated in vacuo. The crude residue was subjected to column chromatography (SiO₂, 80 g cartridge, 0-40% tert-butyl methyl ether in isohexane) to afford the title compound (2.7 g, 2.80 mmol, 66% yield). ¹H NMR (400 MHz, DMSO-d₆ at 90° C.) δ ppm 7.58-7.13 (m, 6H), 6.45 (s, 1H), 6.02-5.83 (m, 1H), 5.31-4.96 (m, 4H), 4.69 (s, 1H), 4.30-4.19 (m, 1H), 3.96 (dt, J=4.8, 1.7 Hz, 1H), 3.31 (dd, J=13.9, 5.9 Hz, 1H), 3.20-2.96 (m, 3H), 2.86 (t, J=16.7 Hz, 1H), 1.97-1.84 (m, 1H), 1.61-1.44 (m, 10H), 1.44-1.25 (m, 18H), 1.13 (d, J=4.1 Hz, 1H), 0.96-0.77 (m, 7H); MS (ESI⁺) m/z 844 [M+Na]⁺.

### Example II-2N: tert-butyl (2R)-6-(benzyloxy)-2-{[(tert-butoxycarbonyl)(2-methylpropyl)amino]methyl}-4-fluoro-5-(1,1,4-trioxo-1λ⁶,2,5-thiadiazolidin-2-yl)-2,3-dihydro-1H-indole-1-carboxylate-ammonia

To a solution of the product of Example II-2M (2.7 g, 2.8 mmol) and potassium carbonate (1.545 mg, 11.18 mmol) in methanol (15 mL) was added tetrakis(triphenylphosphine)palladium(0) (323 mg, 0.280 mmol). The reaction mixture was stirred at 60° C. for 1 hour. The mixture was cooled to room temperature and diluted with methanol (10 mL). Diatomaceous earth was added, and the mixture was concentrated in vacuo. The crude residue was subjected to column chromatography (Reveleris® 80 g reversed phase (C18) flash cartridge, dry loaded with diatomaceous earth, 5-70% methanol in 10 mM ammonium bicarbonate) to afford the title compound as an ammonium salt (1.50 g, 1.765 mmol, 63% yield). ¹H NMR (400 MHz, DMSO-d₆ at 90° C.) δ ppm 7.72-7.14 (m, 6H), 5.16-5.02 (m, 2H), 4.74-4.62 (m, 1H), 3.95 (d, J=13.0 Hz, 1H), 3.88 (d, J=13.0 Hz, 1H), 3.47-3.25 (m, 2H), 3.18-2.93 (m, 6H), 2.91-2.79 (m, 1H), 1.94 (hept, J=6.9 Hz, 1H), 1.52 (s, 9H), 1.37 (s, 9H), 0.84 (dd, J=6.7, 1.6 Hz, 6H); MS (ESI⁺) m/z 685 [M+Na]⁺.

### Example II-2O: tert-butyl (2R)-2-{[(tert-butoxycarbonyl)(2-methylpropyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a suspension of the product of Example II-2N (500 mg, 0.588 mmol) in degassed water (0.1 mL) and ethanol (4 mL) was added 10% Pd/C (125 mg). The resulting suspension was allowed to stir under hydrogen (5 bar) for 24 hours. The reaction mixture was filtered through a Whatman glass fiber filter paper and washed with ethanol (3×20 mL). The filtrate was concentrated in vacuo. The crude residue was subjected to column chromatography (Reveleris® 40 g reversed phase (C18) flash cartridge, dry loaded with diatomaceous earth, 5-55% methanol in 10 mM ammonium bicarbonate) to afford the title compound as an ammonium salt (188 mg, 0.303 mmol, 52% yield). ¹H NMR (400 MHz, DMSO-d₆ at 90° C.) ppm 7.77-7.02 (br s, 4H) 7.00 (s, 1H), 4.64 (dddd, J=9.2, 7.4, 5.6, 2.0 Hz, 1H), 3.99-3.82 (m, 2H), 3.35 (qd, J=14.1, 6.4 Hz, 2H), 3.13-2.93 (m, 3H), 2.82 (dd, J=15.7, 2.0 Hz, 1H), 2.04 (s, 1H), 1.94 (hept, J=6.9 Hz, 1H), 1.53 (s, 9H), 1.37 (s, 9H), 0.84 (dd, J=6.7, 1.8 Hz, 6H); MS (ESI⁺) m/z 595 [M+Na]⁺.

### Example II-2P: 5-[(2R)-4-fluoro-6-hydroxy-2-{[(2-methylpropyl)amino]methyl}-2,3-dihydro-1H-indol-5-yl]-1λ⁶,2,5-thiadiazolidine-1,1,3-trione

To a solution of the product of Example II-20 (80 mg, 0.129 mmol) in dichloromethane (1 mL) at room temperature was added trifluoroacetic acid (0.1 mL, 1.298 mmol). The reaction mixture was stirred at room temperature for 2 hours. A second aliquot of trifluoroacetic acid (0.1 mL, 1.298 mmol) was added and the reaction stirred for a further 2 hours. A third aliquot of trifluoroacetic acid (0.1 mL, 1.298 mmol) was added and the reaction stirred for a further 30 minutes. The reaction mixture was neutralized with 0.7 M NH₃ in methanol:water (95:5) (7 mL) and concentrated under reduced pressure onto diatomaceous earth (1 g). The crude residue was subjected to column chromatography (Reveleris® 24 g, reversed phase (C18) flash cartridge, dry loaded with diatomaceous earth, 5-20% methanol in 10 mM ammonium bicarbonate) to afford the title compound (34 mg, 0.089 mmol, 69% yield). ¹H NMR (400 MHz, DMSO-d₆) δ ppm 8.68 (s, 1H), 8.11 (br s, 2H), 6.02 (s, 1H), 5.92 (s, 1H), 4.26-4.04 (m, 1H), 3.84 (s, 2H), 3.10 (dd, J=15.5, 9.3 Hz, 1H), 3.06-2.93 (m, 2H), 2.83-2.76 (m, 2H), 2.73 (dd, J=15.5, 6.8 Hz, 1H), 1.96 (hept, J=6.4 Hz, 1H), 0.95 (d, J=6.7 Hz, 6H); MS (ESI⁺) m/z 373 [M+H]⁺.

### Example II-3: 5-[(2R)-2-{[(2-ethylbutyl)amino]methyl}-4-fluoro-6-hydroxy-2,3-dihydro-1H-indol-5-yl]-1λ⁶,2,5-thiadiazolidine-1,1,3-trione (Compound 134)

### Example II-3A: tert-butyl (2R)-6-(benzyloxy)-5-[(2-tert-butoxy-2-oxoethyl)(trifluoroacetyl)amino]-2-{[(2-ethylbutyl)amino]methyl}-4-fluoro-2,3-dihydro-1H-indole-1-carboxylate

Acetic acid (0.25 mL, 4.37 mmol) was added to a solution of 2-ethylbutan-1-amine (0.59 mL, 4.43 mmol) and the product of Example II-2I (1.06 g, 1.475 mmol) in 1,2-dichloroethane (6 mL). The reaction mixture was stirred at room temperature for 40 minutes. Sodium triacetoxyborohydride (0.47 g, 2.218 mmol) was added, and the reaction mixture was stirred for 30 minutes. The reaction mixture was quenched with a saturated aqueous solution of sodium bicarbonate (3 mL) and stirred for 10 minutes. The reaction was diluted with water (20 mL) and extracted with ethyl acetate (3×20 mL). The combined organic layers were washed with brine (20 mL), dried over sodium sulfate, filtered, and concentrated under reduced pressure to afford the title compound (0.95 g, 1.282 mmol, 87% yield, 92% purity). ¹H NMR (400 MHz, DMSO-d₆) δ ppm 7.52-7.21 (m, 6H), 5.26-5.02 (m, 2H), 4.55-4.31 (m, 2H), 4.11-3.83 (m, 2H), 3.22-2.87 (m, 2H), 2.83-2.67 (m, 1H), 2.45-2.27 (m, 2H), 2.04-1.93 (m, 1H), 1.56-1.44 (m, 9H), 1.42-1.33 (m, 9H), 1.26-1.15 (m, 4H), 0.89-0.68 (m, 6H); MS (ESI⁺) m/z 683 [M+H]⁺.

### Example II-3B: tert-butyl (2R)-6-(benzyloxy)-2-{[(tert-butoxycarbonyl)(2-ethylbutyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-3A (0.95 g, 1.282 mmol) in acetonitrile (6 mL) at room temperature was added N,N-diisopropylethylamine (0.672 mL, 3.85 mmol) and di-tert-butyl dicarbonate (0.560 g, 2.56 mmol). The mixture was stirred at room temperature for 1 hour. The reaction was quenched with half saturated brine (30 mL). The mixture was diluted with ethyl acetate (20 mL), and the layers were separated. The aqueous layer was further extracted with ethyl acetate (2×20 mL). The combined organic layers were washed with brine (10 mL), dried over sodium sulfate, filtered, and concentrated under reduced pressure. The crude residue was subjected to column chromatography (SiO₂, 80 g cartridge, 0-40% ethyl acetate in isohexane, 60 mL/minute) to afford the title compound (927 mg, 1.126 mmol, 88% yield). ¹H NMR (400 MHz, DMSO-d₆) δ ppm 7.46-7.27 (m, 6H), 5.23-5.02 (m, 2H), 4.77-4.60 (m, 1H), 4.44-4.37 (m, 1H), 4.00-3.90 (m, 1H), 3.55-2.71 (m, 7H), 1.54-1.44 (m, 9H), 1.42-1.36 (m, 9H), 1.36-1.11 (m, 13H), 0.89-0.70 (m, 6H).

### Example II-3C: tert-butyl (2R)-6-(benzyloxy)-2-{[(tert-butoxycarbonyl)(2-ethylbutyl)amino]methyl}-5-[(2-tert-butoxy-2-oxoethyl)amino]-4-fluoro-2,3-dihydro-1H-indole-1-carboxylate

To a solution of the product of Example II-3B (927 mg, 1.126 mmol) in methanol (2.8 mL) and tetrahydrofuran (2.80 mL) at room temperature was added 2 M aqueous lithium hydroxide (1.689 mL, 3.38 mmol). The reaction mixture was then stirred at room temperature for 1 hour. The reaction mixture was neutralized to pH 7 with 1 M aqueous hydrochloric acid and diluted with water (10 mL). The aqueous layer was extracted with ethyl acetate (3×10 mL). The combined organic layers were washed with brine (10 mL), dried over sodium sulfate, filtered, and concentrated in vacuo to afford the title compound (747 mg, 0.980 mmol, 87% yield, 90% purity). ¹H NMR (400 MHz, DMSO-d₆ at 90° C.) δ ppm 7.55-7.27 (m, 5H), 7.16 (s, 1H), 5.24-5.03 (m, 2H), 4.80-4.59 (m, 1H), 4.55-4.37 (m, 1H), 3.95-3.78 (m, 2H), 3.44-3.24 (m, 2H), 3.21-3.03 (m, 3H), 2.91-2.75 (m, 1H), 1.70-1.46 (m, 9H), 1.46-1.32 (m, 18H), 1.32-1.13 (m, 5H), 0.84 (tt, J=7.5, 1.9 Hz, 6H); MS (ESI⁺) m/z 587 [M+H—C(O)OC(CH₃)₃]⁺

### Example II-3D: tert-butyl (2R)-6-(benzyloxy)-2-{[(tert-butoxycarbonyl)(2-ethylbutyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.16 mL, 1.843 mmol) in dichloromethane (4.901 mL) at 0° C. was added allyl alcohol (0.13 mL, 1.912 mmol) dropwise. The resulting solution was stirred for 20 minutes at 0° C. before a prepared solution of the product of Example II-3C (747 mg, 0.980 mmol) and triethylamine (0.34 mL, 2.439 mmol) in dichloromethane (4.90 mL) was added dropwise. The resulting solution was stirred at 0° C. for 30 minutes. The reaction mixture was quenched with water (50 mL) and diluted with dichloromethane (50 mL). The layers were separated, and the aqueous layer was extracted with dichloromethane (3×50 mL). The combined organic layers were dried over sodium sulfate, filtered, and concentrated under reduced pressure. The crude residue was subjected to column chromatography (SiO₂, 40 g cartridge, 0-40% tert-butyl methyl ether in isohexane, 90 mL/minute) to afford the title compound (667 mg, 0.707 mmol, 72% yield, 90% purity). ¹H NMR (400 MHz, DMSO-d₆) δ ppm 11.67-11.44 (m, 1H), 7.67-7.00 (m, 6H), 5.80-5.61 (m, 1H), 5.27-4.99 (m, 4H), 4.77-4.56 (m, 3H), 4.32-4.07 (m, 3H), 3.27-2.87 (m, 4H), 2.87-2.64 (m, 1H), 1.47 (s, 9H), 1.44-1.05 (m, 23H), 0.89-0.73 (m, 6H); MS (ESI⁻) m/z 848 [M−H]⁻.

### Example II-3E: tert-butyl (2R)-6-(benzyloxy)-2-{[(tert-butoxycarbonyl)(2-ethylbutyl)amino]methyl}-4-fluoro-5-(1,1,4-trioxo-1λ⁶,2,5-thiadiazolidin-2-yl)-2,3-dihydro-1H-indole-1-carboxylate-ammonia

A solution of the product of Example II-3D (0.667 g, 0.707 mmol) and potassium carbonate (0.4 g, 2.89 mmol) in methanol (7 mL) was degassed under vacuum and backfilled with nitrogen three times. To the mixture was added tetrakis(triphenylphosphine)palladium(0) (0.082 g, 0.071 mmol), and the reaction mixture was degassed under vacuum and backfilled with nitrogen three times. The reaction mixture was stirred at 60° C. for 4 hours. The mixture was cooled to room temperature. C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 0.1% ammonium hydroxide, 40 mL/minute) to afford the title compound as an ammonium salt (444 mg, 0.572 mmol, 81% yield, 89% purity). ¹H NMR (400 MHz, DMSO-d₆) δ ppm 7.57-7.44 (m, 2H), 7.43-7.25 (m, 3H), 7.20 (s, 1H), 5.17-5.05 (m, 2H), 4.78-4.56 (m, 1H), 3.94 (d, J=13.0 Hz, 1H), 3.87 (d, J=13.0 Hz, 1H), 3.45-3.27 (m, 2H), 3.21 (s, 1H), 3.15-2.92 (m, 3H), 2.91-2.77 (m, 1H), 1.65-1.55 (m, 1H), 1.51 (s, 9H), 1.37 (s, 9H), 1.32-1.03 (m, 4H), 0.93-0.78 (m, 6H); MS (ESI⁺) m/z 714 [M+Na]⁺.

### Example II-3F: tert-butyl (2R)-2-{[(tert-butoxycarbonyl)(2-ethylbutyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a suspension of the product of Example II-3E (444 mg, 0.558 mmol) in deoxygenated water (0.1 mL) and ethanol (4 mL) was added 10% Pd/C (120 mg). The resulting suspension was allowed to stir under hydrogen (5 bar) for 18 hours. The reaction mixture was filtered through a Whatman glass fiber filter paper and washed with ethanol (3×20 mL). The filtrate was concentrated under reduced pressure onto C18 silica (1.5 g). The crude residue was subjected to column chromatography (Reveleris® 40 g reversed phase (C18) flash cartridge, dry loaded with C18 silica, 5-55% methanol in 10 mM ammonium bicarbonate, 45 mL/minute) to afford the title compound (270 mg, 0.427 mmol, 76% yield) as an ammonium salt. ¹H NMR (400 MHz, DMSO-d₆ at 90° C.) δ ppm 6.93 (s, 1H), 4.70-4.58 (m, 1H), 3.95 (d, J=13.0 Hz, 1H), 3.89 (d, J=13.0 Hz, 1H), 3.41-3.26 (m, 2H), 3.17-3.00 (m, 3H), 2.78 (dd, J=15.6, 2.0 Hz, 1H), 1.60 (dt, J=13.0, 6.9 Hz, 1H), 1.53 (s, 9H), 1.38 (s, 9H), 1.33-1.16 (m, 4H), 0.89-0.78 (m, 6H); MS (ESI⁺) m/z 624 [M+Na]⁺.

### Example II-3G: 5-[(2R)-2-{[(2-ethylbutyl)amino]methyl}-4-fluoro-6-hydroxy-2,3-dihydro-1H-indol-5-yl]-1λ⁶,2,5-thiadiazolidine-1,1,3-trione

To a solution of the product of Example II-3F (106 mg, 0.163 mmol) in dichloromethane (1 mL) at room temperature was added trifluoroacetic acid (0.188 mL, 2.445 mmol). The reaction mixture was stirred at room temperature for 5 hours. A second aliquot of trifluoroacetic acid (0.188 mL, 2.445 mmol) was added and reaction mixture was stirred at room temperature for a further 1 hour. The reaction mixture was neutralized with 0.7 M NH₃ in methanol:water (95:5) (7 mL) and concentrated under reduced pressure. The crude residue was subjected to column chromatography (Reveleris® 24 g reversed phase (C18) flash cartridge, dry loaded with C18 silica, 5-20% methanol in 0.1% ammonium hydroxide, 35 mL/minute) to afford the title compound (59 mg, 0.147 mmol, 84% yield). ¹H NMR (400 MHz, DMSO-d₆) δ ppm 8.63 (s, 1H), 8.01 (s, 1H), 5.99 (s, 1H), 5.90 (s, 1H), 4.08 (s, 1H), 3.83 (s, 2H), 3.06 (dd, J=15.5, 9.3 Hz, 1H), 2.92 (s, 2H), 2.82-2.64 (m, 3H), 1.91 (s, 1H), 1.53 (s, 1H), 1.45-1.21 (m, 4H), 0.85 (t, J=7.4 Hz, 6H); MS (ESI⁺) m/z 401 [M+H]⁺.

### Example II-4: 5-{(2R)-4-fluoro-6-hydroxy-2-[(propylamino)methyl]-2,3-dihydro-1H-indol-5-yl}-1λ⁶,2,5-thiadiazolidine-1,1,3-trione (Compound 135)

### Example II-4A: tert-butyl (2R)-6-(benzyloxy)-5-[(2-tert-butoxy-2-oxoethyl)(trifluoroacetyl)amino]-4-fluoro-2-[(propylamino)methyl]-2,3-dihydro-1H-indole-1-carboxylate

Acetic acid (0.252 mL, 4.40 mmol) was added to a solution of the product of Example II-2I (0.92 g, 1.465 mmol) and propan-1-amine (0.36 mL; 4.4 mmol) in 1,2-dichloroethane (6 mL). The reaction mixture was stirred at room temperature for 1 hour. Sodium triacetoxyborohydride (0.466 g, 2.198 mmol) was added, and the reaction mixture was stirred for 1 hour. The reaction mixture was quenched with a saturated aqueous solution of sodium bicarbonate (20 mL), and the resultant mixture was stirred for 10 minutes. The mixture was diluted with water (20 mL) and extracted with ethyl acetate (3×30 mL). The combined organic layers were washed with brine (20 mL), dried over sodium sulfate, filtered, and concentrated under reduced pressure to afford the title compound (983 mg, 1.46 mmol, 99% yield). ¹H NMR (400 MHz, DMSO-d₆) δ ppm 7.48-7.28 (m, 6H), 5.22-5.04 (m, 2H), 4.52-4.44 (m, 1H), 4.40 (dd, J=16.7, 4.0 Hz, 1H), 3.97 (dd, J=16.5, 3.0 Hz, 1H), 3.22-3.08 (m, 1H), 3.06-2.94 (m, 1H), 2.80-2.70 (m, 1H), 2.60-2.38 (m, 3H), 1.50 (s, 9H), 1.38 (s, 11H), 0.87-0.77 (m, 3H); MS (ESI⁺) m/z 640 [M+H]⁺.

### Example II-4B: tert-butyl (2R)-6-(benzyloxy)-2-{[(tert-butoxycarbonyl)(propyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-4A (983 mg, 1.460 mmol) in acetonitrile (6 mL) at room temperature was added N,N-diisopropylethylamine (0.765 mL, 4.38 mmol) and di-tert-butyl dicarbonate (637 mg, 2.92 mmol). The mixture was stirred at room temperature for 1 hour. The reaction was quenched with half saturated brine (30 mL). The mixture was diluted with ethyl acetate (20 mL), and the layers were separated. The aqueous layer was extracted with ethyl acetate (2×20 mL). The combined organic layers were washed with brine (10 mL), dried over sodium sulfate, filtered, and concentrated under reduced pressure. The crude residue was subjected to column chromatography (SiO₂, 80 g cartridge, 0-40% ethyl acetate in isohexane, 60 mL/minute) to afford the title compound (831 mg, 1.090 mmol, 75% yield). ¹H NMR (400 MHz, DMSO-d₆) δ ppm 7.46-7.29 (m, 6H), 5.21-5.04 (m, 2H), 4.74-4.59 (m, 1H), 4.40 (d, J=16.8 Hz, 1H), 3.98 (d, J=17.2 Hz, 1H), 3.29-3.01 (m, 4H), 3.01-2.77 (m, 2H), 1.56-1.43 (m, 11H), 1.38 (s, 9H), 1.36-1.24 (m, 9H), 0.81 (t, J=7.3 Hz, 3H).

### Example II-4C: tert-butyl (2R)-6-(benzyloxy)-2-{[(tert-butoxycarbonyl)(propyl)amino]methyl}-5-[(2-tert-butoxy-2-oxoethyl)amino]-4-fluoro-2,3-dihydro-1H-indole-1-carboxylate

To a solution of the product of Example II-4B (894 mg, 1.172 mmol) in methanol (4 mL) and tetrahydrofuran (4 mL) at room temperature was added a solution of lithium hydroxide monohydrate (246 mg, 5.86 mmol) in water (4 mL). The reaction mixture was then stirred at room temperature for 40 minutes. The reaction mixture was neutralized to pH 7 with 1 M hydrogen chloride (5.86 mL, 5.86 mmol) and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (778 mg, 1.088 mmol, 93% yield, 90% purity). ¹H NMR (400 MHz, DMSO-d₆) δ ppm 7.56-7.18 (m, 6H), 5.22-4.95 (m, 2H), 4.63-4.51 (m, 1H), 3.50-3.16 (m, 2H), 3.16-2.67 (m, 4H), 1.56-1.42 (m, 14H), 1.42-1.20 (m, 17H), 0.86-0.75 (m, 3H).

### Example II-4D: tert-butyl (2R)-6-(benzyloxy)-2-{[(tert-butoxycarbonyl)(propyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.189 mL, 2.175 mmol) in dichloromethane (3 mL) at 0° C. was added allyl alcohol (0.148 mL, 2.175 mmol) dropwise. The resulting solution was stirred for 20 minutes at 0° C. before a prepared solution of the product of Example II-4C (778 mg, 1.088 mmol) and triethylamine (0.379 mL, 2.72 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-100% ethyl acetate in isohexane, 60 mL/minute) to afford the title compound (394 mg, 0.391 mmol, 36% yield, 80% purity). MS (ESI⁻) m/z 805 [M−H]⁻.

### Example II-4E: tert-butyl (2R)-6-(benzyloxy)-2-{[(tert-butoxycarbonyl)(propyl)amino]methyl}-4-fluoro-5-(1,1,4-trioxo-1λ⁶,2,5-thiadiazolidin-2-yl)-2,3-dihydro-1H-indole-1-carboxylate-ammonia

To a solution of the product of Example II-4D (394 mg, 0.391 mmol) and potassium carbonate (220 mg, 1.592 mmol) in methanol (5 mL) was added tetrakis(triphenylphosphine)palladium(0) (45 mg, 0.039 mmol). The reaction mixture was stirred at 60° C. for 1 hour. The mixture was cooled to room temperature and diluted with methanol (10 mL). C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 10 mM ammonium bicarbonate, 60 mL/minute) to afford the title compound as an ammonium salt (125 mg, 0.169 mmol, 43% yield, 90% purity). ¹H NMR (400 MHz, DMSO-d₆) δ ppm 7.75-7.00 (m, 10H), 5.09 (s, 2H), 4.70-4.56 (m, 1H), 4.39-4.30 (m, 1H), 3.99-3.82 (m, 1H), 3.49-3.39 (m, 2H), 3.26-3.20 (m, 1H), 3.16-2.99 (m, 3H), 2.91-2.71 (m, 1H), 1.49 (s, 9H), 1.38 (s, 4H), 1.30 (s, 5H), 0.80 (t, J=7.4 Hz, 3H); MS (ESI⁺) m/z 671 [M+Na]⁺.

### Example II-4F: tert-butyl (2R)-2-{[(tert-butoxycarbonyl)(propyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia (partial)

To a solution of the product of Example II-4E (125 mg, 0.173 mmol) in degassed water (0.5 mL) and ethanol (4 mL) was added 10% Pd/C (25 mg). The resulting suspension was allowed to stir under hydrogen (5 bar) for 18 hours. The suspension was concentrated under reduced pressure onto C18 silica gel (2 g). The crude residue was subjected to column chromatography (Reveleris® 24 g reversed phase (C18) flash cartridge, dry loaded with C18 silica, 10-60% methanol in 10 mM ammonium bicarbonate, 40 mL/minute) to afford the title product as a partial ammonium salt (44 mg, 0.062 mmol, 36% yield, 80% purity). ¹H NMR (400 MHz, DMSO-d₆) δ ppm 9.36 (s, 1H), 7.27-6.59 (m, 3H), 4.65-4.51 (m, 1H), 3.95-3.80 (m, 2H), 3.48-3.39 (m, 2H), 3.24-3.15 (m, 2H), 3.15-2.98 (m, 3H), 2.84-2.69 (m, 1H), 1.50 (s, 9H), 1.37 (s, 4H), 1.32 (s, 5H), 0.81 (t, J=7.4 Hz, 3H); MS (ESI⁺) m/z 581 [M+Na]⁺.

### Example II-4G: 5-{(2R)-4-fluoro-6-hydroxy-2-[(propylamino)methyl]-2,3-dihydro-1H-indol-5-yl}-1λ⁶,2,5-thiadiazolidine-1,1,3-trione

To a solution of the product of Example II-4F (44 mg, 0.079 mmol) in dichloromethane (3 mL) at room temperature was added trifluoroacetic acid (0.073 mL, 0.945 mmol). The reaction mixture was stirred at 25° C. for 4 hours. The mixture was neutralized with 0.7 M ammonia in methanol:water (95:5) (4 mL) and concentrated under reduced pressure. The crude residue was subjected to column chromatography (Reveleris® 12 g reversed phase (C18) flash cartridge, dry load on C18 silica gel, 10-50% methanol in 10 mM ammonium bicarbonate, 30 mL/minute) to afford the title compound (11 mg, 0.031 mmol, 39% yield, 90% purity). ¹H NMR (400 MHz, DMSO-d₆) δ ppm 8.69 (s, 1H), 7.12 (br s, 6H), 6.04 (s, 1H), 5.92 (s, 1H), 4.18-4.04 (m, 1H), 3.84 (s, 2H), 3.19-2.95 (m, 3H), 2.90 (dd, J=9.4, 6.4 Hz, 2H), 2.73 (dd, J=15.6, 7.0 Hz, 1H), 1.70-1.54 (m, 2H), 0.91 (t, J=7.4 Hz, 3H); MS (ESI⁺) m/z 359 [M+H]f.

### Example II-5: 5-[(2R)-4-fluoro-6-hydroxy-2-{[(2-methoxyethyl)amino]methyl}-2,3-dihydro-1H-indol-5-yl]-1λ⁶,2,5-thiadiazolidine-1,1,3-trione (Compound 136)

### Example II-5A: tert-butyl (2R)-6-(benzyloxy)-5-[(2-tert-butoxy-2-oxoethyl)(trifluoroacetyl)amino]-4-fluoro-2-{[(2-methoxyethyl)amino]methyl}-2,3-dihydro-1H-indole-1-carboxylate

Acetic acid (0.252 mL, 4.40 mmol) was added to a solution of the product of Example II-2I (0.92 g, 1.465 mmol) and 2-methoxyethanamine (0.38 mL, 4.4 mmol) in 1,2-dichloroethane (6 mL). The reaction mixture was stirred at room temperature for 1 hour. Sodium triacetoxyborohydride (0.466 g, 2.198 mmol) was added, and the reaction mixture was stirred for 1 hour. The reaction mixture was quenched with a saturated aqueous solution of sodium bicarbonate (20 mL) and stirred for 10 minutes. The mixture was diluted with water (20 mL) and extracted with ethyl acetate (3×30 mL). The combined organic layers were washed with brine (20 mL), dried over sodium sulfate, filtered, and concentrated under reduced pressure to afford the title compound (992 mg, 1.437 mmol, 98% yield). ¹H NMR (400 MHz, DMSO-d₆) δ ppm 7.46-7.29 (m, 6H), 5.21-5.03 (m, 2H), 4.53-4.44 (m, 1H), 4.40 (dd, J=16.7, 2.0 Hz, 1H), 3.99-3.89 (m, 1H), 3.42-3.25 (m, 2H), 3.22 (2 s, J=4.7 Hz, 3H), 3.19-3.10 (m, 1H), 3.03-2.92 (m, 1H), 2.77 (dd, J=11.9, 3.8 Hz, 1H), 2.73-2.53 (m, 3H), 1.51 (s, 9H), 1.38 (s, 9H); MS (ESI⁺) m/z 656 [M+H]⁺.

### Example II-5B: tert-butyl (2R)-6-(benzyloxy)-2-{[(tert-butoxycarbonyl)(2-methoxyethyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-5A (992 mg, 1.437 mmol) in acetonitrile (6 mL) at room temperature was added N,N-diisopropylethylamine (0.753 mL, 4.31 mmol) and di-tert-butyl dicarbonate (627 mg, 2.87 mmol). The mixture was stirred at room temperature for 1 hour. The reaction was quenched with half saturated brine (30 mL). Then the mixture was diluted with ethyl acetate (20 mL), and the layers were separated. The aqueous layer was further extracted with ethyl acetate (2×20 mL). The combined organic layers were washed with brine (10 mL), dried over sodium sulfate, filtered, and concentrated under reduced pressure. The crude residue was subjected to column chromatography (SiO₂, 80 g cartridge, 0-40% ethyl acetate in isohexane, 60 mL/minute) to afford the title compound (894 mg, 1.136 mmol, 79% yield). ¹H NMR (400 MHz, DMSO-d₆) δ ppm 7.44-7.29 (m, 6H), 5.24-5.04 (m, 2H), 4.69 (s, 1H), 4.40 (d, J=16.8 Hz, 1H), 4.03-3.93 (m, 1H), 3.45-3.20 (m, 9H), 3.19-3.07 (m, 1H), 2.96-2.74 (m, 1H), 1.51 (s, 9H), 1.38 (s, 9H), 1.37-1.23 (m, 9H); MS (ESI⁺) m/z 778 [M+Na]⁺.

### Example II-5C: tert-butyl (2R)-6-(benzyloxy)-2-{[(tert-butoxycarbonyl)(2-methoxyethyl)amino]methyl}-5-[(2-tert-butoxy-2-oxoethyl)amino]-4-fluoro-2,3-dihydro-1H-indole-1-carboxylate

To a solution of the product of Example II-5B (831 mg, 1.056 mmol) in methanol (4 mL) and tetrahydrofuran (4 mL) at room temperature was added a solution of lithium hydroxide monohydrate (221 mg, 5.28 mmol) in water (4.00 mL). The reaction mixture was then stirred at room temperature for 40 minutes. The reaction mixture was neutralized to pH 7 with 1 M hydrogen chloride (5.28 mL, 5.28 mmol) and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (811 mg, 1.045 mmol, 99% yield, 85% purity). ¹H NMR (400 MHz, DMSO-d₆) δ ppm 7.58-7.15 (m, 6H), 5.13-5.03 (m, 2H), 4.74-4.50 (m, 1H), 3.84 (s, 2H), 3.51-3.35 (m, 3H), 3.35-3.13 (m, 6H), 3.12-3.00 (m, 1H), 2.86-2.64 (m, 1H), 1.57-1.43 (m, 9H), 1.43-1.20 (m, 18H).

### Example II-5D: tert-butyl (2R)-6-(benzyloxy)-2-{[(tert-butoxycarbonyl)(2-methoxyethyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.181 mL, 2.090 mmol) in dichloromethane (3 mL) at 0° C. was added allyl alcohol (0.142 mL, 2.090 mmol) dropwise. The resulting solution was stirred for 20 minutes at 0° C. before a prepared solution of the product of Example II-5C (811 mg, 1.045 mmol) and triethylamine (0.364 mL, 2.61 mmol) in dichloromethane (3.00 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-100% ethyl acetate in isohexane, 60 mL/minute) to afford the title compound (618 mg, 0.676 mmol, 65% yield, 90% purity). ¹H NMR (400 MHz, DMSO-d₆) δ ppm 11.64-11.48 (m, 1H), 7.53-7.17 (m, 6H), 5.78-5.63 (m, 1H), 5.30-5.01 (m, 4H), 4.72-4.59 (m, 2H), 4.32-4.08 (m, 3H), 3.47-3.37 (m, 2H), 3.36-3.19 (m, 7H), 3.15-3.02 (m, 1H), 2.87-2.70 (m, 1H), 1.56-1.43 (m, 9H), 1.43-1.21 (m, 18H); MS (ESI⁻) m/z 821 [M−H]⁻.

### Example II-5E: tert-butyl (2R)-6-(benzyloxy)-2-{[(tert-butoxycarbonyl)(2-methoxyethyl)amino]methyl}-4-fluoro-5-(1,1,4-trioxo-lf, 2,5-thiadiazolidin-2-yl)-2,3-dihydro-1H-indole-1-carboxylate-ammonia

To a solution of the product of Example II-5D (600 mg, 0.656 mmol) and potassium carbonate (363 mg, 2.62 mmol) in methanol (15 mL) was added tetrakis(triphenylphosphine)palladium(0) (76 mg, 0.066 mmol). The reaction mixture was stirred at 60° C. for 1 hour. The mixture was cooled to room temperature and diluted with methanol (10 mL). C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 10 mM ammonium bicarbonate, 60 mL/minute) to afford the title compound as an ammonium salt (214 mg, 0.232 mmol, 35% yield, 74% purity). ¹H NMR (400 MHz, DMSO-d₆) δ ppm 7.67-7.46 (m, 3H), 7.38-7.26 (m, 3H), 7.06 (s, 3H), 5.17-5.04 (m, 2H), 4.72-4.59 (m, 1H), 4.34 (t, J=5.1 Hz, 1H), 3.98-3.82 (m, 2H), 3.50-3.36 (m, 5H), 3.24 (s, 3H), 3.15-3.02 (m, 1H), 2.94-2.68 (m, 1H), 1.49 (s, 9H), 1.38 (s, 4H), 1.30 (s, 5H); MS (ESI⁺) m/z 687 [M+Na]⁺.

### Example II-5F: tert-butyl (2R)-2-{[(tert-butoxycarbonyl)(2-methoxyethyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a solution of the product of Example II-5E (214 mg, 0.238 mmol) in degassed water (0.5 mL) and ethanol (4 mL) was added 10% Pd/C (25 mg). The resulting suspension was allowed to stir under hydrogen (5 bar) for 18 hours. The suspension was concentrated under reduced pressure onto C18 silica gel (3 g). The crude residue was subjected to column chromatography (Reveleris® 24 g reversed phase (C18) flash cartridge, dry loaded with C18 silica, 10-60% methanol in 10 mM ammonium bicarbonate, 40 mL/minute) to afford the title product as an ammonium salt(100 mg, 0.152 mmol, 64% yield, 90% purity). ¹H NMR (400 MHz, DMSO-d₆) δ ppm 9.26 (s, 1H), 7.27-6.76 (m, 4H), 4.67-4.54 (m, 1H), 3.95-3.80 (m, 2H), 3.49-3.37 (m, 3H), 3.32 (s, 3H), 3.30-3.25 (m, 1H), 3.24 (s, 3H), 3.11-2.95 (m, 1H), 2.82-2.63 (m, 1H), 1.50 (s, 9H), 1.38 (s, 4H), 1.32 (s, 5H); MS (ESI⁺) m/z 597 [M+Na]⁺.

### Example II-5G: 5-[(2R)-4-fluoro-6-hydroxy-2-{[(2-methoxyethyl)amino]methyl}-2,3-dihydro-1H-indol-5-yl]-1λ⁶,2,5-thiadiazolidine-1,1,3-trione

To a solution of the product of Example II-5F (100 mg, 0.152 mmol) in dichloromethane (4 mL) at room temperature was added trifluoroacetic acid (0.181 mL, 2.349 mmol). The reaction mixture was stirred at 25° C. for 20 hours. The reaction mixture was neutralized with 0.7 M NH₃ in methanol:water (95:5) (6 mL) and concentrated under reduced pressure. The crude residue was subjected to column chromatography (Reveleris® 12 g reversed phase (C18) flash cartridge, dry load on C18 silica, 10-50% methanol in 10 mM ammonium bicarbonate, 30 mL/minute) to afford the title compound (29 mg, 0.077 mmol, 51% yield). ¹H NMR (400 MHz, DMSO-d₆) δ ppm 8.68 (s, 1H), 8.48 (br s, 1.5H), 6.01 (s, 1H), 5.92 (s, 1H), 4.18-4.06 (m, 1H), 3.84 (s, 2H), 3.59 (t, J=5.1 Hz, 2H), 3.32 (s, 3H), 3.16 (t, J=5.2 Hz, 2H), 3.13-2.98 (m, 3H), 2.72 (dd, J=15.6, 6.7 Hz, 1H); MS (ESI⁺) m/z 375 [M+H]⁺.

### Example II-6: 5-[(2R)-2-{[(cyclobutylmethyl)amino]methyl}-4-fluoro-6-hydroxy-2,3-dihydro-1H-indol-5-yl]-1λ⁶,2,5-thiadiazolidine-1,1,3-trione (Compound 137)

### Example II-6A: tert-butyl (2R)-6-(benzyloxy)-5-[(2-tert-butoxy-2-oxoethyl)(trifluoroacetyl)amino]-2-{[(cyclobutylmethyl)amino]methyl}-4-fluoro-2,3-dihydro-1H-indole-1-carboxylate

Acetic acid (0.25 mL, 4.37 mmol) was added to a solution of cyclobutylmethanamine, hydrochloric acid (0.54 g, 4.44 mmol) and the product of Example II-2I (1.06 g, 1.475 mmol) in 1,2-dichloroethane (6 mL). The reaction mixture was stirred at room temperature for 1 hour. Triethylamine (0.6 mL, 4.30 mmol) was added, and the reaction mixture was stirred at room temperature for 1 hour. Sodium triacetoxyborohydride (470 mg, 2.218 mmol) was added, and the reaction mixture was stirred for 30 minutes. The reaction mixture was quenched with a saturated aqueous solution of sodium bicarbonate (3 mL) and stirred for 10 minutes. The reaction was diluted with water (20 mL) and extracted with ethyl acetate (3×20 mL). The combined organic layers were washed with brine (20 mL), dried over sodium sulfate, filtered, and concentrated under reduced pressure to afford the title compound (0.89 g, 1.341 mmol, 91% yield). ¹H NMR (400 MHz, DMSO-d₆) δ ppm 7.49-7.25 (m, 6H), 5.23-5.02 (m, 2H), 4.52-4.31 (m, 2H), 3.96 (dd, J=16.6, 2.0 Hz, 1H), 3.21-3.06 (m, 1H), 2.99 (ddd, J=16.0, 7.3, 2.6 Hz, 1H), 2.78-2.69 (m, 1H), 2.61-2.52 (m, 2H), 2.42-2.23 (m, 1H), 1.98-1.87 (m, 2H), 1.87-1.68 (m, 2H), 1.67-1.54 (m, 3H), 1.51 (s, 9H), 1.38 (s, 9H); MS (ESI⁺) m/z 667 [M+H]⁺.

### Example II-6B: tert-butyl (2R)-6-(benzyloxy)-2-{[(tert-butoxycarbonyl)(cyclobutylmethyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-6A (1.05 g, 1.341 mmol) in acetonitrile (6 mL) at room temperature was added N,N-diisopropylethylamine (0.702 mL, 4.02 mmol) and di-tert-butyl dicarbonate (0.585 g, 2.68 mmol). The mixture was stirred at room temperature for 1 hour. The reaction was quenched with half saturated brine (30 mL). The mixture was diluted with ethyl acetate (20 mL) and the layers were separated. The aqueous layer was extracted with ethyl acetate (2×20 mL). The combined organic layers were washed with brine (10 mL), dried over sodium sulfate, filtered, and concentrated under reduced pressure. The crude residue was subjected to column chromatography (SiO₂, 80 g cartridge, 0-40% ethyl acetate in isohexane, 60 mL/minute) to afford the title compound (957 mg, 1.187 mmol, 89% yield). ¹H NMR (400 MHz, DMSO-d₆) δ ppm 7.49-7.26 (m, 6H), 5.25-5.05 (m, 2H), 4.66 (s, 1H), 4.40 (d, J=16.7 Hz, 1H), 3.96 (s, 1H), 3.54-2.73 (m, 7H), 1.98-1.87 (m, 2H), 1.87-1.73 (m, 2H), 1.73-1.59 (m, 2H), 1.54-1.46 (m, 9H), 1.41-1.37 (m, 9H), 1.37-1.22 (m, 9H).

### Example II-6C: tert-butyl (2R)-6-(benzyloxy)-2-{[(tert-butoxycarbonyl)(cyclobutylmethyl)amino]methyl}-5-[(2-tert-butoxy-2-oxoethyl)amino]-4-fluoro-2,3-dihydro-1H-indole-1-carboxylate

To a solution of the product of Example II-6B (957 mg, 1.187 mmol) in methanol (2.8 mL) and tetrahydrofuran (2.8 mL) at room temperature was added 2 M aqueous lithium hydroxide (1.781 mL, 3.56 mmol). The reaction mixture was then stirred at room temperature for 1 hour. The reaction mixture was neutralized to pH 7 with 1 M aqueous hydrochloric acid and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (735 mg, 0.988 mmol, 83% yield, 90% purity). ¹H NMR (400 MHz, DMSO-d₆ at 90° C.) δ ppm 7.53-7.44 (m, 2H), 7.44-7.28 (m, 3H), 7.17 (s, 1H), 5.09 (s, 2H), 4.65-4.55 (m, 1H), 4.54-4.46 (m, 1H), 3.84 (dd, J=6.8, 2.0 Hz, 2H), 3.42-3.14 (m, 4H), 3.06 (dd, J=15.9, 9.2 Hz, 1H), 2.82 (dd, J=15.9, 2.1 Hz, 1H), 2.62-2.52 (m, 1H), 2.07-1.91 (m, 2H), 1.91-1.74 (m, 2H), 1.74-1.60 (m, 2H), 1.51 (s, 9H), 1.46-1.25 (m, 18H); MS (ESI⁺) m/z 570 [M+H—C(O)OC(CH₃)₃]⁺.

### Example II-6D: tert-butyl (2R)-6-(benzyloxy)-2-{[(tert-butoxycarbonyl)(cyclobutylmethyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.16 mL, 1.843 mmol) in dichloromethane (4.94 mL) at 0° C. was added allyl alcohol (0.13 mL, 1.912 mmol) dropwise. The resulting solution was stirred for 20 minutes at 0° C. before a prepared solution of the product of Example II-6C (0.735 g, 0.988 mmol) and triethylamine (0.35 mL, 2.51 mmol) in dichloromethane (5 mL) was added dropwise. The resulting solution was stirred at 0° C. for 30 minutes. The reaction mixture was quenched with water (50 mL) and diluted with dichloromethane (50 mL). The layers were separated, and the aqueous layer was extracted with dichloromethane (3×50 mL). The combined organic layers were dried over sodium sulfate, filtered, and concentrated under reduced pressure. The crude residue was subjected to column chromatography (SiO₂, 40 g cartridge, 0-40% tert-butyl methyl ether in isohexane, 90 mL/minute) to afford the title compound (665 mg, 0.798 mmol, 81% yield). ¹H NMR (400 MHz, DMSO-d₆) δ ppm 11.69-11.44 (m, 1H), 7.56-7.06 (m, 6H), 5.75-5.63 (m, 1H), 5.27-4.95 (m, 4H), 4.73-4.56 (m, 2H), 4.33-4.19 (m, 1H), 4.19-4.07 (m, 2H), 3.44-3.02 (m, 5H), 2.83 (d, J=15.9 Hz, 1H), 1.97-1.87 (m, 2H), 1.87-1.73 (m, 2H), 1.73-1.60 (m, 2H), 1.48 (s, 9H), 1.42-1.21 (m, 19H); MS (ESI⁻) m/z 831 [M−H]⁻.

### Example II-6E: tert-butyl (2R)-6-(benzyloxy)-2-{[(tert-butoxycarbonyl)(cyclobutylmethyl)amino]methyl}-4-fluoro-5-(1,1,4-trioxo-1λ⁶,2,5-thiadiazolidin-2-yl)-2,3-dihydro-1H-indole-1-carboxylate-ammonia

A solution of the product of Example II-6D (0.782 g, 0.798 mmol) and potassium carbonate (0.44 g, 3.18 mmol) in methanol (8 mL) was degassed under vacuum and backfilled with nitrogen three times. To the mixture was added tetrakis(triphenylphosphine)palladium(0) (0.092 g, 0.080 mmol), and the reaction mixture was degassed under vacuum and backfilled with nitrogen three times. The reaction mixture was stirred at 60° C. for 4 hours. The mixture was cooled to room temperature and left overnight. The reaction mixture was diluted with methanol (10 mL). Diatomaceous earth (3 g) was added, and the mixture was concentrated in vacuo. The crude residue was subjected to column chromatography (Reveleris® 40 g reversed phase (C18) flash cartridge, dry loaded with diatomaceous earth, 5-70% methanol in 10 mM ammonium bicarbonate, 15 mL/minute) to afford the title compound as an ammonium salt (608 mg, 0.677 mmol, 85% yield, 80% purity). ¹H NMR (400 MHz, DMSO-d₆ at 90° C.) δ ppm 7.69-7.58 (m, 1H), 7.58-7.45 (m, 3H), 7.41-7.26 (m, 3H), 7.21 (s, 1H), 5.11 (s, 2H), 4.65 (dddd, J=9.4, 7.5, 5.7, 2.1 Hz, 1H), 3.94 (d, J=13.0 Hz, 1H), 3.87 (d, J=13.1 Hz, 1H), 3.39-3.25 (m, 2H), 3.07 (ddd, J=24.4, 12.3, 8.0 Hz, 5H), 2.84 (dt, J=15.5, 4.0 Hz, 1H), 2.63-2.52 (m, 1H), 2.06-1.91 (m, 2H), 1.90-1.76 (m, 2H), 1.76-1.61 (m, 2H), 1.55-1.46 (m, 9H), 1.37 (s, 9H); MS (ESI⁺) m/z 697 [M+Na]⁺.

### Example II-6F: tert-butyl (2R)-2-{[(tert-butoxycarbonyl)(cyclobutylmethyl)amino]methyl}-4-fluoro-6-hydroxy-5-(1,1,4-trioxo-1λ⁶,2,5-thiadiazolidin-2-yl)-2,3-dihydro-1H-indole-1-carboxylate

To a suspension of the product of Example II-6E (100 mg, 0.111 mmol) in degassed water (0.1 mL) and ethanol (4 mL) was added 10% Pd/C (25 mg). The resulting suspension was allowed to stir under hydrogen (5 bar) for 18 hours. The reaction mixture was filtered through a Whatman glass fiber filter paper and washed with ethanol (3×10 mL). The filtrate was concentrated under reduced pressure onto C18 silica (1 g). The crude residue was subjected to column chromatography (Reveleris® 24 g reversed phase (C18) flash cartridge, dry loaded with diatomaceous earth, 5-55% methanol in 10 mM ammonium bicarbonate, 50 mL/minute) to afford the title compound (44 mg, 0.070 mmol, 63% yield). ¹H NMR (400 MHz, DMSO-d₆ at 90° C.) δ ppm 8.65 (s, 1H), 6.99 (s, 1H), 4.69-4.54 (m, 1H), 3.97-3.84 (m, 2H), 3.39-3.17 (m, 4H), 3.11-2.95 (m, 1H), 2.81 (dd, J=15.7, 2.0 Hz, 1H), 2.63-2.52 (m, 1H), 2.06-1.92 (m, 2H), 1.89-1.76 (m, 2H), 1.76-1.63 (m, 2H), 1.54 (s, 9H), 1.38 (s, 9H); MS (ESI⁺) m/z 607 [M+Na]⁺.

### Example II-6G: 5-[(2R)-2-{[(cyclobutylmethyl)amino]methyl}-4-fluoro-6-hydroxy-2,3-dihydro-1H-indol-5-yl]-1λ⁶,2,5-thiadiazolidine-1,1,3-trione

To a solution of the product of Example II-6F (96 mg, 0.148 mmol) in dichloromethane (1 mL) at room temperature was added trifluoroacetic acid (0.171 mL, 2.226 mmol). The reaction mixture was stirred at room temperature for 2 hours. The reaction mixture was neutralized with 0.7 M NH₃ in methanol:water (95:5) (7 mL) and concentrated under reduced pressure. The crude residue was subjected to column chromatography (Reveleris® 24 g reversed phase (C18) flash cartridge, dry loaded with C18 silica, 5-20% methanol in 0.1% ammonium hydroxide, 35 mL/minute) to afford the title compound (56 mg, 0.146 mmol, 98% yield). ¹H NMR (400 MHz, DMSO-d₆) δ ppm 8.65 (s, 1H), 7.87 (s, 2H), 6.00 (s, 1H), 5.91 (s, 1H), 4.16-3.99 (m, 1H), 3.84 (s, 2H), 3.07 (dd, J=15.5, 9.3 Hz, 1H), 3.01-2.83 (m, 4H), 2.71 (dd, J=15.5, 6.7 Hz, 1H), 2.65-2.54 (m, 1H), 2.13-1.97 (m, 2H), 1.95-1.66 (m, 4H); MS (ESI⁺) m/z 385 [M+H]⁺.

### Example II-7: 5-[(2R)-4-fluoro-6-hydroxy-2-({[(oxan-4-yl)methyl]amino}methyl)-2,3-dihydro-1H-indol-5-yl]-1λ⁶,2,5-thiadiazolidine-1,1,3-trione (Compound 138)

### Example II-7A: tert-butyl (2R)-6-(benzyloxy)-5-[(2-tert-butoxy-2-oxoethyl)(trifluoroacetyl)amino]-4-fluoro-2-({[(oxan-4-yl)methyl]amino}methyl)-2,3-dihydro-1H-indole-1-carboxylate

Acetic acid (0.252 mL, 4.40 mmol) was added to a solution of (tetrahydro-2H-pyran-4-yl)methanamine (0.496 mL, 4.40 mmol) and the product of Example II-2I (0.92 g, 1.465 mmol) in 1,2-dichloroethane (6 mL). The reaction mixture was stirred at room temperature for 1 hour. Sodium triacetoxyborohydride (0.466 g, 2.198 mmol) was added, and the reaction mixture was stirred for 1 hour. The reaction mixture was quenched with a saturated aqueous solution of sodium bicarbonate (20 mL) and stirred for 10 minutes. The mixture was diluted with water (20 mL) and extracted with ethyl acetate (3×30 mL). The combined organic layers were washed with brine (20 mL), dried over sodium sulfate, filtered, and concentrated under reduced pressure to afford the title compound (1.0345 g, 1.472 mmol, 100% yield). ¹H NMR (400 MHz, DMSO-d₆) δ ppm 7.44-7.27 (m, 6H), 5.22-5.02 (m, 2H), 4.51-4.44 (m, 1H), 4.40 (dd, J=16.6, 5.8 Hz, 1H), 3.96 (dd, J=16.6, 3.4 Hz, 1H), 3.84-3.76 (m, 2H), 3.27-3.07 (m, 3H), 3.07-2.93 (m, 1H), 2.79-2.69 (m, 1H), 2.63-2.52 (m, 1H), 2.45-2.30 (m, 2H), 1.59-1.52 (m, 3H), 1.50 (s, 9H), 1.38 (s, 9H), 1.14-0.98 (m, 2H); MS (ESI⁺) m/z 696 [M+H]⁺.

### Example II-7B: tert-butyl (2R)-6-(benzyloxy)-2-({(tert-butoxycarbonyl)[(oxan-4-yl)methyl]amino}methyl)-5-[(2-tert-butoxy-2-oxoethyl)(trifluoroacetyl)amino]-4-fluoro-2,3-dihydro-1H-indole-1-carboxylate

To a solution of the product of Example II-7A (1.0345 g, 1.472 mmol) in acetonitrile (6 mL) at room temperature was added N,N-diisopropylethylamine (0.771 mL, 4.42 mmol) and di-tert-butyl dicarbonate (0.643 g, 2.94 mmol). The mixture was stirred at room temperature for 1 hour. The reaction was quenched with half saturated brine (30 mL). The mixture was diluted with ethyl acetate (20 mL), and the layers were separated. The aqueous layer was extracted with ethyl acetate (2×20 mL). The combined organic layers were washed with brine (10 mL), dried over sodium sulfate, filtered, and concentrated under reduced pressure. The crude residue was subjected to column chromatography (SiO₂, 80 g cartridge, 0-40% ethyl acetate in isohexane, 60 mL/minute) to afford the title compound (1.05 g, 1.253 mmol, 85% yield). ¹H NMR (400 MHz, DMSO-d₆) δ ppm 7.46-7.29 (m, 6H), 5.22-5.05 (m, 2H), 4.76-4.63 (m, 1H), 4.41 (d, J=16.8 Hz, 1H), 3.98 (d, J=16.5 Hz, 1H), 3.89-3.78 (m, 2H), 3.33-2.64 (m, 9H), 1.55-1.47 (m, 9H), 1.47-1.40 (m, 2H), 1.40-1.37 (m, 9H), 1.37-1.20 (m, 9H), 1.20-1.06 (m, 2H).

### Example II-7C: tert-butyl (2R)-6-(benzyloxy)-2-({(tert-butoxycarbonyl)[(oxan-4-yl)methyl]amino}methyl)-5-[(2-tert-butoxy-2-oxoethyl)amino]-4-fluoro-2,3-dihydro-1H-indole-1-carboxylate

To a solution of the product of Example II-7B (1.05 g, 1.253 mmol) in methanol (3 mL) and tetrahydrofuran (3 mL) at room temperature was added 2 M aqueous lithium hydroxide (1.880 mL, 3.76 mmol). The reaction mixture was then stirred at room temperature for 30 minutes. The reaction mixture was quenched with saturated ammonium chloride (20 mL). The aqueous layer was extracted with ethyl acetate (3×50 mL). The combined organic layers were washed with brine (70 mL), dried over sodium sulfate, filtered, and concentrated in vacuo. The residue was triturated in isohexanes. The solid was separated by decanting to afford the title compound (0.472 g, 0.682 mmol, 54% yield). ¹H NMR (400 MHz, DMSO-d₆) δ ppm 7.53-7.44 (m, 2H), 7.40 (t, J=7.3 Hz, 2H), 7.34 (t, J=7.2 Hz, 1H), 6.85 (s, 1H), 5.08 (s, 2H), 4.70 (s, 1H), 4.60 (s, 1H), 3.88-3.78 (m, 4H), 3.28-3.16 (m, 4H), 3.14-2.90 (m, 3H), 2.84-2.68 (m, 1H), 1.91-1.73 (m, 1H), 1.48 (s, 9H), 1.44-1.22 (m, 20H), 1.20-1.05 (m, 2H); MS (ESI⁺) m/z 700 [M+H]⁺.

### Example II-7D: tert-butyl (2R)-6-(benzyloxy)-2-({(tert-butoxycarbonyl)[(oxan-4-yl)methyl]amino}methyl)-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.155 mL, 1.785 mmol) in dichloromethane (7 mL) at 0° C. was added allyl alcohol (0.125 mL, 1.838 mmol) dropwise. The resulting solution was stirred for 20 minutes at 0° C. before a prepared solution of the product of Example II-7C (0.530 g, 0.682 mmol) and triethylamine (0.310 mL, 2.224 mmol) in dichloromethane (7 mL) was added dropwise. The resulting solution was stirred at 0° C. for 50 minutes. The reaction mixture was quenched with saturated solution of ammonium chloride (30 mL) and diluted with dichloromethane (50 mL). The layers were separated, and the aqueous layer was extracted with dichloromethane (2×50 mL). The combined organic layers were dried over sodium sulfate, filtered, and concentrated under reduced pressure to afford the title compound (0.628 g, 0.655 mmol, 96% yield). ¹H NMR (400 MHz, DMSO-d₆) δ ppm 7.53-6.81 (m, 7H), 6.07-5.64 (m, 2H), 5.55-5.00 (m, 6H), 4.82-4.39 (m, 4H), 4.31-4.07 (m, 2H), 3.88-3.74 (m, 2H), 3.28-3.01 (m, 4H), 1.95-1.72 (m, 1H), 1.53-1.42 (m, 9H), 1.42-0.99 (m, 22H); MS (ESI⁻) m/z 861 [M−H]⁻.

### Example II-7E: tert-butyl (2R)-6-(benzyloxy)-2-({(tert-butoxycarbonyl)[(oxan-4-yl)methyl]amino}methyl)-4-fluoro-5-(1,1,4-trioxo-1λ⁶,2,5-thiadiazolidin-2-yl)-2,3-dihydro-1H-indole-1-carboxylate-ammonia (partial)

To a solution of the product of Example II-7D (0.628 g, 0.655 mmol) and potassium carbonate (0.402 g, 2.91 mmol) in methanol (10 mL) was added tetrakis(triphenylphosphine)palladium(0) (0.084 g, 0.073 mmol). The reaction mixture was stirred at 60° C. for 1 hour. The mixture was cooled to room temperature and diluted with methanol (10 mL). Diatomaceous earth (2 g) was added, and the mixture was concentrated in vacuo. The crude residue was subjected to column chromatography (Reveleris® 24 g reversed phase (C18) flash cartridge, dry loaded with diatomaceous earth, 5-70% methanol in 10 mM ammonium hydroxide, 15 mL/minute) to afford the title compound as a partial ammonium salt (0.246 g, 0.314 mmol, 48% yield, 92% purity). ¹H NMR (400 MHz, DMSO-d₆) δ ppm 7.56-7.45 (m, 2H), 7.42-7.26 (m, 3H), 7.25-6.77 (m, 2H), 5.10 (s, 2H), 4.70-4.64 (m, 1H), 3.98-3.77 (m, 4H), 3.28-2.96 (m, 7H), 2.91-2.63 (m, 1H), 1.86-1.81 (m, 1H), 1.63-1.20 (m, 20H), 1.19-1.03 (m, 2H); MS (ESI⁻) m/z 703 [M−H]⁻.

### Example II-7F: tert-butyl (2R)-2-({(tert-butoxycarbonyl)[(oxan-4-yl)methyl]amino}methyl)-4-fluoro-6-hydroxy-5-(1,1,4-trioxo-1λ⁶,2,5-thiadiazolidin-2-yl)-2,3-dihydro-1H-indole-1-carboxylate

To a suspension of the product of Example II-7E (246 mg, 0.314 mmol) in water (2 mL) and 1,4-dioxane (2 mL) was added 10% Pd/C (49 mg). The resulting suspension was allowed to stir under hydrogen (5 bar) for 2 hours. Additional 10% Pd/C (50 mg) was added, and the mixture was allowed to stir under hydrogen (5 bar) for 18 hours. The reaction mixture was filtered through a Whatman glass fiber filter paper and washed with ethanol (3×20 mL). The filtrate was concentrated under reduced pressure onto diatomaceous earth (4 g). The crude residue was subjected to column chromatography (Reveleris® 40 g reversed phase (C18) flash cartridge, dry loaded with diatomaceous earth, 5-55% methanol in 10 mM ammonium hydroxide, 25 mL/minute) to afford the title compound (0.123 g, 0.190 mmol, 61% yield). ¹H NMR (400 MHz, DMSO-d₆ at 90° C.) δ ppm 9.20-7.66 (m, 1H), 6.99 (s, 1H), 4.71-4.60 (m, 1H), 3.89 (dd, 2H), 3.84 (ddd, J=11.5, 4.4, 2.3 Hz, 2H), 3.42-3.18 (m, 4H), 3.12-3.01 (m, 3H), 2.81 (dd, J=15.6, 2.0 Hz, 1H), 1.94-1.80 (m, 1H), 1.54 (s, 9H), 1.55-1.43 (m, 2H), 1.38 (s, 9H), 1.25-1.10 (m, 2H); MS (ESI⁻) m/z 613 [M−H]⁻.

### Example II-7G: 5-[(2R)-4-fluoro-6-hydroxy-2-({[(oxan-4-yl)methyl]amino}methyl)-2,3-dihydro-1H-indol-5-yl]-1λ⁶,2,5-thiadiazolidine-1,1,3-trione

To a solution of the product of Example II-7F (116 mg, 0.189 mmol) in dichloromethane (1 mL) at room temperature was added trifluoroacetic acid (0.250 mL, 3.24 mmol). The reaction mixture was stirred at room temperature for 2 hours. Additional trifluoroacetic acid (0.100 mL, 1.298 mmol) was added, and the reaction mixture was stirred overnight. The reaction mixture was neutralized with 0.7 M NH₃ in methanol:water (95:5) (7 mL) and concentrated under reduced pressure. The crude residue was subjected to column chromatography (Reveleris® 24 g reversed phase (C18) flash cartridge, dry loaded with silica gel C18 silica gel, 5-20% methanol in 10 mM ammonium bicarbonate, 25 mL/minute) to afford the title compound (68.6 mg, 0.156 mmol, 82% yield). ¹H NMR (400 MHz, DMSO-d₆) δ ppm 8.64 (s, 1H), 6.00 (t, J=2.2 Hz, 1H), 5.90 (s, 1H), 4.12-4.04 (m, 1H), 3.91-3.75 (m, 4H), 3.33-3.23 (m, 2H), 3.07 (dd, J=15.5, 9.3 Hz, 1H), 2.97-2.86 (m, 2H), 2.81-2.75 (m, 2H), 2.71 (dd, J=15.5, 6.8 Hz, 1H), 1.95-1.77 (m, 1H), 1.63 (dtd, J=13.2, 4.6, 2.3 Hz, 2H), 1.30-1.09 (m, 2H); MS (ESI⁺) m/z 415 [M+H]⁺.

### Example II-8: 5-[(2R)-4-fluoro-6-hydroxy-2-{[(3-methylbutyl)amino]methyl}-2,3-dihydro-1H-indol-5-yl]-1λ⁶,2,5-thiadiazolidine-1,1,3-trione (Compound 139)

### Example II-8A: tert-butyl (2R)-6-(benzyloxy)-5-[(2-tert-butoxy-2-oxoethyl)(trifluoroacetyl)amino]-4-fluoro-2-{[(3-methylbutyl)amino]methyl}-2,3-dihydro-1H-indole-1-carboxylate

Acetic acid (0.25 mL, 4.37 mmol) was added to a solution of 3-methylbutan-1-amine (0.51 mL, 4.39 mmol) and the product of Example II-2I (1.06 g, 1.475 mmol) in 1,2-dichloroethane (6 mL). The reaction mixture was stirred at room temperature for 40 minutes. Sodium triacetoxyborohydride (470 mg, 2.218 mmol) was added, and the reaction mixture was stirred for 30 minutes. The reaction mixture was quenched with a saturated aqueous solution of sodium bicarbonate (3 mL) and stirred for 10 minutes. The reaction was diluted with water (20 mL) and extracted with ethyl acetate (3×20 mL). The combined organic layers were washed with brine (20 mL), dried over sodium sulfate, filtered, and concentrated under reduced pressure to afford the title compound (0.97 g, 1.415 mmol, 96% yield). ¹H NMR (400 MHz, DMSO-d₆) δ ppm 7.52-7.25 (m, 6H), 5.23-5.02 (m, 2H), 4.54-4.33 (m, 2H), 3.96 (d, J=16.6 Hz, 1H), 3.22-3.07 (m, 1H), 3.07-2.92 (m, 1H), 2.80-2.70 (m, 1H), 2.62-2.42 (m, 3H), 1.63-1.45 (m, 10H), 1.38 (s, 9H), 1.30-1.20 (m, 2H), 0.89-0.75 (m, 6H); MS (ESI⁺) m/z 669 [M+H]⁺.

### Example II-8B: tert-butyl (2R)-6-(benzyloxy)-2-{[(tert-butoxycarbonyl)(3-methylbutyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-8A (1.05 g, 1.415 mmol) in acetonitrile (6 mL) at room temperature was added N,N-diisopropylethylamine (0.742 mL, 4.25 mmol) and di-tert-butyl dicarbonate (0.618 g, 2.83 mmol). The mixture was stirred at room temperature for 1 hour. The reaction was quenched with half saturated brine (30 mL). The mixture was diluted with ethyl acetate (20 mL), and the layers were separated. The aqueous layer was extracted with ethyl acetate (2×20 mL). The combined organic layers were washed with brine (10 mL), dried over sodium sulfate, filtered, and concentrated under reduced pressure. The crude residue was subjected to column chromatography (SiO₂, 80 g cartridge, 0-40% ethyl acetate in isohexane, 60 mL/minute) to afford the title compound (0.995 g, 1.296 mmol, 92% yield). ¹H NMR (400 MHz, DMSO-d₆) δ ppm 7.47-7.29 (m, 6H), 5.23-5.04 (m, 2H), 4.66 (s, 1H), 4.40 (d, J=16.7 Hz, 1H), 4.01-3.92 (m, 1H), 3.56-2.74 (m, 7H), 1.51 (s, 9H), 1.38 (s, 11H), 1.37-1.21 (m, 9H), 0.93-0.78 (m, 6H).

### Example II-8C: tert-butyl (2R)-6-(benzyloxy)-2-{[(tert-butoxycarbonyl)(3-methylbutyl)amino]methyl}-5-[(2-tert-butoxy-2-oxoethyl)amino]-4-fluoro-2,3-dihydro-1H-indole-1-carboxylate

To a solution of the product of Example II-8B (1.07 g, 1.296 mmol) in methanol (3 mL) and tetrahydrofuran (3 mL) at room temperature was added 2 M aqueous lithium hydroxide (1.98 mL, 3.96 mmol). The reaction mixture was then stirred at room temperature for 30 minutes. The reaction was quenched with a saturated aqueous solution of ammonium chloride (20 mL). The aqueous layer was extracted with ethyl acetate (3×50 mL). The combined organic layers were washed with brine (70 mL), dried over sodium sulfate, filtered, and concentrated in vacuo to afford the title compound (0.896 g, 1.134 mmol, 87% yield, 85% purity). ¹H NMR (400 MHz, DMSO-d₆) δ ppm 7.54-7.28 (m, 6H), 5.21-4.96 (m, 2H), 4.73-4.44 (m, 2H), 3.84 (s, 1H), 3.30-2.94 (m, 6H), 2.94-2.70 (m, 1H), 1.57-1.05 (m, 30H), 0.92-0.80 (m, 6H); MS (ESI⁺) m/z 672 [M+H]⁺.

### Example II-8D: tert-butyl (2R)-6-(benzyloxy)-2-{[(tert-butoxycarbonyl)(3-methylbutyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.2 mL, 2.303 mmol) in dichloromethane (8 mL) at 0° C. was added allyl alcohol (0.155 mL, 2.279 mmol) dropwise. The resulting solution was stirred for 20 minutes at 0° C. before a prepared solution of the product of Example II-8C (0.896 g, 1.134 mmol) and triethylamine (0.4 mL, 2.87 mmol) in dichloromethane (8 mL) was added dropwise. The resulting solution was stirred at 0° C. for 45 minutes. The reaction mixture was quenched with saturated ammonium chloride solution (30 mL) and diluted with dichloromethane (50 mL). The layers were separated, and the aqueous layer was extracted with dichloromethane (2×50 mL). The combined organic layers were dried over sodium sulfate, filtered, and concentrated under reduced pressure. The crude residue was subjected to column chromatography (SiO₂, 24 g cartridge, 0-50% ethyl acetate in isohexane, 35 mL/minute) to afford the title compound (0.331 g, 0.373 mmol, 33% yield). ¹H NMR (400 MHz, DMSO-d₆) δ ppm 11.67-11.43 (m, 1H), 7.58-7.18 (m, 6H), 5.77-5.64 (m, 1H), 5.26-4.98 (m, 4H), 4.70-4.57 (m, 2H), 4.31-4.07 (m, 3H), 3.27-2.74 (m, 6H), 1.57-1.21 (m, 30H), 0.94-0.79 (m, 6H); MS (ESI⁺) m/z 857 [M+Na]⁺.

### Example II-8E: tert-butyl (2R)-6-(benzyloxy)-2-{[(tert-butoxycarbonyl)(3-methylbutyl)amino]methyl}-4-fluoro-5-(1,1,4-trioxo-1λ⁶,2,5-thiadiazolidin-2-yl)-2,3-dihydro-1H-indole-1-carboxylate-ammonia (partial)

To a solution of the product of Example II-8D (0.389 g, 0.354 mmol) and potassium carbonate (0.196 g, 1.416 mmol) in methanol (10 mL) was added tetrakis(triphenylphosphine)palladium(0) (0.041 g, 0.035 mmol). The reaction mixture was stirred at 60° C. for 1 hour. The mixture was cooled to room temperature and diluted with methanol (10 mL). Diatomaceous earth (1 g) was added, and the mixture was concentrated in vacuo. The crude residue was subjected to column chromatography (Reveleris® 24 g reversed phase (C18) flash cartridge, dry loaded with silica, 5-70% methanol in 0.1% ammonium hydroxide, 17 mL/minute) to afford the title compound as a partial ammonium salt (70 mg, 0.088 mmol, 25% yield, 87% purity). ¹H NMR (400 MHz, DMSO-d₆) δ ppm 7.53-7.44 (m, 2H), 7.39-7.30 (m, 2H), 7.33-7.25 (m, 1H), 7.01 (t, J=51.1 Hz, 1H), 5.09 (s, 2H), 4.65-4.61 (m, 1H), 3.98-3.80 (m, 2H), 3.25-2.70 (m, 6H), 1.49 (app s, 10H), 1.40-1.27 (m, 12H), 0.87 (d, J=6.5 Hz, 6H); MS (ESI⁺) m/z 699 [M+Na]⁺.

### Example II-8F: tert-butyl (2R)-2-{[(tert-butoxycarbonyl)(3-methylbutyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a suspension of the product of Example II-8E (70 mg, 0.088 mmol) in water (1 mL) and 1,4-dioxane (1 mL) was added 10% Pd/C (30 mg). The resulting suspension was allowed to stir under hydrogen (5 bar) for 3 hours. Additional 10% Pd/C (10 mg) was added, and the mixture was allowed to stir under hydrogen (5 bar) for 18 hours. The reaction mixture was filtered through a diatomaceous earth pad and washed with methanol (3×10 mL). The filtrate was concentrated under reduced pressure onto reverse layer C18 silica (1 g). The crude residue was subjected to column chromatography (Reveleris® 24 g reversed phase (C18) flash cartridge, dry loaded with reversed phase C18 silica, 5-55% methanol in 10 mM ammonium hydroxide, 18 mL/minute) to afford the title compound as an ammonium salt (33 mg, 0.054 mmol, 62% yield). ¹H NMR (400 MHz, DMSO-d₆) δ ppm 9.26 (s, 1H), 7.31-6.74 (m, 4H), 4.61-4.57 (m, 1H), 3.99-3.77 (m, 2H), 3.24-2.61 (m, 6H), 1.50 (app s, 10H), 1.40-1.29 (m, 12H), 0.87 (d, J=6.5 Hz, 6H).; MS (ESI⁻) m/z 585 [M−H]⁻.

### Example II-8G: 5-[(2R)-4-fluoro-6-hydroxy-2-{[(3-methylbutyl)amino]methyl}-2,3-dihydro-1H-indol-5-yl]-1λ⁶,2,5-thiadiazolidine-1,1,3-trione

To a solution of the product of Example II-8F (56 mg, 0.091 mmol) in 1,4-dioxane (2 mL) at room temperature was added trifluoroacetic acid (150 μl, 1.947 mmol). The reaction mixture was stirred at room temperature for 1 hour. Additional trifluoroacetic acid (200 μl, 2.60 mmol) was added, and the reaction mixture was stirred for 1 hour. 1 M Aqueous hydrochloric acid (1 mL) was added, and the reaction mixture was stirred for one hour. 6 M Aqueous hydrochloric acid (0.5 mL) was added, and the reaction mixture was stirred for 1 hour. The volatiles were removed under reduced pressure. The residue was redissolved in a mixture of 1,4-dioxane (1. mL) and 3 M aqueous hydrochloric acid (0.5 mL) and stirred for 3 hours. The reaction mixture was neutralized with 0.7 M ammonia in methanol) (2.5 mL) and concentrated under reduced pressure. The crude residue was subjected to column chromatography (Reveleris® 12 g reversed phase (C18) flash cartridge, dry loaded onto reversed phase18 silica, 5-20% methanol in 0.1% ammonium hydroxide, 30 mL/minute) to afford the title compound (14.5 mg, 0.036 mmol, 40% yield). ¹H NMR (400 MHz, DMSO-d₆) δ ppm 8.69 (s, 1H), 8.25 (s, 2H), 6.02 (s, 1H), 5.92 (s, 1H), 4.13-4.08 (m, 1H), 3.84 (s, 2H), 3.10 (dd, J=15.5, 9.3 Hz, 1H), 3.06-2.99 (m, 2H), 2.99-2.90 (m, 2H), 2.73 (dd, J=15.5, 6.9 Hz, 1H), 1.67-1.54 (m, 1H), 1.54-1.44 (m, 2H), 0.89 (d, J=6.5 Hz, 6H); MS (ESI⁺) m/z 387 [M+H]⁺.

### Example II-9: 5-[(2R)-2-{[(3,3-difluoropropyl)amino]methyl}-4-fluoro-6-hydroxy-2,3-dihydro-1H-indol-5-yl]-1λ⁶,2,5-thiadiazolidine-1,1,3-trione (Compound 140)

### Example II-9A: tert-butyl (2R)-6-(benzyloxy)-5-[(2-tert-butoxy-2-oxoethyl)(trifluoroacetyl)amino]-2-{[(3,3-difluoropropyl)amino]methyl}-4-fluoro-2,3-dihydro-1H-indole-1-carboxylate

Acetic acid (0.230 mL, 4.02 mmol) was added to a solution of 3,3-difluoropropan-1-amine hydrochloride (0.529 g, 4.02 mmol) and the product of Example II-2I (1 g, 1.341 mmol) in 1,2-dichloroethane (6 mL). The reaction mixture was stirred at room temperature for 40 minutes. Sodium triacetoxyborohydride (0.426 g, 2.012 mmol) was added, and the reaction mixture was stirred for 1 hour. The reaction mixture was quenched with a saturated aqueous solution of sodium bicarbonate (3 mL) and stirred for 10 minutes. The reaction mixture was diluted with water (20 mL) and extracted with ethyl acetate (3×20 mL). The combined organic layers were washed with brine (20 mL), dried over sodium sulfate, filtered, and concentrated under reduced pressure to afford the title compound (1.095 g, 1.297 mmol, 97% yield, 80% purity). MS (ESI⁺) m/z 676 [M+H]⁺.

### Example II-9B: tert-butyl (2R)-6-(benzyloxy)-2-{[(tert-butoxycarbonyl)(3,3-difluoropropyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-9A (1.09 g, 1.291 mmol) in acetonitrile (6 mL) was added N,N-diisopropylethylamine (0.676 mL, 3.87 mmol) and di-tert-butyl dicarbonate (0.563 g, 2.58 mmol). The reaction mixture was stirred at ambient temperature for 1 hour. The reaction was quenched with half saturated brine (10 mL). The mixture was diluted with ethyl acetate (20 mL), and the layers were separated. The aqueous layer was extracted with ethyl acetate (3×15 mL). The combined organic layers were dried with sodium sulfate, filtered, and concentrated under reduced pressure. The crude residue was subjected to column chromatography (silica gel, 80 g cartridge, 20% ethyl acetate in isohexane, 60 mL/minute) to afford the title compound (965 mg, 1.120 mmol, 83% yield). ¹H NMR (400 MHz, DMSO-d₆ at 90° C.) δ ppm 7.44-7.29 (m, 6H), 6.05 (tq, J=56.3, 4.1 Hz, 1H), 5.18 (dd, J=12.0, 3.9 Hz, 1H), 5.11 (dd, J=12.0, 2.1 Hz, 1H), 4.76-4.67 (m, 1H), 4.43 (dd, J=16.5, 9.0 Hz, 1H), 3.87 (dd, J=27.6, 16.5 Hz, 1H), 3.43-3.12 (m, 5H), 2.93-2.84 (m, 1H), 2.16-2.00 (m, 2H), 1.53 (d, J=2.4 Hz, 9H), 1.40 (d, J=2.2 Hz, 9H), 1.36 (d, J=7.5 Hz, 9H); MS (ESI⁺) m/z 798 [M+Na]⁺.

### Example II-9C: tert-butyl (2R)-6-(benzyloxy)-2-{[(tert-butoxycarbonyl)(3,3-difluoropropyl)amino]methyl}-5-[(2-tert-butoxy-2-oxoethyl)amino]-4-fluoro-2,3-dihydro-1H-indole-1-carboxylate

To a solution of the product of Example II-9B (965 mg, 1.182 mmol) in methanol (2.5 mL) and tetrahydrofuran (2.5 mL) at room temperature was added a solution of lithium hydroxide monohydrate (149 mg, 3.55 mmol) in water (2.5 mL). The reaction mixture was then stirred at room temperature for 40 minutes. The reaction mixture was neutralized to pH 7 with 1 M hydrogen chloride (3.55 mL, 3.55 mmol) and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (803 mg, 1.063 mmol, 90% yield, 90% purity). ¹H NMR (400 MHz, DMSO-d₆) δ ppm 7.56-7.22 (m, 6H), 6.09 (t, J=56.2 Hz, 2H), 5.08 (s, 2H), 4.74-4.49 (m, 1H), 3.85 (s, 2H), 3.45-3.15 (m, 4H), 3.15-3.01 (m, 1H), 2.85-2.65 (m, 1H), 2.05 (s, 2H), 1.47 (s, 9H), 1.38 (s, 4H), 1.34 (s, 9H), 1.29 (s, 5H); MS (ESI⁺) m/z 680 [M+H]⁺.

### Example II-9D: tert-butyl (2R)-6-(benzyloxy)-2-{[(tert-butoxycarbonyl)(3,3-difluoropropyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.2 mL, 2.303 mmol) in dichloromethane (3 mL) at 0° C. was added allyl alcohol (0.16 mL, 2.353 mmol) dropwise. The resulting solution was stirred for 20 minutes at 0° C. before a prepared solution of the product of Example II-9C (803 mg, 1.063 mmol) and triethylamine (0.4 mL, 2.87 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (SiO₂, 40 g cartridge, 0-100% ethyl acetate in isohexane, 60 mL/minute) to afford the title compound (737 mg, 0.647 mmol, 61% yield, 74% purity). ¹H NMR (400 MHz, DMSO-d₆) δ ppm 11.55 (s, 1H), 7.53-7.22 (m, 6H), 6.10 (t, J=56.9 Hz, 1H), 5.78-5.62 (m, 1H), 5.25-5.01 (m, 4H), 4.73-4.57 (m, 2H), 4.20-4.10 (m, 1H), 3.44-3.03 (m, 7H), 2.85-2.61 (m, 1H), 2.17-2.00 (m, 2H), 1.56-1.43 (m, 9H), 1.43-1.21 (m, 18H); MS (ESI⁻) m/z 841 [M−H]⁻.

### Example II-9E: tert-butyl (2R)-6-(benzyloxy)-2-{[(tert-butoxycarbonyl)(3,3-difluoropropyl)amino]methyl}-4-fluoro-5-(1,1,4-trioxo-1λ⁶,2,5-thiadiazolidin-2-yl)-2,3-dihydro-1H-indole-1-carboxylate-ammonia

To a solution of the product of Example II-9D (737 mg, 0.647 mmol) and potassium carbonate (358 mg, 2.59 mmol) in methanol (8 mL) was added tetrakis(triphenylphosphine)palladium(0) (74.8 mg, 0.065 mmol). The reaction mixture was stirred at 60° C. for 4 hours. The mixture was cooled to room temperature and diluted with methanol (5 mL). C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 10 mM ammonium bicarbonate, 60 mL/minute) to afford the title compound as an ammonium salt (347 mg, 0.425 mmol, 66% yield, 86% purity). ¹H NMR (400 MHz, DMSO-d₆) δ ppm 7.68-7.24 (m, 6H), 7.11-6.91 (m, 3H), 6.09 (t, J=56.4 Hz, 1H), 5.09 (s, 2H), 4.72-4.61 (m, 1H), 3.98-3.82 (m, 2H), 3.28-3.21 (m, 2H), 3.19-3.06 (m, 1H), 2.86-2.69 (m, 1H), 2.15-1.95 (m, 2H), 1.48 (s, 9H), 1.38 (s, 5H), 1.29 (s, 6H); MS (ESI⁻) m/z 683 [M−H]⁻.

### Example II-9F: tert-butyl (2R)-2-{[(tert-butoxycarbonyl)(3,3-difluoropropyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a solution of the product of Example II-9E (347 mg, 0.436 mmol) in degassed water (0.5 mL) and ethanol (4 mL) was added 10% Pd/C (50 mg). The resulting suspension was allowed to stir under hydrogen (5 bar) for 18 hours. The suspension was concentrated under reduced pressure onto C18 silica gel (3 g). The crude residue was subjected to column chromatography (Reveleris® 24 g reversed phase (C18) flash cartridge, dry loaded with C18 silica, 10-60% methanol in 10 mM ammonium bicarbonate, 40 mL/minute) to afford the title product as an ammonium salt (208 mg, 0.323 mmol, 74% yield). ¹H NMR (400 MHz, DMSO-d₆) δ ppm 9.30 (s, 1H), 7.09 (s, 4H), 6.09 (tt, J=56.3, 4.1 Hz, 1H), 4.67-4.51 (m, 1H), 3.96-3.80 (m, 2H), 3.38-3.19 (m, 4H), 3.07 (dd, J=15.7, 9.1 Hz, 1H), 2.82-2.59 (m, 1H), 2.14-1.96 (m, 2H), 1.49 (s, 9H), 1.38 (s, 4H), 1.32 (s, 5H); MS (ESI⁻) m/z 593 [M−H]⁻.

### Example II-9G: 5-[(2R)-2-{[(3,3-difluoropropyl)amino]methyl}-4-fluoro-6-hydroxy-2,3-dihydro-1H-indol-5-yl]-1λ⁶,2,5-thiadiazolidine-1,1,3-trione

To a solution of the product of Example II-9F (99 mg, 0.158 mmol) in dichloromethane (4 mL) at room temperature was added trifluoroacetic acid (0.18 mL, 2.336 mmol). The reaction mixture was stirred at 25° C. for 7 hours. The reaction mixture was neutralized with 0.7 M NH₃ in methanol:water (95:5) (8 mL) and concentrated under reduced pressure. The crude residue was subjected to column chromatography (Reveleris® 12 g reversed phase (C18) flash cartridge, dry load on C18 silica, 10-50% methanol in 10 mM ammonium bicarbonate, 30 mL/minute) to afford the title compound (43 mg, 0.109 mmol, 69% yield). ¹H NMR (400 MHz, DMSO-d₆) δ ppm 8.70 (s, 1H), 8.41 (s, 1H), 6.22 (tt, J=56.0, 4.1 Hz, 1H), 6.03 (s, 1H), 5.93 (s, 1H), 4.11 (s, 1H), 3.17-2.97 (m, 5H), 2.74 (dd, J=15.6, 7.0 Hz, 1H), 2.32-2.14 (m, 2H); MS (ESI⁺) m/z 395 [M+H]⁺.

### Example II-10: 5-[(2R)-4-fluoro-6-hydroxy-2-{[(3,3,3-trifluoropropyl)amino]methyl}-2,3-dihydro-1H-indol-5-yl]-1λ⁶,2,5-thiadiazolidine-1,1,3-trione (Compound 141)

### Example II-10A: tert-butyl (2R)-6-(benzyloxy)-5-[(2-tert-butoxy-2-oxoethyl)(trifluoroacetyl)amino]-4-fluoro-2-{[(3,3,3-trifluoropropyl)amino]methyl}-2,3-dihydro-1H-indole-1-carboxylate

Acetic acid (0.21 mL, 3.67 mmol) was added to a solution of the product of Example II-2I (0.85 g, 1.282 mmol) and 3,3,3-trifluoropropan-1-amine (0.41 mL, 3.63 mmol) in 1,2-dichloroethane (6 mL). The reaction mixture was stirred at room temperature for 1 hour. Sodium triacetoxyborohydride (0.408 g, 1.924 mmol) was added, and the reaction mixture was stirred for 1 hour. The reaction mixture was quenched with a saturated aqueous solution of sodium bicarbonate (20 mL) and stirred for 10 minutes. The mixture was diluted with water (20 mL) and extracted with ethyl acetate (3×30 mL). The combined organic layers were washed with brine (20 mL), dried over sodium sulfate, filtered, and concentrated under reduced pressure to afford the title compound (1.0577 g, 1.128 mmol, 88% yield, 74% purity). ¹H NMR (400 MHz, DMSO-d₆) δ ppm 7.48-7.28 (m, 6H), 5.24-5.01 (m, 3H), 4.53-4.44 (m, 1H), 4.44-4.34 (m, 1H), 3.99-3.93 (m, 1H), 3.63-3.54 (m, 1H), 3.23-3.09 (m, 1H), 3.01-2.93 (m, 1H), 2.82-2.64 (m, 2H), 2.64-2.52 (m, 1H), 2.42-2.23 (m, 2H), 1.51 (s, 7H), 1.47 (s, 2H), 1.38 (s, 9H); MS (ESI⁺) m/z 695 [M+H]⁺.

### Example II-10B: tert-butyl (2R)-6-(benzyloxy)-2-{[(tert-butoxycarbonyl)(3,3,3-trifluoropropyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-10A (1.05 g, 1.211 mmol) in acetonitrile (6 mL) were added N,N-diisopropylethylamine (0.635 mL, 3.63 mmol) and di-tert-butyl dicarbonate (0.529 g, 2.422 mmol). The reaction mixture was stirred at room temperature for 1 hour. The reaction was quenched with half saturated brine (10 mL). The resultant mixture was diluted with ethyl acetate (20 mL), and the layers were separated. The aqueous layer was extracted with ethyl acetate (3×15 mL). The combined organic layers were dried with sodium sulfate, filtered, and concentrated under reduced pressure. The crude residue was subjected to column chromatography (silica gel, 80 g cartridge, 20% ethyl acetate in isohexane, 60 mL/minute) to afford the title compound (688 mg, 0.823 mmol, 68% yield). ¹H NMR (400 MHz, DMSO-d₆ at 90° C.) δ ppm 7.74-7.38 (m, 4H), 7.38-7.25 (m, 2H), 5.18 (dd, J=12.0, 2.7 Hz, 1H), 5.11 (dd, J=12.0, 4.9 Hz, 1H), 4.77-4.67 (m, 1H), 4.44 (dd, J=16.4, 9.5 Hz, 1H), 3.88 (dd, J=28.6, 16.5 Hz, 1H), 3.46-3.28 (m, 4H), 3.19 (dt, J=16.6, 8.6 Hz, 1H), 2.99-2.84 (m, 1H), 2.57-2.42 (m, 2H), 1.53 (d, J=2.4 Hz, 9H), 1.40 (d, J=1.8 Hz, 9H), 1.36 (d, J=6.9 Hz, 9H); MS (ESI⁺) m/z 816 [M+Na]⁺.

### Example II-10C: tert-butyl (2R)-6-(benzyloxy)-2-{[(tert-butoxycarbonyl)(3,3,3-trifluoropropyl)amino]methyl}-5-[(2-tert-butoxy-2-oxoethyl)amino]-4-fluoro-2,3-dihydro-1H-indole-1-carboxylate

To a solution of the product of Example II-10B (688 mg, 0.823 mmol) in methanol (2.5 mL) and tetrahydrofuran (2.5 mL) at room temperature was added a solution of lithium hydroxide monohydrate (104 mg, 2.470 mmol) in water (2.5 mL). The reaction mixture was then stirred at room temperature for 45 minutes. The reaction mixture was neutralized to pH 7 with 1 M aqueous hydrochloric acid (2.470 mL) and diluted with water (10 mL). The aqueous layer was extracted with ethyl acetate (3×10 mL). The combined organic layers were washed with brine (10 mL), dried over sodium sulfate, filtered, and concentrated in vacuo to afford the title compound (632 mg, 0.543 mmol, 66% yield, 60% purity). MS (ESI⁺) m/z 599 [M-C(O)OC(CH₃)₃+H]⁺.

### Example II-10D: tert-butyl (2R)-6-(benzyloxy)-2-{[(tert-butoxycarbonyl)(3,3,3-trifluoropropyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.15 mL, 1.728 mmol) in dichloromethane (3 mL) at 0° C. was added allyl alcohol (0.12 mL, 1.764 mmol) dropwise. The resulting solution was stirred for 20 minutes at 0° C. before a prepared solution of the product of Example II-10C (632 mg, 0.543 mmol) and triethylamine (0.30 mL, 2.152 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-100% ethyl acetate in isohexane, 40 mL/minute) to afford the title compound (242 mg, 0.143 mmol, 26% yield, 51% purity). MS (ESI⁺) m/z 765 [M—CH₂CH═CH₂—C(CH₃)₃+H]⁺.

### Example II-10E: tert-butyl (2R)-6-(benzyloxy)-2-{[(tert-butoxycarbonyl)(3,3,3-trifluoropropyl)amino]methyl}-4-fluoro-5-(1,1,4-trioxo-1λ⁶,2,5-thiadiazolidin-2-yl)-2,3-dihydro-1H-indole-1-carboxylate

To a solution of the product of Example II-10D (242 mg, 0.143 mmol) and potassium carbonate (79 mg, 0.573 mmol) in methanol (4 mL) was added tetrakis(triphenylphosphine)palladium(0) (16.57 mg, 0.014 mmol). The reaction mixture was stirred at 60° C. for 4 hours. The mixture was cooled to room temperature and diluted with methanol (5 mL). C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 10 mM ammonium bicarbonate, 60 mL/minute) to afford the title compound (37.7 mg, 0.029 mmol, 20% yield, 54% purity). MS (ESI⁻) m/z 701 [M−H]⁻.

### Example II-10F: tert-butyl (2R)-2-{[(tert-butoxycarbonyl)(3,3,3-trifluoropropyl)amino]methyl}-4-fluoro-6-hydroxy-5-(1,1,4-trioxo-1λ⁶,2,5-thiadiazolidin-2-yl)-2,3-dihydro-1H-indole-1-carboxylate

To a solution of the product of Example II-10E (37.7 mg, 0.029 mmol) in degassed water (0.5 mL) and ethanol (4 mL) was added 10% Pd/C (10 mg). The resulting suspension was allowed to stir under hydrogen (5 bar) for 18 hours. The suspension was concentrated under reduced pressure onto C18 silica gel (2 g). The crude residue was subjected to column chromatography (Reveleris® 24 g reversed phase (C18) flash cartridge, dry loaded with C18 silica, 10-60% methanol in 10 mM ammonium bicarbonate, 40 mL/minute) to afford the title compound (14.5 mg, 0.017 mmol, 58% yield, 71% purity). MS (ESI⁻) m/z 611 [M−H]⁻. Example II-10G: 5-[(2R)-4-fluoro-6-hydroxy-2-{[(3,3,3-trifluoropropyl)amino]methyl}-2,3-dihydro-1H-indol-5-yl]-1λ⁶,2,5-thiadiazolidine-1,1,3-trione

To a solution of the product of Example II-10F (14.5 mg, 0.017 mmol) in dichloromethane (2 mL) at room temperature was added trifluoroacetic acid (0.02 mL, 0.260 mmol). The reaction mixture was stirred at 25° C. for 7 hours. The reaction mixture was neutralized with 0.7 M NH₃ in methanol:water (95:5) (6 mL) and concentrated under reduced pressure. The crude residue was subjected to column chromatography (Reveleris® 12 g reversed phase (C18) flash cartridge, dry load on C18 silica, 10-60% methanol in 10 mM ammonium bicarbonate, 30 mL/minute) to afford the title compound (8.5 mg, 0.020 mmol, quantitative yield). ¹H NMR (400 MHz, DMSO-d₆) δ ppm 8.68 (s, 1H), 6.03 (s, 1H), 5.93 (s, 1H), 4.14-4.04 (m, 1H), 3.84 (s, 2H), 3.19 (s, 2H), 3.15-2.99 (m, 3H), 2.77-2.61 (m, 3H); MS (ESI⁺) m/z 413 [M+H]⁺.

### Example II-11: 5-[(2R)-4-fluoro-6-hydroxy-2-{[(2-methylbutyl)amino]methyl}-2,3-dihydro-1H-indol-5-yl]-1λ⁶,2,5-thiadiazolidine-1,1,3-trione (Compound 142)

### Example II-11A: tert-butyl (2R)-6-(benzyloxy)-5-[(2-tert-butoxy-2-oxoethyl)(trifluoroacetyl)amino]-4-fluoro-2-{[(2-methylbutyl)amino]methyl}-2,3-dihydro-1H-indole-1-carboxylate

Acetic acid (0.23 mL, 4.02 mmol) was added to a solution of 2-methylbutan-1-amine (350 mg, 4.02 mmol) and the product of Example II-2I (0.885 g, 1.335 mmol) in 1,2-dichloroethane (3 mL). The reaction mixture was stirred at room temperature for 2 hours. Additional 2-methylbutan-1-amine (175 mg, 2.008 mmol) and acetic acid (120 mg, 2.003 mmol) were added, and the reaction mixture was stirred at room temperature for 1 hour. Sodium triacetoxyborohydride (420 mg, 1.982 mmol) was added, and the reaction mixture was stirred for 30 minutes. The reaction mixture was quenched with a saturated aqueous solution of sodium bicarbonate (6 mL) and stirred for 10 minutes. The reaction mixture was diluted with water (20 mL) and extracted with ethyl acetate (3×20 mL). The combined organic layers were washed with brine (20 mL), dried over sodium sulfate, filtered, and concentrated under reduced pressure to afford the title compound (0.934 g, 1.189 mmol, 89% yield, 85% purity). ¹H NMR (400 MHz, DMSO-d₆) δ ppm 7.50-7.20 (m, 6H), 5.23-4.99 (m, 2H), 4.56-4.31 (m, 2H), 4.09-3.85 (m, 1H), 3.15 (dt, J=15.8, 10.0 Hz, 1H), 3.01 (dt, J=17.1, 4.0 Hz, 1H), 2.79-2.70 (m, 1H), 2.64-2.21 (m, 6H), 1.50 (s, 9H), 1.38 (s, 9H), 1.12-0.96 (m, 1H), 0.92-0.71 (m, 6H); MS (ESI⁺) m/z 669 [M+H]⁺.

### Example II-11B: tert-butyl (2R)-6-(benzyloxy)-2-{[(tert-butoxycarbonyl)(2-methylbutyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-11A (935 mg, 1.190 mmol) in acetonitrile (6 mL) at room temperature was added N,N-diisopropylethylamine (0.63 mL, 3.61 mmol) and di-tert-butyl dicarbonate (520 mg, 2.380 mmol). The mixture was stirred at room temperature for 30 minutes. The reaction was quenched with water (10 mL). The aqueous layer was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-30% ethyl acetate in isohexane, 32 mL/minute) to afford the title compound (770 mg, 0.903 mmol, 76% yield). ¹H NMR (400 MHz, DMSO-d₆) δ ppm 7.51-7.25 (m, 6H), 5.23-5.04 (m, 2H), 4.69 (s, 1H), 4.48-4.33 (m, 1H), 4.04-3.92 (m, 1H), 3.58-2.73 (m, 7H), 1.78-1.58 (m, 1H), 1.50 (d, J=4.6 Hz, 9H), 1.43-1.19 (m, 18H), 1.13-0.98 (m, 1H), 0.92-0.73 (m, 6H).

### Example II-11C: tert-butyl (2R)-6-(benzyloxy)-2-{[(tert-butoxycarbonyl)(2-methylbutyl)amino]methyl}-5-[(2-tert-butoxy-2-oxoethyl)amino]-4-fluoro-2,3-dihydro-H-indole-1-carboxylate

To a solution of the product of Example II-11B (770 mg, 0.903 mmol) in methanol (4 mL) and tetrahydrofuran (4 mL) at room temperature was added a solution of lithium hydroxide monohydrate (115 mg, 2.74 mmol) in water (4.00 mL). The reaction mixture was then stirred at room temperature for 1 hour. The reaction mixture was neutralized to pH 7 with 1 M hydrogen chloride solution and diluted with water (10 mL). The aqueous layer was extracted with ethyl acetate (3×10 mL). The combined organic layers were washed with brine (10 mL), dried over sodium sulfate, filtered, and concentrated in vacuo to afford the title compound (586 mg, 0.785 mmol, 87% yield). ¹H NMR (400 MHz, DMSO-d₆) δ ppm 7.58-7.12 (m, 6H), 5.08 (s, 2H), 4.77-4.64 (m, 1H), 4.58 (s, 1H), 3.84 (dd, J=7.1, 2.2 Hz, 2H), 3.50-3.28 (m, 1H), 3.27-2.89 (m, 5H), 2.88-2.61 (m, 1H), 1.81-1.57 (m, 1H), 1.47 (s, 9H), 1.34 (s, 18H), 1.04 (dt, J=10.7, 6.8 Hz, 1H), 0.90-0.69 (m, 6H).

### Example II-11D: tert-butyl (2R)-6-(benzyloxy)-2-{[(tert-butoxycarbonyl)(2-methylbutyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.14 mL, 1.612 mmol) in dichloromethane (3 mL) at 0° C. was added allyl alcohol (0.11 mL, 1.617 mmol) dropwise. The resulting solution was stirred for 30 minutes at 0° C. before a prepared solution of the product of Example II-11C (586 mg, 0.785 mmol) and triethylamine (0.27 mL, 1.937 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-40% ethyl acetate in isohexane, 40 mL/minute) to afford the title compound (530 mg, 0.597 mmol, 76% yield). ¹H NMR (400 MHz, DMSO-d₆) δ ppm 11.68-11.46 (m, 1H), 7.57-7.07 (m, 6H), 6.09-5.80 (m, 0.5H), 5.79-5.62 (m, 0.5H), 5.57-5.39 (m, 0.5H), 5.35-4.95 (m, 4.5H), 4.78 (dt, J=6.3, 1.2 Hz, 0.5H), 4.65 (dd, J=17.6, 10.8 Hz, 2H), 4.42 (dt, J=5.1, 1.6 Hz, 0.5H), 4.33-4.05 (m, 2H), 3.31-2.71 (m, 6H), 1.66 (s, 1H), 1.47 (s, 9H), 1.42-1.17 (m, 18H), 1.12-0.96 (m, 1H), 0.93-0.68 (m, 6H).

### Example II-11E: tert-butyl (2R)-6-(benzyloxy)-2-{[(tert-butoxycarbonyl)(2-methylbutyl)amino]methyl}-4-fluoro-5-(1,1,4-trioxo-1λ⁶,2,5-thiadiazolidin-2-yl)-2,3-dihydro-1H-indole-1-carboxylate-ammonia (Partial)

To a solution of the product of Example II-11D (623 mg, 0.597 mmol) and potassium carbonate (330 mg, 2.388 mmol) in methanol (4 mL) was added tetrakis(triphenylphosphine)palladium(0) (69.0 mg, 0.060 mmol). The reaction mixture was stirred at 60° C. for 5 hours. The mixture was cooled to room temperature and concentrated under reduced pressure. The crude residue was subjected to column chromatography (Reveleris® 40 g reversed phase (C18) flash cartridge, dry loaded with C18 silica gel, 5-80% methanol in 10 mM ammonium bicarbonate, 40 mL/minute) to afford the title compound as a partial ammonium salt (279 mg, 0.365 mmol, 61% yield, 90% purity). ¹H NMR (400 MHz, DMSO-d₆) δ ppm 7.67-7.45 (m, 3H), 7.40-7.24 (m, 3H), 7.09 (s, 2H), 5.09 (s, 2H), 4.66 (d, J=7.9 Hz, 1H), 3.99-3.80 (m, 2H), 3.42 (s, 0.5H), 3.37-2.70 (m, 7.5H), 1.79-1.58 (m, 1H), 1.49 (s, 9H), 1.43-1.19 (m, 9H), 1.12-0.97 (m, 1H), 0.93-0.72 (m, 6H); MS (ESI⁻) m/z 675 [M−H]⁻.

### Example II-11F: tert-butyl (2R)-2-{[(tert-butoxycarbonyl)(2-methylbutyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a solution of the product of Example II-11E (270 mg, 0.354 mmol) in degassed water (0.5 mL) and ethanol (4 mL) was added 10% Pd/C (36 mg). The resulting suspension was allowed to stir under hydrogen (5 bar) for 18 hours. The reaction mixture was filtered through a diatomaceous earth pad and concentrated under reduced pressure. The crude residue was subjected to column chromatography (Reveleris® 24 g reversed phase (C18) flash cartridge, dry loaded with C18 silica, 5-65% methanol in 10 mM ammonium bicarbonate, 32 mL/minute) to afford the title compound as an ammonium salt (185 mg, 0.291 mmol, 82% yield). ¹H NMR (400 MHz, DMSO-d₆) δ ppm 9.26 (s, 1H), 7.29-6.75 (m, 4H), 4.61 (s, 1H), 3.98-3.76 (m, 2H), 3.52-3.16 (m, 3H), 3.04 (dd, J=15.7, 9.1 Hz, 3H), 2.84-2.63 (m, 1H), 1.79-1.56 (m, 1H), 1.50 (s, 9H), 1.34 (d, J=16.5 Hz, 9H), 1.12-0.95 (m, 1H), 0.93-0.69 (m, 6H); MS (ESI⁺) m/z 587 [M+H]⁺.

### Example II-11G: 5-[(2R)-4-fluoro-6-hydroxy-2-{[(2-methylbutyl)amino]methyl}-2,3-dihydro-1H-indol-5-yl]-1λ⁶,2,5-thiadiazolidine-1,1,3-trione

To a solution of the product of Example II-11F (165 mg, 0.246 mmol) in dichloromethane (4 mL) at room temperature was added trifluoroacetic acid (0.284 mL, 3.69 mmol). The reaction mixture was stirred at room temperature for 3 hours. Additional trifluoroacetic acid (0.095 mL, 1.230 mmol) was added, and the reaction mixture was stirred at room temperature for 1 hour. Additional trifluoroacetic acid (0.095 mL, 1.230 mmol) was added, and the reaction mixture was stirred at room temperature for 1 hour. The reaction mixture was neutralized with 0.7 M NH₃ in methanol:water (95:5) (15 mL) and concentrated under reduced pressure. The crude residue was subjected to column chromatography (Reveleris® 12 g reversed phase (C18) flash cartridge, dry load on C18 silica, 10-35% methanol in 10 mM ammonium bicarbonate, 30 mL/minute) to afford the title compound (72.74 mg, 0.178 mmol, 72% yield). ¹H NMR (400 MHz, DMSO-d₆) δ ppm 8.68 (s, 1H), 8.29-8.01 (m, 2H), 6.02 (d, J=6.3 Hz, 1H), 5.93 (s, 1H), 4.13 (d, J=8.1 Hz, 1H), 3.84 (s, 2H), 3.17-2.95 (m, 3H), 2.91 (dt, J=13.0, 6.7 Hz, 1H), 2.74 (dt, J=15.8, 6.4 Hz, 2H), 1.75 (h, J=6.7 Hz, 1H), 1.48-1.35 (m, 1H), 1.27-1.10 (m, 1H), 0.99-0.80 (m, 6H); MS (ESI⁺) m/z 387 [M+H]⁺.

### Example II-12: 5-[(2R)-2-({[(3,3-difluorocyclobutyl)methyl]amino}methyl)-4-fluoro-6-hydroxy-2,3-dihydro-1H-indol-5-yl]-1λ⁶,2,5-thiadiazolidine-1,1,3-trione (Compound 143)

### Example II-12A: tert-butyl (2R)-6-(benzyloxy)-5-[(2-tert-butoxy-2-oxoethyl)(trifluoroacetyl)amino]-2-({[(3,3-difluorocyclobutyl)methyl]amino}methyl)-4-fluoro-2,3-dihydro-1H-indole-1-carboxylate

Acetic acid (0.217 mL, 3.78 mmol) was added to a solution of (3,3-difluorocyclobutyl)methanamine, hydrochloric acid (0.596 g, 3.78 mmol) and the product of Example II-2I (0.885 g, 1.261 mmol) in 1,2-dichloroethane (3 mL). Triethylamine (0.527 mL, 3.78 mmol) was added, and the reaction mixture was stirred at room temperature for 40 minutes. Sodium triacetoxyborohydride (0.401 g, 1.891 mmol) was added, and the reaction mixture was stirred for 30 minutes. The reaction mixture was quenched with a saturated aqueous solution of sodium bicarbonate (6 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (0.734 g, 1.008 mmol, 80% yield). ¹H NMR (400 MHz, DMSO-d₆) δ ppm 7.47-7.26 (m, 6H), 5.23-5.02 (m, 1H), 4.52-4.33 (m, 1H), 4.08-3.87 (m, 1H), 3.22-3.07 (m, 1H), 2.99 (d, J=16.3 Hz, 1H), 2.76 (d, J=12.0 Hz, 1H), 2.71-2.43 (m, 8H), 2.25-2.07 (m, 3H), 1.50 (s, 9H), 1.38 (s, 9H); MS (ESI⁺) m/z 703 [M+H]⁺.

### Example II-12B: tert-butyl (2R)-6-(benzyloxy)-2-({(tert-butoxycarbonyl)[(3,3-difluorocyclobutyl)methyl]amino}methyl)-5-[(2-tert-butoxy-2-oxoethyl)(trifluoroacetyl)amino]-4-fluoro-2,3-dihydro-1H-indole-1-carboxylate

To a solution of the product of Example II-12A (884 mg, 1.008 mmol) in acetonitrile (4 mL) at room temperature were added N,N-diisopropylethylamine (0.56 mL, 3.21 mmol) and di-tert-butyl dicarbonate (470 mg, 2.154 mmol). The mixture was stirred at room temperature for 30 minutes. The reaction was quenched with water (10 mL). The aqueous layer was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-40% ethyl acetate in isohexane, 32 mL/minute) to afford the title compound (630 mg, 0.742 mmol, 74% yield). ¹H NMR (400 MHz, DMSO-d₆) δ ppm 7.48-7.27 (m, 6H), 5.25-5.04 (m, 2H), 4.69 (s, 1H), 4.40 (d, J=16.7 Hz, 1H), 4.08-3.91 (m, 1H), 3.15 (dd, J=16.3, 9.3 Hz, 3H), 2.70-2.54 (m, 3H), 2.46-2.16 (m, 5H), 1.50 (s, 9H), 1.42-1.20 (m, 18H).

### Example II-12C: tert-butyl (2R)-6-(benzyloxy)-2-({(tert-butoxycarbonyl)[(3,3-difluorocyclobutyl)methyl]amino}methyl)-5-[(2-tert-butoxy-2-oxoethyl)amino]-4-fluoro-2,3-dihydro-1H-indole-1-carboxylate

To a solution of the product of Example II-12B (700 mg, 0.742 mmol) in methanol (4 mL) and tetrahydrofuran (4 mL) at room temperature was added a solution of lithium hydroxide monohydrate (93 mg, 2.226 mmol) in water (4.00 mL). The reaction mixture was then stirred at room temperature for 40 minutes. The reaction mixture was neutralized to pH 7 with 1 M hydrogen chloride (2.25 mL, 2.250 mmol) and diluted with water (10 mL). The aqueous layer was extracted with ethyl acetate (3×10 mL). The combined organic layers were washed with brine (10 mL), dried over sodium sulfate, filtered, and concentrated in vacuo to afford the title compound (535 mg, 0.720 mmol, 97% yield). ¹H NMR (400 MHz, DMSO-d₆) δ ppm 7.56-7.19 (m, 6H), 5.08 (s, 2H), 4.70 (s, 1H), 4.59 (s, 1H), 3.84 (dd, J=7.2, 2.2 Hz, 2H), 3.45-3.16 (m, 3H), 3.15-2.97 (m, 1H), 2.84-2.54 (m, 3H), 2.45-2.16 (m, 4H), 1.47 (s, 9H), 1.42-1.20 (m, 18H).

### Example II-12D: tert-butyl (2R)-6-(benzyloxy)-2-({(tert-butoxycarbonyl)[(3,3-difluorocyclobutyl)methyl]amino}methyl)-5-[(2-tert-butoxy-2-oxoethyl)(({[prop-2-en-1-yl)oxy]carbonyl}sulfamoyl)amino]-4-fluoro-2,3-dihydro-1H-indole-1-carboxylate

To a solution of chlorosulfonyl isocyanate (0.125 mL, 1.438 mmol) in dichloromethane (3 mL) at 0° C. was added allyl alcohol (0.1 mL, 1.470 mmol) dropwise. The resulting solution was stirred for 30 minutes at 0° C. before a prepared solution of the product of Example II-12C (534 mg, 0.719 mmol) and triethylamine (0.250 mL, 1.797 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-40% ethyl acetate in isohexane, 40 mL/minute) to afford the title compound (574 mg, 0.661 mmol, 92% yield). ¹H NMR (400 MHz, DMSO-d₆) δ ppm 11.68-11.46 (m, 1H), 7.60-7.01 (m, 6H), 6.07-5.94 (m, 0.5H), 5.93-5.80 (m, 0.5H), 5.79-5.64 (m, 1H), 5.56-5.41 (m, 1H), 5.35-5.01 (m, 4H), 4.78 (dt, J=6.2, 1.2 Hz, 1H), 4.65 (dd, J=17.5, 8.3 Hz, 2H), 4.42 (dt, J=5.1, 1.6 Hz, 1H), 4.32-4.04 (m, 3H), 3.18-3.03 (m, 1H), 2.88-2.54 (m, 3H), 2.46-2.20 (m, 3H), 1.56-1.15 (m, 27H).

### Example II-12E: tert-butyl (2R)-6-(benzyloxy)-2-({(tert-butoxycarbonyl)[(3,3-difluorocyclobutyl)methyl]amino}methyl)-4-fluoro-5-(1,1,4-trioxo-1λ⁶,2,5-thiadiazolidin-2-yl)-2,3-dihydro-1H-indole-1-carboxylate-ammonia

To a solution of the product of Example II-12D (718 mg, 0.661 mmol) and potassium carbonate (365 mg, 2.64 mmol) in methanol (3 mL) was added tetrakis(triphenylphosphine)palladium(0) (76 mg, 0.066 mmol). The reaction mixture was stirred at 60° C. for 5 hours. The mixture was cooled to room temperature and concentrated under reduced pressure. The crude material was diluted with methanol (8 mL), C18 silica gel (3 g) was added, and the mixture was concentrated in vacuo. The crude residue was subjected to column chromatography (Reveleris® 40 g reversed phase (C18) flash cartridge, dry loaded with C18 silica gel, 5-75% methanol in 10 mM ammonium bicarbonate, 40 mL/minute) to afford the title compound as an ammonium salt (195 mg, 0.228 mmol, 35% yield, 90% purity). ¹H NMR (400 MHz, DMSO-d₆) δ ppm 7.59-7.45 (m, 3H), 7.41-7.22 (m, 3H), 7.07 (s, 3H), 5.09 (s, 2H), 4.67 (s, 1H), 3.90 (q, J=13.2 Hz, 2H), 3.30 (d, J=9.6 Hz, 3H), 3.19-3.03 (m, 1H), 2.89-2.55 (m, 5H), 2.43-2.22 (m, 3H), 1.49 (s, 9H), 1.34 (d, J=39.1 Hz, 9H); MS (ESI⁻) m/z 709 [M−H]⁻.

### Example II-12F: tert-butyl (2R)-2-({(tert-butoxycarbonyl)[(3,3-difluorocyclobutyl)methyl]amino}methyl)-4-fluoro-6-hydroxy-5-(1,1,4-trioxo-1λ⁶,2,5-thiadiazolidin-2-yl)-2,3-dihydro-1H-indole-1-carboxylate-ammonia

To a solution of the product of Example II-12E (190 mg, 0.222 mmol) in degassed water (0.5 mL) and ethanol (4 mL) was added 10% Pd/C (23.6 mg). The resulting suspension was allowed to stir under hydrogen (5 bar) for 18 hours. The reaction mixture was filtered through a diatomaceous earth pad, and the filtrate was concentrated under reduced pressure. The crude residue was subjected to column chromatography (Reveleris® 24 g reversed phase (C18) flash cartridge, dry loaded with C18 silica, 5-65% methanol in 10 mM ammonium bicarbonate, 32 mL/minute) to afford the title compound as an ammonium salt (115 mg, 0.171 mmol, 77% yield). ¹H NMR (400 MHz, DMSO-d₆) δ ppm 9.27 (s, 1H), 7.28-6.81 (m, 4H), 4.62 (d, J=8.7 Hz, 1H), 3.97-3.75 (m, 2H), 3.49-3.13 (m, 4H), 3.05 (dd, J=15.6, 9.2 Hz, 1H), 2.82-2.55 (m, 3H), 2.44-2.21 (m, 4H), 1.50 (s, 9H), 1.35 (d, J=27.7 Hz, 9H); MS (ESI⁻) m/z 620 [M−H]⁻.

### Example II-12G: 5-[(2R)-2-({[(3,3-difluorocyclobutyl)methyl]amino}methyl)-4-fluoro-6-hydroxy-2,3-dihydro-1H-indol-5-yl]-1λ⁶,2,5-thiadiazolidine-1,1,3-trione

To a solution of the product of Example II-12F (115 mg, 0.171 mmol) in dichloromethane (4 mL) at room temperature was added trifluoroacetic acid (0.198 mL, 2.57 mmol). The reaction mixture was stirred at room temperature for 3 hours. The reaction mixture was neutralized with 0.7 M NH₃ in methanol:water (95:5) (5 mL) and concentrated under reduced pressure. The crude residue was subjected to column chromatography (Reveleris® 12 g reversed phase (C18) flash cartridge, dry load on C18 silica, 10-35% methanol in 10 mM ammonium bicarbonate, 30 mL/minute) to afford the title compound (39.31 mg, 0.089 mmol, 52% yield). ¹H NMR (400 MHz, DMSO-d₆) δ ppm 8.69 (d, J=2.4 Hz, 1H), 8.35 (s, 2H), 6.01 (s, 1H), 5.93 (s, 1H), 4.11 (s, 1H), 3.84 (s, 2H), 3.20-2.92 (m, 5H), 2.81-2.64 (m, 3H), 2.57-2.36 (m, 3H); MS (ESI⁺) m/z 421 [M+H]⁺.

### Example II-13: 5-[4-fluoro-6-hydroxy-2-({[2-(oxetan-3-yl)ethyl]amino}methyl)-2,3-dihydro-1H-indol-5-yl]-1λ⁶,2,5-thiadiazolidine-1,1,3-trione (Compound 144)

### Example II-13A: tert-butyl (2R)-6-(benzyloxy)-5-[(2-tert-butoxy-2-oxoethyl)(trifluoroacetyl)amino]-4-fluoro-2-({[2-(oxetan-3-yl)ethyl]amino}methyl)-2,3-dihydro-1H-indole-1-carboxylate

Acetic acid (0.22 mL, 3.84 mmol) was added to a solution of 2-(oxetan-3-yl)ethanamine (0.38 g, 3.76 mmol) and the product of Example II-2I (0.885 g, 1.335 mmol) in 1,2-dichloroethane (3 mL). The reaction mixture was stirred at room temperature for 2 hours. Additional 2-(oxetan-3-yl)ethanamine (0.19 g, 1.878 mmol) and acetic acid (0.11 g, 1.832 mmol) were added, and the reaction mixture was stirred at room temperature for 1 hour. Sodium triacetoxyborohydride (0.4 g, 1.887 mmol) was added, and the reaction mixture was stirred for 40 minutes. The reaction mixture was quenched with a saturated aqueous solution of sodium bicarbonate (6 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (0.93 g, 1.228 mmol, 92% yield, 90% purity). ¹H NMR (400 MHz, DMSO-d₆) δ ppm 7.49-7.21 (m, 6H), 5.25-5.01 (m, 2H), 4.64-4.33 (m, 3H), 4.06-3.88 (m, 1H), 3.32 (s, 3H), 3.04-2.88 (m, 1H), 2.69-2.31 (m, J=1.9 Hz, 7H), 2.29-2.10 (m, 1H), 1.76 (dd, J=18.3, 11.1 Hz, 1H), 1.50 (d, J=3.0 Hz, 9H), 1.38 (s, 9H); MS (ESI⁺) m/z 683 [M+H]⁺.

### Example II-13B: tert-butyl (2R)-6-(benzyloxy)-2-({(tert-butoxycarbonyl)[2-(oxetan-3-yl)ethyl]amino}methyl)-5-[(2-tert-butoxy-2-oxoethyl)(trifluoroacetyl)amino]-4-fluoro-2,3-dihydro-1H-indole-1-carboxylate

To a solution of the product of Example II-13A (846 mg, 1.241 mmol) in acetonitrile (6 mL) were added N,N-diisopropylethylamine (0.650 mL, 3.72 mmol), 4-dimethylaminopyridine (152 mg, 1.241 mmol) and di-tert-butyl dicarbonate (542 mg, 2.482 mmol) successively. The reaction mixture was stirred at ambient temperature for 2 hours. Additional portions of di-tert-butyl dicarbonate (271 mg, 1.241 mmol) and 4-dimethylaminopyridine (152 mg, 1.241 mmol) were added, and the reaction mixture was stirred for another 30 minutes. The reaction was quenched with half saturated brine (10 mL). The mixture was diluted with ethyl acetate (20 mL), and the layers were separated. The aqueous layer was extracted with ethyl acetate (3×15 mL). The combined organic layers were dried with sodium sulfate, filtered, and concentrated under reduced pressure. The crude residue was subjected to column chromatography (silica gel, 80 g cartridge, isocratic 20% ethyl acetate in isohexane, 60 mL/minute) to afford the title compound (442 mg, 0.565 mmol, 45% yield). ¹H NMR (400 MHz, DMSO-d₆ at 90° C.) δ ppm 7.43-7.35 (m, 5H), 7.37-7.30 (m, 1H), 5.22-5.06 (m, 2H), 4.60-4.49 (m, 1H), 4.48-4.38 (m, 1H), 3.97-3.77 (m, 3H), 3.26-3.13 (m, 1H), 3.00-2.92 (m, 1H), 2.73-2.51 (m, 5H), 2.44-2.28 (m, 2H), 1.93-1.75 (m, 1H), 1.54 (s, 9H), 1.47-1.34 (m, 19H); MS (ESI⁺) m/z 782 [M+H]⁺.

### Example II-13C: tert-butyl (2R)-6-(benzyloxy)-2-({(tert-butoxycarbonyl)[2-(oxetan-3-yl)ethyl]amino}methyl)-5-[(2-tert-butoxy-2-oxoethyl)amino]-4-fluoro-2,3-dihydro-1H-indole-1-carboxylate

To a solution of the product of Example II-13B (490 mg, 0.564 mmol) in methanol (1.5 mL) and tetrahydrofuran (1.5 mL) at room temperature was added a solution of lithium hydroxide monohydrate (71.0 mg, 1.692 mmol) in water (1.5 mL). The reaction mixture was then stirred at room temperature for 45 minutes. The reaction mixture was neutralized to pH 7 with 1 M hydrogen chloride (1.7 mL, 1.7 mmol) and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (371 mg, 0.541 mmol, 96% yield). ¹H NMR (400 MHz, DMSO-d₆ at 90° C.) δ ppm 7.50-7.43 (m, 2H), 7.43-7.31 (m, 3H), 7.20 (s, 1H), 5.08 (s, 2H), 4.48-4.44 (m, 1H), 3.96-3.88 (m, 2H), 3.85 (dd, J=6.7, 2.0 Hz, 2H), 3.12 (ddd, J=15.9, 9.2, 2.7 Hz, 1H), 2.94 (dt, J=15.9, 2.7 Hz, 1H), 2.74-2.51 (m, 5H), 2.43-2.27 (m, 2H), 1.94-1.79 (m, 1H), 1.51 (s, 9H), 1.45-1.41 (m, 10H), 1.37 (s, 9H); MS (ESI⁺) m/z 687 [M+H]⁺.

### Example II-13D: tert-butyl (2R)-6-(benzyloxy)-2-({(tert-butoxycarbonyl)[2-(oxetan-3-yl)ethyl]amino}methyl)-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

A solution of chlorosulfonyl isocyanate (0.094 mL, 1.082 mmol) in dichloromethane (2 mL) was cooled to 0° C. and allyl alcohol (0.074 mL, 1.082 mmol) was added dropwise. The resulting solution was stirred for 20 minutes at 0° C. before a solution of the product of Example II-13C (464 mg, 0.541 mmol) and triethylamine (0.189 mL, 1.353 mmol) in dichloromethane (3 mL) was added dropwise. The resulting solution was stirred at 0° C. for 30 minutes. The reaction was diluted with dichloromethane (10 mL) and quenched with water. The layers were separated, and the aqueous layer was extracted with dichloromethane (3×10 mL). The combined organic layers were dried with sodium sulfate, filtered, and concentrated under reduced pressure. The crude residue was subjected to column chromatography (silica gel, 40 g cartridge, 0-100% (3:1 ethyl acetate: ethanol) in isohexane, 40 mL/minute) to afford the title compound (370 mg, 0.446 mmol, 82% yield). ¹H NMR (400 MHz, DMSO-d₆ at 90° C.) δ ppm 9.65 (s, 1H), 7.50 (d, J=6.9 Hz, 2H), 7.44-7.32 (m, 3H), 7.17 (s, 1H), 6.49 (s, 2H), 5.93 (ddt, J=17.3, 10.0, 4.8 Hz, 1H), 5.25-5.08 (m, 3H), 5.02 (dq, J=10.4, 1.8 Hz, 1H), 4.87-4.76 (m, 1H), 4.38-4.22 (m, 1H), 4.11-4.01 (m, 1H), 3.99-3.93 (m, 2H), 3.59-3.08 (m, 6H), 2.95-2.58 (m, 2H), 2.38-2.04 (m, 1H), 1.88-1.64 (m, 1H), 1.59-1.50 (m, 9H), 1.45 (s, 9H), 1.42-1.38 (m, 1H), 1.35 (s, 9H); MS (ESI⁺) m/z 849 [M+H]⁺.

### Example II-13E: tert-butyl (2R)-6-(benzyloxy)-2-({(tert-butoxycarbonyl)[2-(oxetan-3-yl)ethyl]amino}methyl)-4-fluoro-5-(1,1,4-trioxo-1λ⁶,2,5-thiadiazolidin-2-yl)-2,3-dihydro-1H-indole-1-carboxylate

To a solution of the product of Example II-13D (435 mg, 0.436 mmol) in methanol (4 mL) were added tetrakis(triphenylphosphine)palladium(0) (50.3 mg, 0.044 mmol) and potassium carbonate (241 mg, 1.742 mmol). The reaction mixture was stirred at 60° C. for 4 hours. The reaction mixture was concentrated under reduced pressure. The crude residue was subjected to column chromatography (Reveleris® 40 g reversed phase (C18) flash cartridge, 10-100% methanol in 0.1% ammonium hydroxide, 40 mL/minute) to afford the title compound (104 mg, 0.120 mmol, 28% yield, 80% purity). ¹H NMR (400 MHz, DMSO-d₆ at 90° C.) δ ppm 7.69-7.16 (m, 6H), 5.10 (s, 2H), 4.57-4.40 (m, 1H), 3.99-3.86 (m, 4H), 3.23-3.10 (m, 1H), 2.99-2.92 (m, 2H), 2.78-2.52 (m, 5H), 2.44-2.37 (m, 1H), 1.89-1.84 (m, 1H), 1.56-1.48 (m, 9H), 1.46-1.41 (m, 10H); MS (ESI⁺) m/z 691 [M+H]⁺.

### Example II-13F: tert-butyl (2R)-2-({(tert-butoxycarbonyl)[2-(oxetan-3-yl)ethyl]amino}methyl)-4-fluoro-6-hydroxy-5-(1,1,4-trioxo-1λ⁶,2,5-thiadiazolidin-2-yl)-2,3-dihydro-1H-indole-1-carboxylate

To a solution of the product of Example II-13E (104 mg, 0.120 mmol) in ethanol (1.3 mL) and water (0.13 mL) was added 10% palladium on carbon (28.5 mg). The resulting suspension was allowed to stir under hydrogen (5 bar) for 16 hours. The reaction mixture was filtered through a Whatman glass fiber filter paper and washed with ethanol (3×15 mL). The filtrate was concentrated under reduced pressure, and the crude residue was subjected to column chromatography (Reveleris® 12 g reversed phase (C18) flash cartridge, dry loaded with C18 silica, 10-50% methanol in 0.1% ammonium hydroxide, 30 mL/minute) to afford the title compound (27 mg, 0.042 mmol, 35% yield). MS (ESI⁺) m/z 601 [M+H]⁺.

### Example II-13G: 5-[4-fluoro-6-hydroxy-2-({[2-(oxetan-3-yl)ethyl]amino}methyl)-2,3-dihydro-1H-indol-5-yl]-1λ⁶,2,5-thiadiazolidine-1,1,3-trione

To a solution of the product of Example II-13F (25 mg, 0.039 mmol) in dichloromethane (0.7 mL) at room temperature was added trifluoroacetic acid (0.045 mL, 0.587 mmol). The reaction mixture was stirred at ambient temperature for 3 hours, then cooled to 0° C. and quenched with 0.7 M ammonia in methanol (1.5 mL). The reaction mixture was concentrated under reduced pressure. The crude residue was subjected to column chromatography (Reveleris® 12 g reversed phase (C18) flash cartridge, dry loaded with C18 silica, isocratic 5% methanol in 0.1% ammonium hydroxide, 30 mL/minute) and then a second column chromatography (Reveleris® 12 g reversed phase (C18) flash cartridge, isocratic 0% methanol in 0.1% ammonium hydroxide, 30 mL/minute) to afford the title compound (8 mg, 0.019 mmol, 49% yield). ¹H NMR (400 MHz, DMSO-d₆ at 90° C.) δ ppm 8.23 (s, 1H), 5.96 (s, 1H), 5.81 (s, 1H), 4.22-4.10 (m, 1H), 3.87 (s, 2H), 3.51-3.32 (m, 3H), 3.31-3.11 (m, 5H), 2.69 (dd, J=15.7, 7.1 Hz, 1H), 2.48-2.41 (m, 1H), 2.09-1.95 (m, 1H), 1.82-1.62 (m, 1H); MS (ESI⁺) m/z 401 [M+H]⁺.

### Example II-14: 5-(2-{[(4,4-difluorobutyl)amino]methyl}-4-fluoro-6-hydroxy-2,3-dihydro-1H-indol-5-yl)-1λ⁶,2,5-thiadiazolidine-1,1,3-trione (Compound 145)

### Example II-14A: tert-butyl (2R)-6-(benzyloxy)-5-[(2-tert-butoxy-2-oxoethyl)(trifluoroacetyl)amino]-2-{[(4,4-difluorobutyl)amino]methyl}-4-fluoro-2,3-dihydro-1H-indole-1-carboxylate

Acetic acid (0.220 mL, 3.85 mmol) was added to a solution of 4,4-difluorobutan-1-amine, hydrochloric acid (0.560 g, 3.85 mmol) and the product of Example II-2I (0.85 g, 1.282 mmol) in 1,2-dichloroethane (6 mL). The reaction mixture was stirred at room temperature for 1 hour. Sodium triacetoxyborohydride (0.408 g, 1.924 mmol) was added, and the reaction mixture was stirred for 1 hour. The reaction mixture was quenched with a saturated aqueous solution of sodium bicarbonate (20 mL) and stirred for 10 minutes. The mixture was diluted with water (20 mL) and extracted with ethyl acetate (3×30 mL). The combined organic layers were washed with brine (20 mL), dried over sodium sulfate, filtered, and concentrated under reduced pressure to afford the title compound (1.0144 g, 1.309 mmol, 100% yield). ¹H NMR (400 MHz, DMSO-d₆) δ ppm 7.46-7.29 (m, 6H), 6.21-5.87 (m, 1H), 5.21-5.03 (m, 2H), 4.47 (s, 1H), 4.40 (dd, J=16.6, 2.7 Hz, 1H), 3.96 (dd, J=16.6, 3.3 Hz, 1H), 3.22-3.09 (m, 1H), 3.04-2.95 (m, 1H), 2.79-2.70 (m, 1H), 2.62-2.52 (m, 2H), 1.88-1.68 (m, 2H), 1.50 (s, 9H), 1.48-1.41 (m, 4H), 1.38 (s, 9H); MS (ESI⁺) m/z 691 [M+H]⁺.

### Example II-14B: tert-butyl (2R)-6-(benzyloxy)-2-{[(tert-butoxycarbonyl)(4,4-difluorobutyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-14A (1.01 g, 1.172 mmol) in acetonitrile (6 mL) were added N,N-diisopropylethylamine (0.614 mL, 3.51 mmol) and di-tert-butyl dicarbonate (0.511 g, 2.343 mmol). The reaction mixture was stirred at ambient temperature for 1 hour. The reaction was quenched with half saturated brine (10 mL). The mixture was diluted with ethyl acetate (20 mL), and the layers were separated. The aqueous layer was extracted with ethyl acetate (3×15 mL). The combined organic layers were dried with sodium sulfate, filtered, and concentrated under reduced pressure. The crude residue was subjected to column chromatography (silica gel, 80 g cartridge, isocratic 20% ethyl acetate in isohexane, 60 mL/minute) to afford the title compound (790 mg, 1.001 mmol, 83% yield). ¹H NMR (400 MHz, DMSO-d₆ at 90° C.) δ ppm 7.44-7.29 (m, 6H), 6.03 (tq, J=56.9, 4.2 Hz, 1H), 5.18 (dd, J=12.0, 3.9 Hz, 1H), 5.11 (d, J=11.9 Hz, 1H), 4.75-4.65 (m, 1H), 4.43 (dd, J=16.5, 9.0 Hz, 1H), 3.87 (dd, J=26.1, 16.5 Hz, 1H), 3.45-3.30 (m, 2H), 3.27-3.09 (m, 3H), 2.99-2.85 (m, 1H), 1.89-1.70 (m, 1H), 1.70-1.59 (m, 2H), 1.59-1.47 (m, 10H), 1.42-1.38 (m, 9H), 1.36 (d, J=7.5 Hz, 9H); MS (ESI⁺) m/z 812 [M+Na]⁺.

### Example II-14C: tert-butyl (2R)-6-(benzyloxy)-2-{[(tert-butoxycarbonyl)(4,4-difluorobutyl)amino]methyl}-5-[(2-tert-butoxy-2-oxoethyl)amino]-4-fluoro-2,3-dihydro-1H-indole-1-carboxylate

To a solution of the product of Example II-14B (930 mg, 1.001 mmol) in methanol (2.5 mL) and tetrahydrofuran (2.5 mL) at room temperature was added a solution of lithium hydroxide monohydrate (126 mg, 3.00 mmol) in water (2.5 mL). The reaction mixture was then stirred at ambient temperature for 45 minutes. The reaction mixture was neutralized to pH 7 with 1 M hydrogen chloride (3.00 mL, 3.00 mmol) and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (800 mg, 0.997 mmol, 100% yield, 90% purity). ¹H NMR (400 MHz, DMSO-d₆ at 90° C.) δ ppm 7.54-7.43 (m, 2H), 7.47-7.35 (m, 2H), 7.38-7.29 (m, 1H), 7.17 (s, 1H), 6.02 (tt, J=56.9, 4.3 Hz, 1H), 5.09 (s, 2H), 4.66-4.56 (m, 1H), 4.51 (td, J=6.7, 2.6 Hz, 1H), 3.85 (dd, J=6.7, 2.0 Hz, 2H), 3.40-3.24 (m, 2H), 3.26-3.14 (m, 2H), 3.10 (dd, J=15.9, 9.2 Hz, 1H), 2.81 (dd, J=15.9, 2.1 Hz, 1H), 1.86-1.52 (m, 4H), 1.51 (s, 9H), 1.38 (s, 18H); MS (ESI⁺) m/z 717 [M+Na]⁺.

### Example II-14D: tert-butyl (2R)-6-(benzyloxy)-2-{[(tert-butoxycarbonyl)(4,4-difluorobutyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

A solution of chlorosulfonyl isocyanate (0.153 mL, 1.759 mmol) in dichloromethane (3 mL) was cooled to 0° C. and allyl alcohol (0.120 mL, 1.759 mmol) was added dropwise. The resulting solution was stirred for 20 minutes at 0° C. before a solution of the product of Example II-14C (803 mg, 0.880 mmol) and triethylamine (0.307 mL, 2.199 mmol) in dichloromethane (5 mL) was added dropwise. The resulting solution was stirred at 0° C. for 30 minutes. The mixture was diluted with dichloromethane (10 mL), and the reaction was quenched with water (10 mL). The layers were separated, and the aqueous layer was extracted with dichloromethane (3×10 mL). The combined organic layers were dried with sodium sulfate, filtered, and concentrated under reduced pressure. The crude residue was subjected to column chromatography (silica gel, 40 g cartridge, 0-100% ethyl acetate in isohexane, 40 mL/minute) to afford the title compound (549 mg, 0.640 mmol, 64% yield). ¹H NMR (400 MHz, DMSO-d₆ at 90° C.) δ ppm 7.54-7.43 (m, 2H), 7.43-7.27 (m, 3H), 7.24 (s, 1H), 6.45 (s, 2H), 6.21-5.85 (m, 2H), 5.26-5.07 (m, 3H), 5.02 (dq, J=10.5, 1.7 Hz, 1H), 4.72-4.65 (m, 1H), 4.29-4.20 (m, 1H), 3.96 (dt, J=4.8, 1.7 Hz, 1H), 3.48-3.05 (m, 5H), 2.90-2.77 (m, 1H), 1.85-1.58 (m, 4H), 1.56-1.47 (m, 9H), 1.44-1.29 (m, 18H).

### Example II-14E: tert-butyl (2R)-6-(benzyloxy)-2-{[(tert-butoxycarbonyl)(4,4-difluorobutyl)amino]methyl}-4-fluoro-5-(1,1,4-trioxo-1λ⁶,2,5-thiadiazolidin-2-yl)-2,3-dihydro-1H-indole-1-carboxylate

A solution of the product of Example II-14D (686 mg, 0.640 mmol) in methanol (6 mL) was deoxygenated for 20 minutes (nitrogen gas bleed). Tetrakis(triphenylphosphine)palladium(0) (74.0 mg, 0.064 mmol) and potassium carbonate (354 mg, 2.56 mmol) were added. The reaction mixture was stirred at 60° C. for 4 hours. The reaction mixture was concentrated under reduced pressure. The crude residue was subjected to column chromatography (Reveleris® 40 g reversed phase (C18) flash cartridge, dry loaded with C18 silica, 10-100% methanol in 0.1% ammonium hydroxide, 40 mL/minute) to afford the title compound (315 mg, 0.419 mmol, 66% yield). ¹H NMR (400 MHz, DMSO-d₆ at 90° C.) δ ppm 7.69-7.25 (m, 5H), 7.23 (s, 1H), 6.03 (tt, J=56.9, 4.3 Hz, 1H), 5.11 (s, 2H), 4.72-4.64 (m, 1H), 4.05-3.91 (m, 2H), 3.42-3.29 (m, 2H), 3.26-3.05-(m, 3H), 2.88-2.77 (m, 1H), 1.87-1.69 (m, 2H), 1.67-1.57 (m, 2H), 1.51 (d, J=6.1 Hz, 9H), 1.37 (s, 9H); MS (ESI⁻) m/z 697 [M−H]⁻.

### Example II-14F: tert-butyl (2R)-2-{[(tert-butoxycarbonyl)(4,4-difluorobutyl)amino]methyl}-4-fluoro-6-hydroxy-5-(1,1,4-trioxo-1λ⁶,2,5-thiadiazolidin-2-yl)-2,3-dihydro-1H-indole-1-carboxylate

To a solution of the product of Example II-14E (315 mg, 0.419 mmol) in ethanol (4 mL) and water (0.4 mL) was added 10% Pd/C (99 mg). The resulting suspension was allowed III to stir under hydrogen (5 bar) for 16 hours. The reaction mixture was filtered through a Whatman glass fiber filter paper and washed with ethanol (3×15 mL). The filtrate was concentrated under reduced pressure. The crude residue was subjected to column chromatography (Reveleris® 40 g reversed phase (C18) flash cartridge, dry loaded with C18 silica, 10-100% methanol in 0.1% ammonium hydroxide, 40 mL/minute) to afford the title compound (177 mg, 0.288 mmol, 69% yield). ¹H NMR (400 MHz, DMSO-d₆ at 90° C.) δ ppm 6.75 (s, 1H), 6.03 (tt, J=56.9, 4.3 Hz, 1H), 4.66-4.53 (m, 1H), 3.98 (d, J=13.0 Hz, 1H), 3.89 (d, J=12.9 Hz, 1H), 3.43-3.31 (m, 1H), 3.31-3.18 (m, 4H), 2.77-2.68 (m, 1H), 1.86-1.70 (m, 2H), 1.68-1.59 (m, 2H), 1.52 (s, 9H), 1.39 (s, 9H); MS (ESI⁺) m/z 631 [M+Na]⁺.

### Example II-14G: 5-(2-{[(4,4-difluorobutyl)amino]methyl}-4-fluoro-6-hydroxy-2,3-dihydro-1H-indol-5-yl)-1λ⁶,2,5-thiadiazolidine-1,1,3-trione

To a solution of the product of Example II-14F (170 mg, 0.277 mmol) in dichloromethane (2 mL) at room temperature was added trifluoroacetic acid (0.33 mL, 4.28 mmol). The reaction mixture was stirred at ambient temperature for 3 hours. An additional portion of trifluoroacetic acid (0.11 mL, 1.43 mmol) was added, and the reaction mixture was stirred at ambient temperature for 1 hour before cooling to 0° C. Then the reaction was quenched with 0.7 M ammonia in methanol (8.5 mL). The mixture was concentrated under reduced pressure. The crude residue was subjected to column chromatography (Reveleris® 12 g reversed phase (C18) flash cartridge, dry loaded with C18 silica, 0-20% methanol in 0.1% ammonium hydroxide, 30 mL/minute) to afford the title compound (81 mg, 0.190 mmol, 69% yield). ¹H NMR (400 MHz, DMSO-d₆) δ ppm 8.69 (s, 1H), 8.34 (s, 2H), 6.14 (tt, J=56.5, 4.1 Hz, 1H), 6.02 (s, 1H), 5.93 (s, 1H), 4.13-4.08 (m, 1H), 3.84 (s, 2H), 3.20-3.05 (m, 1H), 3.08-2.96 (m, 4H), 2.74 (dd, J=15.8, 6.8 Hz, 1H), 2.00-1.81 (m, 2H), 1.80-1.67 (m, 2H); MS (ESI⁺) m/z 409 [M+H]⁺.

### Example II-15: 5-[(2R)-2-{[(cyclopentylmethyl)amino]methyl}-4-fluoro-6-hydroxy-2,3-dihydro-1H-indol-5-yl]-1λ⁶,2,5-thiadiazolidine-1,1,3-trione (Compound 146)

### Example II-15A: tert-butyl (2R)-6-(benzyloxy)-5-[(2-tert-butoxy-2-oxoethyl)(trifluoroacetyl)amino]-2-{[(cyclopentylmethyl)amino]methyl}-4-fluoro-2,3-dihydro-1H-indole-1-carboxylate

Acetic acid (0.230 mL, 4.02 mmol) was added to a solution of cyclopentylmethanamine hydrochloride (0.546 g, 4.02 mmol) and the product of Example II-2I (1 g, 1.341 mmol) in 1,2-dichloroethane (6 mL). The reaction mixture was stirred at room temperature for 40 minutes. Sodium triacetoxyborohydride (0.426 g, 2.012 mmol) was added, and the reaction mixture was stirred for 1 hour. The reaction mixture was quenched with a saturated aqueous solution of sodium bicarbonate (3 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (1.06 g, 1.326 mmol, 99% yield, 85% purity). ¹H NMR (400 MHz, DMSO-d₆ at 90° C.) δ ppm 7.49-7.28 (m, 6H), 5.28-5.03 (m, 2H), 4.57-4.48 (m, 1H), 4.43 (dd, J=16.4, 6.8 Hz, 1H), 3.94-3.82 (m, 1H), 3.17 (dt, J=15.8, 9.9 Hz, 1H), 3.06-2.95 (m, 2H), 2.83 (dt, J=12.3, 3.8 Hz, 1H), 2.76-2.60 (m, 1H), 1.99-1.87 (m, 1H), 1.75-1.59 (m, 2H), 1.59-1.43 (m, 13H), 1.40 (s, 9H), 1.23-1.07 (m, 3H); MS (ESI⁺) m/z 680 [M+H]⁺.

### Example II-15B: tert-butyl (2R)-6-(benzyloxy)-2-{[(tert-butoxycarbonyl)(cyclopentylmethyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-15A (1.06 g, 1.326 mmol) in acetonitrile (6 mL) were added N,N-diisopropylethylamine (0.695 mL, 3.98 mmol) and di-tert-butyl dicarbonate (0.579 g, 2.65 mmol). The reaction mixture was stirred at ambient temperature for 1 hour. The reaction was quenched with half saturated brine (10 mL). The mixture was diluted with ethyl acetate (20 mL), and the layers were separated. The aqueous layer was extracted with ethyl acetate (3×15 mL). The combined organic layers were dried with sodium sulfate, filtered, and concentrated under reduced pressure. The crude residue was subjected to column chromatography (silica gel, 80 g cartridge, 20% ethyl acetate in isohexane, 60 mL/minute) to afford the title compound (0.80 g, 1.026 mmol, 76% yield). ¹H NMR (400 MHz, DMSO-d₆ at 90° C.) δ ppm 7.44-7.29 (m, 6H), 5.18 (dd, J=12.0, 5.2 Hz, 1H), 5.11 (d, J=12.1 Hz, 1H), 4.75-4.67 (m, 1H), 4.44 (dd, J=16.5, 9.6 Hz, 1H), 3.95-3.76 (m, 1H), 3.47-3.33 (m, 2H), 3.19-3.05 (m, 3H), 2.96-2.87 (m, 1H), 2.25-2.10 (m, 1H), 1.71-1.57 (m, 5H), 1.57-1.47 (m, 10H), 1.45-1.31 (m, 20H); MS (ESI⁺) m/z 803 [M+Na]⁺.

### Example II-15C: tert-butyl (2R)-6-(benzyloxy)-2-{[(tert-butoxycarbonyl)(cyclopentylmethyl)amino]methyl}-5-[(2-tert-butoxy-2-oxoethyl)amino]-4-fluoro-2,3-dihydro-1H-indole-1-carboxylate

To a solution of the product of Example II-15B (1 g, 1.026 mmol) in methanol (2.8 mL) and tetrahydrofuran (2.8 mL) at room temperature was added 2 M aqueous lithium hydroxide (1.5 mL, 3.08 mmol). The reaction mixture was then stirred at room temperature for 1 hour. The reaction mixture was neutralized to pH 7 with 1 M aqueous hydrochloric acid and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (819 mg, 0.958 mmol, 93% yield, 80% purity). MS (ESI⁺) m/z 708 [M+Na]⁺.

### Example II-15D: tert-butyl (2R)-6-(benzyloxy)-2-{[(tert-butoxycarbonyl)(cyclopentylmethyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.16 mL, 1.843 mmol) in dichloromethane (5 mL) at 0° C. was added allyl alcohol (0.13 mL, 1.912 mmol) dropwise. The resulting solution was stirred for 20 minutes at 0° C. before a prepared solution of the product of Example II-15C (0.819 g, 0.958 mmol) and triethylamine (0.35 mL, 2.51 mmol) in dichloromethane (5 mL) was added dropwise. The resulting solution was stirred at 0° C. for 30 minutes. The reaction mixture was quenched with water (50 mL) and diluted with dichloromethane (50 mL). The layers were separated, and the aqueous layer was extracted with dichloromethane (3×50 mL). The combined organic layers were dried over sodium sulfate, filtered, and concentrated under reduced pressure. The crude residue was subjected to column chromatography (SiO₂, 40 g cartridge, 0-40% tert-butyl methyl ether in isohexane, 90 mL/minute) to afford the title compound (0.605 g, 0.714 mmol, 75% yield). ¹H NMR (400 MHz, DMSO-d₆) δ ppm 11.75-11.42 (m, 1H), 7.59-7.14 (m, 6H), 5.83-5.62 (m, 1H), 5.29-4.98 (m, 4H), 4.65 (dd, J=17.7, 11.2 Hz, 2H), 4.37-4.21 (m, 1H), 4.21-4.11 (m, 2H), 3.28-3.06 (m, 3H), 2.85 (d, J=15.9 Hz, 1H), 2.24-2.09 (m, 1H), 1.71-1.53 (m, 5H), 1.47 (s, 12H), 1.41-1.23 (m, 20H); MS (ESI⁺) m/z 747 [M-C(O)OC(CH₃)₃+H]⁺.

### Example II-15E: tert-butyl (2R)-6-(benzyloxy)-2-{[(tert-butoxycarbonyl)(cyclopentylmethyl)amino]methyl}-4-fluoro-5-(1,1,4-trioxo-1λ⁶,2,5-thiadiazolidin-2-yl)-2,3-dihydro-1H-indole-1-carboxylate

A solution of the product of Example II-15D (0.756 g, 0.714 mmol) and potassium carbonate (0.4 g, 2.89 mmol) in methanol (7 mL) was degassed under vacuum and backfilled with nitrogen three times. To the mixture was added tetrakis(triphenylphosphine)palladium(0) (0.083 g, 0.071 mmol), and the reaction mixture was degassed under vacuum and backfilled with nitrogen three times. The reaction mixture was stirred at 60° C. for 4 hours and then was cooled to room temperature. C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 0.10% ammonium hydroxide, 40 mL/minute) to afford the title compound (615 mg, 0.774 mmol, 100% yield, 80% purity). ¹H NMR (400 MHz, DMSO-d₆ at 90° C.) δ ppm 7.69-7.57 (m, 2H), 7.43-7.25 (m, 3H), 7.21 (s, 1H), 5.11 (s, 2H), 4.76-4.59 (m, 1H), 3.94 (d, J=13.0 Hz, 1H), 3.87 (d, J=13.0 Hz, 1H), 3.47-3.29 (m, 2H), 3.17-3.06 (m, 3H), 2.86 (dd, J=15.8, 2.2 Hz, 1H), 2.19 (hept, J=7.4 Hz, 1H), 1.73-1.57 (m, 4H), 1.52 (s, 11H), 1.37 (s, 9H), 1.26-1.10 (m, 2H); MS (ESI⁺) m/z 711 [M+Na]⁺.

### Example II-15F: tert-butyl (2R)-2-{[(tert-butoxycarbonyl)(cyclopentylmethyl)amino]methyl}-4-fluoro-6-hydroxy-5-(1,1,4-trioxo-1λ⁶,2,5-thiadiazolidin-2-yl)-2,3-dihydro-1H-indole-1-carboxylate

To a suspension of the product of Example II-15E (300 mg, 0.340 mmol) in degassed water (0.4 mL) and ethanol (4 mL) was added 10% Pd/C (72 mg). The resulting suspension was allowed to stir under hydrogen (5 bar) for 24 hours. The reaction mixture was filtered through a Whatman glass fiber filter paper and washed with ethanol (3×10 mL). The filtrate was concentrated under reduced pressure onto C18 silica (2 g). The crude residue was subjected to column chromatography (Reveleris® 24 g reversed phase (C18) flash cartridge, dry loaded with C18 silica, 10-50% methanol in 0.1% ammonium hydroxide, 35 mL/minute) to afford the title compound (66 mg, 0.105 mmol, 31% yield). ¹H NMR (400 MHz, DMSO-d₆ at 90° C.) δ ppm 6.66 (s, 1H), 4.67-4.49 (m, 1H), 4.01 (d, J=13.0 Hz, 1H), 3.89 (d, J=13.0 Hz, 1H), 3.39 (dd, J=13.9, 7.7 Hz, 1H), 3.27 (dd, J=13.9, 5.4 Hz, 1H), 3.18-3.08 (m, 3H), 2.93 (dd, J=15.1, 9.1 Hz, 2H), 2.72 (d, J=14.7 Hz, 1H), 2.20 (hept, J=7.4 Hz, 1H), 1.71-1.54 (m, 5H), 1.51 (s, 10H), 1.39 (s, 9H), 1.27-1.12 (m, 2H); MS (ESI⁺) m/z 621 [M+Na]⁺.

### Example II-15G: 5-[(2R)-2-{[(cyclopentylmethyl)amino]methyl}-4-fluoro-6-hydroxy-2,3-dihydro-1H-indol-5-yl]-1λ⁶,2,5-thiadiazolidine-1,1,3-trione

To a solution of the product of Example II-15F (60 mg, 0.095 mmol) in dichloromethane (1 mL) at room temperature was added trifluoroacetic acid (0.110 mL, 1.432 mmol). The reaction mixture was stirred at room temperature for 20 hours. The reaction mixture was neutralized with 0.7 M NH₃ in methanol:water (95:5) (7 mL) and concentrated under reduced pressure. The crude residue was subjected to column chromatography (Reveleris® 24 g reversed phase (C18) flash cartridge, dry loaded with C18 silica, 5-20% methanol in 0.1% ammonium hydroxide, 35 mL/minute) followed by a second column chromatography (Teledyne ISCO RediSep Rf Gold® 12 g reversed phase (C18) flash cartridge, dry loaded with C18 silica, 0-20% methanol in 0.1% ammonium hydroxide, 35 mL/minute) to afford the title compound (33 mg, 0.076 mmol, 80% yield). ¹H NMR (400 MHz, DMSO-d₆) δ ppm 8.68 (s, 1H), 8.16 (s, 2H), 6.02 (s, 1H), 5.92 (s, 1H), 4.22-4.02 (m, 1H), 3.84 (s, 2H), 3.15-2.95 (m, 3H), 2.92 (dd, J=7.3, 1.8 Hz, 2H), 2.73 (dd, J=15.6, 6.8 Hz, 1H), 2.14 (hept, J=7.7 Hz, 1H), 1.77 (td, J=11.3, 6.0 Hz, 2H), 1.68-1.44 (m, 4H), 1.22 (td, J=11.3, 9.7, 4.3 Hz, 2H); MS (ESI⁺) m/z 399 [M+H]⁺.

### Example II-16: 5-[(2R)-2-{[(cyclopropylmethyl)amino]methyl}-4-fluoro-6-hydroxy-2,3-dihydro-1H-indol-5-yl]-1λ⁶,2,5-thiadiazolidine-1,1,3-trione (Compound 147)

### Example II-16A: tert-butyl (2R)-6-(benzyloxy)-5-[(2-tert-butoxy-2-oxoethyl)(trifluoroacetyl)amino]-2-{[(cyclopropylmethyl)amino]methyl}-4-fluoro-2,3-dihydro-1H-indole-1-carboxylate

Acetic acid (0.230 mL, 4.02 mmol) was added to a solution of cyclopropylmethanamine (0.349 mL, 4.02 mmol) and the product of Example II-2I (1 g, 1.341 mmol) in 1,2-dichloroethane (6 mL). The reaction mixture was stirred at room temperature for 2 hours. Sodium triacetoxyborohydride (0.426 g, 2.012 mmol) was added, and the reaction mixture was stirred for 1 hour. The reaction mixture was quenched with a saturated aqueous solution of sodium bicarbonate (3 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (987 mg, 1.287 mmol, 96% yield, 85% purity). ¹H NMR (400 MHz, DMSO-d₆ at 90° C.) δ ppm 7.57-7.24 (m, 6H), 5.26-5.01 (m, 2H), 4.61-4.48 (m, 1H), 4.43 (dd, J=16.4, 5.9 Hz, 1H), 3.96-3.85 (m, 1H), 3.25-3.13 (m, 1H), 3.09-2.93 (m, 2H), 2.89 (dd, J=12.0, 3.9 Hz, 1H), 2.70 (dt, J=12.0, 7.5 Hz, 1H), 2.47 (dd, J=6.6, 2.3 Hz, 2H), 1.53 (s, 9H), 1.40 (s, 9H), 0.96-0.79 (m, 1H), 0.50-0.35 (m, 2H), 0.21-0.05 (m, 2H); MS (ESI⁺) m/z 652 [M+H]⁺.

### Example II-16B: tert-butyl (2R)-6-(benzyloxy)-2-{[(tert-butoxycarbonyl)(cyclopropylmethyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-16A (0.987 g, 1.287 mmol) in acetonitrile (6 mL) at room temperature were added N,N-diisopropylethylamine (0.675 mL, 3.86 mmol) and di-tert-butyl dicarbonate (0.562 g, 2.57 mmol). The reaction mixture was stirred at room temperature for 1 hour. The reaction was quenched with half saturated brine (10 mL). The mixture was diluted with ethyl acetate (20 mL), and the layers were separated. The aqueous layer was extracted with ethyl acetate (3×15 mL). The combined organic layers were dried with sodium sulfate, filtered, and concentrated under reduced pressure. The crude residue was subjected to column chromatography (silica gel, 40 g cartridge, 0-40% tert-butyl methyl ether in isohexane, 35 mL/minute) to afford the title compound (920 mg, 1.187 mmol, 93% yield). ¹H NMR (400 MHz, DMSO-d₆ at 90° C.) δ ppm 7.52-7.18 (m, 6H), 5.27-5.06 (m, 2H), 4.82-4.66 (m, 1H), 4.44 (dd, J=16.5, 10.4 Hz, 1H), 3.88 (dd, J=25.3, 16.5 Hz, 1H), 3.54-3.34 (m, 2H), 3.25-3.07 (m, 2H), 3.07-2.85 (m, 2H), 1.62-1.45 (m, 9H), 1.45-1.30 (m, 18H), 1.08-0.89 (m, 1H), 0.55-0.38 (m, 2H), 0.28-0.15 (m, 2H); MS (ESI⁺) m/z 775 [M+Na]⁺.

### Example II-16C: tert-butyl (2R)-6-(benzyloxy)-2-{[(tert-butoxycarbonyl)(cyclopropylmethyl)amino]methyl}-5-[(2-tert-butoxy-2-oxoethyl)amino]-4-fluoro-2,3-dihydro-1H-indole-1-carboxylate

To a solution of the product of Example II-16B (920 mg, 1.187 mmol) in methanol (2.8 mL) and tetrahydrofuran (2.8 mL) at room temperature was added 2 M aqueous lithium hydroxide (1.781 mL, 3.56 mmol). The reaction mixture was then stirred at room temperature for 1 hour. The reaction mixture was neutralized to pH 7 with 1 M aqueous hydrochloric acid and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (762 mg, 1.162 mmol, 98% yield). ¹H NMR (400 MHz, DMSO-d₆ at 90° C.) δ ppm 7.55-7.43 (m, 2H), 7.43-7.29 (m, 3H), 7.16 (s, 1H), 5.09 (s, 2H), 4.70-4.57 (m, 1H), 4.57-4.44 (m, 1H), 3.85 (dd, J=6.7, 2.0 Hz, 2H), 3.46-3.28 (m, 2H), 3.19-2.95 (m, 3H), 2.85 (dd, J=15.9, 2.0 Hz, 1H), 1.50 (s, 9H), 1.38 (s, 18H), 1.09-0.88 (m, 1H), 0.55-0.37 (m, 2H), 0.33-0.08 (m, 2H).

### Example II-16D: tert-butyl (2R)-6-(benzyloxy)-2-{[(tert-butoxycarbonyl)(cyclopropylmethyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.192 mL, 2.209 mmol) in dichloromethane (5 mL) at 0° C. was added allyl alcohol (0.158 mL, 2.325 mmol) dropwise. The resulting solution was stirred for 20 minutes at 0° C. before a prepared solution of the product of Example II-16C (0.847 g, 1.162 mmol) and triethylamine (0.405 mL, 2.91 mmol) in dichloromethane (5 mL) was added dropwise. The resulting solution was stirred at 0° C. for 30 minutes. The reaction mixture was quenched with water (50 mL) and diluted with dichloromethane (50 mL). The layers were separated, and the aqueous layer was extracted with dichloromethane (3×50 mL). The combined organic layers were dried over sodium sulfate, filtered, and concentrated under reduced pressure. The crude residue was subjected to column chromatography (SiO₂, 40 g cartridge, 0-40% tert-butyl methyl ether in isohexane, 90 mL/minute) to afford the title compound (600 mg, 0.696 mmol, 60% yield). ¹H NMR (400 MHz, DMSO-d₆) δ ppm 11.73-11.36 (m, 1H), 7.53-7.28 (m, 6H), 5.71 (ddt, J=16.0, 10.7, 5.6 Hz, 1H), 5.26-4.99 (m, 4H), 4.73-4.55 (m, 2H), 4.34-4.20 (m, 1H), 4.20-4.06 (m, 2H), 3.23-2.91 (m, 3H), 2.91-2.72 (m, 1H), 1.47 (s, 9H), 1.42-1.20 (m, 20H), 1.01 (s, 1H), 0.54-0.35 (m, 2H), 0.29-0.11 (m, 2H); MS (ESI⁺) m/z 663 [M-C(O)OC(CH₃)₃—C(CH₃)₃+H]⁺.

### Example II-16E: tert-butyl (2R)-6-(benzyloxy)-2-{[(tert-butoxycarbonyl)(cyclopropylmethyl)amino]methyl}-4-fluoro-5-(1,1,4-trioxo-R 2,5-thiadiazolidin-2-yl)-2,3-dihydro-1H-indole-1-carboxylate

A solution of the product of Example II-16D (0.6 g, 0.696 mmol) and potassium carbonate (0.4 g, 2.89 mmol) in methanol (7 mL) was degassed under vacuum and backfilled with nitrogen three times. To the mixture was added tetrakis(triphenylphosphine)palladium(0) (0.080 g, 0.070 mmol), and the reaction mixture was degassed under vacuum and backfilled with nitrogen three times. The reaction mixture was stirred at 60° C. for 4 hours. The mixture was cooled to room temperature. C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 0.1% ammonium hydroxide, 40 mL/minute) to afford the title compound (492 mg, 0.653 mmol, 94% yield, 90% purity). ¹H NMR (400 MHz, DMSO-d₆) δ ppm 7.58-7.46 (m, 2H), 7.43-7.26 (m, 3H), 7.20 (s, 1H), 5.11 (s, 2H), 4.78-4.64 (m, 1H), 3.94 (d, J=13.0 Hz, 1H), 3.87 (d, J=13.0 Hz, 1H), 3.52-3.33 (m, 2H), 3.18-3.04 (m, 3H), 2.87 (dd, J=15.8, 2.1 Hz, 1H), 1.51 (s, 9H), 1.38 (s, 9H), 1.09-0.91 (m, 1H), 0.46 (dt, J=9.7, 2.8 Hz, 2H), 0.25-0.17 (m, 2H); MS (ESI⁺) m/z 683 [M+Na]⁺.

### Example II-16F: tert-butyl (2R)-2-{[(tert-butoxycarbonyl)(cyclopropylmethyl)amino]methyl}-4-fluoro-6-hydroxy-5-(1,1,4-trioxo-1λ⁶,2,5-thiadiazolidin-2-yl)-2,3-dihydro-1H-indole-1-carboxylate

To a suspension of the product of Example II-16E (250 mg, 0.341 mmol) in deoxygenated water (0.3 mL) and ethanol (3 mL) was added 10% Pd/C (64 mg). The resulting suspension was allowed to stir under hydrogen (5 bar) for 2 hours. The reaction mixture was filtered through a Whatman glass fiber filter paper and washed with ethanol (3×10 mL). The filtrate was concentrated under reduced pressure onto C18 silica (2 g). The crude residue was subjected to column chromatography (Reveleris® 24 g reversed phase (C18) flash cartridge, dry loaded with C18 silica 10-50% methanol in 0.1% ammonium hydroxide, 35 mL/minute) to afford the title compound (168 mg, 0.280 mmol, 82% yield). ¹H NMR (400 MHz, DMSO-d₆ at 90° C.) δ ppm 6.93 (s, 1H), 4.65 (dddd, J=9.3, 7.5, 5.8, 2.0 Hz, 1H), 3.99-3.84 (m, 2H), 3.38 (dt, J=14.0, 6.7 Hz, 2H), 3.14 (dd, J=14.5, 6.6 Hz, 2H), 3.09-2.98 (m, 2H), 2.82 (dd, J=15.6, 2.0 Hz, 1H), 1.52 (s, 9H), 1.38 (s, 9H), 1.08-0.90 (m, 1H), 0.52-0.38 (m, 2H), 0.27-0.15 (m, 2H); MS (ESI⁺) m/z 593 [M+Na]⁺.

### Example II-16G: 5-[(2R)-2-{[(cyclopropylmethyl)amino]methyl}-4-fluoro-6-hydroxy-2,3-dihydro-1H-indol-5-yl]-1λ⁶,2,5-thiadiazolidine-1,1,3-trione

To a solution of the product of Example II-16F (100 mg, 0.167 mmol) in dichloromethane (1 mL) at room temperature was added trifluoroacetic acid (0.193 mL, 2.501 mmol). The reaction mixture was stirred at room temperature for 20 hours. The reaction mixture was neutralized with 0.7 M NH₃ in methanol:water (95:5) (7 mL) and concentrated under reduced pressure. The crude residue was subjected to column chromatography (Reveleris® 24 g reversed phase (C18) flash cartridge, dry loaded with C18 silica, 5-20% methanol in 0.1% ammonium hydroxide, 35 mL/minute) followed by a second column chromatography (Teledyne ISCO RediSep Rf Gold® 12 g reversed phase (C18) flash cartridge, dry loaded with C18 silica, 0-20% methanol in 0.1% ammonium hydroxide, 35 mL/minute) to afford the title compound (28 mg, 0.0756 mmol, 45% yield). ¹H NMR (400 MHz, DMSO-d₆) δ ppm 8.68 (s, 1H), 8.29 (s, 2H), 6.03 (s, 1H), 5.92 (s, 1H), 4.23-4.05 (m, 1H), 3.84 (s, 2H), 3.19-2.94 (m, 3H), 2.85 (d, J=7.3 Hz, 2H), 2.74 (dd, J=15.6, 6.8 Hz, 1H), 1.04 (tt, J=7.9, 4.6 Hz, 1H), 0.67-0.50 (m, 2H), 0.46-0.29 (m, 2H); MS (ESI⁺) m/z 371 [M+H]⁺.

### Example II-17: 5-[(2R)-4-fluoro-6-hydroxy-2-{[(pentan-2-yl)amino]methyl}-2,3-dihydro-1H-indol-5-yl]-1λ⁶,2,5-thiadiazolidine-1,1,3-trione (Compound 148)

### Example II-17A: tert-butyl (2R)-6-(benzyloxy)-5-[(2-tert-butoxy-2-oxoethyl)(trifluoroacetyl)amino]-4-fluoro-2-{[(pentan-2-yl)amino]methyl}-2,3-dihydro-1H-indole-1-carboxylate

Acetic acid (0.273 mL, 4.78 mmol) was added to a solution of pentan-2-amine (0.566 mL, 4.78 mmol) and the product of Example II-2I (1.0 g, 1.592 mmol) in 1,2-dichloroethane (4 mL). The reaction mixture was stirred at room temperature for 1.5 hours. Sodium triacetoxyborohydride (0.506 g, 2.389 mmol) was added, and the reaction mixture was stirred for 3 hours at ambient temperature and at 35° C. for 20 minutes. The reaction mixture was quenched with a saturated aqueous solution of sodium bicarbonate (12 mL), diluted with dichloromethane (50 mL), and stirred for 10 minutes. The layers were separated, and the aqueous layer extracted with dichloromethane (2×25 mL). The combined organic layers were washed with brine (100 mL), dried over sodium sulfate, filtered, and concentrated under reduced pressure to afford the title compound (1.2 g, 1.581 mmol, 99% yield). ¹H NMR (400 MHz, DMSO-d₆) δ ppm 7.45-7.29 (m, 4H), 7.37-7.30 (m, 2H), 5.21-5.04 (m, 2H), 4.46-4.35 (m, 2H), 3.96 (dd, J=16.6, 5.4 Hz, 1H), 3.21-3.07 (m, 1H), 3.06-2.98 (m, 1H), 2.85-2.71 (m, 1H), 2.62-2.51 (m, 2H), 1.50 (s, 9H), 1.38 (s, 9H), 1.33-1.09 (m, 4H), 1.02-0.76 (m, 6H); MS (ESI⁺) m/z 668 [M+H]⁺.

### Example II-17B: tert-butyl (2R)-6-(benzyloxy)-2-{[(tert-butoxycarbonyl)(pentan-2-yl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-17A (1.2 g, 1.581 mmol) in dichloromethane (5 mL) at room temperature was added N,N-diisopropylethylamine (0.846 μL, 4.84 μmol) and di-tert-butyl dicarbonate (0.690 g, 3.16 mmol). The mixture was stirred at room temperature for 3 hours. The reaction was quenched with water (10 mL). The aqueous mixture was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-40% ethyl acetate in isohexane, 35 mL/minute) to afford the title compound (1.44 g, 1.575 mmol, 100% yield). ¹H NMR (400 MHz, DMSO-d₆) δ ppm 7.65-7.00 (m, 6H), 5.27-5.02 (m, 2H), 4.74-4.52 (m, 1H), 4.52-4.32 (m, 1H), 4.00-3.70 (m, 3H), 3.25-2.85 (m, 3H), 1.66-1.03 (m, 34H), 0.87 (td, J=7.4, 2.3 Hz, 3H); MS (ESI⁺) m/z 668 [M-C(O)OC(CH₃)₃+H]⁺.

### Example II-17C: tert-butyl (2R)-6-(benzyloxy)-2-{[(tert-butoxycarbonyl)(pentan-2-yl)amino]methyl}-5-[(2-tert-butoxy-2-oxoethyl)amino]-4-fluoro-2,3-dihydro-1H-indole-1-carboxylate

To a solution of the product of Example II-17B (1.44 g, 1.594 mmol) in methanol (3 mL) and tetrahydrofuran (3 mL) at room temperature was added 2 M aqueous lithium hydroxide (2.2 mL, 4.40 mmol). The reaction mixture was then stirred at room temperature for 50 minutes. The reaction mixture was quenched with a saturated aqueous solution of ammonium chloride (20 mL). The aqueous mixture was extracted with ethyl acetate (3×50 mL). The combined organic layers were washed with brine (70 mL), dried over sodium sulfate, filtered, and concentrated in vacuo. The crude residue was subjected to column chromatography (SiO₂, 24 g cartridge, 0-40% ethyl acetate/isohexane) to afford the title compound (0.741 g, 1.048 mmol, 66% yield). ¹H NMR (400 MHz, DMSO-d₆) δ ppm 7.57-7.26 (m, 6H), 5.08 (d, J=5.6 Hz, 2H), 4.77-4.43 (m, 2H), 3.91-3.51 (m, 3H), 3.30-2.80 (m, 4H), 1.49 (s, 9H), 1.44-1.01 (m, 25H), 0.93-0.77 (m, 3H); MS (ESI⁺) m/z 672 [M+H]⁺.

### Example II-17D: tert-butyl (2R)-6-(benzyloxy)-2-{[(tert-butoxycarbonyl)(pentan-2-yl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.182 mL, 2.096 mmol) in dichloromethane (8 mL) at 0° C. was added allyl alcohol (0.143 mL, 2.096 mmol) dropwise. The resulting solution was stirred for 20 minutes at 0° C. before a prepared solution of the product of Example II-17C (0.741 g, 1.048 mmol) and triethylamine (0.365 mL, 2.62 mmol) in dichloromethane (8 mL) was added dropwise. The resulting solution was stirred at 0° C. for 1 hour. The reaction mixture was quenched with a saturated aqueous solution of ammonium chloride (30 mL) and diluted with dichloromethane (50 mL). The layers were separated, and the aqueous layer was extracted with dichloromethane (2×50 mL). The combined organic layers were dried over sodium sulfate, filtered, and concentrated under reduced pressure. The crude residue was subjected to column chromatography (SiO₂, 24 g cartridge, 0-50% ethyl acetate in isohexane, 37 mL/minute) to afford the title compound (674 mg, 0.743 mmol, 71% yield). ¹H NMR (400 MHz, DMSO-d₆) δ ppm 11.52 (s, 1H), 7.50-7.22 (m, 6H), 5.80-5.62 (m, 1H), 5.30-5.00 (m, 4H), 4.74-4.51 (m, 2H), 4.32-4.08 (m, 3H), 3.91-3.37 (m, 2H), 3.21-2.83 (m, 3H), 1.70-1.43 (m, 9H), 1.43-1.00 (m, 25H), 0.87 (dt, J=11.8, 7.4 Hz, 3H); MS (ESI⁺) m/z 857 [M+Na]⁺.

### Example II-17E: tert-butyl (2R)-6-(benzyloxy)-2-{[(tert-butoxycarbonyl)(pentan-2-yl)amino]methyl}-4-fluoro-5-(1,1,4-trioxo-1λ⁶,2,5-thiadiazolidin-2-yl)-2,3-dihydro-1H-indole-1-carboxylate

To a solution of the product of Example II-17D (0.674 g, 0.743 mmol) and potassium carbonate (0.424 g, 3.07 mmol) in methanol (10 mL) was added tetrakis(triphenylphosphine)palladium(0) (0.090 g, 0.078 mmol). The reaction mixture was stirred at 60° C. for 2 hours. The mixture was cooled to room temperature and diluted with methanol (10 mL). C18 Silica (2 g) was added, and the mixture was concentrated in vacuo. The crude residue was subjected to column chromatography (Reveleris® 24 g reversed phase (C18) flash cartridge, dry loaded with silica, 5-70% methanol in 0.1% ammonium hydroxide, 17 mL/minute) to afford the title compound (300 mg, 0.341 mmol, 46% yield, 80% purity). ¹H NMR (400 MHz, DMSO-d₆ at 90° C.) δ ppm 7.52-7.48 (m, 2H), 7.38-7.26 (m, 3H), 7.24-7.18 (m, 1H), 5.20-5.02 (m, 2H), 4.69-4.59 (m, 1H), 3.98-3.80 (m, 2H), 3.78-3.65 (m, 1H), 3.41-3.31 (m, 1H), 3.25-3.14 (m, 1H), 3.13-3.04 (m, 1H), 2.97-2.90 (m, 1H), 1.65-1.49 (m, 10H), 1.49-1.34 (m, 10H), 1.34-1.19 (m, 2H), 1.18-1.09 (m, 3H), 0.93-0.86 (m, 3H); MS (ESI⁻) m/z 675 [M−H]⁻.

### Example II-17F: tert-butyl (2R)-2-{[(tert-butoxycarbonyl)(pentan-2-yl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a suspension of the product of Example II-17E in deoxygenated water (0.4 mL) and ethanol (4 mL) was added 10% Pd/C (73 mg). The resulting suspension was allowed to stir under hydrogen (5 bar) for 24 hours. The reaction mixture was filtered through a Whatman glass fiber filter paper and washed with ethanol (3×10 mL). The filtrate was concentrated onto C18 silica (2 g). The crude residue was subjected to column chromatography (Reveleris® 24 g reversed phase (C18) flash cartridge, dry loaded with C18 silica, 10-50% methanol in 0.1% ammonium hydroxide, 35 mL/minute) to afford the title compound as an ammonium salt as a 1:1 mixture of diastereoisomers (175 mg, 0.284 mmol, 83% yield). ¹H NMR (400 MHz, DMSO-d₆) δ ppm 8.53 (broad s, 1H) 7.06 (broad s, 3H), 6.99 (s, 1H), 4.75-4.52 (m, 1H), 3.93 (d, J=13.0 Hz, 1H), 3.88 (d, J=13.0 Hz, 1H), 3.82-3.62 (m, 1H), 3.35 (dd, J=14.2, 8.1 Hz, 1H), 3.19 (td, J=13.9, 5.1 Hz, 1H), 3.03 (dd, J=15.6, 9.1 Hz, 1H), 2.90 (dd, J=15.6, 2.2 Hz, 1H), 1.67-1.57 (m, 1H), 1.54 (s, 9H), 1.51-1.41 (m, 1H), 1.38 (s, 9H), 1.35-1.19 (m, 2H), 1.15 (dd, J=12.8, 6.8 Hz, 3H), 0.90 (td, J=7.3, 2.7 Hz, 3H); MS (ESI⁺) m/z 609 [M+Na]⁺.

### Example II-17G: 5-[(2R)-4-fluoro-6-hydroxy-2-{[(pentan-2-yl)amino]methyl}-2,3-dihydro-1H-indol-5-yl]-1λ⁶,2,5-thiadiazolidine-1,1,3-trione

To a solution of the product of Example II-17F (169 mg, 0.274 mmol) in dichloromethane (2 mL) at room temperature was added trifluoroacetic acid (0.317 mL, 4.12 mmol). The reaction mixture was stirred at room temperature for 20 hours. The reaction mixture was neutralized with 0.7 M NH₃ in methanol:water (95:5) (7 mL) and concentrated under reduced pressure. The crude residue was subjected to column chromatography (Teledyne ISCO RediSep Rf Gold® 24 g reversed phase (C18) flash cartridge, dry loaded with C18 silica, 0-20% methanol in 0.1% ammonium hydroxide, 35 mL/minute) to afford the title compound as a 1:1 mixture of diastereoisomers (51 mg, 0.132 mmol, 48% yield). ¹H NMR (400 MHz, DMSO-d₆) δ ppm 8.69 (s, 1H), 8.17 (s, 2H), 6.07-5.95 (m, 1H), 5.93 (s, 1H), 4.22-4.03 (m, 1H), 3.84 (s, 2H), 3.27-3.16 (m, 1H), 3.16-2.92 (m, 3H), 2.73 (dd, J=15.4, 7.1 Hz, 1H), 1.76-1.61 (m, 1H), 1.49-1.34 (m, 2H), 1.34-1.22 (m, 1H), 1.20 (d, J=6.5 Hz, 3H), 0.90 (t, J=7.2 Hz, 3H); MS (ESI⁺) m/z 387 [M+H]⁺.

### Example II-18: 5-[(2R)-2-({[(2R)-butan-2-yl]amino}methyl)-4-fluoro-6-hydroxy-2,3-dihydro-1H-indol-5-yl]-1λ⁶,2,5-thiadiazolidine-1,1,3-trione (Compound 149)

### Example II-18A: tert-butyl (2R)-6-(benzyloxy)-2-({[(2R)-butan-2-yl]amino}methyl)-5-[(2-tert-butoxy-2-oxoethyl)(trifluoroacetyl)amino]-4-fluoro-2,3-dihydro-1H-indole-1-carboxylate

Acetic acid (0.273 mL, 4.78 mmol) was added to a solution of (R)-butan-2-amine (0.482 mL, 4.78 mmol) and the product of Example II-2I (1 g, 1.592 mmol) in 1,2-dichloroethane (4 mL). The reaction mixture was stirred at room temperature for 1.5 hours. Sodium triacetoxyborohydride (0.506 g, 2.389 mmol) was added, and the reaction mixture was stirred for 3 hours. Additional sodium triacetoxyhydroborate (0.2 g, 0.944 mmol) was added to mixture. The reaction mixture was stirred for 30 minutes at ambient temperature and then at 35° C. for 20 minutes. The reaction mixture was quenched with a saturated aqueous solution of sodium bicarbonate (12 mL), diluted with dichloromethane (50 mL) and stirred for 10 minutes. The layers were separated, and the aqueous layer was extracted with dichloromethane (2×25 mL). The combined organic layers were washed with brine (100 mL), dried over sodium sulfate, filtered, and concentrated under reduced pressure to afford the title compound (1.196 g, 1.592 mmol, 100% yield, 90% purity). ¹H NMR (400 MHz, DMSO-d₆) δ ppm 7.45-7.38 (m, 1H), 7.42-7.29 (m, 5H), 5.22-5.04 (m, 2H), 4.45-4.40 (m, 1H), 4.46-4.35 (m, 1H), 3.96 (d, J=16.6 Hz, 1H), 3.22-2.98 (m, 2H), 2.86-2.76 (m, 1H), 2.61-2.51 (m, 1H), 2.50-2.39 (m, 1H), 1.50 (s, 9H), 1.43-1.12 (m, 11H), 1.04-0.88 (m, 3H), 0.88-0.71 (m, 3H); MS (ESI⁺) m/z 654 [M+H]⁺.

### Example II-18B: tert-butyl (2R)-6-(benzyloxy)-2-({[(2R)-butan-2-yl](tert-butoxycarbonyl)amino}methyl)-5-[(2-tert-butoxy-2-oxoethyl)(trifluoroacetyl)amino]-4-fluoro-2,3-dihydro-1H-indole-1-carboxylate

To a solution of the product of Example II-18A (1.196 g, 1.592 mmol) in dichloromethane (5 mL) at room temperature was added N,N-diisopropylethylamine (0.834 mL, 4.78 mmol) and di-tert-butyl dicarbonate (0.695 g, 3.18 mmol). The mixture was stirred at room temperature for 2 hours and then concentrated in vacuo. Acetonitrile (5 mL) was added, and the reaction mixture was stirred for 30 minutes at ambient temperature and at up to 40° C. for 20 minutes. The volatiles were removed in vacuo. The residue was dissolved in dichloromethane (50 mL) and stirred with a saturated aqueous solution of sodium bicarbonate (50 mL) for 10 minutes. The aqueous layer was extracted with dichloromethane (2×50 mL). The combined organic layers were washed with brine (100 mL), dried over sodium sulfate, filtered, and concentrated under reduced. The crude residue was subjected to column chromatography (SiO₂, 40 g cartridge, 0-40% ethyl acetate in isohexane, 40 mL/minute) to afford the title compound (1.05 g, 1.379 mmol, 87% yield). ¹H NMR (400 MHz, DMSO-d₆) δ ppm 7.76-6.86 (m, 6H), 5.25-5.02 (m, 2H), 4.73-4.55 (m, 1H), 4.49-4.33 (m, 1H), 4.00-3.36 (m, 3H), 3.30-2.85 (m, 3H), 1.71-1.42 (m, 11H), 1.42-1.06 (m, 21H), 0.88-0.72 (m, 3H); MS (ESI⁺) m/z 776 [M+Na]⁺.

### Example II-18C: tert-butyl (2R)-6-(benzyloxy)-2-({[(2R)-butan-2-yl](tert-butoxycarbonyl)amino}methyl)-5-[(2-tert-butoxy-2-oxoethyl)amino]-4-fluoro-2,3-dihydro-1H-indole-1-carboxylate

To a solution of the product of Example II-18B (1.05 g, 1.379 mmol) in methanol (3 mL) and tetrahydrofuran (3 mL) at room temperature was added 2 M aqueous lithium hydroxide (2 mL, 4.00 mmol). The reaction mixture was then stirred at room temperature for 50 minutes. The reaction mixture was quenched with a saturated aqueous solution of ammonium chloride (20 mL). The aqueous mixture was extracted with ethyl acetate (3×50 mL). The combined organic layers were washed with brine (70 mL), dried over sodium sulfate, filtered, and concentrated in vacuo. The crude residue was subjected to column chromatography (SiO₂, 24 g cartridge, 0-40% ethyl acetate/isohexane) to afford the title compound (0.677 g, 0.906 mmol, 66% yield, 90% purity). ¹H NMR (400 MHz, DMSO-d₆) δ ppm 7.58-7.29 (m, 5H), 7.03-6.77 (m, 1H), 5.15-5.00 (m, 2H), 4.79-4.28 (m, 2H), 3.92-3.34 (m, 3H), 3.30-2.81 (m, 4H), 1.68-1.43 (m, 11H), 1.43-1.03 (m, 21H), 0.88-0.75 (m, 3H); MS (ESI⁺) m/z 658 [M+H]⁺.

### Example II-18D: tert-butyl (2R)-6-(benzyloxy)-2-({[(2R)-butan-2-yl](tert-butoxycarbonyl)amino}methyl)-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.157 mL, 1.811 mmol) in dichloromethane (8 mL) at 0° C. was added allyl alcohol (0.123 mL, 1.811 mmol) dropwise. The resulting solution was stirred for 20 minutes at 0° C. before a prepared solution of the product of Example II-18C (0.677 g, 0.906 mmol) and triethylamine (0.316 mL, 2.264 mmol) in dichloromethane (8 mL) was added dropwise. The resulting solution was stirred at 0° C. for 60 minutes. The reaction mixture was quenched with saturated ammonium chloride solution (30 mL) and diluted with dichloromethane (50 mL). The layers were separated, and the aqueous layer was extracted with dichloromethane (2×50 mL). The combined organic layers were dried over sodium sulfate, filtered, and concentrated under reduced pressure to afford the title compound (867 mg, 0.877 mmol, 97% yield, 85% purity). ¹H NMR (400 MHz, DMSO-d₆) δ ppm 11.77-11.06 (m, 1H), 7.55-7.23 (m, 6H), 6.07-5.42 (m, 2H), 5.41-4.99 (m, 4H), 4.82-4.51 (m, 2H), 4.45-3.56 (m, 5H), 3.22-2.83 (m, 4H), 1.76-0.97 (m, 30H), 0.81 (dt, J=12.0, 7.4 Hz, 3H); MS (ESI⁻) m/z 819 [M−H]⁻.

### Example II-18E: tert-butyl (2R)-6-(benzyloxy)-2-({[(2R)-butan-2-yl](tert-butoxycarbonyl)amino}methyl)-4-fluoro-5-(1,1,4-trioxo-lf, 2,5-thiadiazolidin-2-yl)-2,3-dihydro-1H-indole-1-carboxylate-ammonia

A solution of the product of Example II-18D (0.867 g, 0.877 mmol) and potassium carbonate (0.48 g, 3.47 mmol) in methanol (9 mL) was degassed under vacuum and backfilled with nitrogen three times. To the mixture was added tetrakis(triphenylphosphine)palladium(0) (0.101 g, 0.088 mmol), and the reaction mixture was degassed under vacuum and backfilled with nitrogen three times. The reaction mixture was stirred at 60° C. for 24 hours. The mixture was cooled to room temperature. C18 Silica gel (3 g) was added, and the mixture was concentrated in vacuo. The crude residue was subjected to column chromatography (Reveleris® 40 g reversed phase (C18) flash cartridge, dry loaded with C18 silica gel, 10-70% methanol in 0.1% ammonium hydroxide, 40 mL/minute) to afford the title compound (590 mg, 0.347 mmol, 40% yield, 40% purity) as an ammonium salt. ¹H NMR (400 MHz, DMSO-d₆ at 90° C.) δ ppm 7.93-7.09 (m, 6H), 5.21-5.04 (m, 2H), 4.73-4.52 (m, 1H), 3.94 (d, J=13.0 Hz, 1H), 3.87 (d, J=13.0 Hz, 1H), 3.68-3.52 (m, 1H), 3.37 (dd, J=14.0, 9.1 Hz, 1H), 3.29-3.15 (m, 1H), 3.13-2.88 (m, 2H), 1.73-1.57 (m, 1H), 1.57-1.44 (m, 10H), 1.39 (s, 9H), 1.23-1.07 (m, 3H), 0.93-0.75 (m, 3H); MS (ESI⁺) m/z 685 [M+Na]⁺.

### Example II-18F: tert-butyl (2R)-2-({[(2R)-butan-2-yl](tert-butoxycarbonyl)amino}methyl)-4-fluoro-6-hydroxy-5-(1,1,4-trioxo-1λ⁶,2,5-thiadiazolidin-2-yl)-2,3-dihydro-1H-indole-1-carboxylate-ammonia

To a suspension of the product of Example II-18E in degassed water (0.4 mL) and ethanol (4 mL) was added 10% Pd/C (109 mg). The resulting suspension was allowed to stir under hydrogen (5 bar) for 24 hours. The reaction mixture was filtered through a Whatman glass fiber filter paper and washed with ethanol (3×10 mL). The filtrate was concentrated under reduced pressure onto C18 silica (3 g). The crude residue was subjected to column chromatography (Reveleris® 24 g reversed phase (C18) flash cartridge, dry loaded with C18 silica, 20-80% methanol in 0.1% ammonium hydroxide, 35 mL/minute) to afford the title compound as an ammonium salt (134 mg, 0.193 mmol, 75% yield). ¹H NMR (400 MHz, DMSO-d₆ at 90° C.) δ ppm 8.62 (s, 1H), 7.00 (s, 4H), 4.70-4.51 (m, 1H), 3.93 (d, J=13.0 Hz, 1H), 3.88 (d, J=13.0 Hz, 1H), 3.60 (q, J=7.0 Hz, 1H), 3.36 (dd, J=14.0, 9.0 Hz, 1H), 3.21 (dd, J=14.1, 5.0 Hz, 1H), 3.09-3.04 (m, 1H), 2.91 (dd, J=15.5, 2.2 Hz, 1H), 1.72-1.57 (m, 1H), 1.54 (s, 9H), 1.48 (dd, J=13.6, 7.3 Hz, 1H), 1.39 (s, 9H), 1.21-1.05 (m, 3H), 0.85 (t, J=7.4 Hz, 3H); MS (ESI⁺) m/z 595 [M+Na]⁺.

### Example II-18G: 5-[(2R)-2-({[(2R)-butan-2-yl]amino}methyl)-4-fluoro-6-hydroxy-2,3-dihydro-1H-indol-5-yl]-1λ⁶,2,5-thiadiazolidine-1,1,3-trione

To a solution of the product of Example II-18F (70 mg, 0.101 mmol) in dichloromethane (1 mL) at room temperature was added trifluoroacetic acid (0.117 mL, 1.514 mmol). The reaction mixture was stirred at room temperature for 20 hours. The reaction mixture was neutralized with 0.7 M NH₃ in methanol:water (95:5) (7 mL) and concentrated under reduced pressure. The crude residue was subjected to column chromatography (Teledyne ISCO RediSep Rf Gold® 12 g reversed phase (C18) flash cartridge, dry loaded with C18 silica, 0-20% methanol in 0.1% ammonium hydroxide, 35 mL/minute) to afford the title compound (27 mg, 0.072 mmol, 72% yield). ¹H NMR (400 MHz, DMSO-d₆) δ ppm 8.69 (s, 1H), 6.00 (s, 1H), 5.93 (s, 1H), 4.10 (d, J=6.8 Hz, 1H), 3.84 (s, 2H), 3.21-2.92 (m, 4H), 2.72 (dd, J=15.6, 7.0 Hz, 1H), 1.84-1.67 (m, 1H), 1.53-1.36 (m, 1H), 1.20 (d, J=6.5 Hz, 3H), 0.90 (t, J=7.4 Hz, 3H); MS (ESI⁺) m/z 373 [M+H]⁺.

### Example II-19: 5-{(2R)-2-[(butylamino)methyl]-4-fluoro-6-hydroxy-2,3-dihydro-1H-indol-5-yl}-1λ⁶,2,5-thiadiazolidine-1,1,3-trione (Compound 150)

### Example II-19A: tert-butyl (2R)-6-(benzyloxy)-5-[(2-tert-butoxy-2-oxoethyl)(trifluoroacetyl)amino]-2-[(butylamino)methyl]-4-fluoro-2,3-dihydro-1H-indole-1-carboxylate

Acetic acid (0.273 mL, 4.78 mmol) was added to a solution of butan-1-amine (0.472 mL, 4.78 mmol) and the product of Example II-2I (1 g, 1.592 mmol) in 1,2-dichloroethane (6 mL). The reaction mixture was stirred at room temperature for 2 hours. Sodium triacetoxyborohydride (0.506 g, 2.389 mmol) was added, and the reaction mixture was stirred for 1 hour. The reaction mixture was quenched with a saturated aqueous solution of sodium bicarbonate (3 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (1.168 g, 1.608 mmol, 100% yield, 90% purity). ¹H NMR (400 MHz, DMSO-d₆) δ ppm 7.51-7.28 (m, 6H), 5.26-5.03 (m, 2H), 4.56-4.47 (m, 1H), 4.43 (dd, J=16.4, 6.6 Hz, 1H), 3.89 (d, J=16.4 Hz, 2H), 3.23-3.10 (m, 1H), 3.06-2.96 (m, 2H), 2.81 (dt, J=12.2, 3.6 Hz, 1H), 2.71-2.53 (m, 2H), 1.53 (s, 9H), 1.40 (s, 9H), 1.37-1.25 (m, 4H), 0.86 (td, J=7.2, 3.1 Hz, 3H); MS (ESI⁺) m/z 654 [M+H]⁺.

### Example II-19B: tert-butyl (2R)-6-(benzyloxy)-2-{[(tert-butoxycarbonyl)(butyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-19A (1.168 g, 1.608 mmol) in acetonitrile (8 mL) at room temperature was added N,N-diisopropylethylamine (0.85 mL, 4.87 mmol) and di-tert-butyl dicarbonate (0.700 g, 3.21 mmol). The mixture was stirred at room temperature for 24 hours. The reaction was quenched with water (10 mL). The aqueous mixture was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-40% ethyl acetate in isohexane, 35 mL/minute) to afford the title compound (1.08 g, 1.404 mmol, 87% yield). ¹H NMR (400 MHz, DMSO-d₆ at 90° C.) δ ppm 7.51-7.26 (m, 6H), 5.26-5.03 (m, 2H), 4.78-4.63 (m, 1H), 4.44 (dd, J=16.5, 9.2 Hz, 1H), 3.88 (dd, J=24.3, 16.5 Hz, 1H), 3.48-3.27 (m, 2H), 3.23-3.07 (m, 2H), 2.92 (dt, J=15.9, 3.0 Hz, 1H), 1.54 (d, J=2.0 Hz, 9H), 1.51-1.42 (m, 2H), 1.42-1.31 (m, 18H), 1.31-1.16 (m, 3H), 0.89 (td, J=7.3, 4.3 Hz, 3H); MS (ESI⁺) m/z 776 [M+Na]⁺.

### Example II-19C: tert-butyl (2R)-6-(benzyloxy)-2-{[(tert-butoxycarbonyl)(butyl)amino]methyl}-5-[(2-tert-butoxy-2-oxoethyl)amino]-4-fluoro-2,3-dihydro-1H-indole-1-carboxylate

To a solution of the product of Example II-19B (1.08 g, 1.404 mmol) in methanol (4 mL) and tetrahydrofuran (4 mL) at room temperature was added a solution of lithium hydroxide monohydrate (0.177 g, 4.21 mmol) in water (4.00 mL). The reaction mixture was then stirred at room temperature for 1 hour. The reaction mixture was neutralized to pH 7 with 1 M hydrogen chloride solution and diluted with water (10 mL). The aqueous mixture was extracted with ethyl acetate (3×15 mL). The combined organic layers were washed with brine (10 mL), dried over sodium sulfate, filtered, and concentrated in vacuo to afford the title compound (0.93 g, 1.272 mmol, 91% yield). ¹H NMR (400 MHz, DMSO-d₆) δ ppm 7.60-7.15 (m, 5H), 5.08 (s, 2H), 4.69 (s, 1H), 4.56 (s, 1H), 3.84 (d, J=6.8 Hz, 2H), 3.48-2.95 (m, 6H), 2.78 (s, 1H), 1.48 (broad s, 11H), 1.41-1.09 (m, 20H), 0.88 (t, J=7.3 Hz, 3H).

### Example II-19D: tert-butyl (2R)-6-(benzyloxy)-2-{[(tert-butoxycarbonyl)(butyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.221 mL, 2.54 mmol) in dichloromethane (3 mL) at 0° C. was added allyl alcohol (0.173 mL, 2.54 mmol) dropwise. The resulting solution was stirred for 30 minutes at 0° C. before a prepared solution of the product of Example II-19C (930 mg, 1.272 mmol) and triethylamine (0.443 mL, 3.18 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-40% ethyl acetate in isohexane, 40 mL/minute) to afford the title compound (836 mg, 0.984 mmol, 77% yield). ¹H NMR (400 MHz, DMSO-d₆) δ ppm 11.66-11.46 (m, 1H), 7.66-6.89 (m, 6H), 5.75 (s, 1H), 5.28-4.97 (m, 4H), 4.73-4.52 (m, 2H), 4.33-4.05 (m, 3H), 3.49-3.01 (m, 5H), 2.91-2.70 (m, 1H), 1.47 (s, 10H), 1.43-1.12 (m, 21H), 0.88 (q, J=7.6 Hz, 3H); MS (ESI⁻) m/z 819 [M−H]⁻.

### Example II-19E: tert-butyl (2R)-6-(benzyloxy)-2-{[(tert-butoxycarbonyl)(butyl)amino]methyl}-4-fluoro-5-(1,1,4-trioxo-1λ⁶,2,5-thiadiazolidin-2-yl)-2,3-dihydro-1H-indole-1-carboxylate-ammonia

To a solution of the product of Example II-19D (950 mg, 0.984 mmol) and potassium carbonate (544 mg, 3.93 mmol) in methanol (3 mL) was added tetrakis(triphenylphosphine)palladium(0) (114 mg, 0.098 mmol). The reaction mixture was stirred at 60° C. for 5 hours. The mixture was cooled to room temperature and concentrated under reduced pressure. The crude residue was subjected to column chromatography (Reveleris® 40 g reversed phase (C18) flash cartridge, dry loaded with C18 silica gel, 5-70% methanol in 10 mM ammonium bicarbonate, 40 mL/minute) to afford the title compound as an ammonium salt (560 mg, 0.700 mmol, 71% yield, 90% purity). ¹H NMR (400 MHz, DMSO-d₆) δ ppm 7.68-7.43 (m, 3H), 7.41-7.26 (m, 3H), 7.26-6.88 (m, 3H), 5.09 (s, 2H), 4.63 (s, 1H), 4.00-3.80 (m, 2H), 3.49-2.97 (m, 6H), 2.91-2.71 (m, 1H), 1.59-1.12 (m, 22H), 0.88 (t, J=7.3 Hz, 3H); MS (ESI⁻) m/z 661 [M−H]⁻.

### Example II-19F: tert-butyl (2R)-2-{[(tert-butoxycarbonyl)(butyl)amino]methyl}-4-fluoro-6-hydroxy-5-(1,1,4-trioxo-1λ⁶,2,5-thiadiazolidin-2-yl)-2,3-dihydro-H-indole-1-carboxylate-ammonia

To a solution of the product of Example II-19E (250 mg, 0.313 mmol) in degassed water (0.5 mL) and ethanol (3 mL) was added 10% Pd/C (34 mg). The resulting suspension was allowed to stir under hydrogen (5 bar) for 2 hours. Additional 10% Pd/C (34 mg) was added, and the resulting suspension was allowed to stir under hydrogen (5 bar) for 18 hours. Additional 10% Pd/C (34 mg) was added, and the resulting suspension was allowed to stir under hydrogen (5 bar) for 4 hours. Additional 10% Pd/C (34 mg) was added, and the resulting suspension was allowed to stir under hydrogen (5 bar) for 18 hours. The reaction mixture was filtered through a pad of diatomaceous earth and concentrated under reduced pressure. The material was dissolved in ethanol (2 mL) and water (0.5 mL) and 10% Pd/C (34 mg) was added. The resulting suspension was allowed to stir under hydrogen (5 bar) for 1 hour. Additional 10% Pd/C (34 mg) was added, and the resulting suspension was allowed to stir under hydrogen (5 bar) for 1 hour. Additional 10% Pd/C (34 mg) was added, and the resulting suspension was allowed to stir under hydrogen (5 bar) for 80 hours. The reaction mixture was filtered through a pad of diatomaceous earth and concentrated under reduced pressure. The crude residue was subjected to column chromatography (Reveleris® 24 g reversed phase (C18) flash cartridge, dry loaded with C18 silica, 5-70% methanol in 10 mM ammonium bicarbonate, 32 mL/minute) to afford the title compound as an ammonium salt (135 mg, 0.213 mmol, 68% yield). ¹H NMR (400 MHz, DMSO-d₆) δ ppm 9.27 (s, 1H), 7.36-6.65 (m, 4H), 4.59 (s, 1H), 3.98-3.78 (m, 2H), 3.53-2.95 (m, 6H), 2.85-2.63 (m, 1H), 1.57-1.41 (m, 11H), 1.41-1.28 (m, 9H), 1.27-1.16 (m, 2H), 0.88 (t, J=7.3 Hz, 3H); MS (ESI⁻) m/z 571 [M−H]⁻.

### Example II-19G: 5-{(2R)-2-[(butylamino)methyl]-4-fluoro-6-hydroxy-2,3-dihydro-1H-indol-5-yl}-1λ⁶,2,5-thiadiazolidine-1,1,3-trione

To a solution of the product of Example II-19F (133 mg, 0.232 mmol) in dichloromethane (2 mL) at room temperature was added trifluoroacetic acid (0.25 mL, 3.24 mmol). The reaction mixture was stirred at room temperature for 5 hours. The reaction mixture was neutralized with 0.7 M NH₃ in methanol:water (95:5) (10 mL) and concentrated under reduced pressure. The crude residue was subjected to column chromatography (Teledyne ISCO RediSep Rf Gold® 12 g reversed phase (C18) flash cartridge, dry loaded with C18 silica, 0-20% methanol in 10 mM ammonium bicarbonate, 30 mL/minute) to afford the title compound (20 mg, 0.054 mmol, 28% yield). ¹H NMR (400 MHz, DMSO-d₆) δ ppm 8.69 (s, 1H), 8.26 (s, 2H), 6.01 (s, 1H), 5.92 (s, 1H), 4.11 (td, J=6.5, 3.4 Hz, 1H), 3.84 (s, 2H), 3.16-2.87 (m, 5H), 2.73 (dd, J=15.6, 7.0 Hz, 1H), 1.57 (tt, J=7.9, 6.4 Hz, 2H), 1.33 (h, J=7.4 Hz, 2H), 0.90 (t, J=7.4 Hz, 3H); MS (ESI⁻) m/z 371 [M−H]⁻.

### Example II-20: 5-[(2R)-4-fluoro-6-hydroxy-2-(hydroxymethyl)-2,3-dihydro-1H-indol-5-yl]-1λ⁶,2,5-thiadiazolidine-1,1,3-trione (Compound 151)

### Example II-20A: tert-butyl (2R)-6-(benzyloxy)-5-[(2-tert-butoxy-2-oxoethyl)amino]-4-fluoro-2-({[tri(propan-2-yl)silyl]oxy}methyl)-2,3-dihydro-1H-indole-1-carboxylate

A solution of lithium hydroxide monohydrate (0.787 g, 18.77 mmol) in water (8 mL) was added to a solution of the product of Example II-2G (3.08 g, 3.75 mmol) in tetrahydrofuran (10 mL) and methanol (10 mL). The reaction mixture was rapidly stirred at room temperature for 30 minutes and then 1 M aqueous hydrochloric acid was added until the mixture was neutralized. Ethyl acetate (50 mL) and water (50 mL) were added, and the layers were separated. The aqueous layer was extracted with ethyl acetate (2×25 mL). The combined organic layers were washed with brine (40 mL), dried over magnesium sulfate, filtered, and concentrated under reduced pressure to afford the title compound (1.33 g, 1.81 mmol, 48% yield). ¹H NMR (400 MHz, DMSO-d₆) δ ppm 7.57-7.15 (m, 6H), 5.06 (s, 2H), 4.63 (s, 1H), 4.41 (s, 1H), 3.82 (dd, J=6.9, 2.1 Hz, 4H), 3.23-3.09 (m, 1H), 2.94 (d, J=15.7 Hz, 1H), 1.46 (s, 9H), 1.34 (s, 9H), 1.07-0.76 (m, 21H).

### Example II-20B: tert-butyl (2R)-6-(benzyloxy)-5-[(2-tert-butoxy-2-oxoethyl)({[(prop-2-en-1-yl)oxy]carbonyl}sulfamoyl)amino]-4-fluoro-2-({[tri(propan-2-yl)silyl]oxy}methyl)-2,3-dihydro-1H-indole-1-carboxylate

To a solution of chlorosulfonyl isocyanate (0.8 mL, 9.21 mmol) in dichloromethane (5 mL) at 0° C. was added allyl alcohol (0.62 mL, 9.12 mmol) dropwise. The resulting solution was stirred for 30 minutes at 0° C. before a prepared solution of the product of Example II-20A (2.66 g, 3.63 mmol) and triethylamine (1.7 mL, 12.20 mmol) in dichloromethane (5 mL) was added dropwise. The reaction mixture was stirred at 0° C. for 15 minutes. The reaction mixture was quenched with water (20 mL) and diluted with dichloromethane (20 mL). The layers were separated, and the aqueous layer was extracted with dichloromethane (3×20 mL). The combined organic layers were dried over magnesium sulfate, filtered, and concentrated under reduced. The crude residue was subjected to column chromatography (SiO₂, dry loaded with silica, 220 g cartridge, 0-30% Ethyl acetate/isohexane) to afford the title compound (1.46 g, 1.69 mmol, 47% yield). ¹H NMR (400 MHz, DMSO-d₆) δ ppm 11.50 (d, J=16.7 Hz, 1H), 7.52-7.12 (m, 6H), 5.75 (s, 3H), 5.33-4.98 (m, 3H), 4.66 (t, J=17.0 Hz, 1H), 4.50 (d, J=9.8 Hz, 1H), 4.40-4.03 (m, 2H), 4.03-3.59 (m, 2H), 3.21 (dd, J=16.0, 9.5 Hz, 1H), 2.94 (t, J=16.9 Hz, 1H), 1.63-1.17 (m, 18H), 0.92 (dd, J=49.8, 6.3 Hz, 21H); MS (ESI⁻) m/z 820 [M−H]⁻.

### Example II-20C: tert-butyl (2R)-6-(benzyloxy)-4-fluoro-5-(1,1,4-trioxo-1λ⁶,2,5-thiadiazolidin-2-yl)-2-({[tri(propan-2-yl)silyl]oxy}methyl)-2,3-dihydro-1H-indole-1-carboxylate

A solution of the product of Example II-20B (5.1 g, 5.89 mmol) in methanol (5 mL) was purged with nitrogen for 30 minutes. Tetrakis(triphenylphosphine)palladium(0) (0.34 g, 0.294 mmol) and sodium methoxide (5.4 mL, 29.2 mmol) were sequentially added. The reaction mixture was stirred at 60° C. for 30 minutes. 1 M Aqueous hydrochloric acid (30 mL) was added. The mixture was diluted with water (30 mL) and ethyl acetate (100 mL). The aqueous layer was separated and extracted with ethyl acetate (2×50 mL). The organic layers were combined, washed with brine (50 mL), dried over magnesium sulfate, filtered, and concentrated under reduced pressure. The crude residue was subjected to column chromatography (SiO₂, dry loaded with silica, 120 g cartridge, 0-15% methanol in dichloromethane) to afford the title compound (1.81 g, 2.45 mmol, 25% yield). ¹H NMR (400 MHz, DMSO-d₆) δ ppm 7.51-7.20 (m, 6H), 5.11 (d, J=2.3 Hz, 2H), 4.50 (d, J=9.7 Hz, 1H), 4.35-4.15 (m, 2H), 3.98-3.69 (m, 2H), 3.29-3.13 (m, 1H), 2.94 (d, J=15.8 Hz, 1H), 1.48 (s, 9H), 1.11-0.77 (m, 21H); MS (ESI⁻) m/z 662 [M−H]⁻.

### Example II-20D: tert-butyl (2R)-6-(benzyloxy)-4-fluoro-2-(hydroxymethyl)-5-(1,1,4-trioxo-1λ⁶,2,5-thiadiazolidin-2-yl)-2,3-dihydro-H-indole-1-carboxylate-ammonia-triethylamine

Triethylamine trihydrofluoride (0.33 mL, 2.027 mmol) was added to a solution of the product of Example II-20C (1 g, 1.356 mmol) in tetrahydrofuran (5 mL) at room temperature. The reaction mixture was stirred at room temperature for 20 hours. Additional triethylamine trihydrofluoride (0.35 mL, 2.149 mmol) was added, and the reaction mixture was stirred at room temperature for 24 hours. Sodium bicarbonate was added until the mixture was neutralized, and the mixture was concentrated under reduced pressure. Tetrahydrofuran (10 mL) was added to the solid and the suspension was sonicated. The mixture was filtered. The filtrate was concentrated under reduced pressure. The crude residue was subjected to column chromatography (Reveleris® 40 g reversed phase (C18) flash cartridge, dry loaded with diatomaceous earth, 5-60% methanol in 10 mM ammonium bicarbonate) to afford the title compound as an ammonium, triethylamine salt (580 mg, 1.050 mmol, 77% yield). ¹H NMR (400 MHz, DMSO-d₆) δ ppm 7.50 (d, J=7.4 Hz, 2H), 7.41-7.22 (m, 3H), 7.09 (s, 3H), 5.08 (s, 2H), 4.94 (t, J=5.7 Hz, 1H), 4.43-4.35 (m, 1H), 4.09 (q, J=5.2 Hz, 1H), 3.91 (s, 2H), 3.57-3.38 (m, 3H), 3.21-3.01 (m, 2H), 3.00-2.89 (m, 1H), 1.49 (s, 9H), 1.16 (t, J=7.3 Hz, 2H); MS (ESI⁻) m/z 506 [M−H]⁻.

### Example II-20E: tert-butyl (2R)-4-fluoro-6-hydroxy-2-(hydroxymethyl)-5-(1,1,4-trioxo-1λ⁶,2,5-thiadiazolidin-2-yl)-2,3-dihydro-1H-indole-1-carboxylate-ammonia

To a suspension of the product of Example II-20D (315 mg, 0.570 mmol) in degassed water (0.1 mL) and ethanol (3 mL) was added 10% Pd/C (40 mg). The resulting suspension was allowed to stir under hydrogen (5 bar) for 3 hours. The reaction mixture was filtered through a diatomaceous earth pad and that was washed with ethanol (3×20 mL). The filtrate was concentrated under reduced pressure. The crude residue was subjected to column chromatography (Reveleris® 12 g reversed phase (C18) flash cartridge, dry loaded with C18 silica, 10-40% methanol in 10 mM ammonium bicarbonate, 30 mL/minute) to afford the title compound as an ammonium salt (180 mg, 0.394 mmol, 69% yield). ¹H NMR (400 MHz, DMSO-d₆) δ ppm 9.23 (s, 1H), 7.25-6.85 (m, 4H), 4.93 (s, 1H), 4.41-4.29 (m, 1H), 3.91 (s, 2H), 3.53 (dd, J=10.6, 3.4 Hz, 1H), 3.48-3.17 (m, 1H), 3.14-3.00 (m, 1H), 2.90 (dd, J=15.8, 2.9 Hz, 1H), 1.50 (s, 9H); MS (ESI⁻) m/z 416 [M−H]⁻.

### Example II-20F: 5-[(2R)-4-fluoro-6-hydroxy-2-(hydroxymethyl)-2,3-dihydro-1H-indol-5-yl]-1λ⁶,2,5-thiadiazolidine-1,1,3-trione

To a solution of the product of Example II-20E (160 mg, 0.383 mmol) in dichloromethane (4 mL) at room temperature was added trifluoroacetic acid (0.4 mL, 5.19 mmol). The reaction mixture was stirred at room temperature for 1 hour. The reaction mixture was neutralized with 0.7 M NH₃ in methanol:water (95:5) (15 mL) and concentrated under reduced pressure. The crude residue was subjected to column chromatography (Reveleris® 24 g reversed phase (C18) flash cartridge, dry loaded with C18 silica, 5% methanol in 10 mM ammonium bicarbonate, 30 mL/minute) to afford the title compound (60 mg, 0.189 mmol, 49% yield). ¹H NMR (400 MHz, DMSO-d₆) δ ppm 8.46 (s, 1H), 7.07 (s, 4H), 5.91 (s, 1H), 5.82 (s, 1H), 4.78 (t, J=5.5 Hz, 1H), 3.86-3.75 (m, 3H), 2.91 (dd, J=15.4, 9.3 Hz, 1H), 2.58 (dd, J=15.4, 6.2 Hz, 1H); MS (ESI⁻) m/z 316 [M−H]⁻.

### Example II-21: 5-[(2R)-2-{[(2-cyclopropylethyl)amino]methyl}-4-fluoro-6-hydroxy-2,3-dihydro-1H-indol-5-yl]-1λ⁶,2,5-thiadiazolidine-1,1,3-trione (Compound 152)

### Example II-21A: tert-butyl (2R)-6-(benzyloxy)-5-[(2-tert-butoxy-2-oxoethyl)(trifluoroacetyl)amino]-2-{[(2-cyclopropylethyl)amino]methyl}-4-fluoro-2,3-dihydro-1H-indole-1-carboxylate

Acetic acid (0.273 mL, 4.78 mmol) was added to a solution of 2-cyclopropylethanamine (0.4 g, 4.70 mmol) and the product of Example II-2I (1 g, 1.592 mmol) in 1,2-dichloroethane (6 mL). The reaction mixture was stirred at room temperature for 2 hours. Sodium triacetoxyborohydride (0.5 g, 2.359 mmol) was added, and the reaction mixture was stirred for 1 hour. The reaction mixture was quenched with a saturated aqueous solution of sodium bicarbonate (3 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (1.19 g, 1.430 mmol, 90% yield, 80% purity). ¹H NMR (400 MHz, DMSO-d₆) δ ppm 7.50-7.28 (m, 6H), 5.26-5.03 (m, 2H), 4.59-4.47 (m, 1H), 4.43 (dd, J=16.4, 6.5 Hz, 1H), 3.95-3.85 (m, 1H), 3.25-3.10 (m, 1H), 3.07-2.89 (m, 2H), 2.82 (ddd, J=12.2, 3.7, 2.3 Hz, 1H), 2.73-2.55 (m, 3H), 1.53 (s, 9H), 1.40 (s, 9H), 1.34-1.24 (m, 2H), 0.76-0.61 (m, 1H), 0.46-0.31 (m, 2H), 0.08-−0.09 (m, 2H); MS (ESI⁺) m/z 666 [M+H]⁺.

### Example II-21B: tert-butyl (2R)-6-(benzyloxy)-2-{[(tert-butoxycarbonyl)(2-cyclopropylethyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-21A (1.19 g, 1.430 mmol) in acetonitrile (3 mL) at room temperature was added N,N-diisopropylethylamine (075 mL, 429 mmol) and di-tert-butyl dicarbonate (0.62 g, 2.84 mmol). The mixture was stirred at room temperature for 18 hours. The mixture was quenched with water (10 mL). The aqueous mixture was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-40% ethyl acetate in isohexane, 50 mL/minute) to afford the title compound (1.11 g, 1.377 mmol, 96% yield). ¹H NMR (400 MHz, DMSO-d₆ at 90° C.) δ ppm 7.48-7.24 (m, 6H), 5.25-5.05 (m, 2H), 4.78-4.61 (m, 1H), 4.43 (dd, J=16.4, 8.8 Hz, 1H), 3.88 (dd, J=24.4, 16.5 Hz, 1H), 3.48-3.28 (m, 2H), 3.28-3.07 (m, 3H), 2.94 (ddd, J=19.0, 15.3, 2.2 Hz, 1H), 1.63-1.48 (m, 9H), 1.47-1.26 (m, 20H), 0.70-0.53 (m, 1H), 0.46-0.32 (m, 2H), 0.06-−0.01 (m, 2H); MS (ESI⁺) m/z 788 [M+Na]⁺.

### Example II-21C: tert-butyl (2R)-6-(benzyloxy)-2-{[(tert-butoxycarbonyl)(2-cyclopropylethyl)amino]methyl}-5-[(2-tert-butoxy-2-oxoethyl)amino]-4-fluoro-2,3-dihydro-1H-indole-1-carboxylate

To a solution of the product of Example II-21B (1.11 g, 1.377 mmol) in methanol (4 mL) and tetrahydrofuran (4 mL) at room temperature was added a solution of lithium hydroxide monohydrate (0.173 g, 4.13 mmol) in water (4.00 mL). The reaction mixture was then stirred at room temperature for 1 hour. The reaction mixture was neutralized to pH 7 with 1 M aqueous hydrochloric acid and diluted with water (10 mL). The aqueous mixture was extracted with ethyl acetate (3×15 mL). The combined organic layers were washed with brine (10 mL), dried over sodium sulfate, filtered, and concentrated in vacuo to afford the title compound (0.96 g, 1.290 mmol, 94% yield). ¹H NMR (400 MHz, DMSO-d₆) δ ppm 7.57-7.18 (m, 6H), 5.08 (s, 2H), 4.69 (s, 1H), 4.56 (s, 1H), 3.84 (d, J=6.2 Hz, 2H), 3.48-3.00 (m, 5H), 2.79 (s, 1H), 1.48 (s, 9H), 1.32 (d, J=17.5 Hz, 20H), 0.56 (s, 1H), 0.39 (d, J=7.7 Hz, 2H), 0.00 (d, J=4.9 Hz, 2H).

### Example II-21D: tert-butyl (2R)-6-(benzyloxy)-2-{[(tert-butoxycarbonyl)(2-cyclopropylethyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.224 mL, 2.58 mmol) in dichloromethane (3 mL) at 0° C. was added allyl alcohol (0.175 mL, 2.58 mmol) dropwise. The resulting solution was stirred for 30 minutes at 0° C. before a prepared solution of the product of Example II-21C (960 mg, 1.290 mmol) and triethylamine (0.449 mL, 3.22 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-40% ethyl acetate in isohexane, 40 mL/minute) to afford the title compound (850 mg, 0.998 mmol, 77% yield). ¹H NMR (400 MHz, DMSO-d₆) δ ppm 11.66-11.46 (m, 1H), 7.70-6.93 (m, 6H), 6.08-5.80 (m, 0.5H), 5.79-5.63 (m, 0.5H), 5.57-5.42 (m, 0.5H), 5.36-4.98 (m, 3.5H), 4.78 (dt, J=6.3, 1.2 Hz, 0.5H), 4.65 (dd, J=17.3, 10.5 Hz, 2H), 4.42 (dt, J=5.2, 1.6 Hz, 0.5H), 4.32-4.07 (m, 3H), 3.50-3.02 (m, 4H), 2.89-2.71 (m, 1H), 1.48 (s, 9H), 1.35 (d, J=7.2 Hz, 20H), 0.57 (d, J=10.2 Hz, 1H), 0.46-0.29 (m, 2H), 0.08-−0.07 (m, 2H); MS (ESI⁻) m/z 831 [M−H]⁻.

### Example II-21E: tert-butyl (2R)-6-(benzyloxy)-2-{[(tert-butoxycarbonyl)(2-cyclopropylethyl)amino]methyl}-4-fluoro-5-(1,1,4-trioxo-1λ⁶,2,5-thiadiazolidin-2-yl)-2,3-dihydro-1H-indole-1-carboxylate-ammonia

To a solution of the product of Example II-21D (924 mg, 0.998 mmol) and potassium carbonate (552 mg, 3.99 mmol) in methanol (3 mL) was added tetrakis(triphenylphosphine)palladium(0) (115 mg, 0.100 mmol). The reaction mixture was stirred at 60° C. for 5 hours. The mixture was cooled to room temperature and concentrated under reduced pressure. The crude residue was subjected to column chromatography (Reveleris® 40 g reversed phase (C18) flash cartridge, dry loaded with C18 silica gel, 5-70% methanol in 10 mM ammonium bicarbonate, 40 mL/minute) to afford the title compound as an ammonium salt (445 mg, 0.547 mmol, 55% yield, 85% purity). ¹H NMR (400 MHz, DMSO-d₆) δ ppm 7.67-7.45 (m, 3H), 7.39-7.26 (m, 3H), 7.26-6.90 (m, 3H), 5.09 (d, J=2.9 Hz, 2H), 4.34 (s, 1H), 3.99-3.83 (m, 2H), 3.50-3.40 (m, 2H), 3.39-3.03 (m, 4H), 2.90-2.71 (m, 1H), 1.50 (s, 9H), 1.44-1.18 (m, 11H), 0.57 (s, 1H), 0.39 (d, J=7.7 Hz, 2H), 0.06-−0.04 (m, 2H); MS (ESI⁺) m/z 697 [M+Na]⁺.

### Example II-21F: tert-butyl (2R)-2-{[(tert-butoxycarbonyl)(2-cyclopropylethyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a solution of the product of Example II-21E (200 mg, 0.246 mmol) in degassed water (0.5 mL) and ethanol (2 mL) was added 10% Pd/C (26 mg). The resulting suspension was allowed to stir under hydrogen (5 bar) for 4 hours. Additional 10% Pd/C (26 mg) was added, and the resulting suspension was allowed to stir under hydrogen (5 bar) for 18 hours. The reaction mixture was filtered through a diatomaceous earth pad, and the filtrate was concentrated under reduced pressure. The crude residue was dissolved in ethanol (2 mL) and water (0.5 mL). 10% Pd/C (26 mg) was added, and the resulting suspension was allowed to stir under hydrogen (5 bar) for 1 hour. Additional 10% Pd/C (26 mg) was added, and the resulting suspension was allowed to stir under hydrogen (5 bar) for 1 hour. The reaction mixture was filtered through a diatomaceous earth pad, and the filtrate was concentrated under reduced pressure. The crude residue was subjected to column chromatography (Reveleris® 24 g reversed phase (C18) flash cartridge, dry loaded with C18 silica, 5-65% methanol in 10 mM ammonium bicarbonate, 32 mL/minute) to afford the title compound as an ammonium salt (155 mg, 0.232 mmol, 94% yield). ¹H NMR (400 MHz, DMSO-d₆) δ ppm 9.28 (s, 1H), 7.37-6.84 (m, 4H), 4.58 (s, 1H), 4.03-3.76 (m, 2H), 3.48-2.96 (m, 6H), 2.85-2.65 (m, 1H), 1.50 (s, 9H), 1.43-1.18 (m, 11H), 0.57 (s, 1H), 0.39 (d, J=7.7 Hz, 2H), 0.09-−0.07 (m, 2H); MS (ESI⁻) m/z 583 [M−H]⁻.

### Example II-21G: 5-[(2R)-2-{[(2-cyclopropylethyl)amino]methyl}-4-fluoro-6-hydroxy-2,3-dihydro-1H-indol-5-yl]-1λ⁶,2,5-thiadiazolidine-1,1,3-trione

To a solution of the product of Example II-21F (139 mg, 0.208 mmol) in dichloromethane (2 mL) at room temperature was added trifluoroacetic acid (0.25 mL, 3.24 mmol). The reaction mixture was stirred at room temperature for 5 hours. The reaction mixture was neutralized with 0.7 M NH₃ in methanol:water (95:5) (10 mL) and concentrated under reduced pressure. The crude residue was subjected to column chromatography (Teledyne ISCO RediSep Rf Gold® 12 g reversed phase (C18) flash cartridge, dry loaded with C18 silica, 0-20% methanol in 10 mM ammonium bicarbonate, 30 mL/minute) to afford the title compound (50 mg, 0.124 mmol, 59% yield). ¹H NMR (400 MHz, DMSO-d₆) δ ppm 8.68 (s, 1H), 8.26 (s, 2H), 6.02 (t, J=2.4 Hz, 1H), 5.92 (s, 1H), 4.11 (ddd, J=9.0, 6.3, 2.8 Hz, 1H), 3.84 (s, 2H), 3.19-2.92 (m, 5H), 2.73 (dd, J=15.6, 6.9 Hz, 1H), 1.50 (dt, J=10.0, 7.3 Hz, 2H), 0.78-0.62 (m, 1H), 0.51-0.36 (m, 2H), 0.19-0.03 (m, 2H); MS (ESI⁺) m/z 385 [M+H]⁺.

### Example II-22: 5-[(2R)-2-{[(3,3-dimethylbutyl)amino]methyl}-4-fluoro-6-hydroxy-2,3-dihydro-1H-indol-5-yl]-1λ⁶,2,5-thiadiazolidine-1,1,3-trione (Compound 153)

### Example II-22A: tert-butyl (2R)-6-(benzyloxy)-5-[(2-tert-butoxy-2-oxoethyl)(trifluoroacetyl)amino]-2-{[(3,3-dimethylbutyl)amino]methyl}-4-fluoro-2,3-dihydro-1H-indole-1-carboxylate

Acetic acid (0.273 mL, 4.78 mmol) was added to a solution of 3,3-dimethylbutan-1-amine (0.645 mL, 4.78 mmol) and the product of Example II-2I (1 g, 1.592 mmol) in 1,2-dichloroethane (6 mL). The reaction mixture was stirred at room temperature for 2 hours. Sodium triacetoxyborohydride (0.506 g, 2.389 mmol) was added. More 1,2-dichloroethane (3 mL) was added, and the reaction mixture was stirred for 1 hour. The reaction mixture was quenched with a saturated aqueous solution of sodium bicarbonate (3 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (1.21 g, 1.580 mmol, 99% yield, 90% purity). ¹H NMR (400 MHz, DMSO-d₆) δ ppm 7.48-7.22 (m, 6H), 5.26-4.99 (m, 2H), 4.57-4.47 (m, 1H), 4.43 (dd, J=16.4, 7.4 Hz, 1H), 3.95-3.83 (m, 1H), 3.24-3.10 (m, 1H), 3.06-2.96 (m, 2H), 2.81 (ddd, J=12.3, 3.7, 1.8 Hz, 1H), 2.73-2.52 (m, 3H), 1.54 (d, J=0.6 Hz, 9H), 1.40 (s, 9H), 1.33-1.25 (m, 2H), 0.96-0.82 (m, 9H); MS (ESI⁺) m/z 683 [M+H]⁺.

### Example II-22B: tert-butyl (2R)-6-(benzyloxy)-2-{[(tert-butoxycarbonyl)(3,3-dimethylbutyl)amino]methyl}-5-[(2-tert-butoxy-2-oxoethyl)(trifluoroacetyl)amino]-4-fluoro-2,3-dihydro-1H-indole-1-carboxylate

To a solution of the product of Example II-22A (1.21 g, 1.580 mmol) in acetonitrile (7 mL) at room temperature was added N,N-diisopropylethylamine (0.85 mL, 4.87 mmol) and di-tert-butyl dicarbonate (0.7 g, 3.21 mmol). The mixture was stirred at room temperature for 30 minutes. The reaction was quenched with water (10 mL). The aqueous mixture was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-40% ethyl acetate in isohexane, 35 mL/minute) to afford the title compound (1.1 g, 1.322 mmol, 84% yield). ¹H NMR (400 MHz, DMSO-d₆ at 90° C.) δ ppm 7.49-7.26 (m, 6H), 5.27-5.04 (m, 2H), 4.80-4.61 (m, 1H), 4.44 (dd, J=16.5, 8.9 Hz, 1H), 3.87 (dd, J=28.2, 16.5 Hz, 1H), 3.47-3.26 (m, 2H), 3.23-3.07 (m, 3H), 2.91 (ddd, J=15.9, 7.2, 2.1 Hz, 1H), 1.63-1.47 (m, 9H), 1.47-1.27 (m, 20H), 0.96-0.79 (m, 9H); MS (ESI⁺) m/z 804 [M+Na]⁺.

### Example II-22C: tert-butyl (2R)-6-(benzyloxy)-2-{[(tert-butoxycarbonyl)(3,3-dimethylbutyl)amino]methyl}-5-[(2-tert-butoxy-2-oxoethyl)amino]-4-fluoro-2,3-dihydro-1H-indole-1-carboxylate

To a solution of the product of Example II-22B (1.1 g, 1.322 mmol) in methanol (4 mL) and tetrahydrofuran (4 mL) at room temperature was added a solution of lithium hydroxide monohydrate (0.166 g, 3.97 mmol) in water (4 mL). The reaction mixture was then stirred at room temperature for 1 hour. The reaction mixture was neutralized to pH 7 with 1 M aqueous hydrochloric acid solution and diluted with water (10 mL). The aqueous mixture was extracted with ethyl acetate (3×10 mL). The combined organic layers were washed with brine (10 mL), dried over sodium sulfate, filtered, and concentrated in vacuo to afford the title compound (866 mg, 1.136 mmol, 86% yield, 90% purity). ¹H NMR (400 MHz, DMSO-d₆) δ ppm 7.65-6.83 (m, 6H), 5.08 (s, 2H), 4.70 (s, 1H), 4.57 (s, 1H), 3.91-3.70 (m, 2H), 3.45-2.97 (m, 5H), 2.76 (t, J=18.9 Hz, 1H), 1.48 (s, 9H), 1.43-1.11 (m, 20H), 0.95-0.72 (m, 9H).

### Example II-22D: tert-butyl (2R)-6-(benzyloxy)-2-{[(tert-butoxycarbonyl)(3,3-dimethylbutyl)amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.197 mL, 2.273 mmol) in dichloromethane (3 mL) at 0° C. was added allyl alcohol (0.155 mL, 2.273 mmol) dropwise. The resulting solution was stirred for 30 minutes at 0° C. before a prepared solution of the product of Example II-22C (866 mg, 1.136 mmol) and triethylamine (0.396 mL, 2.84 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-40% ethyl acetate in isohexane, 40 mL/minute) to afford the title compound (870 mg, 0.922 mmol, 81% yield, 90% purity). ¹H NMR (400 MHz, DMSO-d₆) δ ppm 11.65-11.50 (m, 1H), 7.57-7.18 (m, 6H), 5.81-5.59 (m, 1H), 5.29-4.96 (m, 4H), 4.73-4.58 (m, 2H), 4.35-4.01 (m, 3H), 3.44-3.28 (m, 1H), 3.27-2.99 (m, 4H), 2.81 (d, J=16.5 Hz, 1H), 1.48 (s, 9H), 1.43-1.18 (m, 20H), 0.95-0.72 (m, 9H); MS (ESI⁻) m/z 848 [M−H]⁻.

### Example II-22E: tert-butyl (2R)-6-(benzyloxy)-2-{[(tert-butoxycarbonyl)(3,3-dimethylbutyl)amino]methyl}-4-fluoro-5-(1,1,4-trioxo-lf, 2,5-thiadiazolidin-2-yl)-2,3-dihydro-1H-indole-1-carboxylate-ammonia

To a solution of the product of Example II-22D (870 mg, 0.922 mmol) and potassium carbonate (510 mg, 3.69 mmol) in methanol (3 mL) was added tetrakis(triphenylphosphine)palladium(0) (107 mg, 0.092 mmol). The reaction mixture was stirred at 60° C. for 5 hours. The mixture was cooled to room temperature and concentrated under reduced pressure. The crude residue was subjected to column chromatography (Reveleris® 40 g reversed phase (C18) flash cartridge, dry loaded with C18 silica gel, 5-70% methanol in 10 mM ammonium bicarbonate, 40 mL/minute) to afford the title compound as an ammonium salt (474 mg, 0.633 mmol, 69% yield). ¹H NMR (400 MHz, DMSO-d₆) δ ppm 7.54-7.46 (m, 2H), 7.40-7.25 (m, 4H), 7.24-6.85 (m, 3H), 5.09 (s, 2H), 4.64 (s, 1H), 3.98-3.79 (m, 2H), 3.49-3.38 (m, 1H), 3.27-3.02 (m, 5H), 2.88-2.70 (m, 1H), 1.50 (s, 9H), 1.44-1.22 (m, 11H), 0.87 (s, 9H); MS (ESI⁺) m/z 713 [M+Na]⁺.

### Example II-22F: tert-butyl (2R)-2-{[(tert-butoxycarbonyl)(3,3-dimethylbutyl)amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a solution of the product of Example II-22E (250 mg, 0.300 mmol) in degassed water (0.5 mL) and ethanol (3 mL) was added 10% Pd/C (31.9 mg). The resulting suspension was allowed to stir under hydrogen (5 bar) for 4 hours. The reaction mixture was filtered through a diatomaceous earth pad, washed with ethanol (3×20 mL) and concentrated under reduced pressure. The crude residue was subjected to column chromatography (Reveleris® 24 g reversed phase (C18) flash cartridge, dry loaded with C18 silica, 5-70% methanol in 10 mM ammonium bicarbonate, 32 mL/minute) to afford the title compound (207 mg, 0.302 mmol, 100% yield, 90% purity) as an ammonium salt. ¹H NMR (400 MHz, DMSO-d₆) δ ppm 9.27 (s, 1H), 7.08 (s, 4H), 4.59 (s, 1H), 3.98-3.77 (m, 2H), 3.26-2.95 (m, 6H), 2.84-2.61 (m, 1H), 1.50 (s, 9H), 1.43-1.25 (m, 11H), 0.88 (s, 9H); MS (ESI⁺) m/z 623 [M+Na]⁺.

### Example II-22G: 5-[(2R)-2-{[(3,3-dimethylbutyl)amino]methyl}-4-fluoro-6-hydroxy-2,3-dihydro-1H-indol-5-yl]-1λ⁶,2,5-thiadiazolidine-1,1,3-trione

To a solution of the product of Example II-22F (185 mg, 0.270 mmol) in dichloromethane (2 mL) at room temperature was added trifluoroacetic acid (0.311 mL, 4.04 mmol). The reaction mixture was stirred at room temperature for 5 hours. The reaction mixture was neutralized with 0.7 M NH₃ in methanol:water (95:5) (10 mL) and concentrated under reduced pressure. The crude residue was subjected to column chromatography (Teledyne ISCO RediSep Rf Gold® 12 g reversed phase (C18) flash cartridge, dry loaded with C18 silica, 0-30% methanol in 10 mM ammonium bicarbonate, 30 mL/minute) to afford the title compound (70 mg, 0.166 mmol, 62% yield). ¹H NMR (400 MHz, DMSO-d₆) δ ppm 8.68 (s, 1H), 8.20 (s, 2H), 6.01 (s, 1H), 5.92 (s, 1H), 4.17-4.04 (m, 1H), 3.84 (s, 2H), 3.16-2.86 (m, 5H), 2.73 (dd, J=15.6, 6.9 Hz, 1H), 1.56-1.44 (m, 2H), 0.90 (s, 9H); MS (ESI⁺) m/z 401 [M+H]⁺.

### Example II-23: 5-{(2R)-4-fluoro-6-hydroxy-2-[({[3-(propan-2-yl)cyclobutyl]methyl}amino)methyl]-2,3-dihydro-1H-indol-5-yl}-1λ⁶,2,5-thiadiazolidine-1,1,3-trione (Compound 154)

### Example II-23A: tert-butyl (2R)-6-(benzyloxy)-5-[(2-tert-butoxy-2-oxoethyl)(trifluoroacetyl)amino]-4-fluoro-2-[({[3-(propan-2-yl)cyclobutyl]methyl}amino)methyl]-2,3-dihydro-1H-indole-1-carboxylate

Acetic acid (0.168 mL, 2.94 mmol) was added to a solution of (3-isopropylcyclobutyl)methanamine, hydrochloric acid (0.482 g, 2.94 mmol) and the product of Example II-2I (0.900 g, 0.981 mmol) in 1,2-dichloroethane (3 mL). Triethylamine (0.410 mL, 2.94 mmol) was added, and the reaction mixture was stirred at room temperature for 1 hour. Sodium triacetoxyborohydride (0.312 g, 1.471 mmol) was added, and the reaction mixture was stirred for 30 minutes. The reaction mixture was quenched with a saturated aqueous solution of sodium bicarbonate (6 mL) and stirred for 10 minutes. The mixture was diluted with water (20 mL) and extracted with ethyl acetate (3×20 mL). The combined organic layers were washed with brine (20 mL), dried over sodium sulfate, filtered, and concentrated under reduced pressure to afford the title compound (1.14 g, 0.966 mmol, 99% yield, 60% purity). ¹H NMR (400 MHz, DMSO-d₆) δ ppm 7.53-7.24 (m, 6H), 5.22-5.03 (m, 2H), 4.52-4.32 (m, 2H), 4.03-3.91 (m, 1H), 3.21-3.07 (m, 1H), 3.05-2.93 (m, 1H), 2.79-2.69 (m, 1H), 2.69-2.53 (m, 1H), 2.45-2.36 (m, 1H), 2.19-1.93 (m, 2H), 1.91-1.79 (m, 1H), 1.75-1.59 (m, 4H), 1.55-1.43 (m, 9H), 1.42-1.29 (m, 9H), 1.28-1.10 (m, 2H), 0.81-0.67 (m, 6H); MS (ESI⁺) m/z 709 [M+H]⁺.

### Example II-23B: tert-butyl (2R)-6-(benzyloxy)-2-{[(tert-butoxycarbonyl){[3-(propan-2-yl)cyclobutyl]methyl}amino]methyl}-5-[(2-tert-butoxy-2-oxoethyl)(trifluoroacetyl)amino]-4-fluoro-2,3-dihydro-1H-indole-1-carboxylate

To a solution of the product of Example II-23A (1.14 g, 0.966 mmol) in acetonitrile (4 mL) at room temperature was added N,N-diisopropylethylamine (0.506 mL, 2.90 mmol) and di-tert-butyl dicarbonate (0.422 g, 1.933 mmol). The mixture was stirred at room temperature for 1 hour. The reaction was quenched with water (10 mL). The aqueous mixture was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-30% ethyl acetate in isohexane, 32 mL/minute) to afford the title compound (779 mg, 0.916 mmol, 95% yield). ¹H NMR (400 MHz, DMSO-d₆) δ ppm 7.59-7.25 (m, 6H), 5.25-5.01 (m, 2H), 4.66 (s, 1H), 4.55-4.31 (m, 1H), 3.44 (s, 1H), 3.21-3.05 (m, 3H), 3.03-2.74 (m, 1H), 2.40-2.21 (m, 2H), 2.07-1.82 (m, 2H), 1.79-1.61 (m, 3H), 1.51 (s, 9H), 1.44-1.22 (m, 20H), 0.83-0.70 (m, 6H); MS (ESI⁺) m/z 831 [M+Na]⁺.

### Example II-23C: tert-butyl (2R)-6-(benzyloxy)-2-{[(tert-butoxycarbonyl){[3-(propan-2-yl)cyclobutyl]methyl}amino]methyl}-5-[(2-tert-butoxy-2-oxoethyl)amino]-4-fluoro-2,3-dihydro-1H-indole-1-carboxylate

To a solution of the product of Example II-23B (779 mg, 0.916 mmol) in methanol (4 mL) and tetrahydrofuran (4 mL) at room temperature was added a solution of lithium hydroxide monohydrate (115 mg, 2.75 mmol) in water (4 mL). The reaction mixture was then stirred at room temperature for 1 hour. The reaction mixture was neutralized to pH 7 with 1 M aqueous hydrochloric acid and diluted with water (10 mL). The aqueous layer was extracted with ethyl acetate (3×10 mL). The combined organic layers were washed with brine (10 mL), dried over sodium sulfate, filtered, and concentrated in vacuo to afford the title compound (674 mg, 0.805 mmol, 88% yield). ¹H NMR (400 MHz, DMSO-d₆) δ ppm 7.63-7.17 (m, 6H), 5.08 (s, 2H), 4.69 (s, 1H), 4.55 (s, 1H), 3.91-3.76 (m, 2H), 3.46-3.26 (m, 1H), 3.27-2.94 (m, 4H), 2.90-2.68 (m, 1H), 2.43-2.17 (m, 1H), 2.08-1.85 (m, 1H), 1.77-1.62 (m, 3H), 1.54-1.42 (m, 9H), 1.43-1.21 (m, 20H), 0.82-0.67 (m, 6H).

### Example II-23D: tert-butyl (2R)-6-(benzyloxy)-2-{[(tert-butoxycarbonyl){[3-(propan-2-yl)cyclobutyl]methyl}amino]methyl}-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.140 mL, 1.609 mmol) in dichloromethane (3 mL) at 0° C. was added allyl alcohol (0.109 mL, 1.609 mmol) dropwise. The resulting solution was stirred for 30 minutes at 0° C. before a prepared solution of the product of Example II-23C (674 mg, 0.805 mmol) and triethylamine (0.280 mL, 2.012 mmol) in dichloromethane (5 mL) was added dropwise. The resulting solution was stirred at 0° C. for 30 minutes. The reaction mixture was quenched with water (10 mL) and diluted with dichloromethane (10 mL). The layers were separated, and the aqueous layer was extracted with dichloromethane (3×10 mL). The combined organic layers were dried over sodium sulfate, filtered, and concentrated under reduced pressure. The crude residue was subjected to column chromatography (silica gel, 40 g cartridge, 0-40% ethyl acetate in isohexane, 40 mL/minute) to afford the title compound (609 mg, 0.680 mmol, 84% yield). ¹H NMR (400 MHz, DMSO-d₆) δ ppm 11.69-11.46 (m, 1H), 7.59-6.85 (m, 6H), 6.11-5.78 (m, 1H), 5.79-5.64 (m, 1H), 5.56-5.26 (m, 1H), 5.26-5.01 (m, 3H), 4.74-4.55 (m, 2H), 4.31-4.20 (m, 1H), 4.20-4.08 (m, 1H), 3.47-3.36 (m, 1H), 3.26-3.00 (m, 4H), 2.91-2.70 (m, 1H), 2.43-2.19 (m, 1H), 2.09-1.85 (m, 1H), 1.78-1.61 (m, 3H), 1.48 (s, 9H), 1.43-1.22 (m, 20H), 0.82-0.70 (m, 6H); MS (ESI⁻) m/z 873 [M−H]⁻.

### Example II-23E: tert-butyl (2R)-6-(benzyloxy)-2-{[(tert-butoxycarbonyl){[3-(propan-2-yl)cyclobutyl]methyl}amino]methyl}-4-fluoro-5-(1,1,4-trioxo-1λ⁶,2,5-thiadiazolidin-2-yl)-2,3-dihydro-1H-indole-1-carboxylate-ammonia

To a solution of the product of Example II-23D (700 mg, 0.680 mmol) and potassium carbonate (376 mg, 2.72 mmol) in methanol (3 mL) was added tetrakis(triphenylphosphine)palladium(0) (79 mg, 0.068 mmol). The reaction mixture was stirred at 60° C. for 7 hours. The mixture was cooled to room temperature and concentrated under reduced pressure. The crude residue was subjected to column chromatography (Reveleris® 40 g reversed phase (C18) flash cartridge, dry loaded with C18 silica gel, 5-70% methanol in 10 mM ammonium bicarbonate, 40 mL/minute) to afford the title compound as an ammonium salt (185 mg, 0.239 mmol, 35% yield). ¹H NMR (400 MHz, DMSO-d₆ at 90° C.) 8 ppm 7.56-7.46 (m, 2H), 7.39-7.24 (m, 3H), 7.21 (s, 1H), 7.01 (s, 3H), 5.11 (s, 2H), 4.71-4.60 (m, 1H), 4.00-3.81 (m, 2H), 3.42-3.23 (m, 3H), 3.19-2.94 (m, 2H), 2.85 (dd, J=15.5, 2.1 Hz, 1H), 2.47-2.36 (m, 1H), 2.35-2.21 (m, 1H), 2.09-1.90 (m, 1.5H), 1.82-1.68 (m, 2.5H), 1.59-1.46 (m, 9H), 1.45-1.24 (m, 11H), 0.88-0.72 (m, 6H); MS (ESI⁻) m/z 715 [M−H]⁻.

### Example II-23F: tert-butyl (2R)-2-{[(tert-butoxycarbonyl){[3-(propan-2-yl)cyclobutyl]methyl}amino]methyl}-4-fluoro-6-hydroxy-5-(1,1,4-trioxo-1λ⁶,2,5-thiadiazolidin-2-yl)-2,3-dihydro-1H-indole-1-carboxylate-ammonia

To a solution of the product of Example II-23E (180 mg, 0.233 mmol) in degassed water (0.5 mL) and ethanol (3 mL) was added 10% Pd/C (24.8 mg, 0.023 mmol). The resulting suspension was allowed to stir under hydrogen (5 bar) for 4 hours. The reaction mixture was filtered through a diatomaceous earth pad, washed with ethanol (3×20 mL) and concentrated under reduced pressure. The crude residue was subjected to column chromatography (Reveleris® 24 g reversed phase (C18) flash cartridge, dry loaded with C18 silica, 5-70% methanol in 10 mM ammonium bicarbonate, 32 mL/minute) to afford the title compound as an ammonium salt (115 mg, 0.161 mmol, 69% yield). ¹H NMR (400 MHz, DMSO-d₆ at 90° C.) δ ppm 8.64 (s, 1H), 7.32-6.78 (m, 4H), 4.70-4.52 (m, 1H), 3.99-3.79 (m, 2H), 3.39-3.22 (m, 3H), 3.21-3.10 (m, 1H), 3.09-2.94 (m, 1H), 2.81 (dd, J=15.7, 2.1 Hz, 1H), 2.46-2.36 (m, 0.5H), 2.35-2.21 (m, 0.5H), 2.10-1.89 (m, 1.5H), 1.83-1.68 (m, 2.5H), 1.60-1.47 (m, 10H), 1.46-1.25 (m, 11H), 0.84-0.73 (m, 6H); MS (ESI⁻) m/z 625 [M−H]⁻.

### Example II-23G: 5-{(2R)-4-fluoro-6-hydroxy-2-[({[3-(propan-2-yl)cyclobutyl]methyl}amino)methyl]-2,3-dihydro-1H-indol-5-yl}-1λ⁶,2,5-thiadiazolidine-1,1,3-trione

To a solution of the product of Example II-23F (105 mg, 0.147 mmol) in dichloromethane (2 mL) at room temperature was added trifluoroacetic acid (0.170 mL, 2.202 mmol). The reaction mixture was stirred at room temperature for 3 hours. The reaction mixture was neutralized with 0.7 M NH₃ in methanol:water (95:5) (10 mL) and concentrated under reduced pressure. The crude residue was subjected to column chromatography (Teledyne ISCO RediSep Rf Gold® 12 g reversed phase (C18) flash cartridge, dry loaded with C18 silica, 0-30% methanol in 10 mM ammonium bicarbonate, 30 mL/minute) to afford the title compound (51 mg, 0.114 mmol, 77% yield). ¹H NMR (400 MHz, DMSO-d₆) δ ppm 8.67 (s, 1H), 8.17 (s, 2H), 6.00 (s, 1H), 5.92 (s, 1H), 4.18-4.02 (m, 1H), 3.83 (s, 2H), 3.15-3.04 (m, 2H), 3.04-2.88 (m, 3H), 2.77-2.68 (m, 1H), 2.46-2.28 (m, 1H), 2.17-2.06 (m, 1H), 2.00-1.89 (m, 0.5H), 1.89-1.71 (m, 2.5H), 1.58-1.46 (m, 0.5H), 1.45-1.31 (m, 1.5H), 0.84-0.70 (m, 6H); MS (ESI⁺) m/z 427 [M+H]⁺.

### Example II-24: 5-[(2R)-4-fluoro-6-hydroxy-2-{[methyl(2-methylpropyl)amino]methyl}-2,3-dihydro-1H-indol-5-yl]-1λ⁶,2,5-thiadiazolidine-1,1,3-trione (Compound 155)

### Example II-24A-1: tert-butyl (2R)-4-fluoro-6-hydroxy-2-{[(2-methylpropyl)amino]methyl}-5-(1,1,4-trioxo-1λ⁶,2,5-thiadiazolidin-2-yl)-2,3-dihydro-1H-indole-1-carboxylate and

### Example II-24A-2: tert-butyl {[(2R)-4-fluoro-6-hydroxy-5-(1,1,4-trioxo-1λ⁶,2,5-thiadiazolidin-2-yl)-2,3-dihydro-1H-indol-2-yl]methyl}(2-methylpropyl)carbamate

Trifluoroacetic acid (1.252 mL, 16.25 mmol) was added to a solution of the product of Example II-20 (653 mg, 1.083 mmol) in dichloromethane (10 mL). The resulting solution was stirred at room temperature for 30 minutes. The reaction was then neutralized with 0.7 M NH₃ in methanol (15 mL) and concentrated under reduced pressure. The crude residue was dissolved in dimethyl sulfoxide (8 mL), filtered and purified by reversed phase preparative HPLC on a Phenomenex® Gemini® NX—C18 prep column, 110 Å, 5 μm, 30 mm×150 mm, flow rate 40 mL/minute eluted with 0.3% ammonia in water-methanol over 15 minutes using UV detection at 220 nm. At-column dilution pump gives 2 mL/minute methanol over the first minute. Gradient information: 0.0-0.5 minute, 5% methanol; 0.5-1.0 minute, ramped from 5% methanol to 30% methanol; 1.0-16.0 minutes, isocratic at 30% methanol, ramped from 30% methanol to 100% acetonitrile; 16.0-16.5 minutes, held at 100% acetonitrile. This afforded the two regioisomeric title compounds, Example II-24A-1 (first to elute) and Example II-24A-2 (second to elute):

Example II-24A-1 (216 mg, 0.434 mmol, 40% yield). ¹H NMR (400 MHz, DMSO-d₆ at 90° C.) 5 ppm 8.75 (s, 1H), 6.96 (s, 1H), 4.80-4.62 (m, 1H), 3.93 (s, 2H), 3.23 (dd, J=16.0, 9.7 Hz, 1H), 3.10 (dd, J=12.5, 5.3 Hz, 2H), 3.03-2.91 (m, 2H), 2.80-2.65 (m, 2H), 1.92 (hept, J=6.8 Hz, 1H), 1.55 (s, 9H), 0.95 (d, J=6.6 Hz, 6H); MS (ESI⁺) m/z 473 [M+H]⁺.

### And

Example II-24A-2 (57 mg, 0.121 mmol, 11% yield). ¹H NMR (400 MHz, DMSO-d₆) δ ppm 8.52 (s, 1H), 7.08 (s, 4H), 5.97 (s, 1H), 5.83 (s, 1H), 4.01 (s, 1H), 3.82 (s, 2H), 3.32-3.26 (m, 1H), 3.11 (s, 2H), 2.97 (s, 1H), 2.91 (dd, J=15.3, 9.0 Hz, 1H), 2.57 (dd, J=15.4, 6.9 Hz, 1H), 1.89 (dt, J=13.7, 6.9 Hz, 1H), 1.39 (s, 9H), 0.82 (t, J=6.0 Hz, 6H); MS (ESI⁻) m/z 471 [M−H]⁻.

### Example II-24B: tert-butyl (2R)-4-fluoro-6-hydroxy-2-{[methyl(2-methylpropyl)amino]methyl}-5-(1,1,4-trioxo-1λ⁶,2,5-thiadiazolidin-2-yl)-2,3-dihydro-1H-indole-1-carboxylate

Acetic acid (0.035 mL, 0.603 mmol) was added to a solution of formaldehyde (50% in water) (0.033 mL, 0.603 mmol) and the product of Example II-24A-1 (100 mg, 0.201 mmol) in 1,2-dichloroethane (1 mL), followed by sodium triacetoxyborohydride (63.9 mg, 0.302 mmol). The reaction mixture was stirred at room temperature for 1 hour. The reaction mixture was then neutralized with 0.7 M NH₃ in methanol (2 mL) and concentrated onto C18 silica gel (1 g). The crude residue was subjected to column chromatography (Teledyne ISCO RediSep Rf Gold® 12 g reversed phase (C18) flash cartridge, 0-30% methanol in 0.1% ammonium hydroxide, 15 mL/minute) to afford the title compound (77 mg, 0.158 mmol, 79% yield). ¹H NMR (400 MHz, DMSO-d₆ at 90° C.) δ ppm 8.87 (s, 1H), 6.98 (s, 1H), 4.69 (s, 1H), 3.97 (s, 2H), 3.28-3.10 (m, 2H), 3.03-2.79 (m, 3H), 2.69-2.41 (m, 4H), 1.92 (s, 1H), 1.62-1.48 (m, 9H), 0.98-0.87 (m, 6H); MS (ESI⁺) m/z 487 [M+H]⁺.

### Example II-24C: 5-[(2R)-4-fluoro-6-hydroxy-2-{[methyl(2-methylpropyl)amino]methyl}-2,3-dihydro-1H-indol-5-yl]-1λ⁶,2,5-thiadiazolidine-1,1,3-trione

Trifluoroacetic acid (0.152 mL, 1.972 mmol) was added to a solution of the product of Example II-24B (77 mg, 0.158 mmol) in dichloromethane (2 mL). The resulting solution stirred at room temperature for 20 hours. The reaction was then neutralized with 0.7 M NH₃ in methanol (2 mL) and concentrated onto C18 silica gel (1 g). The crude residue was subjected to column chromatography (Teledyne ISCO RediSep Rf Gold® 12 g reversed phase (C18) flash cartridge, 0-30% methanol in 0.1% ammonium hydroxide, 15 mL/minute) to afford the title compound (48 mg, 0.124 mmol, 79% yield). ¹H NMR (400 MHz, DMSO-d₆ at 90° C.) 8 ppm 8.15 (s, 1H), 7.08 (s, 3H), 5.94 (s, 1H), 4.17-4.05 (m, 1H), 3.87 (s, 2H), 3.21 (s, 1H), 3.10 (dd, J=15.5, 9.3 Hz, 1H), 2.87-2.69 (m, 2H), 2.66 (dd, J=15.5, 6.6 Hz, 2H), 2.52 (s, 3H), 1.92 (s, 1H), 1.01-0.88 (m, 6H); MS (ESI⁺) m/z 387 [M+H]⁺.

### Example II-25: 5-[(2R)-4-fluoro-6-hydroxy-1-methyl-2-{[(2-methylpropyl)amino]methyl}-2,3-dihydro-1H-indol-5-yl]-1λ⁶,2,5-thiadiazolidine-1,1,3-trione (Compound 156)

### Example II-25A: tert-butyl {[(2R)-4-fluoro-6-hydroxy-1-methyl-5-(1,1,4-trioxo-1λ⁶,2,5-thiadiazolidin-2-yl)-2,3-dihydro-1H-indol-2-yl]methyl}(2-methylpropyl)carbamate

Acetic acid (20 μL, 0.349 mmol) was added to a solution of formaldehyde (50% in water) (20 μL, 0.726 mmol) and the product of Example II-24A-2 (57 mg, 0.115 mmol) in dichloroethane (1 mL), followed by sodium triacetoxyborohydride (37 mg, 0.175 mmol). The reaction mixture was stirred at room temperature for 1 hour. The reaction was then neutralized with 0.7 M NH₃ in methanol (2 mL) and concentrated onto C18 silica gel (1 g). The crude residue was subjected to column chromatography (Teledyne ISCO RediSep Rf Gold® 12 g reversed phase (C18) flash cartridge, 0-30% methanol in 0.1% ammonium hydroxide, 15 mL/minute) to afford the title compound (10 mg, 0.011 mmol, 10% yield). MS (ESI⁻) m/z 486 [M−H]⁻.

### Example II-25B: 5-[(2R)-4-fluoro-6-hydroxy-1-methyl-2-{[(2-methylpropyl)amino]methyl}-2,3-dihydro-1H-indol-5-yl]-1λ⁶,2,5-thiadiazolidine-1,1,3-trione

Trifluoroacetic acid (20 μL, 0.260 mmol) was added to a solution of the product of Example II-25A (10 mg, 0.011 mmol) in dichloromethane (1 mL) and the reaction mixture was stirred at room temperature for 8 hours. The reaction was then neutralized with 0.7 M NH₃ in methanol (1 mL) and concentrated and stored at −18° C. overnight. The crude residue was redissolved in dichloromethane (1 mL) and trifluoroacetic acid (300 μL, 3.89 mmol) was added. The resulting solution was stirred at room temperature for 3 hours. The reaction was then neutralized with 0.7 M NH₃ in methanol (2 mL) and concentrated onto C18 silica gel (1 g). The crude residue was subjected to column chromatography (Teledyne ISCO RediSep Rf Gold® 12 g reversed phase (C18) flash cartridge, 0-30% methanol in 0.1% ammonium hydroxide, 15 mL/minute) to afford the title compound (2 mg, 5.2 μmol, 47% yield). ¹H NMR (400 MHz, DMSO-d₆) δ ppm 8.73 (s, 1H), 7.17 (s, 4H), 5.92 (s, 1H), 3.85 (s, 2H), 3.71 (q, J=8.2, 7.4 Hz, 1H), 3.25 (d, J=12.7 Hz, 1H), 3.19-3.03 (m, 2H), 2.88-2.72 (m, 3H), 2.70 (s, 3H), 2.06-1.92 (m, 1H), 0.95 (t, J=6.2 Hz, 6H); MS (ESI⁺) m/z 387 [M+H]⁺.

### Example II-26: 5-[(2R)-2-{[(3,3-difluoro-2-hydroxypropyl)amino]methyl}-4-fluoro-6-hydroxy-2,3-dihydro-1H-indol-5-yl]-1λ⁶,2,5-thiadiazolidine-1,1,3-trione (Compound 157)

### Example II-26A: N-(3,3-difluoro-2-hydroxypropyl)-2,2,2-trifluoroacetamide

To a stirred solution of 3-amino-1,1-difluoropropan-2-ol (0.49 g, 4.19 mmol) in methanol (5 mL) at 0° C. was added ethyl 2,2,2-trifluoroacetate (0.540 mL, 4.54 mmol) dropwise. After completion of the addition, the cooling was removed, and the reaction mixture was stirred at room temperature for 24 hours. The reaction mixture was carefully concentrated under reduced pressure to afford the title compound (0.9699 g, 4.17 mmol, 99% yield). ¹H NMR (400 MHz, CDCl₃) δ ppm 6.74 (s, 1H), 5.74 (td, J=55.3, 4.0 Hz, 1H), 4.00 (dddd, J=14.6, 10.6, 7.6, 3.9 Hz, 1H), 3.79 (ddd, J=14.4, 6.8, 3.8 Hz, 1H), 3.49 (ddd, J=13.5, 7.5, 5.3 Hz, 1H), 2.10 (d, J=40.4 Hz, 1H); MS (ESI⁻) m/z 206 [M−H]⁻.

### Example II-26B: N-{3,3-difluoro-2-[(2-methoxyethoxy)methoxy]propyl}-2,2,2-trifluoroacetamide

To a stirred solution of the product of Example II-26A (0.9799 g, 4.17 mmol) in dichloromethane (4 mL) at 0° C. were added N,N-diisopropylethylamine (1.829 mL, 10.42 mmol) and then 2-methoxyethoxymethyl chloride (technical grade purity) (0.885 mL, 6.25 mmol) dropwise. After completion of the addition, the cooling was removed, and the reaction mixture was stirred at room temperature for 20 hours. More N,N-diisopropylethylamine (1.463 mL, 8.34 mmol) and 2-methoxyethoxymethyl chloride (0.885 mL, 6.25 mmol) were added. The reaction mixture was stirred at room temperature for 20 hours. More N,N-diisopropylethylamine (0.732 mL, 4.17 mmol) and 2-methoxyethoxymethyl chloride (0.590 mL, 4.17 mmol) were added. The reaction mixture was stirred at room temperature for 2 days. More N,N-diisopropylethylamine (1.097 mL, 6.25 mmol) and 2-methoxyethoxymethyl chloride (0.590 mL, 4.17 mmol) were added. The reaction mixture was stirred at room temperature for 24 hours. More N,N-diisopropylethylamine (1.097 mL, 6.25 mmol) and 2-methoxyethoxymethyl chloride (0.590 mL, 4.17 mmol) were added. The reaction mixture was stirred at room temperature for 24 hours. The reaction mixture was diluted with dichloromethane (15 mL). A saturated aqueous solution of ammonium chloride (20 mL) was added. The organic layer was separated. The aqueous layer was extracted with dichloromethane (3×20 mL). The combined organic layers were washed with brine (20 mL), dried over sodium sulfate, filtered, and concentrated under reduced pressure. The crude residue was subjected to column chromatography (silica gel, 24 g cartridge, 0-100% tert-butyl methyl ether: isohexane, 60 mL/minute) to afford the title compound with unreacted starting material. To a solution of this residue in dichloromethane (4 mL) at 0° C. were added N,N-diisopropylethylamine (1.407 mL, 8.02 mmol) and then 2-methoxyethoxymethyl chloride (technical grade purity) (0.681 mL, 4.81 mmol) dropwise. After completion of the addition, the cooling was removed, and the reaction mixture was stirred at room temperature for 2 hours. More 2-methoxyethoxymethyl chloride (technical grade purity) (0.681 mL, 4.81 mmol) and N,N-diisopropylethylamine (1.407 mL, 8.02 mmol) were added. The reaction mixture was stirred at room temperature for 2 hours. More 2-methoxyethoxymethyl chloride (technical grade purity) (0.681 mL, 4.81 mmol) and N,N-diisopropylethylamine (1.407 mL, 8.02 mmol) were added. The reaction mixture was stirred at room temperature for 20 hours. More 2-methoxyethoxymethyl chloride (technical grade purity) (0.681 mL, 4.81 mmol) and N,N-diisopropylethylamine (1.407 mL, 8.02 mmol) were added. The reaction mixture was stirred at room temperature for 2 hours. More 2-methoxyethoxymethyl chloride (technical grade purity) (0.681 mL, 4.81 mmol) and N,N-diisopropylethylamine (1.407 mL, 8.02 mmol) were added. The reaction mixture was stirred at room temperature for 20 hours. The reaction mixture was diluted with dichloromethane (15 mL). A saturated aqueous solution of ammonium chloride (20 mL) was added. The organic layer was separated. The aqueous layer was extracted with dichloromethane (3×20 mL). The combined organic layers were washed with brine (20 mL), dried over sodium sulfate, filtered, and concentrated under reduced pressure. The crude residue was subjected to column chromatography (silica gel, 40 g cartridge, 0-100% tert-butyl methyl ether: isohexane, 60 mL/minute) to afford the title compound (2.5 g, 1.694 mmol, 53% yield, 20% purity). ¹H NMR (400 MHz, CDCl₃) δ ppm 7.84 (s, 1H), 5.97-5.60 (m, 1H), 4.85-4.78 (m, 2H), 3.89-3.47 (m, 6H), 3.40-3.30 (m, 3H), 3.22-3.14 (m, 1H); MS (ESI⁻) m/z 294 [M−H]⁻.

### Example II-26C: 3,3-difluoro-2-[(2-methoxyethoxy)methoxy]propan-1-amine

To a stirred solution of the product of Example II-26B (2.5 g, 1.694 mmol) in methanol (4 mL) was added sodium hydroxide (2 M aqueous) (2.5 mL, 5.0 mmol). The reaction mixture was stirred at room temperature for 24 hours. An additional aliquot of sodium hydroxide (2 M aqueous) (2.5 mL, 5.0 mmol) was added, and the reaction mixture was stirred for a further 2 hours. An additional aliquot of sodium hydroxide (2 M aqueous) (2 mL, 4.0 mmol) was added, and the reaction mixture was stirred for a further 2 hours. The reaction mixture was concentrated with a stream of compressed air and then diluted with dichloromethane (10 mL) and water (10 mL). The organic layer was separated. The aqueous layer was extracted with dichloromethane (3×10 mL). The combined organic layers were washed with brine (10 mL), dried over sodium sulfate, filtered and carefully concentrated under reduced pressure to afford the title compound (2.38 g, 1.195 mmol, 71% yield, 10% purity). MS (ESI⁺) m/z 200 [M+H]⁺.

### Example II-26D: tert-butyl (2R)-6-(benzyloxy)-5-[(2-tert-butoxy-2-oxoethyl)(trifluoroacetyl)amino]-2-[8-(difluoromethyl)-2,5,7-trioxa-10-azaundecan-11-yl]-4-fluoro-2,3-dihydro-1H-indole-1-carboxylate

Acetic acid (0.07 mL, 1.20 mmol) was added to a solution of the product of Example II-26C (2.38 g, 1.195 mmol, 10% purity) and the product of Example II-2I (0.852 g, 1 mmol) in 1,2-dichloroethane (5 mL). The reaction mixture was stirred at room temperature for 5 hours. Sodium triacetoxyborohydride (0.318 g, 1.500 mmol) was added, and the reaction mixture was stirred for 20 hours. The reaction mixture was quenched with a saturated aqueous solution of sodium bicarbonate (3 mL) and stirred for 10 minutes. The reaction mixture was diluted with water (20 mL) and extracted with ethyl acetate (3×20 mL). The combined organic layers were washed with brine (20 mL), dried over sodium sulfate, filtered, and concentrated under reduced pressure to afford the title compound (1.45 g, 0.930 mmol, 93% yield, 50% purity). MS (ESI⁺) m/z 780 [M+H]⁺.

### Example II-26E: tert-butyl (2R)-6-(benzyloxy)-2-[0-(tert-butoxycarbonyl)-8-(difluoromethyl)-2,5,7-trioxa-10-azaundecan-11-yl]-5-[(2-tert-butoxy-2-oxoethyl)(trifluoroacetyl)amino]-4-fluoro-2,3-dihydro-1H-indole-1-carboxylate

To a solution of the product of Example II-26D (1.45 g, 0.930 mmol) in acetonitrile (6 mL) at room temperature was added N,N-diisopropylethylamine (0.487 mL, 2.79 mmol) and di-tert-butyl dicarbonate (0.406 g, 1.860 mmol). The mixture was stirred at room temperature for 16 hours. The reaction was quenched with water (10 mL). The aqueous layer was extracted with ethyl acetate (3×15 mL). The combined organic layers were washed with brine (10 mL), dried over sodium sulfate, filtered, and concentrated under reduced pressure. The crude residue was subjected to column chromatography (SiO₂, 24 g cartridge, 0-40% ethyl acetate in isohexane, 50 mL/minute) to afford the title compound (1.13 g, 0.642 mmol, 69% yield, 50% purity). ¹H NMR (400 MHz, DMSO-d₆ at 90° C.) δ ppm 7.48-7.28 (m, 6H), 6.23-5.87 (m, 1H), 5.29-5.06 (m, 2H), 4.84-4.67 (m, 5H), 4.64 (s, 2H), 4.57-4.36 (m, 1H), 4.02-3.77 (m, 1H), 3.70-3.57 (m, 4H), 3.56-3.37 (m, 4H), 3.16 (dd, J=16.7, 9.7 Hz, 1H), 2.87 (d, J=7.5 Hz, 1H), 1.66 (s, 2H), 1.58-1.48 (m, 7H), 1.46-1.30 (m, 18H); MS (ESI⁺) m/z 902 [M+Na]⁺.

### Example II-26F: tert-butyl (2R)-6-(benzyloxy)-2-[0-(tert-butoxycarbonyl)-8-(difluoromethyl)-2,5,7-trioxa-10-azaundecan-11-yl]-5-[(2-tert-butoxy-2-oxoethyl)amino]-4-fluoro-2,3-dihydro-1H-indole-1-carboxylate

To a solution of the product of Example II-26E (1.13 g, 0.642 mmol) in methanol (2.8 mL) and tetrahydrofuran (2.8 mL) at room temperature was added 2 M aqueous lithium hydroxide (0.96 mL, 1.93 mmol). The reaction mixture was then stirred at room temperature for 1 hour. The reaction mixture was neutralized to pH 7 with 1 M aqueous hydrochloric acid and diluted with water (10 mL). The aqueous layer was extracted with ethyl acetate (3×10 mL). The combined organic layers were washed with brine (10 mL), dried over sodium sulfate, filtered, and concentrated in vacuo to afford the title compound (0.794 g, 0.506 mmol, 79% yield, 50% purity). ¹H NMR (400 MHz, DMSO-d₆ at 90° C.) δ ppm 7.56-7.43 (m, 2H), 7.43-7.28 (m, 3H), 7.23-7.12 (m, 1H), 6.22-5.81 (m, 1H), 5.14-5.03 (m, 2H), 4.82-4.68 (m, 3H), 4.64 (s, 3H), 4.55-4.35 (m, 1H), 4.16-4.00 (m, 1H), 3.91-3.80 (m, 2H), 3.71-3.56 (m, 2H), 3.54-3.44 (m, 2H), 3.44-3.34 (m, 2H), 3.32-3.23 (m, 2H), 3.17-3.05 (m, 1H), 2.84-2.73 (m, 1H), 1.73-1.17 (m, 27H); MS (ESI⁺) m/z 785 [M+H]⁺.

### Example II-26G: tert-butyl (2R)-6-(benzyloxy)-2-[10-(tert-butoxycarbonyl)-8-(difluoromethyl)-2,5,7-trioxa-10-azaundecan-11-yl]-5-[(2-tert-butoxy-2-oxoethyl)({[(prop-2-en-1-yl)oxy]carbonyl}sulfamoyl)amino]-4-fluoro-2,3-dihydro-1H-indole-1-carboxylate

To a solution of chlorosulfonyl isocyanate (0.084 mL, 0.962 mmol) in dichloromethane (5 mL) at 0° C. was added allyl alcohol (0.069 mL, 1.013 mmol) dropwise. The resulting solution was stirred for 20 minutes at 0° C. before a prepared solution of the product of Example II-26F (0.794 g, 0.506 mmol) and triethylamine (0.176 mL, 1.266 mmol) in dichloromethane (5 mL) was added dropwise. The resulting solution was stirred at 0° C. for 30 minutes. The reaction mixture was quenched with water (50 mL) and diluted with dichloromethane (50 mL). The layers were separated, and the aqueous layer was extracted with dichloromethane (3×50 mL). The combined organic layers were dried over sodium sulfate, filtered, and concentrated under reduced pressure. The crude residue was subjected to column chromatography (SiO₂, 40 g cartridge, 0-80% tert-butyl methyl ether in isohexane, 90 mL/minute) to afford the title compound (490 mg, 0.259 mmol, 51% yield, 50% purity). ¹H NMR (400 MHz, DMSO-d₆) δ ppm 11.74-11.41 (m, 1H), 7.76-7.09 (m, 6H), 6.12 (t, J=54.5 Hz, 1H), 5.72 (ddd, J=16.5, 10.7, 5.4 Hz, 1H), 5.31-4.99 (m, 4H), 4.90-4.59 (m, 7H), 4.36-3.95 (m, 4H), 3.68-3.48 (m, 2H), 3.48-3.37 (m, 2H), 3.38-3.29 (m, 2H), 3.27-3.19 (m, 2H), 3.19-3.04 (m, 1H), 2.79 (t, J=18.8 Hz, 1H), 1.66-1.08 (m, 27H); MS (ESI⁻) m/z 946 [M−H]⁻.

### Example II-26H: tert-butyl (2R)-6-(benzyloxy)-2-[10-(tert-butoxycarbonyl)-8-(difluoromethyl)-2,5,7-trioxa-10-azaundecan-11-yl]-4-fluoro-5-(1,1,4-trioxo-1λ⁶,2,5-thiadiazolidin-2-yl)-2,3-dihydro-1H-indole-1-carboxylate

A solution of the product of Example II-26G (0.49 g, 0.259 mmol) and potassium carbonate (0.143 g, 1.035 mmol) in methanol (3 mL) was degassed under vacuum and backfilled with nitrogen three times. To the mixture was added tetrakis(triphenylphosphine)palladium(0) (0.030 g, 0.026 mmol), and the reaction mixture was degassed under vacuum and backfilled with nitrogen three times. The reaction mixture was stirred at 60° C. for 24 hours. The mixture was cooled to room temperature and C18 silica gel (3 g) was added, and the mixture was concentrated in vacuo. The crude residue was subjected to column chromatography (Reveleris® 40 g reversed phase (C18) flash cartridge, dry loaded with C18 silica gel 3 g, 10-70% methanol in 0.10% ammonium hydroxide, 40 mL/minute) to afford the title compound (220 mg, 0.251 mmol, 97% yield, 90% purity). ¹H NMR (400 MHz, DMSO-d₆ at 90° C.) δ ppm 7.58-7.44 (m, 2H), 7.43-7.25 (m, 3H), 7.25-7.18 (m, 1H), 6.04 (tt, J=54.6, 2.5 Hz, 1H), 5.09 (s, 2H), 4.77 (dd, J=9.0, 6.6 Hz, 1H), 4.74-4.64 (m, 2H), 4.14-3.98 (m, 1H), 3.94 (d, J=13.0 Hz, 1H), 3.88 (dd, J=13.0, 2.3 Hz, 1H), 3.71-3.55 (m, 2H), 3.54-3.42 (m, 4H), 3.37 (dd, J=14.2, 6.1 Hz, 1H), 3.33-3.20 (m, 4H), 3.20-3.08 (m, 1H), 2.90-2.73 (m, 1H), 1.60-1.46 (m, 9H), 1.46-1.29 (m, 9H); MS (ESI⁻) m/z 787 [M−H]⁻.

### Example II-26I: tert-butyl (2R)-2-[10-(tert-butoxycarbonyl)-8-(difluoromethyl)-2,5,7-trioxa-10-azaundecan-11-yl]-4-fluoro-6-hydroxy-5-(1,1,4-trioxo-1λ⁶,2,5-thiadiazolidin-2-yl)-2,3-dihydro-1H-indole-1-carboxylate

To a suspension of the product of Example II-26H (220 mg, 0.251 mmol) in degassed water (0.1 mL) and ethanol (4 mL) was added 10% Pd/C (53 mg). The resulting suspension was allowed to stir under hydrogen (5 bar) for 18 hours. The reaction mixture was filtered through a Whatman glass fiber filter paper and washed with ethanol (3×10 mL). The filtrate was concentrated under reduced pressure onto C18 silica (1 g). The crude residue was subjected to column chromatography (Reveleris® 24 g reversed phase (C18) flash cartridge, dry loaded with diatomaceous earth, 5-55% methanol in 10 mM ammonium bicarbonate, 50 mL/minute) to afford the title compound (146 mg, 0.209 mmol, 83% yield). ¹H NMR (400 MHz, DMSO-d₆ at 90° C.) δ ppm 6.82 (s, 1H), 6.04 (tt, J=54.7, 3.0 Hz, 1H), 4.76 (dd, J=7.9, 6.6 Hz, 1H), 4.70 (dd, J=6.6, 3.9 Hz, 1H), 4.67-4.54 (m, 1H), 4.15-4.01 (m, 1H), 3.97 (dd, J=13.0, 1.3 Hz, 1H), 3.89 (dd, J=13.0, 2.8 Hz, 1H), 3.71-3.55 (m, 2H), 3.55-3.41 (m, 4H), 3.41-3.32 (m, 1H), 3.32-3.17 (m, 5H), 3.01 (ddd, J=15.7, 9.0, 4.3 Hz, 1H), 2.78-2.65 (m, 1H), 1.61-1.47 (m, 9H), 1.47-1.34 (m, 9H); MS (ESI⁻) m/z 697 [M−H]⁻.

### Example II-26J: 5-[(2R)-2-{[(3,3-difluoro-2-hydroxypropyl)amino]methyl}-4-fluoro-6-hydroxy-2,3-dihydro-1H-indol-5-yl]-1λ⁶,2,5-thiadiazolidine-1,1,3-trione

Trifluoroacetic acid (0.885 mL, 11.49 mmol) was added to a solution of the product of Example II-26I (146 mg, 0.209 mmol) and propan-1-amine (0.259 mL, 3.13 mmol) in dichloromethane (2 mL) at 0° C. The resulting solution was stirred at room temperature for 3 hours. The reaction formed a suspension during this time. A second aliquot of trifluoroacetic acid (0.885 mL, 11.49 mmol) and acetonitrile (1 mL) was added to aid solubility. The resulting solution was stirred at room temperature for 20 hours. The reaction was then neutralized with 0.7 M ammonia in methanol (20 mL) and concentrated onto C18 silica gel. The crude residue was subjected to column chromatography (Teledyne ISCO RediSep Rf Gold® 12 g reversed phase (C18) flash cartridge, 0-30% methanol in 0.1% ammonium hydroxide, 15 mL/minute) followed by another column chromatography (Teledyne ISCO RediSep Rf Gold® 12 g reversed phase (C18) flash cartridge, 0-20% methanol in 0.1% ammonium hydroxide, 15 mL/minute) to afford the title compound (1 mg, 2.4 μmol, 1% yield). ¹H NMR (400 MHz, DMSO-d₆) δ ppm 7.80 (s, 2H), 7.12 (s, 4H), 6.04 (d, J=2.4 Hz, 1H), 5.83 (td, J=55.4, 3.8 Hz, 1H), 4.30-4.11 (m, 3H), 4.11-3.93 (m, 1H), 3.31-3.11 (m, 2H), 3.11-2.97 (m, 3H), 2.77 (dd, J=15.6, 7.1 Hz, 1H), 1.32 (s, 2H); MS (ESI⁺) m/z 411 [M+H]⁺.

### Biological Assays

### Abbreviations

DMEM for Dulbecco's modified Eagle's medium; DMSO for dimethyl sulfoxide; DTT for dithiothreitol; EDTA for ethylenediaminetetraacetic acid; EGTA for ethylene glycol-bis(2-aminoethylether)-N,N,N′,N′-tetraacetic acid; HEPES for 4-(2-hydroxyethyl)piperazine-1-ethanesulfonic acid; IFNγ for interferon gamma; and Tween® 20 for polyethylene glycol sorbitan monolaurate.

### Example III-1: Mobility Shift Assay (MSA) Used to Determine Potency of PTPN2 Inhibitors

Compound activity was determined using in house His tagged PTPN2 (TC45) protein (SEQ ID NO: 1) in an in vitro enzymatic reaction. The enzymatic assay used to determine activity was a mobility shift assay using a LabChip EZ Reader by Caliper Life Sciences. The enzymatic reaction was carried out in assay buffer (50 mM HEPES pH 7.5, 1 mM EGTA, 10 mM EDTA, 0.01% Tween® 20, and 2 mM DTT). The compounds were dispensed on a white 384 well ProxiPlate™ (PerkinElmer Catalog #6008289) plate using the Labcyte Echo at varying concentrations (12 point, 1:3 dilution). The enzyme (at 0.5 nM) was incubated with compound for 10 minutes at room temperature. Thereafter, the substrate (phosphorylated insulin receptor probe sequence: ((OG488)-(NH—CH₂—CH₂—O—CH₂—CH₂—O—CH₂—CO)-T-R-D-I—(PY)-E-T-D-Y—Y—R—K—K—NH₂) (SEQ ID NO: 2) was added at 2 μM to the plates and incubated for another 10 minutes at room temperature. Finally, a quench solution (water and 4-bromo-3-(2-oxo-2-propoxyethoxy)-5-(3-{[1-(phenylmethanesulfonyl)piperidin-4-yl]amino}phenyl)thiophene-2-carboxylic acid) was added to the plates, which were then run on the EZ Reader (excitation 488 nm, emission 530 nm) to measure % conversion (the amount of phosphorylated substrate which was de-phosphorylated by PTPN2). Each plate had a 100% control (inhibitor: 4-bromo-3-(2-oxo-2-propoxyethoxy)-5-(3-{[1-(phenylmethanesulfonyl)piperidin-4-yl]amino}phenyl)thiophene-2-carboxylic acid) and 0% control (DMSO), which were used to calculate % inhibition. The % inhibition was then used to calculate the IC₅₀ values.

### Example III-2: Mobility Shift Assay (MSA) Used to Determine Potency of PTPN1 Inhibitors

Compound activity was determined using in house His tagged full-length PTPN1 protein (SEQ ID NO: 3) in an in vitro enzymatic reaction. The enzymatic assay used to determine activity is a mobility shift assay using a LabChip EZ Reader by Caliper Life Sciences. The enzymatic reaction was carried out in assay buffer (50 mM HEPES pH 7.5, 1 mM EGTA, 10 mM EDTA, 0.01% Tween® 20, and 2 mM DTT). The compounds were dispensed on a white 384 well ProxiPlate™ (PerkinElmer Cat ^{#}6008289) plate using a Labcyte Echo® liquid handler at varying concentrations (12 point, 1:3 dilution). The enzyme (at 0.5 nM) was incubated with compound for 10 minutes at room temperature. Thereafter, the substrate (phosphorylated insulin receptor probe sequence: ((OG488)-(NH—CH₂—CH₂—O—CH₂—CH₂—O—CH₂—CO)-T-R-D-I—(PY)-E-T-D-Y—Y—R—K—K—NH₂) (SEQ ID NO: 2) was added at 2 μM to the plates and incubated for another 10 minutes at room temperature. Finally, a quench solution (water and 4-bromo-3-(2-oxo-2-propoxyethoxy)-5-(3-{[1-(phenylmethanesulfonyl)piperidin-4-yl]amino}phenyl)thiophene-2-carboxylic acid) was added to the plates, which were then run on the EZ Reader (excitation 488 nm, emission 530 nm) to measure % conversion (the amount of phosphorylated substrate which was de-phosphorylated by PTPN1). Each plate had a 100% control (inhibitor: 4-bromo-3-(2-oxo-2-propoxyethoxy)-5-(3-{[1-(phenylmethanesulfonyl)piperidin-4-yl]amino}phenyl)thiophene-2-carboxylic acid) and 0% control (DMSO), which were used to calculate % inhibition. The % inhibition was then used to calculate the IC₅₀ values.

Table 1 below summarizes the IC₅₀ data obtained using the PTPN2 MSA assay and the PTPN1 MSA assay for exemplary compounds of the disclosure. In this table, “A” represents an IC₅₀ of less than 10 nM; “B” an IC₅₀ of between 10 nM and 100 nM; and “C” an IC₅₀ of greater than 10 nM to 100 nM greater than 100 nM.

TABLE 1
IC₅₀ values of exemplary compounds of the disclosure
in the PTPN2 and PTPN1 Mobility Shift Assays (MSA).

| | PTPN2 | PTPN1 | | PTPN2 | PTPN1 |
|---|---|---|---|---|---|
| Compound | MSA | MSA | Compound | MSA | MSA |
| No. | IC₅₀ | IC₅₀ | No. | IC₅₀ | IC₅₀ |
| 100 | A | B | 101 | C | C |
| 102 | B | B | 103 | A | B |
| 104 | B | B | 105 | B | B |
| 106 | A | B | 107 | B | B |
| 108 | B | B | 109 | B | B |
| 110 | B | B | 111 | B | B |
| 112 | B | B | 113 | B | B |
| 114 | B | B | 115 | B | B |
| 116 | B | B | 117 | B | B |
| 118 | B | B | 119 | B | B |
| 120 | B | B | 121 | B | B |
| 122 | B | B | 123 | B | B |
| 124 | B | B | 125 | A | B |
| 126 | A | B | 127 | A | B |
| 128 | A | B | 129 | A | B |
| 130 | A | B | 131 | B | B |
| 132 | B | C | 133 | B | B |
| 134 | B | B | 135 | B | B |
| 136 | B | B | 137 | B | B |
| 138 | B | B | 139 | B | B |
| 140 | B | B | 141 | B | B |
| 142 | B | B | 143 | B | B |
| 144 | B | C | 145 | B | B |
| 146 | B | B | 147 | B | B |
| 148 | A | B | 149 | A | B |
| 150 | B | B | 151 | B | B |
| 152 | B | B | 153 | B | B |
| 154 | A | B | 155 | B | B |
| 156 | C | C | 157 | C | C |

### Example III-3: B16F10 (Murine Melanoma Cells) Phospho-STAT1 HTRF Proximal Pharmacodynamic (PD) Assay

B16F10 cells were grown and maintained in high glucose DMEM (Gibco, Catalog^{#}11965-092, Dun Laoghaire Co Dublin) supplemented with 10% fetal bovine serum (Gibco, Catalog^{#}10082-139, Dun Laoghaire Co Dublin). The cells were pelleted and resuspended in high glucose DMEM without phenol red (Gibco, Catalog^{#}11054-020, Dun Laoghaire Co Dublin), supplemented with 10% fetal bovine serum and plated in a 384 well Corning plate (product^{#}3765, Corning, NY) at 11,000 cells per well in a volume of 20 μL. The cells were dosed with the compounds of interest using an Echo Liquid Handler (Beckman Coulter, Brea, CA) at 50 μM top dose with 3-fold dilutions down to 0.002679 μM for a 10-point dose response. The plate was incubated for 3 hours at 37° C. and subsequently treated with recombinant mouse IFNγ (R&D Systems, Catalog^{#}485-MI, Minneapolis, MN; 100 nM final concentration) for 10 minutes at 37° C. to induce STAT1 phosphorylation followed by 3.3 μM staurosporine treatment for 1 hour at 37° C. to terminate phosphorylation. Media was then aspirated and 20 μL of 1× lysis buffer/blocking reagent (Cisbio Phospho-STAT1 kit, part^{#} 63ADK026PEH, Bedford, MA) was added. The plate was placed on a plate shaker for 30 minutes at room temperature, sealed, and stored at −80° C. until needed. To thaw, the plate was placed on shaker at room temperature until completely thawed while the antibody master mix was made (1:40 Phospho-STAT1 Eu Cryptate antibody, 1:40 Phospho-STAT d2 antibody, with the detection buffer; Cisbio Phospho-STAT1 kit, part^{#}63ADK026PEH, Bedford, MA). The antibody master mix was then dispensed at 4 μL per well into a 384 ProxiPlate Plus (PerkinElmer, part^{#}6008289, Waltham, MA) and 16 μL of lysate was added from the Corning plate to the Proxiplate using a VIAFLO 384 (INTEGRA). The plate was incubated for 3 hours at room temperature and read on an EnVision® (Perkin Elmer) plate reader with laser excitation at 335 nm and emission at 665 nm. Dotmatics Studies (Bishop's Stortford, UK) was utilized to generate all dose-response curves and calculate EC50s and are shown in Table 2.

TABLE 2
EC₅₀ values in B16F10 IFNγ-induced
STAT1 phosphorylation (pSTAT1) cell assay.

| | B16F10 IFNγ | | B16F10 IFNγ |
|---|---|---|---|
| Compound | Induced pSTAT1 | Compound | Induced pSTAT1 |
| No. | EC₅₀ (μM) | No. | EC₅₀ (μM) |
| 100 | 0.853 | 102 | 3.42 |
| 103 | 1.87 | 104 | 5.11 |
| 105 | 2.95 | 106 | 1.86 |
| 107 | 1.33 | 108 | 1.91 |
| 109 | 1.35 | 110 | 18 |
| 111 | 1.11 | 112 | 0.776 |
| 113 | 0.77 | 114 | 1.46 |
| 115 | 0.835 | 116 | 0.736 |
| 117 | 0.818 | 118 | 20.1 |
| 119 | 0.848 | 120 | 1.61 |
| 121 | 1.74 | 122 | 1.98 |
| 125 | 3.09 | 126 | 3.67 |
| 127 | 10.8 | 128 | >50 |
| 129 | 1.29 | 130 | >50 |
| 131 | 22.1 | 132 | 4.69 |
| 133 | 1.24 | 134 | 0.947 |
| 135 | 0.756 | 136 | 1.23 |
| 137 | 0.904 | 138 | 1.56 |
| 139 | 1.56 | 140 | 0.701 |
| 141 | 0.704 | 142 | 1.14 |
| 143 | 1 | 144 | 7.18 |
| 145 | 1.97 | 146 | 1.83 |
| 147 | 1.84 | 148 | 1.85 |
| 149 | 1.56 | 150 | 2.18 |
| 151 | 2.77 | 152 | 1.17 |
| 153 | 2.01 | 154 | 1.88 |
| 155 | 15.7 | | |

### Example III-4: Comparative Mobility Shift Assay (MSA) and B16F10 pSTAT1 Proximal Pharmacodynamic (PD) Assay for Dihydrobenzofuran and Indoline Compounds of the Disclosure and Corresponding Chromane Analogs

Assay data was obtained using the protocols described in Example III-1 for PTPN2 mobility shift assay data (biochemical potency) and in Example III-3 for B16F10 pSTAT1 proximal pharmacodynamic (PD) assay data (cellular potency). The data comparing dihydrobenzofuran and indoline analogs of the disclosure with corresponding chromane analogs. Ten of the twelve dihydrobenzofuran compounds exceed the corresponding chromane analogs in biochemical potency as shown in the PTPN2 MSA. None of the three indoline compounds exceed the corresponding chromane analogs in biochemical potency as shown in the PTPN2 MSA. Eight of the ten dihydrobenzofuran compounds exceed the corresponding chromane analogs in cellular potency in the pSTAT1 proximal pharmacodynamic assay. All of the indoline compounds exceed the corresponding chromane analogs in cellular potency in the pSTAT1 proximal pharmacodynamic assay. For six of the ten comparisons, the dihydrobenzofuran analogs showed greater potency enhancement in the cellular assay over the potency enhancement observed in the corresponding biochemical comparison. For all comparisons, the indoline analogs showed greater potency enhancement in the cellular assay over the potency enhancement observed in the corresponding biochemical comparison.

TABLE 3
IC₅₀ values of exemplary dihydroxybenzofuran compounds and indoline
compounds of the disclosure in the PTPN2 Mobility Shift Assays (MSA) and EC₅₀ values in
B16F10 IFNγ-induced STAT1 phosphorylation (pSTAT1) cell assays compared with
corresponding chromane analogs.

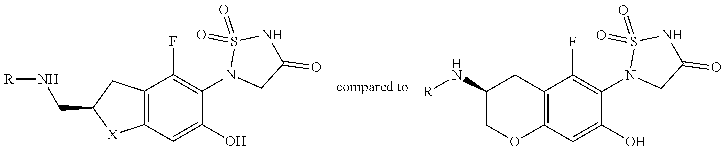

| | | | | | | | Dihydro- | |
|---|---|---|---|---|---|---|---|---|
| | | | | | | | benzofuran | Dihydro- |
| | X = O or | PTPN2 | | | PTPN2 | pSTA | or Indoline | benzofuran |
| | NH | MSA | pSTAT1 | Chromane | MSA | T1 | x- | or Indoline x- |
| | Compound | IC₅₀ | EC₅₀ | Comparator | IC₅₀ | EC₅₀ | fold>potency | fold>potency |
| R | # | (μM) | (μM) | # | (μM) | (μM) | biochemical | cellular |
| (F)₂HC—CH₂CH₂CH₂— | 107, X = | 0.0133 | 1.33 | C-1 | 0.00885 | 3.6 | 0.7 | 2.7 |
| | O | | | | | | | |
| 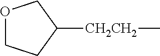 | 108, X = O | 0.0158 | 1.91 | C-2 | 0.0356 | 22.6 | 2.3 | 11.8 |
| (CH₃)₂HC—CH₂CH₂— | 115, X = | 0.0171 | 0.835 | C-3 | 0.0159 | 6.96 | 0.9 | 8.3 |
| | O | | | | | | | |
| (F)₂HC—CH₂ | 123, X = | 0.0112 | | C-4 | 0.0239 | >50 | 2.1 | |
| O—CH₂CH₂CH₂— | O | | | | | | | |
| 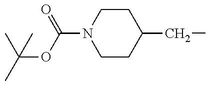 | 124, X = O | 0.0132 | | C-5 | 0.0148 | >50 | 1.1 | |
| 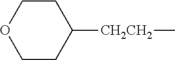 | 125, X = O | 0.00832 | 3.09 | C-6 | 0.0181 | 32.2 | 2.2 | 10.4 |
| 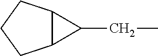 | 126, X = O | 0.00606 | 3.67 | C-7 | 0.0324 | >50 | 5.3 | >13.6 |
| 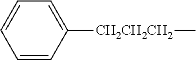 | 127, X = O | 0.00405 | 10.8 | C-8 | 0.0191 | >50 | 4.7 | >4.6 |
| 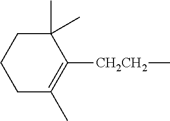 | 128, X = O | 0.00181 | >50 | C-9 | 0.0226 | >50 | 12.5 | ~1.0 |
| 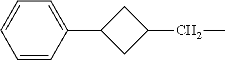 | 129, X = O | 0.00251 | 1.29 | C-10 | 0.00828 | >50 | 3.3 | >38.8 |
| 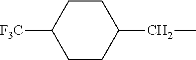 | 130, X = O | 0.00438 | >50 | C-11 | 0.0266 | >50 | 6.1 | ~1.0 |
| 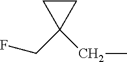 | 131, X = O | 0.0209 | 22.1 | C-12 | 0.0495 | >50 | 2.4 | >2.3 |
| 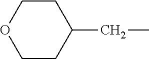 | 138, X —NH | 0.0216 | 156 | C-13 R = 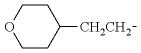 | 0.0181 | 32.2 | 0.8 | 20.6 |
| (CH₃)₂HC—CH₂CH₂— | 139, X = NH | 0.0198 | 1.56 | C-14 | 0.0159 | 6.96 | 0.8 | 4.5 |
| (F)₂HC—CH₂CH₂— | 140, X = NH | 0.0152 | 0.701 | C-15 R = (R)₂HC—CH₂CH₂CH₂— | 0.00885 | 3.6 | 0.6 | 5.1 |

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound represented by Formula (I): 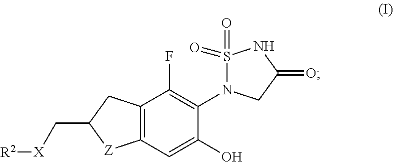
or a pharmaceutically acceptable salt thereof, wherein:
X is —O— or —N(R^{a})—;
Z is —O— or —N(R¹)—;
R¹ is hydrogen or C₁₋₆alkyl;
R² is selected from the group consisting of hydrogen, —C₁₋₆alkyl, —C₁₋₆alkylene-C₃₋₆cycloalkyl, —C₁₋₆alkylene-4-6 membered heterocyclyl, and —C₁₋₆alkylene-phenyl;
wherein —C₁₋₆alkyl, —C₁₋₆alkylene-C₃₋₆cycloalkyl, —C₁₋₆alkylene-4-6 membered heterocyclyl, and —C₁₋₆alkylene-phenyl may optionally be substituted on one or more available carbons by one, two, three or more substituents each independently selected from R^{g};
wherein if —C₁₋₆alkylene-4-6 membered heterocyclyl contains a substitutable ring nitrogen atom, that ring nitrogen atom may optionally be substituted by R^{h};
R^{g} is independently selected, for each occurrence, from the group consisting of halogen, hydroxyl, C₁₋₆alkyl, phenyl, and C₁₋₆alkoxy; wherein C₁₋₆alkyl, phenyl, and C₁₋₆alkoxy may optionally be substituted by one, two three or more substituents each independently selected from R^{P};
R^{h} is C₁₋₆alkyl-O—C(O)—;
R^{P} is independently selected, for each occurrence, from the group consisting of halogen, and hydroxyl; and
R^{a} is selected from the group consisting of hydrogen and C₁₋₆alkyl; wherein C₁₋₆alkyl may optionally be substituted by one or more substituents each independently selected from the group consisting of halogen, cyano, oxo, hydroxyl, and C₃₋₆cycloalkyl.

2. The compound of claim 1, wherein the compound is represented by Formula (Ia) or Formula (Ib): 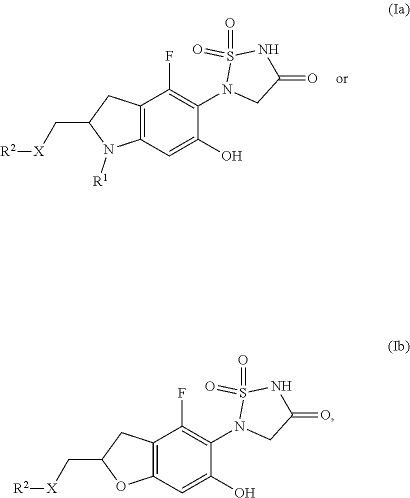
or a pharmaceutically acceptable salt thereof.

3. The compound of claim 2, wherein the compound is represented by Formula (Ia-1) or Formula (Ia-2): 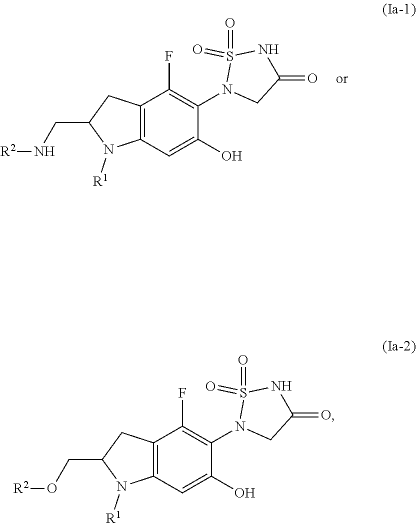
or a pharmaceutically acceptable salt thereof.

4. The compound of claim 3, wherein R¹ is hydrogen.

5. The compound of claim 2, wherein the compound is represented by Formula (Ib-1) or Formula (Ib-2): 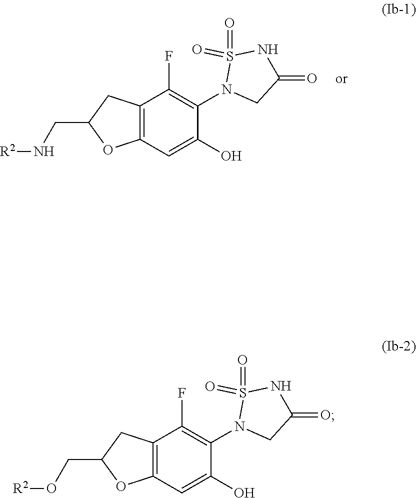
or a pharmaceutically acceptable salt thereof.

6. The compound of any one of claims 1-5, wherein R² is —C₁₋₆alkyl optionally substituted with one, two, or three instances of R^{g}.

7. The compound of claim 6, wherein R^{g} is selected from halogen, hydroxyl, C₁₋₆alkyl, and C₁₋₆alkoxy, wherein C₁₋₆alkyl, and C₁₋₆alkoxy are optionally substituted by one, two, three or more substituents each independently selected from R^{P}; and
R^{P} is independently selected, for each occurrence, from halogen.

8. The compound of claim 6 or 7, wherein R² is selected from the group consisting of

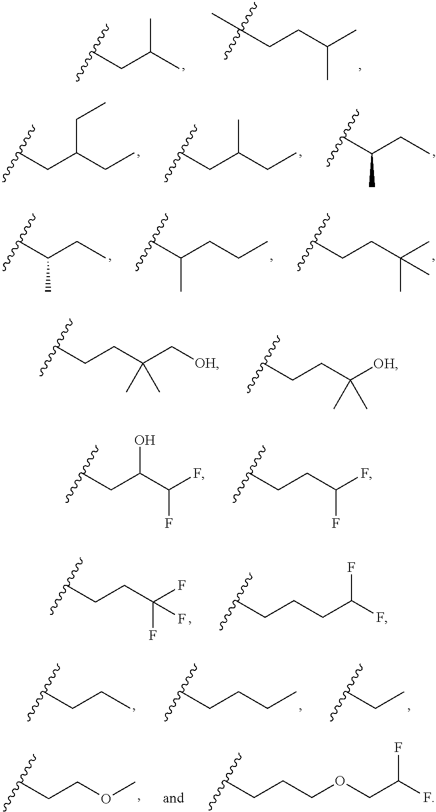

9. The compound of any one of claims 1-5, wherein R² is —C₁₋₆alkylene-C₃₋₆cycloalkyl, wherein —C₁₋₆alkylene-C₃₋₆cycloalkyl may optionally be substituted by one, two, three or more substituents each independently selected from R^{g}.

10. The compound of claim 9, wherein R^{g} is independently selected, for each occurrence, from the group consisting of fluorine, C₁₋₆alkyl, and phenyl, wherein C₁₋₆alkyl and phenyl may optionally be substituted by one, two or three substituents each independently selected from R^{P}; and
R^{P} is independently selected, for each occurrence, from hydroxyl or fluorine.

11. The compound of claim 9 or 10, wherein R² is selected from the group consisting of

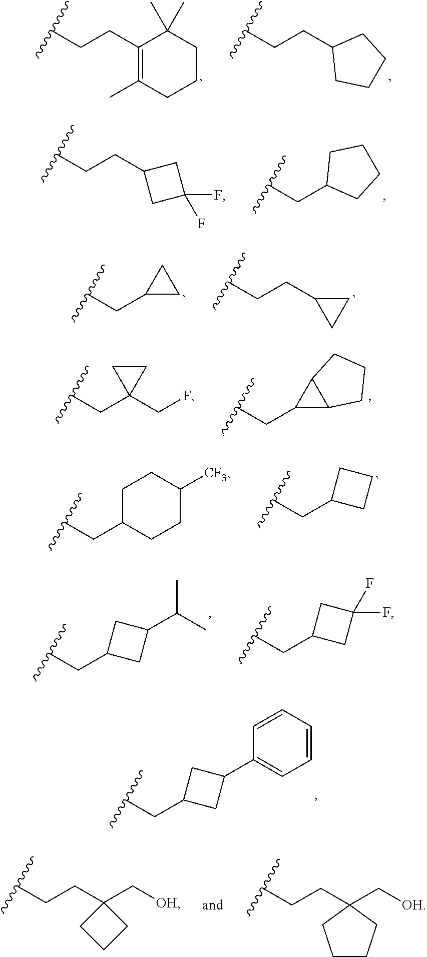

12. The compound of any one of claims 1-5, wherein R² is —C₁₋₆alkylene-4-6 membered heterocyclyl, wherein the —C₁₋₆alkylene-4-6 membered heterocyclyl may optionally be substituted by one, two, three or more substituents each independently selected from R^{g}; and
wherein if —C₁₋₆alkylene-4-6 membered heterocyclyl contains a substitutable ring nitrogen atom, that ring nitrogen atom may optionally be substituted by R^{h};

13. The compound of claim 12, wherein R^{h} is C₁₋₆alkyl-O—C(O)—.

14. The compound of claim 12 or 13, wherein R² is selected from the group consisting of

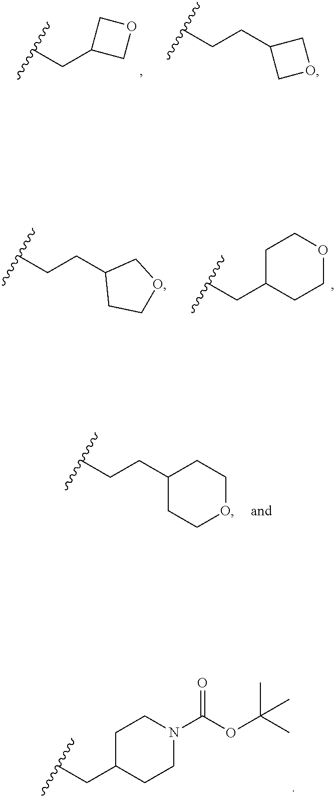 [SMILES: *CC1COC1 ; *CCC1COC1 ; *CCC1CCOC1 ; *CC1CCOCC1 ; *CCC1CCOCC1 ; *CC1CCN(C(=O)OC(C)(C)C)CC1]

15. A compound selected from the group consisting of
5-[(2R)-4-fluoro-6-hydroxy-2-{[(2-methylpropyl)amino]methyl}-2,3-dihydro-1-benzofuran-5-yl]-1λ⁶,2,5-thiadiazolidine-1,1,3-trione;
5-[(2S)-4-fluoro-6-hydroxy-2-{[(2-methylpropyl)amino]methyl}-2,3-dihydro-1-benzofuran-5-yl]-1λ⁶,2,5-thiadiazolidine-1,1,3-trione;
5-(4-fluoro-6-hydroxy-2-{[(2-methylpropyl)amino]methyl}-2,3-dihydro-1-benzofuran-5-yl)-1′,2,5-thiadiazolidine-1,1,3-trione;
5-[(2R)-2-{[(2-cyclopentylethyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-{[(4-hydroxy-3,3-dimethylbutyl)amino]methyl}-2,3-dihydro-1-benzofuran-5-yl]-1λ⁶,2,5-thiadiazolidine-1,1,3-trione;
5-[(2R)-4-fluoro-6-hydroxy-2-{[(3-hydroxy-3-methylbutyl)amino]methyl}-2,3-dihydro-1-benzofuran-5-yl]-1λ⁶,2,5-thiadiazolidine-1,1,3-trione;
5-[(2R)-2-({[2-(3,3-difluorocyclobutyl)ethyl]amino}methyl)-4-fluoro-6-hydroxy-2,3-dihydro-1-benzofuran-5-yl]-1λ⁶,2,5-thiadiazolidine-1,1,3-trione;
5-[(2R)-2-{[(4,4-difluorobutyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-({[2-(oxolan-3-yl)ethyl]amino}methyl)-2,3-dihydro-1-benzofuran-5-yl]-1λ⁶,2,5-thiadiazolidine-1,1,3-trione;
5-{(2R)-4-fluoro-6-hydroxy-2-[(propylamino)methyl]-2,3-dihydro-1-benzofuran-5-yl}-1λ⁶,2,5-thiadiazolidine-1,1,3-trione;
5-{(2R)-2-[(dipropylamino)methyl]-4-fluoro-6-hydroxy-2,3-dihydro-1-benzofuran-5-yl}-1λ⁶,2,5-thiadiazolidine-1,1,3-trione;
5-{(2R)-2-[(butylamino)methyl]-4-fluoro-6-hydroxy-2,3-dihydro-1-benzofuran-5-yl}-1λ⁶,2,5-thiadiazolidine-1,1,3-trione;
5-{(2R)-2-[(ethylamino)methyl]-4-fluoro-6-hydroxy-2,3-dihydro-1-benzofuran-5-yl}-1λ⁶,2,5-thiadiazolidine-1,1,3-trione;
5-[(2R)-2-{[(cyclopentylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-({[(oxan-4-yl)methyl]amino}methyl)-2,3-dihydro-1-benzofuran-5-yl]-1λ⁶,2,5-thiadiazolidine-1,1,3-trione;
5-[(2R)-4-fluoro-6-hydroxy-2-{[(3-methylbutyl)amino]methyl}-2,3-dihydro-1-benzofuran-5-yl]-1λ⁶,2,5-thiadiazolidine-1,1,3-trione;
5-[(2R)-4-fluoro-6-hydroxy-2-{[(2-methylbutyl)amino]methyl}-2,3-dihydro-1-benzofuran-5-yl]-1λ⁶,2,5-thiadiazolidine-1,1,3-trione;
5-[(2R)-2-{[(cyclopropylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-2-{[bis(cyclopropylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-2-{[(cyclobutylmethyl)amino]methyl}-4-fluoro-6-hydroxy-2,3-dihydro-1-benzofuran-5-yl]-1λ⁶,2,5-thiadiazolidine-1,1,3-trione;
5-[(2R)-4-fluoro-6-hydroxy-2-({[(oxetan-3-yl)methyl]amino}methyl)-2,3-dihydro-1-benzofuran-5-yl]-1λ⁶,2,5-thiadiazolidine-1,1,3-trione;
5-{(2R)-4-fluoro-6-hydroxy-2-[({2-[1-(hydroxymethyl)cyclobutyl]ethyl}amino)methyl]-2,3-dihydro-1-benzofuran-5-yl}-1λ⁶,2,5-thiadiazolidine-1,1,3-trione;
5-{(2R)-4-fluoro-6-hydroxy-2-[({2-[1-(hydroxymethyl)cyclopentyl]ethyl}amino)methyl]-2,3-dihydro-1-benzofuran-5-yl}-1λ⁶,2,5-thiadiazolidine-1,1,3-trione;
5-[(2R)-2-({[3-(2,2-difluoroethoxy)propyl]amino}methyl)-4-fluoro-6-hydroxy-2,3-dihydro-1-benzofuran-5-yl]-1λ⁶,2,5-thiadiazolidine-1,1,3-trione;
tert-butyl 4-[({[(2R)-4-fluoro-6-hydroxy-5-(1,1,4-trioxo-1λ⁶,2,5-thiadiazolidin-2-yl)-2,3-dihydro-1-benzofuran-2-yl]methyl}amino)methyl]piperidine-1-carboxylate;
5-[(2R)-4-fluoro-6-hydroxy-2-({[2-(oxan-4-yl)ethyl]amino}methyl)-2,3-dihydro-1-benzofuran-5-yl]-1λ⁶,2,5-thiadiazolidine-1,1,3-trione;
5-{(2R)-2-[({[(1RS,5SR)-bicyclo[3.1.0]hexan-6-yl]methyl}amino)methyl]-4-fluoro-6-hydroxy-2,3-dihydro-1-benzofuran-5-yl}-1λ⁶,2,5-thiadiazolidine-1,1,3-trione;
5-[(2R)-4-fluoro-6-hydroxy-2-{[(3-phenylpropyl)amino]methyl}-2,3-dihydro-1-benzofuran-5-yl]-1λ⁶,2,5-thiadiazolidine-1,1,3-trione;
5-[(2R)-4-fluoro-6-hydroxy-2-({[2-(2,6,6-trimethylcyclohex-1-en-1-yl)ethyl]amino}methyl)-2,3-dihydro-1-benzofuran-5-yl]-1λ⁶,2,5-thiadiazolidine-1,1,3-trione;
5-[(2R)-4-fluoro-6-hydroxy-2-({[(3-phenylcyclobutyl)methyl]amino}methyl)-2,3-dihydro-1-benzofuran-5-yl]-1λ⁶,2,5-thiadiazolidine-1,1,3-trione;
5-{(2R)-4-fluoro-6-hydroxy-2-[({[4-(trifluoromethyl)cyclohexyl]methyl}amino)methyl]-2,3-dihydro-1-benzofuran-5-yl}-1λ⁶,2,5-thiadiazolidine-1,1,3-trione;
5-{(2R)-4-fluoro-2-[({[1-(fluoromethyl)cyclopropyl]methyl}amino)methyl]-6-hydroxy-2,3-dihydro-1-benzofuran-5-yl}-1λ⁶,2,5-thiadiazolidine-1,1,3-trione;
5-[(2S)-4-fluoro-6-hydroxy-2-{[(2-methylpropyl)amino]methyl}-2,3-dihydro-1H-indol-5-yl]-1λ⁶,2,5-thiadiazolidine-1,1,3-trione;
5-[(2R)-4-fluoro-6-hydroxy-2-{[(2-methylpropyl)amino]methyl}-2,3-dihydro-1H-indol-5-yl]-1λ⁶,2,5-thiadiazolidine-1,1,3-trione;
5-[(2R)-2-{[(2-ethylbutyl)amino]methyl}-4-fluoro-6-hydroxy-2,3-dihydro-1H-indol-5-yl]-1λ⁶,2,5-thiadiazolidine-1,1,3-trione;
5-{(2R)-4-fluoro-6-hydroxy-2-[(propylamino)methyl]-2,3-dihydro-1H-indol-5-yl}-1λ⁶,2,5-thiadiazolidine-1,1,3-trione;
5-[(2R)-4-fluoro-6-hydroxy-2-{[(2-methoxyethyl)amino]methyl}-2,3-dihydro-1H-indol-5-yl]-1λ⁶,2,5-thiadiazolidine-1,1,3-trione;
5-[(2R)-2-{[(cyclobutylmethyl)amino]methyl}-4-fluoro-6-hydroxy-2,3-dihydro-1H-indol-5-yl]-1λ⁶,2,5-thiadiazolidine-1,1,3-trione;
5-[(2R)-4-fluoro-6-hydroxy-2-({[(oxan-4-yl)methyl]amino}methyl)-2,3-dihydro-1H-indol-5-yl]-1λ⁶,2,5-thiadiazolidine-1,1,3-trione;
5-[(2R)-4-fluoro-6-hydroxy-2-{[(3-methylbutyl)amino]methyl}-2,3-dihydro-1H-indol-5-yl]-1λ⁶,2,5-thiadiazolidine-1,1,3-trione;
5-[(2R)-2-{[(3,3-difluoropropyl)amino]methyl}-4-fluoro-6-hydroxy-2,3-dihydro-1H-indol-5-yl]-1λ⁶,2,5-thiadiazolidine-1,1,3-trione;
5-[(2R)-4-fluoro-6-hydroxy-2-{[(3,3,3-trifluoropropyl)amino]methyl}-2,3-dihydro-1H-indol-5-yl]-1λ⁶,2,5-thiadiazolidine-1,1,3-trione;
5-[(2R)-4-fluoro-6-hydroxy-2-{[(2-methylbutyl)amino]methyl}-2,3-dihydro-1H-indol-5-yl]-1λ⁶,2,5-thiadiazolidine-1,1,3-trione;
5-[(2R)-2-({[(3,3-difluorocyclobutyl)methyl]amino}methyl)-4-fluoro-6-hydroxy-2,3-dihydro-1H-indol-5-yl]-1λ⁶,2,5-thiadiazolidine-1,1,3-trione;
5-[4-fluoro-6-hydroxy-2-({[2-(oxetan-3-yl)ethyl]amino}methyl)-2,3-dihydro-1H-indol-5-yl]-1λ⁶,2,5-thiadiazolidine-1,1,3-trione;
5-(2-{[(4,4-difluorobutyl)amino]methyl}-4-fluoro-6-hydroxy-2,3-dihydro-1H-indol-5-yl)-1λ⁶,2,5-thiadiazolidine-1,1,3-trione;
5-[(2R)-2-{[(cyclopentylmethyl)amino]methyl}-4-fluoro-6-hydroxy-2,3-dihydro-1H-indol-5-yl]-1λ⁶,2,5-thiadiazolidine-1,1,3-trione;
5-[(2R)-2-{[(cyclopropylmethyl)amino]methyl}-4-fluoro-6-hydroxy-2,3-dihydro-1H-indol-5-yl]-1λ⁶,2,5-thiadiazolidine-1,1,3-trione;
5-[(2R)-4-fluoro-6-hydroxy-2-{[(pentan-2-yl)amino]methyl}-2,3-dihydro-1H-indol-5-yl]-1λ⁶,2,5-thiadiazolidine-1,1,3-trione;
5-[(2R)-2-({[(2R)-butan-2-yl]amino}methyl)-4-fluoro-6-hydroxy-2,3-dihydro-1H-indol-5-yl]-1λ⁶,2,5-thiadiazolidine-1,1,3-trione;
5-{(2R)-2-[(butylamino)methyl]-4-fluoro-6-hydroxy-2,3-dihydro-1H-indol-5-yl}-1λ⁶,2,5-thiadiazolidine-1,1,3-trione;
5-[(2R)-4-fluoro-6-hydroxy-2-(hydroxymethyl)-2,3-dihydro-1H-indol-5-yl]-1λ⁶,2,5-thiadiazolidine-1,1,3-trione;
5-[(2R)-2-{[(2-cyclopropylethyl)amino]methyl}-4-fluoro-6-hydroxy-2,3-dihydro-1H-indol-5-yl]-1λ⁶,2,5-thiadiazolidine-1,1,3-trione;
5-[(2R)-2-{[(3,3-dimethylbutyl)amino]methyl}-4-fluoro-6-hydroxy-2,3-dihydro-1H-indol-5-yl]-1λ⁶,2,5-thiadiazolidine-1,1,3-trione;
5-{(2R)-4-fluoro-6-hydroxy-2-[({[3-(propan-2-yl)cyclobutyl]methyl}amino)methyl]-2,3-dihydro-1H-indol-5-yl}-1λ⁶,2,5-thiadiazolidine-1,1,3-trione;
5-[(2R)-4-fluoro-6-hydroxy-2-{[methyl(2-methylpropyl)amino]methyl}-2,3-dihydro-1H-indol-5-yl]-1λ⁶,2,5-thiadiazolidine-1,1,3-trione;
5-[(2R)-4-fluoro-6-hydroxy-1-methyl-2-{[(2-methylpropyl)amino]methyl}-2,3-dihydro-1H-indol-5-yl]-1λ⁶,2,5-thiadiazolidine-1,1,3-trione; and
5-[(2R)-2-{[(3,3-difluoro-2-hydroxypropyl)amino]methyl}-4-fluoro-6-hydroxy-2,3-dihydro-1H-indol-5-yl]-1λ⁶,2,5-thiadiazolidine-1,1,3-trione;
or a pharmaceutically acceptable salt thereof.

16. A pharmaceutically acceptable composition comprising a compound of any one of claims 1-15 and a pharmaceutically acceptable carrier.
